(19) United States
(12) Patent Application Publication
(10) Pub. No.: US 2009/0104605 A1
(43) Pub. Date: Apr. 23, 2009

(54) DIAGNOSIS OF SEPSIS

(76) Inventors: Gary Siuzdak, San Diego, CA (US); William A. Nussbaumer, Baltimore, MD (US); Craig C. Whiteford, York, PA (US); Richard L. Moore, Glenville, PA (US)

Correspondence Address: David W. Highet, VP & Chief IP Counsel;Becton, Dickinson and Company, (Jones Day), 1 Becton Drive, MC 110, Franklin Lakes, NJ, 07417-1880, US

(21) Appl. No.: 12/098,398

(22) Filed: Apr. 4, 2008

Related U.S. Application Data
(60) Provisional application No. 60/922,247, filed on Apr. 5, 2007.

(30) Foreign Application Priority Data
Dec. 14, 2006 (US) PCT/US2006/047737

(51) Int. Cl. C12Q 1/68; G01N 33/53; G01N 33/567; G06G 7/60

(52) U.S. Cl. 435/6; 435/7.1; 435/7.2; 703/11

(57) ABSTRACT

Methods and apparatus for predicting the development of sepsis in a subject at risk for developing sepsis are provided. Features in a biomarker profile of the subject are evaluated. The subject is likely to develop sepsis if these features satisfy a particular value set. Methods and apparatus for predicting the development of a stage of sepsis in a subject at risk for developing a stage of sepsis are provided. A plurality of features in a biomarker profile of the subject is evaluated. The subject is likely to have the stage of sepsis if these feature values satisfy a particular value set. Methods and apparatus for diagnosing sepsis in a subject are provided. A plurality of features in a biomarker profile of the subject is evaluated. The subject is likely to develop sepsis when the plurality of features satisfies a particular value set.

### CROSS REFERENCE TO RELATED APPLICATIONS

This application claims priority to Provisional Patent Application No. 60/751,197, filed Dec. 15, 2005 which is hereby incorporated by reference herein in its entirety. This application also claims priority to Provisional Patent Application No. 60/819,983, filed Jul. 10, 2006 which is hereby incorporated by reference herein in its entirety. This application also claims priority to PCT Application No. PCT/US2006/047737, filed Dec. 14, 2006, which is hereby incorporated by reference herein in its entirety. This application also claims priority to Provisional Patent Application No. 60/922,247, filed Apr. 5, 2007 which is hereby incorporated by reference herein in its entirety.

### 1. FIELD OF THE INVENTION

The present invention relates to methods and compositions for diagnosing or predicting sepsis and/or its stages of progression in a subject. The present invention also relates to methods and compositions for diagnosing systemic inflammatory response syndrome in a subject.

### 2. BACKGROUND OF THE INVENTION

Early detection of a disease condition typically allows for a more effective therapeutic treatment with a correspondingly more favorable clinical outcome. In many cases, however, early detection of disease symptoms is problematic due to the complexity of the disease; hence, a disease may become relatively advanced before diagnosis is possible. Systemic inflammatory conditions represent one such class of diseases. These conditions, particularly sepsis, typically, but not always, result from an interaction between a pathogenic microorganism and the host's defense system that triggers an excessive and dysregulated inflammatory response in the host. The complexity of the host's response during the systemic inflammatory response has complicated efforts towards understanding disease pathogenesis (reviewed in Healy, 2002, Annul. Pharmacother. 36:648-54). An incomplete understanding of the disease pathogenesis, in turn, contributes to the difficulty in finding useful diagnostic biomarkers. Early and reliable diagnosis is imperative, however, because of the remarkably rapid progression of sepsis into a life-threatening condition.

The development of sepsis in a subject follows a well-described course, progressing from systemic inflammatory response syndrome (“SIRS”)-negative, to SIRS-positive, and then to sepsis, which may then progress to severe sepsis, septic shock, multiple organ dysfunction (“MOD”), and ultimately death. Sepsis may also arise in an infected subject when the subject subsequently develops SIRS. SIRS represents the host response to numerous stimuli including trauma, burns, pancreatitis, transfusion reactions, and major surgery. Unfortunately, in the critically ill patient, sepsis is often difficult to diagnose as patients may already manifest SIRS from other illness. Approximately 70% of SIRS patients admitted to an ICU have a non-infectious etiology. See, Sprung et al., 2006, Intensive Care Med. 32:421-427. However, the prompt diagnosis of sepsis is essential because early treatment is important for improving outcomes (Rivers et al. 2001, N Engl J. Med. 345:1368-77, Dellinger et al. 2004, Crit. Care Med 32:858-73, and sepsis remains the leading cause of death in non-coronary intensive care units (Parrillo et al., 1990, Ann Intern Med. 113:227-242).

“Sepsis” is commonly defined as the systemic host response to infection with SIRS plus a documented infection. “Severe sepsis” is associated with MOD, hypotension, disseminated intravascular coagulation (“DIC”) or hypoperfusion abnormalities, including lactic acidosis, oliguria, and changes in mental status. “Septic shock” is commonly defined as sepsis-induced hypotension that is resistant to fluid resuscitation with the additional presence of hypoperfusion abnormalities.

Documenting the presence of the pathogenic microorganisms that are clinically significant to sepsis has proven difficult. Causative microorganisms typically are detected by culturing a subject's blood, sputum, urine, wound secretion, in-dwelling line catheter surfaces, etc. Unfortunately, cultures can take over 24 hours to obtain results and are neither sensitive nor specific. Causative microorganisms may reside only in certain body microenvironments such that the particular material that is cultured may not contain the contaminating microorganisms. Detection may be complicated further by low numbers of microorganisms at the site of infection. Low numbers of pathogens in blood present a particular problem for diagnosing sepsis by culturing blood. In one study, for example, positive culture results were obtained in only 17% of subjects presenting clinical manifestations of sepsis (Rangel-Frausto et al., 1995, JAMA 273:117-123). Diagnosis can be further complicated by contamination of samples by non-pathogenic microorganisms. For example, only 12.4% of detected microorganisms were clinically significant in a study of 707 subjects with septicemia (Weinstein et al., 1997, Clinical Infectious Diseases 24:584-602). Other biologic markers have been studied as well. Procalcitonin has been considered a potential sepsis biomarker, and demonstrates prognostic capabilities. Widespread use of procalcitonin in the ICU has been limited due to lack of specificity and variable sensitivity. See, Giamarellos-Bourboulis et al., 2002, Intensive Care Med. 28:1351-56. A recent meta-analysis confirmed the superiority of procalcitonin to C-reactive protein, but also identified its weakness as a diagnostic tool, suggesting it be used as a screening test with empiric antibiotics and further testing to accompany positive results. See Uzzan et al., 2006, Crit. Care Med. 34:1996-2003. Given the lack of a gold-standard molecular diagnosis for sepsis, there is an escalating search for biomarkers to help identify sepsis in the critically ill patient.

The difficulty in early diagnosis of sepsis is reflected by the high morbidity and mortality associated with the disease. Sepsis currently is the tenth leading cause of death in the United States and is especially prevalent among hospitalized patients in non-coronary intensive care units (ICUs), where it is the most common cause of death. The overall rate of mortality is as high as 35 percent, with an estimated 750,000 cases per year occurring in the United States alone. The annual cost to treat sepsis in the United States alone is on the order of billions of dollars.

A need, therefore, exists for a method of diagnosing sepsis, using techniques that have satisfactory specificity and sensitivity performance, sufficiently early to allow effective intervention and prevention.

### 3. SUMMARY OF THE INVENTION

The present invention relates to methods and compositions for diagnosing sepsis, including the onset of sepsis, in a test subject. The present invention also relates to methods and compositions for predicting sepsis in a test subject.

The present invention further relates to methods and compositions for diagnosing or predicting stages of sepsis progression in a test subject. The present invention still further relates to methods and compositions for diagnosing systemic inflammatory response syndrome (SIRS) in a test subject.

In one aspect, the present invention provides a method of predicting the development of sepsis in a test subject at risk for developing sepsis. This method comprises evaluating whether a plurality of features in a biomarker profile of the test subject satisfies a value set, wherein satisfying the value set means that the test subject will develop sepsis with a likelihood that is determined by the accuracy of the decision rule to which the plurality of features are applied in order to determine whether they satisfy the value set. In some embodiments, the accuracy of the decision rule is at least 60 percent, at least 70 percent, at least 80 percent, or at least 90 percent. Therefore, correspondingly, the likelihood that the test subject will develop sepsis when the plurality of features satisfies the value set is at least 60 percent, at least 70 percent, at least 80 percent, or at least 90 percent.

Yet another aspect of the invention comprises methods for diagnosing sepsis in a test subject. These methods comprise evaluating whether a plurality of features in a biomarker profile of the test subject satisfies a value set, wherein satisfying the value set predicts that the test subject has sepsis with a likelihood that is determined by the accuracy of the decision rule to which the plurality of features are applied in order to determine whether they satisfy the value set. When the plurality of biomarkers comprises complement component C3 and complement component C4, the plurality of biomarkers comprises three or more biomarkers. In some embodiments, the accuracy of the decision rule is at least 60 percent, at least 70 percent, at least 80 percent, or at least 90 percent. Therefore, correspondingly, the likelihood that the test subject has sepsis when the plurality of features satisfies the value set is at least 60 percent, at least 70 percent, at least 80 percent, or at least 90 percent.

In a particular embodiment, the biomarker profile comprises at least two features, each feature representing a feature of a corresponding biomarker listed in column three or four of Table 1. In one embodiment, the biomarker profile comprises at least two different biomarkers listed in column three or four of Table 1. In such an embodiment, the biomarker profile can comprise a respective corresponding feature for the at least two biomarkers. Generally, the at least two biomarkers are derived from at least two different genes. When the plurality of biomarkers comprises complement component C3 and complement component C4, the plurality of biomarkers comprises three or more biomarkers.

In the case where a biomarker in the at least two different biomarkers is listed in column three of Table 1, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. Further still, the biomarker can be, for example, a protein listed in column four of Table 1 or a discriminating fragment of the protein, or an indication of any of the above. Here, a discriminating molecule or fragment is a molecule or fragment that, when detected, indicates presence or abundance of the above-identified transcript, cDNA, amplified nucleic acid, or protein. In accordance with this embodiment, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in Table 1). In one embodiment, such an assay utilizes a nucleic acid microarray. In some embodiments, the biomarker profile comprises at least 2, 3, 4, 5, 6, 7, 8, 9, 10, 11, 12, 13, 14, 15, 16, 17, 18, 19, 20, 21, 22, 23, 24, 25, 30, 35, 40, 45, or 50 different biomarkers from Table 1.

In a particular embodiment, the biomarker profile comprises at least two features, each feature representing a feature of a corresponding biomarker listed in column three or four of Table 4. In one embodiment, the biomarker profile comprises at least two different biomarkers listed in column three or four of Table 4. In such an embodiment, the biomarker profile can comprise a respective corresponding feature for the at least two biomarkers. Generally, the at least two biomarkers are derived from at least two different genes. In the case where a biomarker in the at least two different biomarkers is listed in column three of Table 4, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. Further still, the biomarker can be, for example, a protein listed in column four of Table 4 or a discriminating fragment of the protein, or an indication of any of the above. Here, a discriminating molecule or fragment is a molecule or fragment that, when detected, indicates presence or abundance of the above-identified transcript, cDNA, amplified nucleic acid, or protein. In accordance with this embodiment, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in Table 4). In one embodiment, such an assay utilizes a nucleic acid microarray. In some embodiments, the biomarker profile comprises at least 2, 3, 4, 5, 6, 7, 8, 9, or 10 different biomarkers from Table 4. In some embodiments, the biomarker profile comprises SERPINC1, APOA2, and CRP. In some embodiments, the biomarker profile comprises at least one of SERPINC1, APOA2, and CRP, and, additionally, at least 1, 2, 3, 4, 5, 6 or 7 other additional biomarkers in Table 4. In some embodiments, the biomarker profile comprises at least one of SERPINC1, APOA2, and CRP and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, and 7. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, and 7. In some embodiments, each of the biomarkers in the profile is a protein. In some embodiments, each of the biomarkers in the profile is a nucleic acid. In some embodiments, some of the biomarkers in the profile are nucleic acids and some of the biomarkers are proteins.

In a particular embodiment, the biomarker profile comprises at least two features, each feature representing a feature of a corresponding biomarker listed in column three or four of Table 5. In one embodiment, the biomarker profile comprises at least two different biomarkers listed in column three or four of Table 5. In such an embodiment, the biomarker profile can comprise a respective corresponding feature for the at least two biomarkers. Generally, the at least two biomarkers are derived from at least two different genes. In the case where a biomarker in the at least two different biomarkers is listed in column three of Table 5, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. Further still, the biomarker can be, for example, a protein listed in column four of Table 5 or a discriminating fragment of the protein, or an indication of any of the above. Here, a discriminating molecule or fragment is a molecule or fragment that, when detected, indicates presence or abundance of the above-identified transcript, cDNA, amplified nucleic acid, or protein. In accordance with this embodiment, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in Table 5). In one embodiment, such an assay utilizes a nucleic acid microarray. In some embodiments, the biomarker profile comprises at least 2, 3, 4, 5, 6, 7, 8, 9, 10, 11, 12, 13, 14, 15, 16, 17, 18, 19, 20, 21, 22, 23, 24, 25, 30, 35, 40, 45, or 50 different biomarkers from Table 5.

In a particular embodiment, the biomarker profile comprises at least two features, each feature representing a feature of a corresponding biomarker listed in column three or four of Table 6. In one embodiment, the biomarker profile comprises at least two different biomarkers listed in column three or four of Table 6. In such an embodiment, the biomarker profile can comprise a respective corresponding feature for the at least two biomarkers. Generally, the at least two biomarkers are derived from at least two different genes. In the case where a biomarker in the at least two different biomarkers is listed in column three of Table 6, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. Further still, the biomarker can be, for example, a protein listed in column four of Table 6 or a discriminating fragment of the protein, or an indication of any of the above. Here, a discriminating molecule or fragment is a molecule or fragment that, when detected, indicates presence or abundance of the above-identified transcript, cDNA, amplified nucleic acid, or protein. In accordance with this embodiment, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in Table 6). In one embodiment, such an assay utilizes a nucleic acid microarray. In some embodiments, the biomarker profile comprises at least 2, 3, 4, 5, 6, 7, 8, 9, 10, 11, 12, 13, 14, 15, 16, 17, 18, 19, 20, 21, 22, 23, 24, or 25 different biomarkers from Table 6.

In a particular embodiment, the biomarker profile comprises at least two features, each feature representing a feature of a corresponding biomarker listed in column three or four of Table 7. In one embodiment, the biomarker profile comprises at least two different biomarkers listed in column three or four of Table 7. In such an embodiment, the biomarker profile can comprise a respective corresponding feature for the at least two biomarkers. Generally, the at least two biomarkers are derived from at least two different genes. In the case where a biomarker in the at least two different biomarkers is listed in column three of Table 7, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. Further still, the biomarker can be, for example, a protein listed in column four of Table 7 or a discriminating fragment of the protein, or an indication of any of the above. Here, a discriminating molecule or fragment is a molecule or fragment that, when detected, indicates presence or abundance of the above-identified transcript, cDNA, amplified nucleic acid, or protein. In accordance with this embodiment, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in Table 7). In one embodiment, such an assay utilizes a nucleic acid microarray. In some embodiments, the biomarker profile comprises at least 2, 3, 4, 5, 6, 7, 8, 9, 10, 11, 12, 13, 14, 15, 16, 17, 18, 19, 20, 21, 22, 23, or 24 different biomarkers from Table 7.

In a particular embodiment, the biomarker profile comprises at least two features, each feature representing a feature of a corresponding biomarker listed in column three or four of Table 15. In one embodiment, the biomarker profile comprises at least two different biomarkers listed in column three or four of Table 15. In such an embodiment, the biomarker profile can comprise a respective corresponding feature for the at least two biomarkers. Generally, the at least two biomarkers are derived from at least two different genes. In the case where a biomarker in the at least two different biomarkers is listed in column three of Table 15, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. Further still, the biomarker can be, for example, a protein listed in column four of Table 15 or a discriminating fragment of the protein, or an indication of any of the above. Here, a discriminating molecule or fragment is a molecule or fragment that, when detected, indicates presence or abundance of the above-identified transcript, cDNA, amplified nucleic acid, or protein. In accordance with this embodiment, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in Table 15). In one embodiment, such an assay utilizes a nucleic acid microarray. In some embodiments, the biomarker profile comprises at least 2, 3, 4, 5, 6, 7, 8, 9, 10, 11, 12, 13, 14, 15, 16, 17, 18, 19, 20, 21, 22, 23, or 24 different biomarkers from Table 15.

In a particular embodiment, the biomarker profile comprises at least two features, each feature representing a feature of a corresponding biomarker listed in column three or four of Table 17. In one embodiment, the biomarker profile comprises at least two different biomarkers listed in column three or four of Table 17. In such an embodiment, the biomarker profile can comprise a respective corresponding feature for the at least two biomarkers. Generally, the at least two biomarkers are derived from at least two different genes. In the case where a biomarker in the at least two different biomarkers is listed in column three of Table 17, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. Further still, the biomarker can be, for example, a protein listed in column four of Table 17 or a discriminating fragment of the protein, or an indication of any of the above. Here, a discriminating molecule or fragment is a molecule or fragment that, when detected, indicates presence or abundance of the above-identified transcript, cDNA, amplified nucleic acid, or protein. In accordance with this embodiment, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in Table 17). In one embodiment, such an assay utilizes a nucleic acid microarray. In some embodiments, the biomarker profile comprises at least 2, 3, 4, 5, 6, 7, 8, 9, 10, 11, 12, 13, 14, 15, 16, 17, 18, 19, 20, 21, 22, 23, or 24 different biomarkers from Table 17.

In a particular embodiment, the biomarker profile comprises at least two features, each feature representing a feature of a corresponding biomarker listed in column three or four of any combination of Tables 1, 4, 5, 6, 7, 12, 13, 14, 15, 16, 17, 18, 19, 20, and 21. In one embodiment, the biomarker profile comprises at least two different biomarkers listed in column three or four of any combination of Tables 1, 4, 5, 6, 7, 12, 13, 14, 15, 16, 17, 18, 19, 20, and 21. In such an embodiment, the biomarker profile can comprise a respective corresponding feature for the at least two biomarkers. Generally, the at least two biomarkers are derived from at least two different genes. In the case where a biomarker in the at least two different biomarkers is listed in column three of any combination of Tables 1, 4, 5, 6, 7, 12, 13, 14, 15, 16, 17, 18, 19, 20, and 21, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. Further still, the biomarker can be, for example, a protein listed in column four of any combination of Tables 1, 4, 5, 6, 7, 12, 13, 14, 15, 16, 17, 18, 19, 20, and 21 or a discriminating fragment of the protein, or an indication of any of the above. Here, a discriminating molecule or fragment is a molecule or fragment that, when detected, indicates presence or abundance of the above-identified transcript, cDNA, amplified nucleic acid, or protein. In accordance with this embodiment, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in any combination of Tables 1, 4, 5, 6, 7, 12, 13, 14, 15, 16, 17, 18, 19, 20, and 21). In one embodiment, such an assay utilizes a nucleic acid microarray. In some embodiments, the biomarker profile comprises at least 2, 3, 4, 5, 6, 7, 8, 9, 10, 11, 12, 13, 14, 15, 16, 17, 18, 19, 20, 21, 22, 23, 24, 25, 30, 35, 40, 45, or 50 different biomarkers from any combination of Tables 1, 4, 5, 6, 7, 12, 13, 14, 15, 16, 17, 18, 19, 20, and 21. In some embodiments, the biomarker profile comprises the biomarkers CRP, APOA2, and SERPINC1 described in Table 4 below. In some embodiments these three biomarkers are proteins. In some embodiments, these three biomarkers are nucleic acids. In some embodiments, these three biomarkers are any combination of proteins and nucleic acids. In some embodiments, the biomarker profile comprise at least one of the biomarkers CRP, APOA2, and SERPINC1, and, additionally, 1, 2, 3, 4, 5, 6, or 7 biomarkers from those set forth in Table 4. In some embodiments, the biomarker profile comprises at least one of the biomarkers CRP, APOA2, and SERPINC1, and, additionally, 1, 2, 3, 4, 5, 6, 7, 8, 9, 10, 11, 12, 13, 14, 15, 16, 17, 18, 19, 20 or more additional biomarkers from those listed in columns 3 and/or 4 of any one of Tables 1, 4, 5, 6, 7, 12, 13, 14, 15, 16, 17, 18, 19, 20, and 21. In some embodiments, the biomarker profile comprises at least one of the biomarkers CRP, APOA2, and SERPINC1, and, additionally, 1, 2, 3, 4, 5, 6, 7, 8, 9, 10, 11, 12, 13, 14, 15, 16, 17, 18, 19, 20 or more additional biomarkers from those listed in columns 3 and/or 4 of any combination of Tables 1, 4, 5, 6, 7, 12, 13, 14, 15, 16, 17, 18, 19, 20, and 21.

In a particular embodiment, the biomarker profile comprises at least four features, each feature representing a feature of a corresponding biomarker listed in column three or four of any combination of Tables 1, 4, 5, 6 7, 12, 13, 14, 15, 16, 17, 18, 19, 20, and 21. In one embodiment, the biomarker profile comprises at least four different biomarkers listed in column three or four of any combination of Tables 1, 4, 5, 6, 7, 12, 13, 14, 15, 16, 17, 18, 19, 20, and 21. In such an embodiment, the biomarker profile can comprise a respective corresponding feature for the at least four biomarkers. Generally, the at least four biomarkers are derived from at least four different genes. In the case where a biomarker in the at least four different biomarkers is listed in column three of any combination of Tables 1, 4, 5, 6, 7, 12, 13, 14, 15, 16, 17, 18, 19, 20, and 21, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. Further still, the biomarker can be, for example, a protein listed in column four of any combination of Tables 1, 4, 5, 6, 7, 12, 13, 14, 15, 16, 17, 18, 19, 20, and 21 or a discriminating fragment of the protein, or an indication of any of the above. Here, a discriminating molecule or fragment is a molecule or fragment that, when detected, indicates presence or abundance of the above-identified transcript, cDNA, amplified nucleic acid, or protein. In accordance with this embodiment, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in any combination of Tables 1, 4, 5, 6, 7, 12, 13, 14, 15, 16, 17, 18, 19, 20, and 21). In one embodiment, such an assay utilizes a nucleic acid microarray. In some embodiments, the biomarker profile comprises at least 4, 5, 6, 7, 8, 9, 10, 11, 12, 13, 14, 15, 16, 17, 18, 19, 20, 21, 22, 23, 24, 25, 30, 35, 40, 45, or 50 different biomarkers from any combination of Tables 1, 4, 5, 6, 7, 12, 13, 14, 15, 16, 17, 18, 19, 20, and 21.

Although the methods of the present invention are particularly useful for detecting or predicting the onset of sepsis in SIRS subjects, one of skill in the art will understand that the present methods may be used for any subject including, but not limited to, subjects suspected of having SIRS or of being at any stage of sepsis. For example, a biological sample can be taken from a subject, and a profile of biomarkers in the sample can be evaluated in light of biomarker profiles obtained from several different types of training populations. Representative training populations variously include, for example, populations that include subjects who are SIRS-negative, populations that include subjects who are SIRS-positive, and/or populations that include subjects at a particular stage of sepsis. Evaluation of the biomarker profile in light of each of these different training populations can be used to determine whether the test subject is SIRS-negative, SIRS-positive, is likely to become septic, or has a particular stage of sepsis. Based on the diagnosis resulting from the methods of the present invention, an appropriate treatment regimen can then be initiated.

In particular embodiments, the invention also provides kits that are useful in diagnosing or predicting the development of sepsis or SIRS in a subject (see Section 5.3, infra). Some such kits of the present invention comprise at least 2, 3, 4, 5, 6, 7, 8, 9, 10, 11, 12, 13, 14, 15, 16, 17, 18, 19, 20, 25, 30, 35, 40, 45, 50, 54, 5, 60, 65, 70, 75, 80, 85, 90, 95, 96 or more biomarkers and/or reagents used to detect presence or abundance of such biomarkers. In some embodiments, each of these biomarkers is from Table 1. In some embodiments, each of these biomarkers is from Table 4. In some of these embodiments three of the biomarkers in the kit are CRP, APOA2, and SERPINC1. In some embodiments, the biomarkers in the kit are at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19, 20, and 21. In some embodiments, the biomarkers in the kit are at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21. In some embodiments, each of these biomarkers is from Table 5. In some embodiments, each of these biomarkers is from Table 6. In some embodiments, each of these biomarkers is from Table 7. In some embodiments, each of these biomarkers is from Table 15. In some embodiments, each of these biomarkers is from Table 17. In some embodiments, each of these biomarkers is from any combination of Tables 1, 4, 5, 6, 7, 12, 13, 14, 15, 16, 17, 18, 19, 20, and 21. In another embodiment, the kits of the present invention comprise at least 2, but as many as one hundred or more biomarkers and/or reagents used to detect the presence or abundance of such biomarkers.

In a specific embodiment, the kits of the present invention comprise at least 2, 3, 4, 5, 6, 7, 8, 9, 10, 11, 12, 13, 14, 15, 16, 17, 18, 19, 20, 25, 30, 35, 40, 45, 50, 54, 5, 60, 65, 70, 75, 80, 85, 90, 95, 96, 100 or 200 or more reagents that specifically bind the biomarkers of the present invention. For example, such kits can comprise nucleic acid molecules and/or antibody molecules that specifically bind to biomarkers of the present invention.

Specific exemplary biomarkers that are useful in the present invention are set forth in Tables 1, 4, 5, 6, 7, 12, 13, 14, 15, 16, 17, 18, 19, 20, and 21 of Section 6. The biomarkers of the kit of the present invention can be used to generate biomarker profiles according to the present invention. Examples of types of biomarkers and/or reagents within such kits include, but are not limited to, proteins and fragments thereof, peptides, polypeptides, antibodies, proteoglycans, glycoproteins, lipoproteins, carbohydrates, lipids, nucleic acids (e.g., mRNA, DNA, cDNA, siRNA), organic and inorganic chemicals, and natural and synthetic polymers or a discriminating molecule or fragment thereof.

Still another aspect of the present invention comprises computers and computer readable media for evaluating whether a test subject is likely to develop sepsis or SIRS. For instance, one embodiment of the present invention provides a computer program product for use in conjunction with a computer system. The computer program product comprises a computer readable storage medium and a computer program mechanism embedded therein. The computer program mechanism comprises instructions for evaluating whether a plurality of features in a biomarker profile of a test subject at risk for developing sepsis satisfies a first value set. Satisfaction of the first value set predicts that the test subject is likely to develop sepsis. In some embodiments, the features are measurable aspects of a plurality of biomarkers comprising at least 2, 3, 4, 5, 6, 7, 8, 9, 10, 11, 12, 13, 14, 15, 16, 17, 18, 19, or biomarkers listed in any one of Tables 1, 4, 5, 6, 7, 12, 13, 14, 15, 16, 17, 18, 19, 20, and 21. When the plurality of biomarkers comprises complement component C3 and complement component C4, the plurality of biomarkers comprises three or more biomarkers. In some embodiments, the features are measurable aspects of a plurality of biomarkers comprising at least 2, 3, 4, 5, 6, 7, 8, 9, 10, 11, 12, 13, 14, 15, 16, 17, 18, 19, or 20 biomarkers listed in any combination of Tables 1, 4, 5, 6, 7, 12, 13, 14, 15, 16, 17, 18, 19, 20, and 21. In some embodiments, the computer program product further comprises instructions for evaluating whether the plurality of features in the biomarker profile of the test subject satisfies a second value set. Satisfaction of the second value set predicts that the test subject is not likely to develop sepsis. In some embodiments, the biomarker profile has between 3 and 50 biomarkers listed in one of Tables 1, 4, 5, 6, 7, 12, 13, 14, 15, 16, 17, 18, 19, 20, and 21, between 3 and 40 biomarkers listed in one of Tables 1, 4, 5, 6, 7, 12, 13, 14, 15, 16, 17, 18, 19, 20, and 21, at least four biomarkers listed in one of Tables 1, 4, 5, 6, 7, 12, 13, 14, 15, 16, 17, 18, 19, 20, and 21, or at least six biomarkers listed in one of Tables 1, 4, 5, 6, 7, 12, 13, 14, 15, 16, 17, 18, 19, 20, and 21. In some embodiments, the biomarker profile has at least 1, 2, 3, 4, 5, 6, 7, 8, 9 or 10 biomarkers from columns 3 and/or 4 of Table 4. In some embodiments, the biomarker profile comprises CRP, APOA2, and SERPINC1. In some embodiments, the biomarker profile comprises at least one of SERPINC1, APOA2, and CRP, and, additionally, at least 1, 2, 3, 4, 5, 6 or 7 other additional biomarkers in Table 4. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19, 20, and 21. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21.

Another computer embodiment of the present invention comprises a central processing unit and a memory coupled to the central processing unit. The memory stores instructions for evaluating whether a plurality of features in a biomarker profile of a test subject at risk for developing sepsis satisfies a first value set. Satisfaction of the first value set predicts that the test subject is likely to develop sepsis. The features are measurable aspects of a plurality of biomarkers. In some embodiments, this plurality of biomarkers comprises at least 2, 3, 4, 5, 6, 7, 8, 9, 10, 11, 12, 13, 14, 15, 16, 17, 18, 19, or 20 biomarkers from anyone of Tables 1, 4, 5, 6, 7, 12, 13, 14, 15, 16, 17, 18, 19, 20, and 21. In some embodiments, this plurality of biomarkers comprises at least 2, 3, 4, 5, 6, 7, 8, 9, 10, 11, 12, 13, 14, 15, 16, 17, 18, 19, or 20 biomarkers from any combination of Tables 1, 4, 5, 6, 7, 12, 13, 14, 15, 16, 17, 18, 19, 20, and 21. In some embodiments, the memory further stores instructions for evaluating whether the plurality of features in the biomarker profile of the test subject satisfies a second value set, where satisfying the second value set predicts that the test subject is not likely to develop sepsis. In some embodiments, the biomarker profile consists of between 3 and 50 biomarkers listed in one of Tables 1, 4, 5, 6, 7, 12, 13, 14, 15, 16, 17, 18, 19, 20, and 21, between 3 and 40 biomarkers listed in one of Tables 1, 4, 5, 6, 7, 12, 13, 14, 15, 16, 17, 18, 19, 20, and 21, at least four biomarkers listed in one of 1, 4, 5, 6, 7, 12, 13, 14, 15, 16, 17, 18, 19, 20, and 21, or at least eight biomarkers listed in one of Tables 1, 4, 5, 6, 7, 12, 13, 14, 15, 16, 17, 18, 19, 20, and 21. In some embodiments, when the plurality of biomarkers comprises complement component C3 and complement component C4, the plurality of biomarkers comprises three or more biomarkers.

Another computer embodiment in accordance with the present invention comprises a computer system for determining whether a subject is likely to develop sepsis. The computer system comprises a central processing unit and a memory, coupled to the central processing unit. The memory stores instructions for obtaining a biomarker profile of a test subject. The biomarker profile comprises a plurality of features. In some embodiments, the plurality of features comprises at least 2, 3, 4, 5, 6, 7, 8, 9, 10, 11, 12, 13, 14, 15, 16, 17, 18, 19, or 20 biomarkers listed in any one of Tables 1, 4, 5, 6, 7, 12, 13, 14, 15, 16, 17, 18, 19, 20, and 21. In some embodiments, the plurality of features comprises at least 2, 3, 4, 5, 6, 7, 8, 9, 10, 11, 12, 13, 14, 15, 16, 17, 18, 19, or 20 biomarkers listed in any combination of Tables 1, 4, 5, 6, 7, 12, 13, 14, 15, 16, 17, 18, 19, 20, and 21. When the plurality of features comprises features for complement component C3 and complement component C4, the plurality of features comprises features for three or more biomarkers. The memory further comprises instructions for transmitting the biomarker profile to a remote computer. The remote computer includes instructions for evaluating whether the plurality of features in the biomarker profile of the test subject satisfies a first value set. Satisfaction of the first value set predicts that the test subject is likely to develop sepsis. The memory further comprises instructions for receiving a determination, from the remote computer, as to whether the plurality of features in the biomarker profile of the test subject satisfies the first value set. The memory also comprises instructions for reporting whether the plurality of features in the biomarker profile of the test subject satisfies the first value set. In some embodiments, the remote computer further comprises instructions for evaluating whether the plurality of features in the biomarker profile of the test subject satisfies a second value set. Satisfaction of the second value set predicts that the test subject is not likely to develop sepsis. In such embodiments, the memory further comprises instructions for receiving a determination, from the remote computer, as to whether the plurality of features in the biomarker profile of the test subject satisfies the second set as well as instructions for reporting whether the plurality of features in the biomarker profile of the test subject satisfies the second value set. In some embodiments, the plurality of biomarkers comprises CRP, APOA2, and SERPINC1. In some embodiments, the biomarker profile comprises at least one of SERPINC1, APOA2, and CRP and, additionally, at least 1, 2, 3, 4, 5, 6 or 7 other additional biomarkers in Table 4. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19, 20, and 21. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21. In some embodiments, the plurality of biomarkers comprises at least two biomarkers from Table 4. In some embodiments, when the plurality of biomarkers comprises complement component C3 and complement component C4, the plurality of biomarkers comprises three or more biomarkers.

Yet another aspect of the present invention provides a computer system for determining whether a subject is likely to develop sepsis. The computer system comprises a central processing unit and a memory, coupled to the central processing unit. The memory stores instructions for obtaining a biomarker profile of a test subject. The biomarker profile comprises a plurality of features. The features are measurable aspects of a plurality of biomarkers. In some embodiments, the plurality of biomarkers comprise at least 2, 3, 4, 5, 6, 7, 8, 9, 10, 11, 12, 13, 14, 15, 16, 17, 18, 19 or 20 biomarkers listed in anyone of Tables 1, 4, 5, 6, 7, 12, 13, 14, 15, 16, 17, 18, 19, 20, and 21. In some embodiments, the plurality of biomarkers comprise at least 2, 3, 4, 5, 6, 7, 8, 9, 10, 11, 12, 13, 14, 15, 16, 17, 18, 19 or 20 biomarkers listed in any combination of Tables 1, 4, 5, 6, 7, 12, 13, 14, 15, 16, 17, 18, 19, 20, and 21. The memory further stores instructions for evaluating whether the plurality of features in the biomarker profile of the test subject satisfies a first value set. Satisfying the first value set predicts that the test subject is likely to develop sepsis. The memory also stores instructions for reporting whether the plurality of features in the biomarker profile of the test subject satisfies the first value set. In some embodiments, the plurality of biomarkers comprises CRP, APOA2, and SERPINC1. In some embodiments, the plurality of biomarkers comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19, 20, and 21. In some embodiments, the plurality of biomarkers comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21. In some embodiments, the plurality of biomarkers comprises at least 2, 3, 4, 5, 6, 7, 8, 9 or 10 biomarkers from Table 4. In some embodiments, when the plurality of biomarkers comprises complement component C3 and complement component C4, the plurality of biomarkers comprises three or more biomarkers.

Each of the methods, computer program products, and computers disclosed herein optionally further comprises a step of, or instructions for, outputting a result (for example, to a monitor, to a user, to computer readable media, e.g., storage media or to a remote computer). Furthermore, in some embodiments, each of the methods, computer program products, and computers disclosed herein can optionally be limited to analysis of human subjects. Furthermore, in some embodiments, each of the methods, computer program products, and computers disclosed herein can optionally be limited to analysis of blood samples from human subjects. Still further, in some embodiments, each of the methods, computer program products, and computers disclosed herein can optionally be limited to analysis of nucleic acids in samples from human subjects. Still further, in some embodiments, each of the methods, computer program products, and computers disclosed herein can optionally be limited to analysis of proteins in samples from human subjects.

### 4. BRIEF DESCRIPTION OF THE FIGURES

FIG. 1 shows a computer system in accordance with the present invention.

FIG. 2 illustrates the involvement of classical and alternative complement cascades in differentiating sepsis from SIRS patients in terms of proteins identified in the present invention.

FIG. 3 illustrates the involvement of Intrinsic Prothrombin Activation pathway in differentiating Sepsis from SIRS patients using proteins identified in the present invention.

FIG. 4 illustrates a time normalization scheme in which samples are evaluated at (i) T₋₆₀=49-72 hours before the T₋₀ time point, (ii) T₋₃₆=25-48 hours prior to T₋₀, and (iii) T₋₁₂=1-24 hours prior to T₋₀, where T₋₀ is considered the time of clinical diagnosis of sepsis for the pre-septic group or time-matched control for the uninfected SIRS group.

### 5. DETAILED DESCRIPTION OF THE PREFERRED EMBODIMENTS

The present invention allows for the rapid and accurate diagnosis or prediction of sepsis by evaluating biomarker features in biomarker profiles. These biomarker profiles can be constructed from one or more biological samples of subjects at a single time point (“snapshot”), or multiple such time points, during the course of time the subject is at risk for developing sepsis. Advantageously, sepsis can be diagnosed or predicted prior to the onset of conventional clinical sepsis symptoms, thereby allowing for more effective therapeutic intervention.

### 5.1 Definitions

“Systemic inflammatory response syndrome,” or “SIRS,” refers to a clinical response that is triggered by infectious or noninfectious conditions such as localized or generalized infection, trauma, thermal injury, or sterile inflammatory processes (e.g., acute pancreatitis). SIRS is considered to be present when a subject is (i) undergoing a response to any of the foregoing infectious or noninfectious conditions, and (ii) is exhibiting some of the following clinical findings:

fever (body temperature greater than 38.3° C.);

hypothermia (body temperature less than 36° C.);

heart rate (HR) greater than 90 beats/minute or >2 standard deviations above the normal value for age;

tachypnea;

altered mental status;

significant edema or positive fluid balance (>20 mL/kg over 24 hours);

hyperglycemia (plasma glucose>120 mg/dL or 7.7 mmol/L) in the absence of diabetes;

leukocytosis (white cell blood count>12,000 μL⁻¹);

leucopenia (white cell blood count<4,000 μL⁻¹);

normal white cell blood count>10% immature forms;

plasma C-reactive protein>2 standard deviations above the normal value;

plasma procalcitonin>2 standard deviations above the normal value;

arterial hypotension (SBP<90 mm Hg, MAP<80, or an SBP decrease>40 mm Hg in adults or <two standard deviations below normal for age);

cardiac index>3.5 L·min⁻¹·M⁻²³;

arterial hypoxemia (PaO₂/FIO₂<300);

acute oliguria (urine output<0.5 mL·kg⁻¹·hr⁻¹ or 45 mmol/L for at least two hours);

creatinine increase>0.5 mg/dL;

coagulation abnormalities (INR>1.5 or aPTT>60 seconds);

Ileus (absent bowel sounds);

Thrombocytopenia (platelet count<100,000 μL⁻¹);

Hyperbilirubinemia (plasma total bilirubin>4 mg/dL or 70 mmol/L);

Hyperlactatemia (>1 mmol/L); and

Decreased capillary refill or mottling.

These symptoms of SIRS represent a consensus definition of SIRS that can be modified or supplanted by other definitions in the future. The present definition is used to clarify current clinical practice and does not represent a critical aspect of the invention. For more information on standards used to define SIRs see, for example, American College of Chest Physicians/Society of Critical Care Medicine Consensus Conference: Definitions for Sepsis and Organ Failure and Guidelines for the Use of Innovative Therapies in Sepsis, 1992, Crit. Care. Med. 20, 864-874; Levy et al., 2003, “2001 SCCM/ESICM/ACCP/ATS/SIS International Sepsis Definitions Conference,” Crit. Care Med. 31, 1250-1256; and Carrigan et al., 2004, “Toward Resolving the Challenges of Sepsis Diagnosis,” 1301-1314, each of which is incorporated by reference herein in its entirety.

A subject with SIRS has a clinical presentation that is classified as SIRS, as defined above, but is not clinically deemed to be septic. Methods for determining which subjects are at risk of developing sepsis are well known to those in the art. Such subjects include, for example, those in an ICU and those who have otherwise suffered from a physiological trauma, such as a burn, surgery or other insult. A hallmark of SIRS is the creation of a proinflammatory state that can be marked by tachycardia, tachypnea or hyperpnea, hypotension, hypoperfusion, oliguria, leukocytosis or leukopenia, pyrexia or hypothermia and the need for volume infusion. SIRS characteristically does not include a documented source of infection (e.g., bacteremia).

“Sepsis” refers to a state in which a subject has both (i) SIRS and (ii) a documented or suspected infection (e.g., a subsequent laboratory confirmation of a clinically significant infection such as a positive culture for an organism). Thus, sepsis refers to the systemic inflammatory response to an infection (see, e.g., American College of Chest Physicians Society of Critical Care Medicine, Chest, 1997, 101:1644-1655, the entire contents of which are herein incorporated by reference). As used here, the term “infection” means a pathological process induced by a microorganism. Such an infection can be caused by pathogenic gram-negative and gram-positive bacteria, anaerobic bacteria, fungi, yeast, or polymicrobial organisms. Exemplary non-limiting sites of such infections are respiratory tract infactions, genitourinary infections, and intraabdoiminal infections. As used herein, “sepsis” includes all stages of sepsis including, but not limited to, the onset of sepsis, severe sepsis, septic shock and multiple organ dysfunction (“MOD”) associated with the end stages of sepsis.

The “onset of sepsis” refers to an early stage of sepsis, e.g., prior to a stage when conventional clinical manifestations are sufficient to support a clinical suspicion of sepsis. Because the methods of the present invention are used to detect sepsis prior to a time that sepsis would be suspected using conventional techniques, the subject's disease status at early sepsis can only be confirmed retrospectively, when the manifestation of sepsis is more clinically obvious. The exact mechanism by which a subject becomes septic is not a critical aspect of the invention. The methods of the present invention can detect the onset of sepsis independent of the origin of the infectious process.

“Severe sepsis” refers to sepsis associated with organ dysfunction, hypoperfusion abnormalities, or sepsis-induced hypotension. Hypoperfusion abnormalities include, but are not limited to, lactic acidosis, oliguria, or an acute alteration in mental status.

“Septic shock” in adults refers to a state of acute circulatory failure characterized by persistent arterial hypotension unexplained by other causes. Hypotension is defined by a systolic arterial pressure below 90 mm Hg (or, in children, <2SD below normal for their age), a MAP<60, or a reduction in systolic blood pressure of >40 mm Hg from baseline, despite adequate volume resuscitation, in the absence of other causes for hypotension. Children and neontates maintain higher vascular tone than adults. Therefore, the shock state occurs long before hypertension in children. Septic shock in pediatric patients is defined as a tachychardia (may be absent in the hypothermic patient) with sings of decreased perfusion including decreased peripheral pulses compared with central pulses, altered alertness, flash capillary refill or capillary refill>2 seconds, mottled or cool extremities, or decreased urine output. Hypotension is a sign of late and decompensated shock in children. See, for example, Levy et al., 2003, “2001 SCCM/ESICM/ACCP/ATS/SIS International Sepsis Definitions Conference,” Crit. Care Med. 31, 1250-1256; and Carrigan et al., 2004, “Toward Resolving the Challenges of Sepsis Diagnosis,” 1301-1314; and Carcillo, 2002, “Clinical Practice Paramaters for Hemodynamic Support of Pediatric and Neonatal Patients in Septic Shock,” Crit. Care Med. 30, pp. 1-13, each of which is hereby incorporated by reference in its entirety.

A “converter” or “converter subject” refers to a SIRS-positive subject who progresses to clinical suspicion of sepsis during the period the subject is monitored, typically during an ICU stay.

A “non-converter” or “non-converter subject” refers to a SIRS-positive subject who does not progress to clinical suspicion of sepsis during the period the subject is monitored, typically during an ICU stay.

A “biomarker” is virtually any detectable compound, such as a protein, a peptide, a proteoglycan, a glycoprotein, a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite), or a discriminating molecule or discriminating fragment of any of the foregoing, that is present in or derived from a biological sample. “Derived from” as used in this context refers to a compound that, when detected, is indicative of a particular molecule being present in the biological sample. For example, detection of a particular cDNA can be indicative of the presence of a particular RNA transcript in the biological sample. As another example, detection of binding to a particular antibody can be indicative of the presence or absence of a particular antigen (e.g., protein) in the biological sample. Here, a discriminating molecule or fragment is a molecule or fragment that, when detected, indicates presence or abundance of an above-identified compound.

A biomarker can, for example, be isolated from the biological sample, directly measured in the biological sample, or detected in or determined to be in the biological sample. A biomarker can, for example, be functional, partially functional, or non-functional. In one embodiment of the present invention, a biomarker is isolated and used, for example, to raise a specifically-binding antibody that can facilitate biomarker detection in a variety of diagnostic assays. Any immunoassay may use any antibodies, antibody fragment or derivative thereof capable of binding the biomarker molecules (e.g., Fab, F(ab′)₂, Fv, or scFv fragments). Such immunoassays are well-known in the art. In addition, if the biomarker is a protein or fragment thereof, it can be sequenced and its encoding gene can be cloned using well-established techniques. When a specific biomarker is listed herein, for example, as part of a biomarker profile, kit, or otherwise, the biomarker can be, for example, the precursor of the listed biomarker, the fully processed version of the listed biomarker, a splice variant of the biomarker, a fragment thereof, an antibody thereof, or a discriminating molecule thereof. For instance, reference to CRP herein is, for example, a reference to C-reactive protein, C-reactive protein precursor, a fragment thereof, an antibody thereof, a nucleic acid encoding all or a fragment thereof, a discriminating molecule thereof, or any other type of biomarker for CRP. Reference to APOA2 herein is, for example, is a reference to apolipoprotein A-II, apolipoprotein A-II precursor, a fragment thereof, an antibody thereof, a nucleic acid encoding all or a fragment thereof, a discriminating molecule thereof, or any other type of biomarker for APOA2. Reference to SERPINC1 herein is, for example, is a reference to serine (or cysteine) proteinase inhibitor (or any of its synonyms including, but not limited to, clade C, antithrombin member 1, antithrombin-III precursor, ATIII, etc.), a fragment thereof, an antibody thereof, a nucleic acid encoding all or a fragment thereof, a discriminating molecule thereof, or any other type of biomarker for SERPINC.

As used herein, the term “a species of a biomarker” refers to any discriminating portion or discriminating fragment of a biomarker described herein, such as a splice variant of a particular gene described herein (e.g., a gene listed in Table 1, 4, 5, 6 and/or 7, below). Here, a discriminating portion or discriminating fragment is a portion or fragment of a molecule that, when detected, indicates presence or abundance of the above-identified transcript, cDNA, amplified nucleic acid, or protein.

As used herein, the terms “protein”, “peptide”, and “polypeptide” are, unless otherwise indicated, interchangeable.

A “biomarker profile” comprises a plurality of one or more types of biomarkers (e.g., an mRNA molecule, a cDNA molecule, a protein and/or a carbohydrate, etc.), or an indication thereof, together with a feature, such as a measurable aspect (e.g., abundance) of the biomarkers. A biomarker profile comprises at least two such biomarkers or indications thereof, where the biomarkers can be in the same or different classes, such as, for example, a nucleic acid and a carbohydrate. A biomarker profile may also comprise at least 3, 4, 5, 6, 7, 8, 9, 10, 11, 12, 13, 14, 15, 16, 17, 18, 19, 20, 21, 22, 23, 24, 25, 30, 35, 40, 45, 50, 54, 5, 60, 65, 70, 75, 80, 85, 90, 95, or 100 or more biomarkers or indications thereof. In one embodiment, a biomarker profile comprises hundreds, or even thousands, of biomarkers or indications thereof. A biomarker profile can further comprise one or more controls or internal standards. In one embodiment, the biomarker profile comprises at least one biomarker, or indication thereof, that serves as an internal standard. In another embodiment, a biomarker profile comprises an indication of one or more types of biomarkers. The term “indication” as used herein in this context merely refers to a situation where the biomarker profile contains symbols, data, abbreviations or other similar indicia for a biomarker, rather than the biomarker molecular entity itself. In some embodiments, the biomarker profile comprises a nominal indication of the quantity of a transcript of a gene from one any one of Tables 1, 4, 5, 6, 7, 12, 13, 14, 15, 16, 17, 18, 19, 20, and 21. Still another exemplary biomarker profile of the present invention comprises a microarray to which a physical quantity of a gene transcript from one of Tables 1, 4, 5, 6, 7, 12, 13, 14, 15, 16, 17, 18, 19, 20, and 21 is taken. In this last exemplary biomarker profile, at least twenty percent, forty percent, or more than forty percent of the probes spots are based on sequences in one of Tables 1, 4, 5, 6, 7, 12, 13, 14, 15, 16, 17, 18, 19, 20, and 21. In another exemplary biomarker profile, at least twenty percent, forty percent, or more than forty percent of the probes spots are based on sequences in probe sets for biomarkers listed in any one of Tables 1, 4, 5, 6, 7, 12, 13, 14, 15, 16, 17, 18, 19, 20, and 21. In some embodiments, when the biomarker profile comprises the biomarkers component C3 and complement component C4, the biomarker profile comprises three or more biomarkers.

In typical embodiments, each biomarker in a biomarker profile includes a corresponding “feature.” A “feature”, as used herein, refers to a measurable aspect of a biomarker. A feature can include, for example, the presence or absence of biomarkers in the biological sample from the subject as illustrated in exemplary biomarker profile 1:

Exemplary Biomarker Profile 1.

| | Feature |
|---|---|
| Biomarker | Presence in sample |
| transcript of gene A | Present |
| transcript of gene B | Absent |

In exemplary biomarker profile 1, the feature value for the transcript of gene A is “presence” and the feature value for the transcript of gene B is “absence.”

A feature can include, for example, the abundance of a biomarker in the biological sample from a subject as illustrated in exemplary biomarker profile 2:

Exemplary Biomarker Profile 2.

| | Feature |
|---|---|
| | Abundance in sample in |
| Biomarker | relative units |
| transcript of gene A | 300 |
| transcript of gene B | 400 |

In exemplary biomarker profile 2, the feature value for the transcript of gene A is 300 units and the feature value for the transcript of gene B is 400 units.

A feature can also be a ratio of two or more measurable aspects of a biomarker as illustrated in exemplary biomarker profile 3:

Exemplary Biomarker Profile 3.

| | Feature |
|---|---|
| | Ratio of abundance of |
| | transcript of gene A/transcript of |
| Biomarker | gene Y |
| transcript of gene A | 300/400 |
| transcript of gene B | |

In exemplary biomarker profile 3, the feature value for the transcript of gene A and the feature value for the transcript of gene B is 0.75 (300/400).

A feature may also be the difference between a measurable aspect of the corresponding biomarker that is taken from two samples, where the two samples are collected from a subject at two different time points. For example, consider the case where the biomarker is a transcript of a gene A and the “measurable aspect” is abundance of the transcript, in samples obtained from a test subject as determined by, e.g., RT-PCR or microarray analysis. In this example, the abundance of the transcript of gene A is measured in a first sample as well as a second sample. The first sample is taken from the test subject a number of hours before the second sample. To compute the feature for gene A, the abundance of the transcript of gene A in one sample is subtracted from the abundance of the transcript of gene A in the second sample. A feature can also be an indication as to whether an abundance of a biomarker is increasing in biological samples obtained from a subject over time and/or an indication as to whether an abundance of a biomarker is decreasing in biological samples obtained from a subject over time.

In some embodiments, there is a one-to-one correspondence between features and biomarkers in a biomarker profile as illustrated in exemplary biomarker profile 1, above. In some embodiments, the relationship between features and biomarkers in a biomarker profile of the present invention is more complex, as illustrated in Exemplary biomarker profile 3, above.

Those of skill in the art will appreciate that other methods of computation of a feature can be devised and all such methods are within the scope of the present invention. For example, a feature can represent the average of an abundance of a biomarker across biological samples collected from a subject at two or more time points. Furthermore, a feature can be the difference or ratio of the abundance of two or more biomarkers from a biological sample obtained from a subject in a single time point. A biomarker profile may also comprise at least three, four, five, 10, 20, 30 or more features. In one embodiment, a biomarker profile comprises hundreds, or even thousands, of features.

In some embodiments, features of biomarkers are measured using microarrays. The construction of microarrays and the techniques used to process microarrays in order to obtain abundance data is well known, and is described, for example, by Draghici, 2003, Data Analysis Tools for DNA Microarrays, Chapman & Hall/CRC, and international publication number WO 03/061564, each of which is hereby incorporated by reference herein in its entirety. A microarray comprises a plurality of probes. In some instances, each probe recognizes, e.g., binds to, a different biomarker. In some instances, two or more different probes on a microarray recognize, e.g., bind to, the same biomarker. Thus, typically, the relationship between probe spots on the microarray and a subject biomarker is a two to one correspondence, a three to one correspondence, or some other form of correspondence. However, it can be the case that there is a unique one-to-one correspondence between probes on a microarray and biomarkers.

A “phenotypic change” is a detectable change in a parameter associated with a given state of the subject. For instance, a phenotypic change can include an increase or decrease of a biomarker in a bodily fluid, where the change is associated with SIRS, sepsis, the onset of sepsis or with a particular stage in the progression of sepsis. A phenotypic change can further include a change in a detectable aspect of a given state of the subject that is not a change in a measurable aspect of a biomarker. For example, a change in phenotype can include a detectable change in body temperature, respiration rate, pulse, blood pressure, or other physiological parameter. Such changes can be determined via clinical observation and measurement using conventional techniques that are well-known to the skilled artisan.

As used herein, the term “complementary,” in the context of a nucleic acid sequence (e.g., a nucleotide sequence encoding a gene described herein), refers to the chemical affinity between specific nitrogenous bases as a result of their hydrogen bonding properties. For example, guanine (G) forms a hydrogen bond with only cytosine (C), while adenine forms a hydrogen bond only with thymine (T) in the case of DNA, and uracil (U) in the case of RNA. These reactions are described as base pairing, and the paired bases (G with C, or A with T/U) are said to be complementary. Thus, two nucleic acid sequences may be complementary if their nitrogenous bases are able to form hydrogen bonds. Such sequences are referred to as “complements” of each other. Such complement sequences can be naturally occurring, or, they can be chemically synthesized by any method known to those skilled in the art, as for example, in the case of antisense nucleic acid molecules which are complementary to the sense strand of a DNA molecule or an RNA molecule (e.g., an mRNA transcript). See, e.g., Lewin, 2002, Genes VII. Oxford University Press Inc., New York, N.Y., which is hereby incorporated by reference herein in its entirety.

As used herein, “conventional techniques” in the context of diagnosing or predicting sepsis or SIRS are those techniques that classify a subject based on phenotypic changes without obtaining a biomarker profile according to the present invention.

A “decision rule” is a method used to evaluate biomarker profiles. Such decision rules can take on one or more forms that are known in the art, as exemplified in Hastie et al., 2001, The Elements of Statistical Learning, Springer-Verlag, New York, which is hereby incorporated by reference in its entirety. A decision rule may be used to act on a data set of features to, inter alia, predict the onset of sepsis, to determine the progression of sepsis, or to diagnose sepsis. Exemplary decision rules that can be used in some embodiments of the present invention are described in further detail in Section 5.5, below.

“Predicting the development of sepsis” is the determination as to whether a subject will develop sepsis. Any such prediction is limited by the accuracy of the means used to make this determination. The present invention provides a method, e.g., by utilizing a decision rule(s), for making this determination with an accuracy that is 60% or greater. As used herein, the terms “predicting the development of sepsis” and “predicting sepsis” are interchangeable. In some embodiments, the act of predicting the development of sepsis (predicting sepsis) is accomplished by evaluating one or more biomarker profiles from a subject using a decision rule that is indicative of the development of sepsis and, as a result of this evaluation, receiving a result from the decision rule that indicates that the subject will become septic. Such an evaluation of one or more biomarker profiles from a test subject using a decision rule uses some or all the features in the one or more biomarker profiles to obtain such a result.

The terms “obtain” and “obtaining,” as used herein, mean “to come into possession of,” or “coming into possession of,” respectively. This can be done, for example, by retrieving data from a data store in a computer system. This can also be done, for example, by direct measurement.

As used herein, the term “specifically,” and analogous terms, in the context of an antibody, refers to peptides, polypeptides, and antibodies or fragments thereof that specifically bind to an antigen or a fragment and do not specifically bind to other antigens or other fragments. A peptide or polypeptide that specifically binds to an antigen may bind to other peptides or polypeptides with lower affinity, as determined by standard experimental techniques, for example, by any immunoassay well-known to those skilled in the art. Such immunoassays include, but are not limited to, radioimmunoassays (RIAs) and enzyme-linked immunosorbent assays (ELISAs). Antibodies or fragments that specifically bind to an antigen may be cross-reactive with related antigens. Preferably, antibodies or fragments thereof that specifically bind to an antigen do not cross-react with other antigens. See, e.g., Paul, ed., 2003, Fundamental Immunology, 5th ed., Raven Press, New York at pages 69-105, which is incorporated by reference herein, for a discussion regarding antigen-antibody interactions, specificity and cross-reactivity, and methods for determining all of the above.

As used herein, a “subject” is an animal, preferably a mammal, more preferably a non-human primate, and most preferably a human. The terms “subject” “individual” and “patient” are used interchangeably herein.

As used herein, a “test subject,” typically, is any subject that is not in a training population used to construct a decision rule. A test subject can optionally be suspected of either having sepsis at risk of developing sepsis.

As used herein, a “tissue type,” is a type of tissue. A tissue is an association of cells of a multicellular organism, with a common embryoloical origin or pathway and similar structure and function. Often, cells of a tissue are contiguous at cell membranes but occasionally the tissue may be fluid (e.g., blood). Cells of a tissue may be all of one type (a simple tissue, e.g., squamous epithelium, plant parentchyma) or of more than one type (a mixed tissue, e.g., connective tissue).

As used herein, a “training population” is a set of samples from a population of subjects used to construct a decision rule, using a data analysis algorithm, for evaluation of the biomarker profiles of subjects at risk for developing sepsis. In a preferred embodiment, a training population includes samples from subjects that are converters and subjects that are nonconverters.

As used herein, a “data analysis algorithm” is an algorithm used to construct a decision rule using biomarker profiles of subjects in a training population. Representative data analysis algorithms are described in Section 5.5. A “decision rule” is the final product of a data analysis algorithm, and is characterized by one or more value sets, where each of these value sets is indicative of an aspect of SIRS, the onset of sepsis, sepsis, or a prediction that a subject will acquire sepsis. In one specific example, a value set represents a prediction that a subject will develop sepsis. In another example, a value set represents a prediction that a subject will not develop sepsis.

As used herein, a “validation population” is a set of samples from a population of subjects used to determine the accuracy of a decision rule. In a preferred embodiment, a validation population includes samples from subjects that are converters and subjects that are nonconverters. In a preferred embodiment, a validation population does not include subjects that are part of the training population used to train the decision rule for which an accuracy measurement is sought.

As used herein, a “value set” is a combination of values, or ranges of values for features in a biomarker profile. The nature of this value set and the values therein is dependent upon the type of features present in the biomarker profile and the data analysis algorithm used to construct the decision rule that dictates the value set. To illustrate, reconsider exemplary biomarker profile 2:

Exemplary Biomarker Profile 2.

| | Feature |
|---|---|
| | Abundance in sample in |
| Biomarker | relative units |
| transcript of gene A | 300 |
| transcript of gene B | 400 |

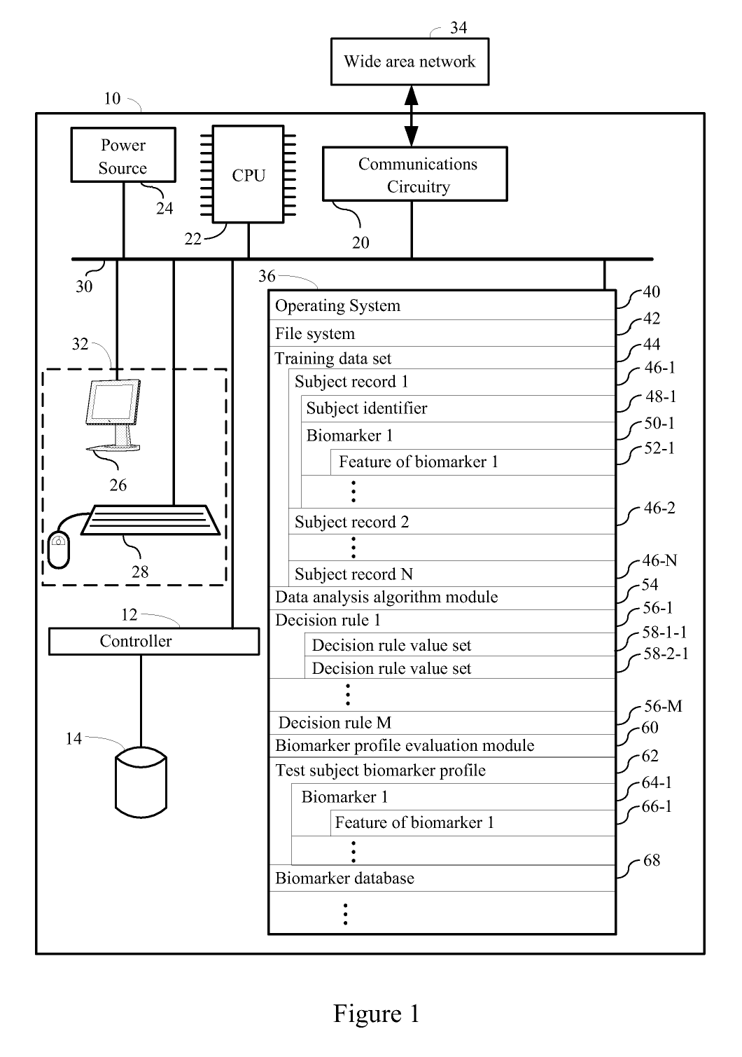

In this example, the biomarker profile of each member of a training population is obtained. Each such biomarker profile includes a measured feature, here abundance, for the transcript of gene A, and a measured feature, here abundance, for the transcript of gene B. These feature values, here abundance values, are used by a data analysis algorithm to construct a decision rule. In this example, the data analysis algorithm is a decision tree, described in Section 5.5.1 and the final product of this data analysis algorithm, the decision rule, is a decision tree. An exemplary decision tree is illustrated in FIG. 1. The decision rule defines value sets. One such value set is predictive of the onset of sepsis. A subject whose biomarker feature values satisfy this value set is likely to become septic. An exemplary value set of this class is exemplary value set 1:

Exemplary Value Set 1.

| | Value set component |
|---|---|
| | (Abundance in sample in |
| Biomarker | relative units) |
| transcript of gene A | <400 |
| transcript of gene B | <600 |

Another such value set is predictive of a septic-free state. A subject whose biomarker feature values satisfy this value set is not likely to become septic. An exemplary value set of this class is exemplary value set 2:

Exemplary Value Set 2.

| | Value set component |
|---|---|
| | (Abundance in sample in |
| Biomarker | relative units) |
| transcript of gene A | >400 |
| transcript of gene B | >600 |

In the case where the data analysis algorithm is a neural network analysis and the final product of this neural network analysis is an appropriately weighted neural network, one value set is those ranges of biomarker profile feature values that will cause the weighted neural network to indicate that onset of sepsis is likely. Another value set is those ranges of biomarker profile feature values that will cause the weighted neural network to indicate that onset of sepsis is not likely.

As used herein, the term “probe spot” in the context of a microarray refers to a single stranded DNA molecule (e.g., a single stranded cDNA molecule or synthetic DNA oligomer), referred to herein as a “probe,” that is used to determine the abundance of a particular nucleic acid in a sample. For example, a probe spot can be used to determine the level of mRNA in a biological sample (e.g., a collection of cells) from a test subject. In a specific embodiment, a typical microarray comprises multiple probe spots that are placed onto a glass slide (or other substrate) in known locations on a grid. The nucleic acid for each probe spot is a single stranded contiguous portion of the sequence of a gene or gene of interest (e.g., a 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer or larger) and is a probe for the mRNA encoded by the particular gene or gene of interest. Each probe spot is characterized by a single nucleic acid sequence, and is hybridized under conditions that cause it to hybridize only to its complementary DNA strand or mRNA molecule. As such, there can be many probe spots on a substrate, and each can represent a unique gene or sequence of interest. In addition, two or more probe spots can represent the same gene sequence. In some embodiments, a labeled nucleic sample is hybridized to a probe spot, and the amount of labeled nucleic acid specifically hybridized to a probe spot can be quantified to determine the levels of that specific nucleic acid (e.g., mRNA transcript of a particular gene) in a particular biological sample. Probes, probe spots, and microarrays, generally, are described in Draghici, 2003, Data Analysis Tools for DNA Microarrays, Chapman & Hall/CRC, Chapter, 2, which is hereby incorporated by reference in its entirety.

As used herein, the term “annotation data” refers to any type of data that describes a property of a biomarker. Annotation data includes, but is not limited to, biological pathway membership, enzymatic class (e.g., phosphodiesterase, kinase, metalloproteinase, etc.), protein domain information, enzymatic substrate information, enzymatic reaction information, protein interaction data, disease association, cellular localization, tissue type localization, and cell type localization.

As used herein, the term “T₋₀” refers to a clinical conversion time point that is defined as the time when: 1) a positive culture is obtained from an otherwise sterile location or direct visualization of perforated or necrotic bowel in a subject; and 2) a clinical treatment (antibiotics and/or surgical procedure) is initiated on the subject for the infection as determined by majority consensus of an infectious disease attending, surgery attending, and critical care attendance. For subjects that do not become septic, such subjects are time matched to similar pre-septic subjects that later acquire sepsis based on demographic information, continued presence of SIRS, and elapsed time in the study.

As used herein, the term “T₋₁₂” refers to samples collected between 1 to 24 hours prior to the T₋₀ time point.

As used herein, the term T₋₃₆” refers to samples collected 25-48 hours prior to the T₋₀ time point.

As used herein, the term “T₋₆₀” refers to samples drawn 49-72 hours prior to the T₋₀ time point.

### 5.2 Methods for Screening Subjects

The present invention allows for accurate, rapid prediction and/or diagnosis of sepsis through detection of 2, 3, 4, 5, 6, 7, 8, 9, 10, 11, 12, 13, 14, 15, 16, 17, 18, 19, 20 or more features of a biomarker profile of a test individual suspected of or at risk for developing sepsis in each of one or more biological samples from a test subject. In one embodiment, only a single biological sample taken at a single point in time from the test subject is needed to construct a biomarker profile that is used to make this prediction or diagnosis of sepsis. In another embodiment, multiple biological samples taken at different points in time from the test subject are used to construct a biomarker profile that is used to make this prediction or diagnosis of sepsis.

In specific embodiments of the invention, subjects at risk for developing sepsis or SIRS are screened using the methods of the present invention. In accordance with these embodiments, the methods of the present invention can be employed to screen, for example, subjects admitted to an ICU and/or those who have experienced some sort of trauma (such as, e.g., surgery, vehicular accident, gunshot wound, etc.).

In specific embodiments, a biological sample such as, for example, blood, is taken upon admission. In some embodiments, a biological sample is blood, plasma, serum, saliva, sputum, urine, cerebral spinal fluid, cells, a cellular extract, a tissue specimen, a tissue biopsy, or a stool specimen. In some embodiments a biological sample is whole blood and this whole blood is used to obtain measurements for a biomarker profile. In some embodiments a biological sample is some component of whole blood. For example, in some embodiments some portion of the mixture of proteins, nucleic acid, and/or other molecules (e.g., metabolites) within a cellular fraction or within a liquid (e.g., plasma or serum fraction) of the blood is resolved as a biomarker profile. This can be accomplished by measuring features of the biomarkers in the biomarker profile. In some embodiments, the biological sample is whole blood but the biomarker profile is resolved from biomarkers in a specific cell type that is isolated from the whole blood. In some embodiments, the biological sample is whole blood but the biomarker profile is resolved from biomarkers expressed or otherwise found in monocytes that are isolated from the whole blood. In some embodiments, the biological sample is whole blood but the biomarker profile is resolved from biomarkers expressed or otherwise found in red blood cells that are isolated from the whole blood. In some embodiments, the biological sample is whole blood but the biomarker profile is resolved from biomarkers expressed or otherwise found in platelets that are isolated from the whole blood. In some embodiments, the biological sample is whole blood but the biomarker profile is resolved from biomarkers expressed or otherwise found in neutriphils that are isolated from the whole blood. In some embodiments, the biological sample is whole blood but the biomarker profile is resolved from biomarkers expressed or otherwise found in eosinophils that are isolated from the whole blood. In some embodiments, the biological sample is whole blood but the biomarker profile is resolved from biomarkers expressed or otherwise found in basophils that are isolated from the whole blood. In some embodiments, the biological sample is whole blood but the biomarker profile is resolved from biomarkers expressed or otherwise found in lymphocytes that are isolated from the whole blood. In some embodiments, the biological sample is whole blood but the biomarker profile is resolved from biomarkers expressed or otherwise found in monocytes that are isolated from the whole blood. In some embodiments, the biological sample is whole blood but the biomarker profile is resolved from one, two, three, four, five, six, or seven cell types from the group of cells types consisting of red blood cells, platelets, neutrophils, eosinophils, basophils, lymphocytes, and monocytes.

In some embodiments, a biomarker profile comprises a plurality of one or more types of biomarkers (e.g., an mRNA molecule, a cDNA molecule, a protein and/or a carbohydrate, etc.), or an indication thereof, together with features, such as a measurable aspect (e.g., abundance) of the biomarkers. A biomarker profile can comprise at least two such biomarkers or indications thereof, where the biomarkers can be in the same or different classes, such as, for example, a nucleic acid and a carbohydrate. In some embodiments, a biomarker profile comprises at least 2, 3, 4, 5, 6, 7, 8, 9, 10, 11, 12, 13, 14, 15, 16, 17, 18, 19, 20, 25, 30, 35, 40, 45, 50, 54, 5, 60, 65, 70, 75, 80, 85, 90, 95, 96, or 100 or more biomarkers or indications thereof. In one embodiment, a biomarker profile comprises hundreds, or even thousands, of biomarkers or indications thereof. In some embodiments, a biomarker profile comprises at least 2, 3, 4, 5, 6, 7, 8, 9, 10, 11, 12, 13, 14, 15, 16, 17, 18, 19, 20, 25, 30, 35, 40, 45, 50, or more biomarkers or indications thereof. In one example, in some embodiments, a biomarker profile comprises at least 2, 3, 4, 5, 6, 7, 8, 9, 10, 11, 12, 13, 14, 15, 16, 17, 18, 19, 20 or more biomarkers selected from Table 1 or indications thereof. In another example, in some embodiments, a biomarker profile comprises at least 2, 3, 4, 5, 6, 7, 8, 9 or more biomarkers selected from Table 4 or indications thereof. In another example, in some embodiments, a biomarker profile comprises at least CRP, APOA2, and SERPINC1, or indications thereof. In some embodiments, the biomarker profile comprises at least one of SERPINC1, APOA2, and CRP, and, additionally, at least 1, 2, 3, 4, 5, 6 or 7 other additional biomarkers in Table 4. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19, 20, and 21. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21. In another example, in some embodiments, a biomarker profile comprises at least 2, 3, 4, 5, 6, 7, 8, 9, 10, 11, 12, 13, 14, 15, 16, 17, 18, 19, 20 or more biomarkers selected from Table 5 or indications thereof. In yet another example, in some embodiments, a biomarker profile comprises at least 2, 3, 4, 5, 6, 7, 8, 9, 10, 11, 12, 13, 14, 15, 16, 17, 18, 19, 20 or more biomarkers selected from Table 6 or indications thereof. In one example, in some embodiments, a biomarker profile comprises at least 2, 3, 4, 5, 6, 7, 8, 9, 10, 11, 12, 13, 14, 15, 16, 17, 18, 19, 20 or more biomarkers selected from Table 7 or indications thereof. In one example, in some embodiments, a biomarker profile comprises at least 2, 3, 4, 5, 6, 7, 8, 9, 10, 11, 12, 13, 14, 15, 16, 17, 18, 19, 20 or more biomarkers selected from Table 15 or indications thereof. In one example, in some embodiments, a biomarker profile comprises at least 2, 3, 4, 5, 6, 7, 8, 9, 10, 11, 12, 13, 14, 15, 16, 17, 18, 19, 20 or more biomarkers selected from Table 17 or indications thereof. In some embodiments, when the biomarker profile comprises complement component C3 and complement component C4, the biomarker profile comprises three or more biomarkers. In typical embodiments, each biomarker in the biomarker profile is represented by a feature. In other words, there is a correspondence between biomarkers and features. In some embodiments, the correspondence between biomarkers and features is 1:1, meaning that for each single biomarker there is a corresponding single feature. In some embodiments, there is more than one feature for each biomarker. In some embodiments the number of features corresponding to one biomarker in the biomarker profile is different than then number of features corresponding to another biomarker in the biomarker profile. As such, in some embodiments, a biomarker profile can include at least 2, 3, 4, 5, 6, 7, 8, 9, 10, 11, 12, 13, 14, 15, 16, 17, 18, 19, 20, 25, 30, 35, 40, 45, 50, 54, 5, 60, 65, 70, 75, 80, 85, 90, 95, 96, 100 or more features, provided that there are at least 2, 3, 4, 5, 6, 7, 8, 9, 10, 11, 12, 13, 14, 15, 16, 17, 18, 19, 20, 25, 30, 35, 40, 45, 50, 54, 5, 60, 65, 70, 75, 80, 85, 90, 95, 96, 100 or more biomarkers in the biomarker profile. In some embodiments, a biomarker profile can include at least 2, 3, 4, 5, 6, 7, 8, 9, 10, 11, 12, 13, 14, 15, 16, 17, 18, 19, 20, 25, 30, 35, 40, 45, 50, or more features.

In some embodiments, a biomarker profile comprises at least 2, 3, 4, 5, 6, 7, 8, 9, 10, 11, 12, 13, 14, 15, 16, 17, 18, 19, 20, 25, 30, 35, 40, 45, 50, 54, 5, 60, 65, 70, 75, 80, 85, 90, 95, 96, or 100 or more biomarkers, where each such biomarker exhibits differential expression between converter and non-converter populations. Such differential expression (e.g., differential expression of nucleic acid or protein) can be in the form of upregulation or downregulation. For example, in some embodiments, a biomarker profile comprises biomarkers that are upregulated or downregulated in a converter population relative to the same biomarkers in a nonconverter population. In some embodiments, a biomarker is deemed to be upregulated in a converter population relative to a nonconverter population when the p-value for a parametric test (e.g., a T-test) of the significance of upregulation of the biomarker in the converter population relative to the nonconverter population is 0.1 or less, 0.05 or less, or 0.001 or less. In some embodiments, a biomarker is deemed to be downregulated in a converter population relative to a nonconverter population when the p-value for a parametric test (e.g., a T-test) of the significance of downregulation of the biomarker in the converter population relative to the nonconverter population is 0.1 or less, 0.05 or less, or 0.001 or less. In some embodiments, such differential expression may occur at any time point prior to conversion to sepsis, e.g., time of entry, T₋₆₀, T₋₃₆, T₋₁₂, etc. In some embodiments, such differential expression must occur at a specific time point prior to conversion to sepsis. For example, in some embodiments, a biomarker profile comprises biomarkers that exhibit differential expression at time of entry. In another example, a biomarker profile comprises biomarkers that exhibit differential expression at T₋₆₀. In another example, a biomarker profile comprises biomarkers that exhibit differential expression at T₋₃₆. In another example, a biomarker profile comprises biomarkers that exhibit differential expression at T₋₁₂. Examples of biomarkers that are differentially expressed includes, but is not limited to those listed in Table 12, 13, 14, 15, 16, 17, and 21, below.

Regardless of embodiment, the aforementioned features can be determined through the use of any reproducible measurement technique or combination of measurement techniques. Such techniques include those that are well known in the art including any technique described herein or, for example, any technique disclosed below. Typically, such techniques are used to measure feature values using a biological sample taken from a subject at a single point in time or multiple samples taken at multiple points in time. In one embodiment, an exemplary technique to obtain a biomarker profile from a sample taken from a subject is a cDNA microarray. In another embodiment, an exemplary technique to obtain a biomarker profile from a sample taken from a subject is a protein-based assay or other form of protein-based technique such as described in the BD Cytometric Bead Array (CBA) Human Inflammation Kit Instruction Manual (BD Biosciences) or the bead assay described in U.S. Pat. No. 5,981,180, each of which is incorporated herein by reference in its entirety, and in particular for their teachings of various methods of assay protein concentrations in biological samples. In still another embodiment, the biomarker profile is mixed, meaning that it comprises some biomarkers that are nucleic acids, or indications thereof, and some biomarkers that are proteins, or indications thereof. In such embodiments, both protein based and nucleic acid based techniques are used to obtain a biomarker profile from one or more samples taken from a subject. In other words, the feature values for the features associated with the biomarkers in the biomarker profile that are nucleic acids are obtained by nucleic acid based measurement techniques (e.g., a nucleic acid microarray) and the feature values for the features associated with the biomarkers in the biomarker profile that are proteins are obtained by protein based measurement techniques. In some embodiments biomarker profiles can be obtained using a kit, such as a kit described in Section 5.3 below.

In specific embodiments, a subject is screened using the methods and compositions of the invention as frequently as necessary (e.g., during their stay in the ICU) to diagnose or predict sepsis or SIRS in a subject. In a preferred embodiment, subjects are screened soon after they arrive in the ICU or other medical establishment. In some embodiments, subjects are screened daily after they arrive in the ICU or other medical establishment. In some embodiments, subjects screened every 1 to 4 hours, 1 to 8 hours, 8 to 12 hours, 12 to 16 hours, or 16 to 24 hours after they arrive in the ICU or other medical establishment.

### 5.3 Kits

The invention also provides kits that are useful in diagnosing or predicting the development of sepsis or diagnosing SIRS in a subject. In some embodiments, the kit comprises any of the biomarker profiles described in Section 5.2 above. In some embodiments, the kits of the present invention comprise at least 2, 3, 4, 5, 6, 7, 8, 9, 10, 11, 12, 13, 14, 15, 16, 17, 18, 19, 20, 25, 30, 35, 40, 45, 50, 54, 5, 60, 65, 70, 75, 80, 85, 90, 95, 96, 100, 105, 110, 115, 120, 125, 130, 135, 140, 145, 150 or more biomarkers. In other embodiments, the kits of the present invention comprise at least 2, but as many as several hundred or more biomarkers. In a specific embodiment, the kits of the present invention comprise at least 2, 3, 4, 5, 6, 7, 8, 9, 10, 11, 12, 13, 14, 15, 16, 17, 18, 19, 20, 25, 30, 35, 40, 45, 50, 54, 5, 60, 65, 70, 75, 80, 85, 90, 95, 96, 100, 105, 110, 115, 120, 125, 130, 135, 140, 145, 150 or more reagents that specifically bind the biomarkers of the present invention. Specific biomarkers that are useful in the present invention are set forth in Section 5.6 as well as Tables 1, 4, 5, 6, 7, 12, 13, 14, 15, 16, 17, 18, 19, 20, and 21 of Section 6. The biomarkers of the kit can be used to generate biomarker profiles according to the present invention. Examples of classes of compounds of the kit include, but are not limited to, proteins and fragments thereof, peptides, proteoglycans, glycoproteins, lipoproteins, carbohydrates, lipids, nucleic acids (e.g., DNA, such as cDNA or amplified DNA, or RNA, such as mRNA), organic or inorganic chemicals, natural or synthetic polymers, small molecules (e.g., metabolites), or discriminating molecules or discriminating fragments of any of the foregoing. Here, a discriminating molecule or fragment is a molecule or fragment that, when detected, indicates presence or abundance of a molecule of interest (e.g., a cDNA, amplified nucleic acid molecule, or protein). In a specific embodiment, a biomarker is of a particular size, (e.g., at least 10, 15, 20, 25, 30, 35, 40, 45, 50, 54, 5, 60, 65, 70, 75, 80, 85, 90, 95, 100, 105, 110, 115, 120, 125, 130, 135, 140, 145, 150, 155, 160, 165, 170, 175, 180, 185, 190, 195 or 200 Da or greater). The biomarker(s) may be part of an array, or the biomarker(s) may be packaged separately and/or individually. The kit may also comprise at least one internal standard to be used in generating the biomarker profiles of the present invention. Likewise, the internal standard or standards can be any of the classes of compounds described above.

In one embodiment, the invention provides kits comprising probes and/or primers that may or may not be immobilized at an addressable position on a substrate, such as found, for example, in a microarray. In a particular embodiment, the invention provides such a microarray.

The kits of the present invention may also contain reagents that can be used to detect biomarkers contained in the biological samples from which the biomarker profiles are generated. In a specific embodiment, the invention provides a kit for predicting the development of sepsis in a test subject comprises a plurality of antibodies that specifically bind a plurality of biomarkers listed in any one of Tables 1, 4, 5, 6, 7, 12, 13, 14, 15, 16, 17, 18, 19, 20, and 21. In another specific embodiment, the invention provides a kit for predicting the development of sepsis in a test subject comprises a plurality of antibodies that specifically bind a plurality of biomarkers listed in any combination of Tables 1, 4, 5, 6 7, 12, 13, 14, 15, 16, 17, 18, 19, 20, and 21. In accordance with this embodiment, the kit may comprise a set of antibodies or functional fragments or derivatives thereof (e.g., Fab, F(ab′)₂, Fv, or scFv fragments) that specifically bind at least 2, 3, 4, 5, 6, 7, 8, 9, 10, 11, 12, 13, 14, 15, 16, 17, 18, 19, 20, 21, 22, 23, 24, 25, or more of the protein-based biomarkers set forth in any one of Tables 1, 4, 5, 6, 7, 12, 13, 14, 15, 16, 17, 18, 19, 20, and 21. In some embodiments, when the kit comprises antibodies to complement component C3 and complement component C4, the kit comprises an antibody to at least one other biomarker in any one of Tables 1, 4, 5, 6, 7, 12, 13, 14, 15, 16, 17, 18, 19, 20, and 21. In accordance with this embodiment, the kit may include antibodies, fragments or derivatives thereof (e.g., Fab, F(ab′)₂, Fv, or scFv fragments) that are specific for the biomarkers of the present invention. In one embodiment, the antibodies may be detectably labeled. In one embodiment, the kit comprises antibodies to any combination of the proteins set forth in Table 4. In one embodiment, the kit comprises antibodies to CRP, APOA2, and SERPINC1. In some embodiments, the biomarker profile comprises antibodies to at least one of SERPINC1, APOA2, and CRP, and, additionally, antibodies to at least 1, 2, 3, 4, 5, 6 or 7 other additional biomarkers in Table 4. In some embodiments, the biomarker profile comprises antibodies to at least one of SERPINC1, APOA2, and CRP, and, additionally, antibodies to at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19, 20, and 21. In some embodiments, the biomarker profile comprises antibodies to at least one of SERPINC1, APOA2, and CRP, and, additionally, antibodies to at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21.

In other embodiments of the invention, a kit may comprise a specific biomarker binding component, such as an aptamer. If the biomarkers comprise a nucleic acid, the kit may provide an oligonucleotide probe that is capable of forming a duplex with the biomarker or with a complementary strand of a biomarker. The oligonucleotide probe may be detectably labeled.

The kits of the present invention may also include reagents such as buffers, or other reagents that can be used in constructing the biomarker profile. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like.

Some kits of the present invention comprise a microarray. In one embodiment this microarray comprises a plurality of probe spots, wherein at least twenty percent of the probe spots in the plurality of probe spots correspond to biomarkers in any one of Tables 1, 4, 5, 6 7, 12, 13, 14, 15, 16, 17, 18, 19, 20, and 21. In some embodiments, at least forty percent, or at least sixty percent, or at least eighty percent of the probe spots in the plurality of probe spots correspond to biomarkers in any one of Tables 1, 4, 5, 6, 7, 12, 13, 14, 15, 16, 17, 18, 19, 20, and 21. In one embodiment this microarray comprises a plurality of probe spots, wherein at least twenty percent of the probe spots in the plurality of probe spots correspond to biomarkers in any combination of Tables 1, 4, 5, 6, 7, 12, 13, 14, 15, 16, 17, 18, 19, 20, and 21. In some embodiments, at least forty percent, or at least sixty percent, or at least eighty percent of the probe spots in the plurality of probe spots correspond to biomarkers in any combination of Tables 1, 4, 5, 6, 7, 12, 13, 14, 15, 16, 17, 18, 19, 20, and 21. In some embodiments, when the plurality of probe spots contain a spot the corresponds to complement component C3 and complement component C4, the plurality of probe spots comprises a probe spot for at least one other biomarker in any of Tables 1, 4, 5, 6, 7, 12, 13, 14, 15, 16, 17, 18, 19, 20, and 21. In some embodiments, the microarray consists of between about three and about one hundred probe spots on a substrate. In some embodiments, the microarray consists of between about three and about one hundred probe spots on a substrate. As used in this context, the term “about” means within five percent of the stated value, within ten percent of the stated value, or within twenty-five percent of the stated value.

Some kits of the invention may further comprise a computer program product for use in conjunction with a computer system. In such kits, the computer program product comprises a computer readable storage medium and a computer program mechanism embedded therein. The computer program mechanism comprises instructions for evaluating whether a plurality of features in a biomarker profile of a test subject at risk for developing sepsis satisfies a first value set. Satisfying the first value set predicts that the test subject is likely to develop sepsis. In one embodiment, the plurality of features corresponds to biomarkers listed in any one of Tables 1, 4, 5, 6, 7, 12, 13, 14, 15, 16, 17, 18, 19, 20, and 21. In one embodiment, the plurality of features corresponds to biomarkers listed in any combination of Tables 1, 4, 5, 6, 7, 12, 13, 14, 15, 16, 17, 18, 19, 20, and 21. In one embodiment, the plurality of features comprises features for CRP, APOA2, and SERPINC1. In some embodiments, the plurality of features comprises features for at least one of SERPINC1, APOA2, and CRP, and, additionally, features for at least 1, 2, 3, 4, 5, 6 or 7 other additional biomarkers in Table 4. In some embodiments, the plurality of features comprises features for at least one of SERPINC1, APOA2, and CRP, and, additionally, features for at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19, 20, and 21. In some embodiments, the plurality of features comprises features for at least one of SERPINC1, APOA2, and CRP, and, additionally, features for at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21. In some embodiments, where the plurality of features comprises features for component C3 and complement component C4, the plurality of features comprises a feature for at least one other biomarker in any of Tables 1, 4, 5, 6, 7, 12, 13, 14, 15, 16, 17, 18, 19, 20, and 21. In some kits, the computer program product further comprises instructions for evaluating whether the plurality of features in the biomarker profile of the test subject satisfies a second value set. Satisfying the second value set predicts that the test subject is not likely to develop sepsis.

Some kits of the present invention comprise a computer having a central processing unit and a memory coupled to the central processing unit. The memory stores instructions for evaluating whether a plurality of features in a biomarker profile of a test subject at risk for developing sepsis satisfies a first value set. Satisfying the first value set predicts that the test subject is likely to develop sepsis. In one embodiment, the plurality of features corresponds to biomarkers listed in any one of Tables 1, 4, 5, 6, 7, 12, 13, 14, 15, 16, 17, 18, 19, 20, and 21. In one embodiment, when the plurality of features includes a feature for complement component C3 and complement component C4, the plurality of features includes a feature for at least one other biomarker in any of Tables 1, 4, 5, 6, 7, 12, 13, 14, 15, 16, 17, 18, 19, 20, and 21. In one embodiment, the plurality of features corresponds to biomarkers listed in any combination of Tables 1, 4, 5, 6, 7, 12, 13, 14, 15, 16, 17, 18, 19, 20, and 21.

FIG. 1 details an exemplary system that supports the functionality described above. The system is preferably a computer system 10 having:
a central processing unit 22;
a main non-volatile storage unit 14, for example, a hard disk drive, for storing software and data, the storage unit 14 controlled by storage controller 12;
a system memory 36, preferably high speed random-access memory (RAM), for storing system control programs, data, and application programs, comprising programs and data loaded from non-volatile storage unit 14;
system memory 36 may also include read-only memory (ROM);
a user interface 32, comprising one or more input devices (e.g., keyboard 28) and a display 26 or other output device;
a network interface card 20 for connecting to any wired or wireless communication network 34 (e.g., a wide area network such as the Internet);
an internal bus 30 for interconnecting the aforementioned elements of the system; and
a power source 24 to power the aforementioned elements.

Operation of computer 10 is controlled primarily by operating system 40, which is executed by central processing unit 22. Operating system 40 can be stored in system memory 36. In addition to operating system 40, in a typical implementation system memory 36 includes:
file system 42 for controlling access to the various files and data structures used by the present invention;
a training data set 44 for use in construction one or more decision rules in accordance with the present invention;
a data analysis algorithm module 54 for processing training data and constructing decision rules;
one or more decision rules 56;
a biomarker profile evaluation module 60 for determining whether a plurality of features in a biomarker profile of a test subject satisfies a first value set or a second value set;
a test subject biomarker profile 62 comprising biomarkers 64 and, for each such biomarkers, features 66; and
a database 68 of select biomarkers of the present invention (e.g., Tables 1, 4, 5, 6, 7, 12, 13, 14, 15, 16, 17, 18, 19, 20 and/or 21).

Training data set 46 comprises data for a plurality of subjects 46. For each subject 46 there is a subject identifier 48 and a plurality of biomarkers 50. For each biomarker 50, there is at least one feature 52. Although not shown in FIG. 1, for each feature 52, there is at least one feature value. For each decision rule 56 constructed using data analysis algorithms, there is at least one decision rule value set 58.

As illustrated in FIG. 1, computer 10 comprises software program modules and data structures. The data structures stored in computer 10 include training data set 44, decision rules 56, test subject biomarker profile 62, and biomarker database 68. Each of these data structures can comprise any form of data storage system including, but not limited to, a flat ASCII or binary file, an Excel spreadsheet, a relational database (SQL), or an on-line analytical processing (OLAP) database (e.g., MDX and/or variants thereof). In some specific embodiments, such data structures are each in the form of one or more databases that include hierarchical structure (e.g., a star schema). In some embodiments, such data structures are each in the form of databases that do not have explicit hierarchy (e.g., dimension tables that are not hierarchically arranged).

In some embodiments, each of the data structures stored or accessible to system 10 are single data structures. In other embodiments, such data structures in fact comprise a plurality of data structures (e.g., databases, files, archives) that may or may not all be hosted by the same computer 10. For example, in some embodiments, training data set 44 comprises a plurality of Excel spreadsheets that are stored either on computer 10 and/or on computers that are addressable by computer 10 across wide area network 34. In another example, training data set 44 comprises a database that is either stored on computer 10 or is distributed across one or more computers that are addressable by computer 10 across wide area network 34.

It will be appreciated that many of the modules and data structures illustrated in FIG. 1 can be located on one or more remote computers. For example, some embodiments of the present application are web service-type implementations. In such embodiments, biomarker profile evaluation module 60 and/or other modules can reside on a client computer that is in communication with computer 10 via network 34. In some embodiments, for example, biomarker profile evaluation module 60 can be an interactive web page.

In some embodiments, training data set 44, decision rules 56, and/or biomarker database 68 illustrated in FIG. 1 are on a single computer (computer 10) and in other embodiments one or more of such data structures and modules are hosted by one or more remote computers (not shown). Any arrangement of the data structures and software modules illustrated in FIG. 1 on one or more computers is within the scope of the present invention so long as these data structures and software modules are addressable with respect to each other across network 34 or by other electronic means. Thus, the present invention fully encompasses a broad array of computer systems.

Still another kit of the present invention comprises computers and computer readable media for evaluating whether a test subject is likely to develop sepsis or SIRS. For instance, one embodiment of the present invention provides a computer program product for use in conjunction with a computer system. The computer program product comprises a computer readable storage medium and a computer program mechanism embedded therein. The computer program mechanism comprises instructions for evaluating whether a plurality of features in a biomarker profile of a test subject at risk for developing sepsis satisfies a first value set. Satisfaction of the first value set predicts that the test subject is likely to develop sepsis. In some embodiments, this plurality of features is measurable aspects of a plurality of biomarkers. The plurality of biomarkers can comprise at least 2, 3, 4, 5, 6, 7, 8, 9, 10, 11, 12, 13, 14, 15, 16, 17, 18, 19, or 20 biomarkers listed in any one of Tables 1, 4, 5, 6, 7, 12, 13, 14, 15, 16, 17, 18, 19, 20, and 21. In certain embodiments, the plurality of biomarkers comprises at least 2, 3, 4, 5, 6, 7, 8, 9, 10, 11, 12, 13, 14, 15, 16, 17, 18, 19, or biomarkers listed in any combination of Tables 1, 4, 5, 6, 7, 12, 13, 14, 15, 16, 17, 18, 19, 20, and 21. In some embodiments, the plurality of biomarkers comprises CRP, APOA2, and SERPINC1. In some embodiments, the plurality of biomarkers comprises at least one of SERPINC1, APOA2, and CRP, and, additionally, at least 1, 2, 3, 4, 5, 6 or 7 other additional biomarkers in Table 4. In some embodiments, the plurality of biomarkers comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19, 20, and 21. In some embodiments, the plurality of biomarkers comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21. In some embodiments, when the plurality of biomarkers comprises complement component C3 and complement component C4, the plurality of biomarkers comprises at least one other biomarker from any one of Tables 1, 4, 5, 6, 7, 12, 13, 14, 15, 16, 17, 18, 19, 20, and 21. In some embodiments, the computer program product further comprises instructions for evaluating whether the plurality of features in the biomarker profile of the test subject satisfies a second value set. Satisfaction of the second value set predicts that the test subject is not likely to develop sepsis. In some embodiments, the biomarker profile has between 3 and 50 biomarkers listed in any one of Tables 1, 4, 5, 6, 7, 12, 13, 14, 15, 16, 17, 18, 19, 20, and 21, between 3 and 40 biomarkers listed in any one of Tables 1, 4, 5, 6, 7, 12, 13, 14, 15, 16, 17, 18, 19, 20, and 21, at least four biomarkers listed in any one of Tables 1, 4, 5, 6, 7, 12, 13, 14, 15, 16, 17, 18, 19, 20, and 21, or at least eight biomarkers listed in any one of Tables 1, 4, 5, 6, 7, 12, 13, 14, 15, 16, 17, 18, 19, 20, and 21. In some embodiments, the biomarker profile has between 3 and 50 biomarkers listed in any combination of Tables 1, 4, 5, 6, 7, 12, 13, 14, 15, 16, 17, 18, 19, 20, and 21, between 3 and 40 biomarkers listed in any combination of Tables 1, 4, 5, 6, 7, 12, 13, 14, 15, 16, 17, 18, 19, 20, and 21, at least four biomarkers listed in any combination of Tables 1, 4, 5, 6, 7, 12, 13, 14, 15, 16, 17, 18, 19, 20, and 21, or at least eight biomarkers listed in any combination of Tables 1, 4, 5, 6, 7, 12, 13, 14, 15, 16, 17, 18, 19, 20, and 21.

Another kit of the present invention comprises a central processing unit and a memory coupled to the central processing unit. The memory stores instructions for evaluating whether a plurality of features in a biomarker profile of a test subject at risk for developing sepsis satisfies a first value set. Satisfaction of the first value set predicts that the test subject is likely to develop sepsis. The plurality of features is measurable aspects of a plurality of biomarkers. In some embodiments, this plurality of biomarkers comprises at least 2, 3, 4, 5, 6, 7, 8, 9, 10, 11, 12, 13, 14, 15, 16, 17, 18, 19, or 20 biomarkers from any one of Tables 1, 4, 5, 6, 7, 12, 13, 14, 15, 16, 17, 18, 19, 20, and 21. In some embodiments, this plurality of biomarkers comprises at least 2, 3, 4, 5, 6, 7, 8, 9, 10, 11, 12, 13, 14, 15, 16, 17, 18, 19, or 20 biomarkers from any combination of Tables 1, 4, 5, 6, 7, 12, 13, 14, 15, 16, 17, 18, 19, 20, and 21. In some embodiments, this plurality of biomarkers comprises CRP, APOA2, and SERPINC1. In some embodiments, the plurality of biomarkers comprises at least one of SERPINC1, APOA2, and CRP, and, additionally, at least 1, 2, 3, 4, 5, 6 or 7 other additional biomarkers in Table 4. In some embodiments, the plurality of biomarkers comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19, 20, and 21. In some embodiments, the plurality of biomarkers comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21. In some embodiments, when the plurality of biomarkers comprises complement component C3 and complement component C4, the plurality of biomarkers comprises at least one other biomarker from any one of Tables 1, 4, 5, 6, 7, 12, 13, 14, 15, 16, 17, 18, 19, 20, and 21. In some embodiments, the memory further stores instructions for evaluating whether the plurality of features in the biomarker profile of the test subject satisfies a second value set. Satisfaction of the second value set predicts that the test subject is not likely to develop sepsis. In some embodiments, the biomarker profile consists of between 3 and 50 biomarkers listed in any one of Tables 1, 4, 5, 6, 7, 12, 13, 14, 15, 16, 17, 18, 19, 20, and 21, between 3 and 40 biomarkers listed in any one of Tables 1, 4, 5, 6, 7, 12, 13, 14, 15, 16, 17, 18, 19, 20, and 21, at least four biomarkers listed in any one of Tables 1, 4, 5, 6, 7, 12, 13, 14, 15, 16, 17, 18, 19, 20, and 21, or at least eight biomarkers listed in any one of Tables 1, 4, 5, 6, 7, 12, 13, 14, 15, 16, 17, 18, 19, 20, and 21 (for example, all found in Table 1, all found in Table 4, all found in Table 5, all found in Table 6, all found in Table 7, all found Table 12, all found in Table 13, all found in Table 14, all found in Table 15, all found in Table 16, all found in Table 17, all found in Table 18, all found in Table 19, or all found in Table 20). In some embodiments, the biomarker profile consists of between 3 and 50 biomarkers listed in any combination of Tables 1, 4, 5, 6, 7, 12, 13, 14, 15, 16, 17, 18, 19, 20, and 21, between 3 and 40 biomarkers listed in any combination of Tables 1, 4, 5, 6, 7, 12, 13, 14, 15, 16, 17, 18, 19, 20, and 21, at least 3, 4, 5, 6, 7, 8, 9, or 10 biomarkers listed in any combination of Tables 1, 4, 5, 6, 7, 12, 13, 14, 15, 16, 17, 18, 19, 20, and 21 (for example, all found in Tables 1 or 4, all found in Table 4 or 5, all found in Tables 1, 5 and 7, all found in Table 15 or 17).

Another kit in accordance with the present invention comprises a computer system for determining whether a subject is likely to develop sepsis. The computer system comprises a central processing unit and a memory, coupled to the central processing unit. The memory stores instructions for obtaining a biomarker profile of a test subject. The biomarker profile comprises a plurality of features. Each feature in the plurality of features is a measurable aspect of a corresponding biomarker in a plurality of biomarkers. In some embodiments, the plurality of biomarkers comprises at least 2, 3, 4, 5, 6, 7, 8, 9, 10, 11, 12, 13, 14, 15, 16, 17, 18, 19, 20 or biomarkers listed in any combination of Tables 1, 4, 5, 6, 7, 12, 13, 14, 15, 16, 17, 18, 19, 20, and 21. In some embodiments, the plurality of biomarkers comprises at least 2, 3, 4, 5, 6, 7, 8, 9, 10, 11, 12, 13, 14, 15, 16, 17, 18, 19, 20 or biomarkers listed in any one of Tables 1, 4, 5, 6, 7, 12, 13, 14, 15, 16, 17, 18, 19, 20, and 21. In some embodiments, the plurality of biomarkers comprises CRP, APOA2, and SERPINC1. In some embodiments, the biomarker profile comprises at least one of SERPINC1, APOA2, and CRP, and, additionally, at least 1, 2, 3, 4, 5, 6 or 7 other additional biomarkers in Table 4. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21. In some embodiments, when the plurality of biomarkers comprises complement component C3 and complement component C4, the plurality of biomarkers comprises at least one other biomarker from any one of Tables 1, 4, 5, 6, 7, 12, 13, 14, 15, 16, 17, 18, 19, 20, and 21. The memory further comprises instructions for transmitting the biomarker profile to a remote computer. The remote computer includes instructions for evaluating whether the plurality of features in the biomarker profile of the test subject satisfies a first value set. Satisfaction of the first value set predicts that the test subject is likely to develop sepsis. The memory further comprises instructions for receiving a determination, from the remote computer, as to whether the plurality of features in the biomarker profile of the test subject satisfies the first value set. The memory also comprises instructions for reporting whether the plurality of features in the biomarker profile of the test subject satisfies the first value set. In some embodiments, the plurality of biomarkers comprises at least 2, 3, 4, 5, 6, 7, 8, 9, 10, 11, 12, 13, 14, 15, 16, 17, 18, 19, or 20 biomarkers listed in any one of Tables 1, 4, 5, 6, 7, 12, 13, 14, 15, 16, 17, 18, 19, 20, and 21. In some embodiments, the plurality of biomarkers comprises at least 2, 3, 4, 5, 6, 7, 8, 9, 10, 11, 12, 13, 14, 15, 16, 17, 18, 19, or 20 biomarkers listed in any combination of Tables 1, 4, 5, 6, 7, 12, 13, 14, 15, 16, 17, 18, 19, 20, and 21. In some embodiments, the remote computer further comprises instructions for evaluating whether the plurality of features in the biomarker profile of the test subject satisfies a second value set. Satisfaction of the second value set predicts that the test subject is not likely to develop sepsis. In such embodiments, the memory further comprises instructions for receiving a determination, from the remote computer, as to whether the plurality of features in the biomarker profile of the test subject satisfies the second set as well as instructions for reporting whether the plurality of features in the biomarker profile of the test subject satisfies the second value set.

Yet another kit of the present invention provides a computer system for determining whether a subject is likely to develop sepsis. The computer system comprises a central processing unit and a memory, coupled to the central processing unit. The memory stores instructions for obtaining a biomarker profile of a test subject. The biomarker profile comprises a plurality of features. The plurality of features is measurable aspects of a plurality of biomarkers. In some embodiments, the plurality of biomarkers comprises at least 2, 3, 4, 5, 6, 7, 8, 9, 10, 11, 12, 13, 14, 15, 16, 17, 18, 19, 20 or biomarkers listed in any one of Tables 1, 4, 5, 6, 7, 12, 13, 14, 15, 16, 17, 18, 19, 20, and 21. In some embodiments, the plurality of biomarkers comprises at least 2, 3, 4, 5, 6, 7, 8, 9, 10, 11, 12, 13, 14, 15, 16, 17, 18, 19, 20 or biomarkers listed in any combination of Tables 1, 4, 5, 6, 7, 12, 13, 14, 15, 16, 17, 18 19, 20, and 21. The memory further stores instructions for evaluating whether the plurality of features in the biomarker profile of the test subject satisfies a first value set. Satisfying the first value set predicts that the test subject is likely to develop sepsis. The memory also stores instructions for reporting whether the plurality of features in the biomarker profile of the test subject satisfies the first value set. In some embodiments, the plurality of biomarkers comprises at least 2, 3, 4, 5, 6, 7, 8, 9, 10, 11, 12, 13, 14, 15, 16, 17, 18, 19, 20, 21, 22, 23, 24, or 25 biomarkers from Table 1. In some embodiments, the plurality of biomarkers comprises at least 2, 3, 4, 5, 6, 7, 8, 9, 10 biomarkers from Table 4. In some embodiments, the plurality of biomarkers comprises at least 2, 3, 4, 5, 6, 7, 8, 9, 10, 11, 12, 13, 14, 15, 16, 17, 18, 19, 20, 21, 22, 23, 24, or 25 biomarkers from Table 5. In some embodiments, the plurality of biomarkers comprises at least 2, 3, 4, 5, 6, 7, 8, 9, 10, 11, 12, 13, 14, 15, 16, 17, 18, 19, 20, 21, 22, 23, 24, or 25 biomarkers from Table 6. In some embodiments, the plurality of biomarkers comprises at least 2, 3, 4, 5, 6, 7, 8, 9, 10, 11, 12, 13, 14, 15, 16, 17, 18, 19, 20, 21, 22, 23, 24, or 25 biomarkers from Table 7. In some embodiments, the plurality of biomarkers comprises at least 2, 3, 4, 5, 6, 7, 8, 9, 10, 11, 12, 13, 14, 15, 16, 17, 18, 19, 20, 21, 22, 23, 24, or 25 biomarkers from Table 15. In some embodiments, the plurality of biomarkers comprises at least 2, 3, 4, 5, 6, 7, 8, 9, 10, 11, 12, 13, 14, 15, 16, 17, 18, 19, 20, 21, 22, 23, 24, or 25 biomarkers from Table 17. In some embodiments, the plurality of biomarkers comprises CRP, APOA2, and SERPINC1. In some embodiments, the plurality of biomarkers comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19, 20, and 21. In some embodiments, the plurality of biomarkers comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19, 20, and 21. In some embodiments, when the plurality of biomarkers comprises complement component C3 and complement component C4, the plurality of biomarkers comprises at least one other biomarker from any one of Tables 1, 4, 5, 6, 7, 12, 13, 14, 15, 16, 17, 18, 19, 20, and 21.

### 5.4 Generation of Biomarker Profiles

According to one embodiment, the methods of the present invention comprise generating a biomarker profile from a biological sample taken from a subject. The biological sample may be, for example, whole blood, plasma, serum, red blood cells, platelets, neutrophils, eosinophils, basophils, lymphocytes, monocytes, saliva, sputum, urine, cerebral spinal fluid, cells, a cellular extract, a tissue sample, a tissue biopsy, a stool sample or any sample that may be obtained from a subject using techniques well known to those of skill in the art. In a specific embodiment, a biomarker profile is determined using samples collected from a subject at one or more separate time points. In another specific embodiment, a biomarker profile is generated using samples obtained from a subject at separate time points. In one example, these samples are obtained from the subject either once or, alternatively, on a daily basis, or more frequently, e.g., every 4, 6, 8 or 12 hours. In some embodiments, these samples are collected from the subject on multiple different time points, but on an irregular time basis. In a specific embodiment, a biomarker profile is determined using samples collected from a single tissue type. In another specific embodiment, a biomarker profile is determined using samples collected from at least 2, 3, 4, 4, 5, 6 or 7 different tissue types.

5.4.1 Methods of Detecting Nucleic Acid Biomarkers

In specific embodiments of the invention, biomarkers in a biomarker profile are nucleic acids. Such biomarkers and corresponding features of the biomarker profile may be generated, for example, by detecting the expression product (e.g., a polynucleotide or polypeptide) of one or more genes described herein (e.g., a gene listed in Tables 1, 4, 5, 6, 7, 12, 13, 14, 15, 16, 17, 18, 19, 20 and/or 21). In a specific embodiment, the biomarkers and corresponding features in a biomarker profile are obtained by detecting and/or analyzing one or more nucleic acids expressed from a gene disclosed herein (e.g., a gene listed in Tables 1, 4, 5, 6, 7, 12, 13, 14, 15, 16, 17, 18, 19, 20 and/or 21) using any method well known to those skilled in the art including, but by no means limited to, hybridization, microarray analysis, RT-PCR, nuclease protection assays and Northern blot analysis.

In certain embodiments, nucleic acids detected and/or analyzed by the methods and compositions of the invention include RNA molecules such as, for example, expressed RNA molecules which include messenger RNA (mRNA) molecules, mRNA spliced variants as well as regulatory RNA, cRNA molecules (e.g., RNA molecules prepared from cDNA molecules that are transcribed in vitro) and discriminating fragments thereof. Nucleic acids detected and/or analyzed by the methods and compositions of the present invention can also include, for example, DNA molecules such as genomic DNA molecules, cDNA molecules, and discriminating fragments thereof (e.g., oligonucleotides, ESTs, STSs, etc.).

The nucleic acid molecules detected and/or analyzed by the methods and compositions of the invention may be naturally occurring nucleic acid molecules such as genomic or extragenomic DNA molecules isolated from a sample, or RNA molecules, such as mRNA molecules, present in, isolated from or derived from a biological sample. The sample of nucleic acids detected and/or analyzed by the methods and compositions of the invention comprise, e.g., molecules of DNA, RNA, or copolymers of DNA and RNA. Generally, these nucleic acids correspond to particular genes or alleles of genes, or to particular gene transcripts (e.g., to particular mRNA sequences expressed in specific cell types or to particular cDNA sequences derived from such mRNA sequences). The nucleic acids detected and/or analyzed by the methods and compositions of the invention may correspond to different exons of the same gene, e.g., so that different splice variants of that gene may be detected and/or analyzed.

In specific embodiments, the nucleic acids are prepared in vitro from nucleic acids present in, or isolated or partially isolated from biological a sample. For example, in one embodiment, RNA is extracted from a sample (e.g., total cellular RNA, poly(A)⁺ messenger RNA, fraction thereof) and messenger RNA is purified from the total extracted RNA. Methods for preparing total and poly(A)⁺ RNA are well known in the art, and are described generally, e.g., in Sambrook et al, 2001, Molecular Cloning: A Laboratory Manual. 3^{rd} edition, Cold Spring Harbor Laboratory Press (Cold Spring Harbor, N.Y.), which is incorporated by reference herein in its entirety. In one embodiment, RNA is extracted from a sample using guanidinium thiocyanate lysis followed by CsCl centrifugation and an oligo dT purification (Chirgwin et al., 1979, Biochemistry 18:5294-5299). In another embodiment, RNA is extracted from a sample using guanidinium thiocyanate lysis followed by purification on RNeasy columns (Qiagen, Valencia, Calif.). cDNA is then synthesized from the purified mRNA using, e.g., oligo-dT or random primers. In specific embodiments, the target nucleic acids are cRNA prepared from purified messenger RNA extracted from a sample. As used herein, cRNA is defined here as RNA complementary to the source RNA. The extracted RNAs are amplified using a process in which doubled-stranded cDNAs are synthesized from the RNAs using a primer linked to an RNA polymerase promoter in a direction capable of directing transcription of anti-sense RNA. Anti-sense RNAs or cRNAs are then transcribed from the second strand of the double-stranded cDNAs using an RNA polymerase (see, e.g., U.S. Pat. Nos. 5,891,636, 5,716,785; 5,545,522 and 6,132,997, which are hereby incorporated by reference). Both oligo-dT primers (U.S. Pat. Nos. 5,545,522 and 6,132,997, hereby incorporated by reference herein) or random primers that contain an RNA polymerase promoter or complement thereof can be used. In some embodiments the target nucleic acids are short and/or fragmented nucleic acid molecules which are representative of the original nucleic acid population of the sample.

In one embodiment, nucleic acids of the invention can be detectably labeled. For example, cDNA can be labeled directly, e.g., with nucleotide analogs, or indirectly, e.g., by making a second, labeled cDNA strand using the first strand as a template. Alternatively, the double-stranded cDNA can be transcribed into cRNA and labeled. In some embodiments the detectable label is a fluorescent label, e.g., by incorporation of nucleotide analogs.

5.4.1.1 Nucleic Acid Arrays

In certain embodiments of the invention, nucleic acid arrays are employed to generate features of biomarkers in a biomarker profile by detecting the expression of any one or more of the genes described herein (e.g., a gene listed in Tables 1, 4, 5, 6, 7, 12, 13, 14, 15, 16, 17, 18, 19 20 and/or 21). In one embodiment of the invention, a microarray, such as a cDNA microarray, is used to determine feature values of biomarkers in a biomarker profile. The diagnostic use of cDNA arrays is well known in the art. (See, e.g., Zou et. al., 2002, Oncogene 21:4855-4862; as well as Draghici, 2003, Data Analysis Tools for DNA Microarrays, Chapman & Hall/CRC, each of which is hereby incorporated by reference herein in its entirety).

In certain embodiments, the feature values for biomarkers in a biomarker profile are obtained by hybridizing to the array detectably labeled nucleic acids representing or corresponding to the nucleic acid sequences in mRNA transcripts present in a biological sample (e.g., fluorescently labeled cDNA synthesized from the sample) to a microarray comprising one or more probe spots.

Nucleic acid arrays, for example, microarrays, can be made in a number of ways, of which several are described herein below. Preferably, the arrays are reproducible, allowing multiple copies of a given array to be produced and results from said microarrays compared with each other. Preferably, the arrays are made from materials that are stable under binding (e.g., nucleic acid hybridization) conditions. Those skilled in the art will know of suitable supports, substrates or carriers for hybridizing test probes to probe spots on an array, or will be able to ascertain the same by use of routine experimentation.

Arrays, for example, microarrays, used can include one or more test probes. In some embodiments each such test probe comprises a nucleic acid sequence that is complementary to a subsequence of RNA or DNA to be detected. Each probe typically has a different nucleic acid sequence, and the position of each probe on the solid surface of the array is usually known or can be determined. Arrays useful in accordance with the invention can include, for example, oligonucleotide microarrays, cDNA based arrays, SNP arrays, spliced variant arrays and any other array able to provide a qualitative, quantitative or semi-quantitative measurement of expression of a gene described herein (e.g., a gene listed in Tables 1, 4, 5, 6, 7, 12, 13, 14, 15, 16, 17, 18, 19 20 and/or 21). Some types of microarrays are addressable arrays. More specifically, some microarrays are positionally addressable arrays. In some embodiments, each probe of the array is located at a known, predetermined position on the solid support so that the identity (e.g., the sequence) of each probe can be determined from its position on the array (e.g., on the support or surface). In some embodiments, the arrays are ordered arrays. Microarrays are generally described in Draghici, 2003, Data Analysis Tools for DNA Microarrays, Chapman & Hall/CRC, which is hereby incorporated by reference herein in its entirety.

In some embodiments of the present invention, an expressed transcript (e.g., a transcript of a gene described herein) is represented in the nucleic acid arrays. In such embodiments, a set of binding sites can include probes with different nucleic acids that are complementary to different sequence segments of the expressed transcript. Exemplary nucleic acids that fall within this class can be of length of 15 to 200 bases, 20 to 100 bases, 25 to 50 bases, 40 to 60 bases or some other range of bases. Each probe sequence can also comprise one or more linker sequences in addition to the sequence that is complementary to its target sequence. As used herein, a linker sequence is a sequence between the sequence that is complementary to its target sequence and the surface of support. For example, the nucleic acid arrays of the invention can comprise one probe specific to each target gene or exon. However, if desired, the nucleic acid arrays can contain at least 2, 5, 10, 100, or 1000 or more probes specific to some expressed transcript (e.g., a transcript of a gene described herein, e.g., in Tables 1, 4, 5, 6, 7, 12, 13, 14, 15, 16, 17, 18, 19 20 and/or 21). For example, the array may contain probes tiled across the sequence of the longest mRNA isoform of a gene.

It will be appreciated that when cDNA complementary to the RNA of a cell, for example, a cell in a biological sample, is made and hybridized to a microarray under suitable hybridization conditions, the level of hybridization to the site in the array corresponding to a gene described herein (e.g., a gene listed in Tables 1, 4, 5, 6, 7, 12, 13, 14, 15, 16, 17, 18, 19 20 and/or 21) will reflect the prevalence in the cell of mRNA or mRNAs transcribed from that gene. Alternatively, in instances where multiple isoforms or alternate splice variants produced by particular genes are to be distinguished, detectably labeled (e.g., with a fluorophore) cDNA complementary to the total cellular mRNA can be hybridized to a microarray, and the site on the array corresponding to an exon of the gene that is not transcribed or is removed during RNA splicing in the cell will have little or no signal (e.g., fluorescent signal), and a site corresponding to an exon of a gene for which the encoded mRNA expressing the exon is prevalent will have a relatively strong signal. The relative abundance of different mRNAs produced from the same gene by alternative splicing is then determined by the signal strength pattern across the whole set of exons monitored for the gene.

In one embodiment, hybridization levels at different hybridization times are measured separately on different, identical microarrays. For each such measurement, at hybridization time when hybridization level is measured, the microarray is washed briefly, preferably in room temperature in an aqueous solution of high to moderate salt concentration (e.g., 0.5 to 3 M salt concentration) under conditions which retain all bound or hybridized nucleic acids while removing all unbound nucleic acids. The detectable label on the remaining, hybridized nucleic acid molecules on each probe is then measured by a method which is appropriate to the particular labeling method used. The resulting hybridization levels are then combined to form a hybridization curve. In another embodiment, hybridization levels are measured in real time using a single microarray. In this embodiment, the microarray is allowed to hybridize to the sample without interruption and the microarray is interrogated at each hybridization time in a non-invasive manner. In still another embodiment, one can use one array, hybridize for a short time, wash and measure the hybridization level, put back to the same sample, hybridize for another period of time, wash and measure again to get the hybridization time curve.

In some embodiments, nucleic acid hybridization and wash conditions are chosen so that the nucleic acid biomarkers to be analyzed specifically bind or specifically hybridize to the complementary nucleic acid sequences of the array, typically to a specific array site, where its complementary DNA is located.

Arrays containing double-stranded probe DNA situated thereon can be subjected to denaturing conditions to render the DNA single-stranded prior to contacting with the target nucleic acid molecules. Arrays containing single-stranded probe DNA (e.g., synthetic oligodeoxyribonucleic acids) may need to be denatured prior to contacting with the target nucleic acid molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. General parameters for specific (i.e., stringent) hybridization conditions for nucleic acids are described in Sambrook et al., (supra), and in Ausubel et al., 1988, Current Protocols in Molecular Biology, Greene Publishing and Wiley-Interscience, New York. When the cDNA microarrays of Shena et al. are used, typical hybridization conditions are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S.A. 93:10614). Useful hybridization conditions are also provided in, e.g., Tijessen, 1993, Hybridization With Nucleic Acid Probes, Elsevier Science Publishers B. V.; Kricka, 1992, Nonisotopic DNA Probe Techniques, Academic Press, San Diego, Calif.; and Zou et. al., 2002, Oncogene 21:4855-4862; and Draghici, Data Analysis Tools for DNA Microanalysis, 2003, CRC Press LLC, Boca Raton, Fla., pp. 342-343, which are hereby incorporated by reference herein in their entirety.

In a specific embodiment, a microarray can be used to sort out RT-PCR products that have been generated by the methods described, for example, below in Section 5.4.1.2.

5.4.1.2 RT-PCR

In certain embodiments, to determine the feature values of biomarkers in a biomarker profile of the invention, the level of expression of one or more of the genes described herein (e.g., a gene listed in Tables 1, 4, 5, 6, 7, 12, 13, 14, 15, 16, 17, 18, 19 20 and/or 21) is measured by amplifying RNA from a sample using reverse transcription (RT) in combination with the polymerase chain reaction (PCR). In accordance with this embodiment, the reverse transcription may be quantitative or semi-quantitative. The RT-PCR methods taught herein may be used in conjunction with the microarray methods described above, for example, in Section 5.4.1.1. For example, a bulk PCR reaction may be performed, the PCR products may be resolved and used as probe spots on a microarray.

Total RNA, or mRNA from a sample is used as a template and a primer specific to the transcribed portion of the gene(s) is used to initiate reverse transcription. Methods of reverse transcribing RNA into cDNA are well known and described in Sambrook et al., 2001, supra. Primer design can be accomplished based on known nucleotide sequences that have been published or available from any publicly available sequence database such as GenBank. For example, primers may be designed for any of the genes described herein (see, e.g., Table 1, 4, 5, 6, 7, 12, 13, 14, 15, 16, 17, 18, 19 20 and/or 21 which provides the GenBank accession numbers of the nucleotide and amino acid sequences of the genes described herein). Further, primer design may be accomplished by utilizing commercially available software (e.g., Primer Designer 1.0, Scientific Software etc.). The product of the reverse transcription is subsequently used as a template for PCR.

PCR provides a method for rapidly amplifying a particular nucleic acid sequence by using multiple cycles of DNA replication catalyzed by a thermostable, DNA-dependent DNA polymerase to amplify the target sequence of interest. PCR requires the presence of a nucleic acid to be amplified, two single-stranded oligonucleotide primers flanking the sequence to be amplified, a DNA polymerase, deoxyribonucleoside triphosphates, a buffer and salts. The method of PCR is well known in the art. PCR is performed, for example, as described in Mullis and Faloona, 1987, Methods Enzymol. 155:335, which is hereby incorporated by reference herein in its entirety.

PCR can be performed using template DNA or cDNA (at least 1 fg; more usefully, 1-1000 ng) and at least 25 pmol of oligonucleotide primers. A typical reaction mixture includes: 2 μl of DNA, 25 pmol of oligonucleotide primer, 2.5 μl of 10 M PCR buffer 1 (Perkin-Elmer, Foster City, Calif.), 0.4 μl of 1.25 M dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

Quantitative RT-PCR (“QRT-PCR”), which is quantitative in nature, can also be performed to provide a quantitative measure of gene expression levels. In QRT-PCR reverse transcription and PCR can be performed in two steps, or reverse transcription combined with PCR can be performed concurrently. One of these techniques, for which there are commercially available kits such as Taqman (Perkin Elmer, Foster City, Calif.) or as provided by Applied Biosystems (Foster City, Calif.) is performed with a transcript-specific antisense probe. This probe is specific for the PCR product (e.g. a nucleic acid fragment derived from a gene) and is prepared with a quencher and fluorescent reporter probe complexed to the 5′ end of the oligonucleotide. Different fluorescent markers are attached to different reporters, allowing for measurement of two products in one reaction. When Taq DNA polymerase is activated, it cleaves off the fluorescent reporters of the probe bound to the template by virtue of its 5′-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions are performed in 96-well plates so that samples derived from many individuals are processed and measured simultaneously. The Taqman system has the additional advantage of not requiring gel electrophoresis and allows for quantification when used with a standard curve.

A second technique useful for detecting PCR products quantitatively without is to use an intercalating dye such as the commercially available QuantiTect SYBR Green PCR (Qiagen, Valencia Calif.). RT-PCR is performed using SYBR green as a fluorescent label which is incorporated into the PCR product during the PCR stage and produces a fluorescence proportional to the amount of PCR product.

Both Taqman and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other systems to quantitatively measure mRNA expression products are known including Molecular Beacons® which uses a probe having a fluorescent molecule and a quencher molecule, the probe capable of forming a hairpin structure such that when in the hairpin form, the fluorescence molecule is quenched, and when hybridized the fluorescence increases giving a quantitative measurement of gene expression.

Additional techniques to quantitatively measure RNA expression include, but are not limited to, polymerase chain reaction, ligase chain reaction, Qbeta replicase (see, e.g., International Application No. PCT/US87/00880, which is hereby incorporated by reference herein in its entirety), isothermal amplification method (see, e.g., Walker et al., 1992, PNAS 89:382-396, which is hereby incorporated by reference herein in its entirety), strand displacement amplification (SDA), repair chain reaction, Asymmetric Quantitative PCR (see, e.g., U.S. Publication No. US 2003/30134307A1, herein incorporated by reference) and the multiplex microsphere bead assay described in Fuja et al., 2004, Journal of Biotechnology 108:193-205, herein incorporated by reference.

The level of expression of one or more of the genes described herein (e.g., the genes listed in Tables 1, 4, 5, 6, 7, 12, 13, 14, 15, 16, 17, 18, 19 20 and/or 21) can, for example, be measured by amplifying RNA from a sample using amplification (NASBA). See, e.g., Kwoh et al., 1989, PNAS USA 86:1173; International Publication No. WO 88/10315; and U.S. Pat. No. 6,329,179, each of which is hereby incorporated by reference. In NASBA, the nucleic acids may be prepared for amplification using conventional methods, e.g., phenol/chloroform extraction, heat denaturation, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once with a polymerase such as T7 or SP6. The resulting products, whether truncated or complete, indicate target specific sequences.

Several techniques may be used to separate amplification products. For example, amplification products may be separated by agarose, agarose-acrylamide or polyacrylamide gel electrophoresis using conventional methods. See Sambrook et al., 2001. Several techniques for detecting PCR products quantitatively without electrophoresis may also be used according to the invention (see, e.g., PCR Protocols, A Guide to Methods and Applications, Innis et al., 1990, Academic Press, Inc. N.Y., which is hereby incorporated by reference). For example, chromatographic techniques may be employed to effect separation. There are many kinds of chromatography which may be used in the present invention: adsorption, partition, ion-exchange and molecular sieve, HPLC, and many specialized techniques for using them including column, paper, thin-layer and gas chromatography (Freifelder, Physical Biochemistry Applications to Biochemistry and Molecular Biology, 2nd ed., Wm. Freeman and Co., New York, N.Y., 1982, which is hereby incorporated by reference).

Another example of a separation methodology is to covalently label the oligonucleotide primers used in a PCR reaction with various types of small molecule ligands. In one such separation, a different ligand is present on each oligonucleotide. A molecule, perhaps an antibody or avidin if the ligand is biotin, that specifically binds to one of the ligands is used to coat the surface of a plate such as a 96 well ELISA plate. Upon application of the PCR reactions to the surface of such a prepared plate, the PCR products are bound with specificity to the surface. After washing the plate to remove unbound reagents, a solution containing a second molecule that binds to the first ligand is added. This second molecule is linked to some kind of reporter system. The second molecule only binds to the plate if a PCR product has been produced whereby both oligonucleotide primers are incorporated into the final PCR products. The amount of the PCR product is then detected and quantified in a commercial plate reader much as ELISA reactions are detected and quantified. An ELISA-like system such as the one described here has been developed by Raggio Italgene (under the C-Track tradename).

Amplification products should be visualized in order to confirm amplification of the nucleic acid sequences of interest, i.e., nucleic acid sequences of one or more of the genes described herein (e.g., a gene listed in Tables 1, 4, 5, 6, 7, 12, 13, 14, 15, 16, 17, 18, 19 20 and/or 21). One typical visualization method involves staining of a gel with ethidium bromide and visualization under UV light. Alternatively, if the amplification products are integrally labeled with radio- or fluorometrically-labeled nucleotides, the amplification products may then be exposed to x-ray film or visualized under the appropriate stimulating spectra, following separation.

In one embodiment, visualization is achieved indirectly. Following separation of amplification products, a labeled, nucleic acid probe is brought into contact with the amplified nucleic acid sequence of interest, i.e., nucleic acid sequences of one or more of the genes described herein (e.g., a gene listed in Tables 1, 4, 5, 6, 7, 12, 13, 14, 15, 16, 17, 18, 19 20 and/or 21). The probe preferably is conjugated to a chromophore but may be radiolabeled. In another embodiment, the probe is conjugated to a binding partner, such as an antibody or biotin, where the other member of the binding pair carries a detectable moiety.

In another embodiment, detection is by Southern blotting and hybridization with a labeled probe. The techniques involved in Southern blotting are well known to those of skill in the art and may be found in many standard books on molecular protocols. See Sambrook et al., 2001. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

5.4.1.3 Nuclease Protection Assays

In particular embodiments, feature values for biomarkers in a biomarker profile can be obtained by performing nuclease protection assays (including both ribonuclease protection assays and S1 nuclease assays) to detect and quantify specific mRNAs (e.g., mRNAs of a gene described in Tables 1, 4, 5, 6, 7, 12, 13, 14, 15, 16, 17, 18, 19 20 and/or 21). Such assays are described in, for example, Sambrook et al., 2001, supra. In nuclease protection assays, an antisense probe (labeled with, e.g., radiolabeled or nonisotopic) hybridizes in solution to an RNA sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 μg maximum of blot hybridizations.

The ribonuclease protection assay, which is the most common type of nuclease protection assay, requires the use of RNA probes. Oligonucleotides and other single-stranded DNA probes can only be used in assays containing S1 nuclease. The single-stranded, antisense probe must typically be completely homologous to target RNA to prevent cleavage of the probe:target hybrid by nuclease.

5.4.1.4 Northern Blot Assays

Any hybridization technique known to those of skill in the art can be used to generate feature values for biomarkers in a biomarker profile. In other particular embodiments, feature values for biomarkers in a biomarker profile can be obtained by Northern blot analysis (to detect and quantify specific RNA molecules (e.g., RNAs of a gene described in Tables 1, 4, 5, 6, 7, 12, 13, 14, 15, 16, 17, 18, 19 20 and/or 21). A standard Northern blot assay can be used to ascertain an RNA transcript size, identify alternatively spliced RNA transcripts, and the relative amounts of one or more genes described herein (in particular, mRNA) in a sample, in accordance with conventional Northern hybridization techniques known to those persons of ordinary skill in the art. In Northern blots, RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes. The labeled probe, e.g., a radiolabelled cDNA, either containing the full-length, single stranded DNA or a fragment of that DNA sequence may be at least 20, at least 30, at least 50, or at least 100 consecutive nucleotides in length. The probe can be labeled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels.

5.4.2 Methods of Detecting Proteins

In specific embodiments of the invention, feature values of biomarkers in a biomarker profile can be obtained by detecting proteins, for example, by detecting the expression product (e.g., a nucleic acid or protein) of one or more genes described herein (e.g., a gene listed in Tables 1, 4, 5, 6, 7, 12, 13, 14, 15, 16, 17, 18, 19 20 and/or 21), or post-translationally modified, or otherwise modified, or processed forms of such proteins. In a specific embodiment, a biomarker profile is generated by detecting and/or analyzing one or more proteins and/or discriminating fragments thereof expressed from a gene disclosed herein (e.g., a gene listed in Tables 1, 4, 5, 6, 7, 12, 13, 14, 15, 16, 17, 18, 19 20 and/or 21) using any method known to those skilled in the art for detecting proteins including, but not limited to protein microarray analysis, immunohistochemistry and mass spectrometry.

Standard techniques may be utilized for determining the amount of the protein or proteins of interest (e.g., proteins expressed from genes listed in Tables 1, 4, 5, 6, 7, 12, 13, 14, 15, 16, 17, 18, 19 20 and/or 21) present in a sample. For example, standard techniques can be employed using, e.g., immunoassays such as, for example Western blot, immunoprecipitation followed by sodium dodecyl sulfate polyacrylamide gel electrophoresis, (SDS-PAGE), immunocytochemistry, and the like to determine the amount of protein or proteins of interest present in a sample. One exemplary agent for detecting a protein of interest is an antibody capable of specifically binding to a protein of interest, preferably an antibody detectably labeled, either directly or indirectly.

For such detection methods, if desired a protein from the sample to be analyzed can easily be isolated using techniques which are well known to those of skill in the art. Protein isolation methods can, for example, be such as those described in Harlow and Lane, 1988, Antibodies: A Laboratory Manual. Cold Spring Harbor Laboratory Press (Cold Spring Harbor, N.Y.), which is incorporated by reference herein in its entirety.

Antibodies, or fragments of antibodies, specific for a protein of interest can be used to quantitatively or qualitatively detect the presence of a protein. This can be accomplished, for example, by immunofluorescence techniques. Antibodies (or fragments thereof) can, additionally, be employed histologically, as in immunofluorescence or immunoelectron microscopy, for in situ detection of a protein of interest. In situ detection can be accomplished by removing a biological sample (e.g., a biopsy specimen) from a patient, and applying thereto a labeled antibody that is directed to a protein of interest (e.g., a protein expressed from a gene in Tables 1, 4, 5, 6, 7, 12, 13, 14, 15, 16, 17, 18, 19 20 and/or 21). The antibody (or fragment) is preferably applied by overlaying the antibody (or fragment) onto a biological sample. Through the use of such a procedure, it is possible to determine not only the presence of the protein of interest, but also its distribution, in a particular sample. A wide variety of well-known histological methods (such as staining procedures) can be utilized to achieve such in situ detection.

Immunoassays for a protein of interest typically comprise incubating a biological sample of a detectably labeled antibody capable of identifying a protein of interest, and detecting the bound antibody by any of a number of techniques well-known in the art. As discussed in more detail, below, the term “labeled” can refer to direct labeling of the antibody via, e.g., coupling (i.e., physically linking) a detectable substance to the antibody, and can also refer to indirect labeling of the antibody by reactivity with another reagent that is directly labeled. Examples of indirect labeling include detection of a primary antibody using a fluorescently labeled secondary antibody.

The biological sample can be brought in contact with and immobilized onto a solid phase support or carrier such as nitrocellulose, or other solid support which is capable of immobilizing cells, cell particles or soluble proteins. The support can then be washed with suitable buffers followed by treatment with the detectably labeled fingerprint gene-specific antibody. The solid phase support can then be washed with the buffer a second time to remove unbound antibody. The amount of bound label on solid support can then be detected by conventional methods.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides and magnetite. The nature of the carrier can be either soluble to some extent or insoluble for the purposes of the present invention. The support material can have virtually any possible structural configuration so long as the coupled molecule is capable of binding to an antigen or antibody. Thus, the support configuration can be spherical, as in a bead, or cylindrical, as in the inside surface of a test tube, or the external surface of a rod. Alternatively, the surface can be flat such as a sheet, test strip, etc. Preferred supports include polystyrene beads. Those skilled in the art will know many other suitable carriers for binding antibody or antigen, or will be able to ascertain the same by use of routine experimentation.

In some embodiments, a bead assay is used to measure feature values for the biomarkers in the biomarker profile. One such bead assay is the Becton Dickinson Cytometric Bead Array (CBA). CBA employs a series of particles with discrete fluorescence intensities to simultaneously detect multiple soluble analytes. CBA is combined with flow cytometry to create a multiplexed assay. The Becton Dickinson CBA system, as embodied for example in the Becton Dickinson Human Inflammation Kit, uses the sensitivity of amplified fluorescence detection by flow cytometry to measure soluble analytes in a particle-based immunoassay. Each bead in a CBA provides a capture surface for a specific protein and is analogous to an individually coated well in an ELISA plate. The BD CBA capture bead mixture is in suspension to allow for the detection of multiple analytes in a small volume sample.

In some embodiments the multiplex analysis method described in U.S. Pat. No. 5,981,180 (“the '180 patent”), hereby incorporated by reference herein in its entirety, and in particular for its teachings of the general methodology, bead technology, system hardware and antibody detection, is used to measure feature values for the biomarkers in a biomarker profile. For this analysis, a matrix of microparticles is synthesized, where the matrix consists of different sets of microparticles. Each set of microparticles can have thousands of molecules of a distinct antibody capture reagent immobilized on the microparticle surface and can be color coded by incorporation of varying amounts of two fluorescent dyes. The ratio of the two fluorescent dyes provides a distinct emission spectrum for each set of microparticles, allowing the identification of a microparticle a set following the pooling of the various sets of microparticles. See also U.S. Pat. Nos. 6,268,222 and 6,599,331, also hereby incorporated by reference herein in their entireties, and in particular for their teachings of various methods of labeling microparticles for multiplex analysis.

5.4.3 Use of Other Methods of Detection

In some embodiments, a separation method may be used determine feature values for biomarkers in a biomarker profile, such that only a subset of biomarkers within the sample is analyzed. For example, the biomarkers that are analyzed in a sample may be mRNA species from a cellular extract which has been fractionated to obtain only the nucleic acid biomarkers within the sample, or the biomarkers may be from a fraction of the total complement of proteins within the sample, which have been fractionated by chromatographic techniques.

Feature values for biomarkers in a biomarker profile can also, for example, be generated by the use of one or more of the following methods described below. For example, methods may include nuclear magnetic resonance (NMR) spectroscopy, a mass spectrometry method, such as electrospray ionization mass spectrometry (ESI-MS), ESI-MS/MS, ESI-MS/(MS)ⁿ (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)ⁿ, atmospheric pressure photoionization mass spectrometry (APPI-MS), APPI-MS/MS, and APPI-(MS)ⁿ. Other mass spectrometry methods may include, inter alia, quadrupole, Fourier transform mass spectrometry (FTMS) and ion trap. Other suitable methods may include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE) or other chromatography, such as thin-layer, gas or liquid chromatography, or any combination thereof. In one embodiment, the biological sample may be fractionated prior to application of the separation method.

In one embodiment, laser desorption/ionization time-of-flight mass spectrometry is used to create determine feature values in a biomarker profile where the biomarkers are proteins or protein fragments that have been ionized and vaporized off an immobilizing support by incident laser radiation and the feature values are the presence or absence of peaks representing these fragments in the mass spectra profile. A variety of laser desorption/ionization techniques are known in the art (see, e.g., Guttman et al., 2001, Anal. Chem. 73:1252-62 and Wei et al., 1999, Nature 399:243-246, each of which is hereby incorporated herein by reference in its entirety).

Laser desorption/ionization time-of-flight mass spectrometry allows the generation of large amounts of information in a relatively short period of time. A biological sample is applied to one of several varieties of a support that binds all of the biomarkers, or a subset thereof, in the sample. Cell lysates or samples are directly applied to these surfaces in volumes as small as 0.5 μL, with or without prior purification or fractionation. The lysates or sample can be concentrated or diluted prior to application onto the support surface. Laser desorption/ionization is then used to generate mass spectra of the sample, or samples, in as little as three hours.

### 5.5 Data Analysis Algorithms

Biomarkers whose corresponding feature values are capable of discriminating between converters and nonconverters are identified in the present invention. The identity of these biomarkers and their corresponding features (e.g., expression levels) can be used to develop a decision rule, or plurality of decision rules, that discriminate between converters and nonconverters. Data analysis algorithms can be used to construct a number of decision rules. Each such data analysis algorithm uses features (e.g., expression values) of a subset of the biomarkers identified in the present invention across a training population that includes converters and nonconverters. Typically, a SIRS subject is considered a nonconverter when the subject does not develop sepsis in a defined time period (e.g., observation period). This defined time period can be, for example, twelve hours, twenty four hours, forty-eight hours, a day, a week, a month, or longer. Specific data analysis algorithms for building a decision rule, or plurality of decision rules, that discriminate between subjects that develop sepsis and subjects that do not develop sepsis during a defined period will be described in the subsections below. Once a decision rule has been built using these exemplary data analysis algorithms or other techniques known in the art, the decision rule can be used to classify a test subject into one of the two or more phenotypic classes (e.g., a converter or a nonconverter). This is accomplished by applying the decision rule to a biomarker profile obtained from the test subject. Such decision rules, therefore, have enormous value as diagnostic indicators.

The present invention provides, in one aspect, for the evaluation of a biomarker profile from a test subject to biomarker profiles obtained from a training population. In some embodiments, each biomarker profile obtained from subjects in the training population, as well as the test subject, comprises a feature for each of a plurality of different biomarkers. In some embodiments, this comparison is accomplished by (i) developing a decision rule using the biomarker profiles from the training population and (ii) applying the decision rule to the biomarker profile from the test subject. As such, the decision rules applied in some embodiments of the present invention are used to determine whether a test subject having SIRS will or will not likely acquire sepsis.

In some embodiments of the present invention, when the results of the application of a decision rule indicate that the subject will likely acquire sepsis, the subject is diagnosed as a “sepsis” subject. If the results of an application of a decision rule indicate that the subject will not acquire sepsis, the subject is diagnosed as a “SIRS” subject. Thus, in some embodiments, the result in the above-described binary decision situation has four possible outcomes:

(i) truly septic, where the decision rule indicates that the subject will acquire sepsis and the subject does in fact acquire sepsis during the definite time period (true positive, TP);

(ii) falsely septic, where the decision rule indicates that the subject will acquire sepsis and the subject, in fact, does not acquire sepsis during the definite time period (false positive, FP);

(iii) truly SIRS, where the decision rule indicates that the subject will not acquire sepsis and the subject, in fact, does not acquire sepsis during the definite time period (true negative, TN); or

(iv) falsely SIRS, where the decision rule indicates that the subject will not acquire sepsis and the subject, in fact, does acquire sepsis during the definite time period (false negative, FN).

It will be appreciated that other definitions for TP, FP, TN, FN can be made. For example, TP could have been defined as instances where the decision rule indicates that the subject will not acquire sepsis and the subject, in fact, does not acquire sepsis during the definite time period. While all such alternative definitions are within the scope of the present invention, for ease of understanding the present invention, the definitions for TP, FP, TN, and FN given by definitions (i) through (iv) above will be used herein, unless otherwise stated.

As will be appreciated by those of skill in the art, a number of quantitative criteria can be used to communicate the performance of the comparisons made between a test biomarker profile and reference biomarker profiles (e.g., the application of a decision rule to the biomarker profile from a test subject). These include positive predicted value (PPV), negative predicted value (NPV), specificity, sensitivity, accuracy, and certainty. In addition, other constructs such a receiver operator curves (ROC) can be used to evaluate decision rule performance. As used herein:

$$P P V = \frac{TP}{TP + FP}$$

$$N P V = \frac{TN}{TN + FN}$$

$$specificity = \frac{TN}{TN + FP}$$

$$sensitivity = \frac{TP}{TP + FN}$$

$$accuracy = certainty = \frac{TP + TN}{N}$$

Here, N is the number of samples compared (e.g., the number of test samples for which a determination of sepsis or SIRS is sought). For example, consider the case in which there are ten subjects for which SIRS/sepsis classification is sought. Biomarker profiles are constructed for each of the ten test subjects. Then, each of the biomarker profiles is evaluated by applying a decision rule, where the decision rule was developed based upon biomarker profiles obtained from a training population. In this example, N, from the above equations, is equal to 10. Typically, N is a number of samples, where each sample was collected from a different member of a population. This population can, in fact, be of two different types. In one type, the population comprises subjects whose samples and phenotypic data (e.g., feature values of biomarkers and an indication of whether or not the subject acquired sepsis) was used to construct or refine a decision rule. Such a population is referred to herein as a training population. In the other type, the population comprises subjects that were not used to construct the decision rule. Such a population is referred to herein as a validation population. Unless otherwise stated, the population represented by N is either exclusively a training population or exclusively a validation population, as opposed to a mixture of the two population types. It will be appreciated that scores such as accuracy will be higher (closer to unity) when they are based on a training population as opposed to a validation population. Nevertheless, unless otherwise explicitly stated herein, all criteria used to assess the performance of a decision rule (or other forms of evaluation of a biomarker profile from a test subject) including certainty (accuracy) refer to criteria that were measured by applying the decision rule corresponding to the criteria to either a training population or a validation population. Furthermore, the definitions for PPV, NPV, specificity, sensitivity, and accuracy defined above can also be found in Draghici, Data Analysis Tools for DNA Microanalysis, 2003, CRC Press LLC, Boca Raton, Fla., pp. 342-343, which is hereby incorporated by reference herein in its entirety.

In some embodiments the training population comprises nonconverters and converters. In some embodiments, biomarker profiles are constructed from this population using biological samples collected from the training population at some time period prior to the onset of sepsis by the converters of the population. As such, for the converters of the training population, a biological sample can be collected two weeks before, one week before, four days before, three days before, one day before, or any other time period before the converters became septic. In practice, such collections are obtained by collecting a biological sample at regular time intervals after admittance into the hospital with a SIRS diagnosis. For example, in one approach, subjects who have been diagnosed with SIRS in a hospital are used as a training population. Once admitted to the hospital with SIRS, the biological samples are collected from the subjects at selected times (e.g., hourly, every eight hours, every twelve hours, daily, etc.). A portion of the subjects acquire sepsis and a portion of the subjects do not acquire sepsis. For the subjects that acquire sepsis, the biological sample taken from the subjects just prior to the onset of sepsis are termed the T₋₁₂ biological samples. All other biological samples from the subjects are retroactively indexed relative to these biological samples. For instance, when a biological sample has been taken from a subject on a daily basis, the biological sample taken the day before the T₋₁₂ sample is referred to as the T₋₃₆ biological sample. Time points for biological samples for a nonconverter in the training population are identified by “time-matching” the nonconverter subject with a converter subject. To illustrate, consider the case in which a subject in the training population became clinically-defined as septic on his sixth day of enrollment. For the sake of illustration, for this subject, T₋₃₆ is day four of the study, and the T₋₃₆ biological sample is the biological sample that was obtained on day four of the study. Likewise, T₋₃₆ for the matched nonconverter subject is deemed to be day four of the study on this paired nonconverter subject.

In some embodiments, N is more than one, more than five, more than ten, more than twenty, between ten and 100, more than 100, or less than 1000 subjects. A decision rule (or other forms of comparison) can have at least about 99% certainty, or even more, in some embodiments, against a training population or a validation population. In other embodiments, the certainty is at least about 97%, at least about 95%, at least about 90%, at least about 85%, at least about 80%, at least about 75%, or at least about 70%, at least about 65%, or at least about 60%, against a training population or a validation population (and therefore against a single subject that is not part of a training population such as a clinical patient). The useful degree of certainty may vary, depending on the particular method of the present invention. As used herein, “certainty” means “accuracy.” In one embodiment, the sensitivity and/or specificity is at is at least about 97%, at least about 95%, at least about 90%, at least about 85%, at least about 80%, at least about 75%, or at least about 70% against a training population or a validation population. In some embodiments, such decision rules are used to predict the development of sepsis with the stated accuracy. In some embodiments, such decision rules are used to diagnoses sepsis with the stated accuracy. In some embodiments, such decision rules are used to determine a stage of sepsis with the stated accuracy.

The number of features that may be used by a decision rule to classify a test subject with adequate certainty is two or more. In some embodiments, it is three or more, four or more, ten or more, or between 10 20, and 210. Depending on the degree of certainty sought, however, the number of features used in a decision rule can be more or less, but in all cases is at least two. In one embodiment, the number of features that may be used by a decision rule to classify a test subject is optimized to allow a classification of a test subject with high certainty.

Relevant data analysis algorithms for developing a decision rule include, but are not limited to, discriminant analysis including linear, logistic, and more flexible discrimination techniques (see, e.g., Gnanadesikan, 1977, Methods for Statistical Data Analysis of Multivariate Observations, New York: Wiley 1977, which is hereby incorporated by reference herein in its entirety); tree-based algorithms such as classification and regression trees (CART) and variants (see, e.g., Breiman, 1984, Classification and Regression Trees, Belmont, Calif.: Wadsworth International Group, which is hereby incorporated by reference herein in its entirety, as well as Section 5.1.3, below); generalized additive models (see, e.g., Tibshirani, 1990, Generalized Additive Models, London: Chapman and Hall, which is hereby incorporated by reference herein in its entirety); and neural networks (see, e.g., Neal, 1996, Bayesian Learning for Neural Networks, New York: Springer-Verlag; and Insua, 1998, Feedforward neural networks for nonparametric regression In: Practical Nonparametric and Semiparametric Bayesian Statistics, pp. 181-194, New York: Springer, which is hereby incorporated by reference herein in its entirety).

In one embodiment, comparison of a test subject's biomarker profile to a biomarker profiles obtained from a training population is performed, and comprises applying a decision rule. The decision rule is constructed using a data analysis algorithm, such as a computer pattern recognition algorithm. Other suitable data analysis algorithms for constructing decision rules include, but are not limited to, logistic regression or a nonparametric algorithm that detects differences in the distribution of feature values (e.g., a Wilcoxon Signed Rank Test (unadjusted and adjusted)). The decision rule can be based upon two, three, four, five, 10, 20 or more features, corresponding to measured observables from one, two, three, four, five, 10, 20 or more biomarkers. In one embodiment, the decision rule is based on hundreds of features or more. Decision rules may also be built using a classification tree algorithm. For example, each biomarker profile from a training population can comprise at least three features, where the features are predictors in a classification tree algorithm. The decision rule predicts membership within a population (or class) with an accuracy of at least about at least about 70%, of at least about 75%, of at least about 80%, of at least about 85%, of at least about 90%, of at least about 95%, of at least about 97%, of at least about 98%, of at least about 99%, or about 100%.

Suitable data analysis algorithms are known in the art, some of which are reviewed in Hastie et al., supra. In a specific embodiment, a data analysis algorithm of the invention comprises Classification and Regression Tree (CART), Multiple Additive Regression Tree (MART), Prediction Analysis for Microarrays (PAM) or Random Forest analysis. Such algorithms classify complex spectra from biological materials, such as a blood sample, to distinguish subjects as normal or as possessing biomarker expression levels characteristic of a particular disease state. In other embodiments, a data analysis algorithm of the invention comprises ANOVA and nonparametric equivalents, linear discriminant analysis, logistic regression analysis, nearest neighbor classifier analysis, neural networks, principal component analysis, quadratic discriminant analysis, regression classifiers and support vector machines. While such algorithms may be used to construct a decision rule and/or increase the speed and efficiency of the application of the decision rule and to avoid investigator bias, one of ordinary skill in the art will realize that computer-based algorithms are not required to carry out the methods of the present invention.

Decision rules can be used to evaluate biomarker profiles, regardless of the method that was used to generate the biomarker profile. For example, suitable decision rules that can be used to evaluate biomarker profiles generated using gas chromatography, as discussed in Harper, “Pyrolysis and GC in Polymer Analysis,” Dekker, New York (1985). Further, Wagner et al., 2002, Anal. Chem. 74:1824-1835 disclose a decision rule that improves the ability to classify subjects based on spectra obtained by static time-of-flight secondary ion mass spectrometry (TOF-SIMS). Additionally, Bright et al., 2002, J. Microbiol. Methods 48:127-38, hereby incorporated by reference herein in its entirety, disclose a method of distinguishing between bacterial strains with high certainty (79-89% correct classification rates) by analysis of MALDI-TOF-MS spectra. Dalluge, 2000, Fresenius J. Anal. Chem. 366:701-711, hereby incorporated by reference herein in its entirety, discusses the use of MALDI-TOF-MS and liquid chromatography-electrospray ionization mass spectrometry (LC/ESI-MS) to classify profiles of biomarkers in complex biological samples.

5.5.1 Decision Trees

One type of decision rule that can be constructed using the feature values of the biomarkers identified in the present invention is a decision tree. Here, the “data analysis algorithm” is any technique that can build the decision tree, whereas the final “decision tree” is the decision rule. A decision tree is constructed using a training population and specific data analysis algorithms. Decision trees are described generally by Duda, 2001, Pattern Classification, John Wiley & Sons, Inc., New York. pp. 395-396, which is hereby incorporated by reference herein. Tree-based methods partition the feature space into a set of rectangles, and then fit a model (like a constant) in each one.

The training population data includes the features (e.g., expression values, or some other observable) for the biomarkers of the present invention across a training set population. One specific algorithm that can be used to construct a decision tree is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

In some embodiments of the present invention, decision trees are used to classify subjects using features for combinations of biomarkers of the present invention. Decision tree algorithms belong to the class of supervised learning algorithms. The aim of a decision tree is to induce a classifier (a tree) from real-world example data. This tree can be used to classify unseen examples that have not been used to derive the decision tree. As such, a decision tree is derived from training data. Exemplary training data contains data for a plurality of subjects (the training population). For each respective subject there is a plurality of features the class of the respective subject (e.g., sepsis/SIRS). In one embodiment of the present invention, the training data is expression data for a combination of biomarkers across the training population.

The following algorithm describes an exemplary decision tree derivation:

```
Tree(Examples,Class,Features)
  Create a root node
  If all Examples have the same Class value, give the root this label
  Else if Features is empty label the root according to the most
  common value
  Else begin
    Calculate the information gain for each Feature
Select the Feature A with highest information gain and make this the
root Feature
    For each possible value, v, of this Feature
      Add a new branch below the root, corresponding to A = v
      Let Examples(v) be those examples with A = v
  If Examples(v) is empty, make the new branch a leaf node labeled
with the most common value among Examples
  Else let the new branch be the tree created by Tree(Examples(v),
Class,Features - {A})
    end
```

A more detailed description of the calculation of information gain is shown in the following. If the possible classes vᵢ of the examples have probabilities P(vᵢ) then the information content I of the actual answer is given by:

$$I ( P ( {v}_{1} ) , … , P ( {v}_{n} ) ) = {\sum }_{i = 1}^{n} - P ( {v}_{i} ) {log}_{2} P ( {v}_{i} )$$

The I— value shows how much information we need in order to be able to describe the outcome of a classification for the specific dataset used. Supposing that the dataset contains p positive (e.g. will develop sepsis) and n negative (e.g. will not develop sepsis) examples (e.g. subjects), the information contained in a correct answer is:

$$I ( \frac{p}{p + n} , \frac{n}{p + n} ) = - \frac{p}{p + n} {log}_{2} \frac{p}{p + n} - \frac{n}{p + n} {log}_{2} \frac{n}{p + n}$$

where log₂ is the logarithm using base two. By testing single features the amount of information needed to make a correct classification can be reduced. The remainder for a specific feature A (e.g. representing a specific biomarker) shows how much the information that is needed can be reduced.

$$Re mainder ( A ) = {\sum }_{i = 1}^{v} \frac{{p}_{i} + {n}_{i}}{p + n} I ( \frac{{p}_{i}}{{p}_{i} + {n}_{i}} , \frac{{n}_{i}}{{p}_{i} + {n}_{i}} )$$

“v” is the number of unique attribute values for feature A in a certain dataset, “i” is a certain attribute value, “pᵢ” is the number of examples for feature A where the classification is positive (e.g. will develop sepsis), “nᵢ” is the number of examples for feature A where the classification is negative (e.g. will not develop sepsis).

The information gain of a specific feature A is calculated as the difference between the information content for the classes and the remainder of feature A:

$$Gain ( A ) = I ( \frac{p}{p + n} , \frac{n}{p + n} ) - Remainder ( A )$$

The information gain is used to evaluate how important the different features are for the classification (how well they split up the examples), and the feature with the highest information.

In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, but are not limited to classification and regression trees (CART), multivariate decision trees, ID3, and C4.5.

In one approach, when a decision tree is used, the gene expression data for a select combination of genes described in the present invention across a training population is standardized to have mean zero and unit variance. The members of the training population are randomly divided into a training set and a test set. For example, in one embodiment, two thirds of the members of the training population are placed in the training set and one third of the members of the training population are placed in the test set. The expression values for a select combination of biomarkers described in the present invention is used to construct the decision tree. Then, the ability for the decision tree to correctly classify members in the test set is determined. In some embodiments, this computation is performed several times for a given combination of biomarkers. In each computational iteration, the members of the training population are randomly assigned to the training set and the test set. Then, the quality of the combination of biomarkers is taken as the average of each such iteration of the decision tree computation.

In addition to univariate decision trees in which each split is based on a feature value for a corresponding biomarker, among the set of biomarkers of the present invention, or the relative feature values of two such biomarkers, multivariate decision trees can be implemented as a decision rule. In such multivariate decision trees, some or all of the decisions actually comprise a linear combination of feature values for a plurality of biomarkers of the present invention. Such a linear combination can be trained using known techniques such as gradient descent on a classification or by the use of a sum-squared-error criterion. To illustrate such a decision tree, consider the expression:

0.04x₁+0.16x₂<500

Here, x₁ and x₂ refer to two different features for two different biomarkers from among the biomarkers of the present invention. To poll the decision rule, the values of features x₁ and x₂ are obtained from the measurements obtained from the unclassified subject. These values are then inserted into the equation. If a value of less than 500 is computed, then a first branch in the decision tree is taken. Otherwise, a second branch in the decision tree is taken. Multivariate decision trees are described in Duda, 2001, Pattern Classification, John Wiley & Sons, Inc., New York, pp. 408-409, which is hereby incorporated by reference.

Another approach that can be used in the present invention is multivariate adaptive regression splines (MARS). MARS is an adaptive procedure for regression, and is well suited for the high-dimensional problems addressed by the present invention. MARS can be viewed as a generalization of stepwise linear regression or a modification of the CART method to improve the performance of CART in the regression setting. MARS is described in Hastie et al., 2001, The Elements of Statistical Learning, Springer-Verlag, New York, pp. 283-295, which is hereby incorporated by reference in its entirety.

5.5.2 Predictive Analysis of Microarrays (PAM)

One approach to developing a decision rule using feature values of biomarkers of the present invention is the nearest centroid classifier. Such a technique computes, for each class (sepsis and SIRS), a centroid given by the average feature levels of the biomarkers in the class, and then assigns new samples to the class whose centroid is nearest. This approach is similar to k-means clustering except clusters are replaced by known classes. This algorithm can be sensitive to noise when a large number of biomarkers are used. One enhancement to the technique uses shrinkage: for each biomarker, differences between class centroids are set to zero if they are deemed likely to be due to chance. This approach is implemented in the Prediction Analysis of Microarray, or PAM. See, for example, Tibshirani et al., 2002, Proceedings of the National Academy of Science USA 99; 6567-6572, which is hereby incorporated by reference in its entirety. Shrinkage is controlled by a threshold below which differences are considered noise. Biomarkers that show no difference above the noise level are removed. A threshold can be chosen by cross-validation. As the threshold is decreased, more biomarkers are included and estimated classification errors decrease, until they reach a bottom and start climbing again as a result of noise biomarkers—a phenomenon known as overfitting.

5.5.3 Bagging, Boosting, and the Random Subspace Method

Bagging, boosting, the random subspace method, and additive trees are data analysis algorithms known as combining techniques that can be used to improve weak decision rules. These techniques are designed for, and usually applied to, decision trees, such as the decision trees described in Section 5.5.1, above. In addition, such techniques can also be useful in decision rules developed using other types of data analysis algorithms such as linear discriminant analysis.

In bagging, one samples the training set, generating random independent bootstrap replicates, constructs the decision rule on each of these, and aggregates them by a simple majority vote in the final decision rule. See, for example, Breiman, 1996, Machine Learning 24, 123-140; and Efron & Tibshirani, An Introduction to Boostrap, Chapman & Hall, New York, 1993, which is hereby incorporated by reference in its entirety.

In boosting, decision rules are constructed on weighted versions of the training set, which are dependent on previous classification results. Initially, all features under consideration have equal weights, and the first decision rule is constructed on this data set. Then, weights are changed according to the performance of the decision rule. Erroneously classified features get larger weights, and the next decision rule is boosted on the reweighted training set. In this way, a sequence of training sets and decision rules is obtained, which is then combined by simple majority voting or by weighted majority voting in the final decision rule. See, for example, Freund & Schapire, “Experiments with a new boosting algorithm,” Proceedings 13th International Conference on Machine Learning, 1996, 148-156, which is hereby incorporated by reference in its entirety.

To illustrate boosting, consider the case where there are two phenotypes exhibited by the population under study, phenotype 1 (e.g., acquiring sepsis during a defined time period), and phenotype 2 (e.g., SIRS only, meaning that the subject does acquire sepsis within a defined time period). Given a vector of predictor biomarkers (e.g., a vector of features that represent such biomarkers) from the training set data, a decision rule G(X) produces a prediction taking one of the type values in the two value set: {phenotype 1, phenotype 2}. The error rate on the training sample is

$$\overline{err} = \frac{1}{N} {\sum }_{i = 1}^{N} I ( {y}_{i} \neq G ( {x}_{i} ) )$$

where N is the number of subjects in the training set (the sum total of the subjects that have either phenotype 1 or phenotype 2). For example, if there are 49 organisms that acquire sepsis and 72 organisms that remain in the SIRS state, N is 121. A weak decision rule is one whose error rate is only slightly better than random guessing. In the boosting algorithm, the weak decision rule is repeatedly applied to modified versions of the data, thereby producing a sequence of weak decision rules Gₘ(x), m, =1, 2, . . . , M. The predictions from all of the decision rules in this sequence are then combined through a weighted majority vote to produce the final decision rule:

$$G ( x ) = sign ( {\sum }_{m = 1}^{M} {α}_{m} {G}_{m} ( x ) )$$

Here α₁, α₂, . . . , α_{M} are computed by the boosting algorithm and their purpose is to weigh the contribution of each respective decision rule Gₘ(x). Their effect is to give higher influence to the more accurate decision rules in the sequence.

The data modifications at each boosting step consist of applying weights w₁, w₂, . . . wₙ to each of the training observations (xᵢ, yᵢ), i=1, 2, . . . , N. Initially all the weights are set to wᵢ=1/N, so that the first step simply trains the decision rule on the data in the usual manner. For each successive iteration m=2, 3, . . . , M the observation weights are individually modified and the decision rule is reapplied to the weighted observations. At step m, those observations that were misclassified by the decision rule Gₘ−1(x) induced at the previous step have their weights increased, whereas the weights are decreased for those that were classified correctly. Thus as iterations proceed, observations that are difficult to correctly classify receive ever-increasing influence. Each successive decision rule is thereby forced to concentrate on those training observations that are missed by previous ones in the sequence.

The exemplary boosting algorithm is summarized as follows:

1. Initialize the observation weights wᵢ = 1/N, i = 1, 2, . . . , N.
2. For m = 1 to M:

(a) Fit a decision rule Gₘ(x) to the training set using weights wᵢ.
(b) Compute

${err}_{m} = \frac{{\sum }_{i = 1}^{N} {w}_{i} I ( {y}_{1} \neq {G}_{m} ( {x}_{i} ) )}{{\sum }_{i = 1}^{N} {w}_{i}}$

(c) Compute αₘ=log((1−errₘ)/errₘ).
(d) Set wᵢ ← wᵢ · exp[αₘ · I(yᵢ ≠ Gₘ(xᵢ))], i = 1,2,K , N.

$3. Output G ( x ) = sign ⌊ {\sum }_{m = 1}^{M} {α}_{m} {G}_{m} ( x ) ⌋$

In one embodiment in accordance with this algorithm, each object is, in fact, a factor. Furthermore, in the algorithm, the current decision rule Gₘ(x) is induced on the weighted observations at line 2a. The resulting weighted error rate is computed at line 2b. Line 2c calculates the weight aₘ given to Gₘ(x) in producing the final classifier G(x) (line

3). The individual weights of each of the observations are updated for the next iteration at line 2d. Observations misclassified by Gₘ(x) have their weights scaled by a factor exp(αₘ), increasing their relative influence for inducing the next classifier Gₘ+1(x) in the sequence. In some embodiments, modifications of the Freund and Schapire, 1997, Journal of Computer and System Sciences 55, pp. 119-139, boosting methods are used. See, for example, Hasti et al., The Elements of Statistical Learning, 2001, Springer, New York, Chapter 10, which is hereby incorporated by reference in its entirety. For example, in some embodiments, feature preselection is performed using a technique such as the nonparametric scoring methods of Park et al., 2002, Pac. Symp. Biocomput. 6, 52-63, which is hereby incorporated by reference in its entirety. Feature preselection is a form of dimensionality reduction in which the genes that discriminate between classifications the best are selected for use in the classifier. Then, the LogitBoost procedure introduced by Friedman et al., 2000, Ann Stat 28, 337-407 is used rather than the boosting procedure of Freund and Schapire. In some embodiments, the boosting and other classification methods of Ben-Dor et al., 2000, Journal of Computational Biology 7, 559-583, hereby incorporated by reference in its entirety, are used in the present invention. In some embodiments, the boosting and other classification methods of Freund and Schapire, 1997, Journal of Computer and System Sciences 55, 119-139, hereby incorporated by reference in its entirety, are used.

In the random subspace method, decision rules are constructed in random subspaces of the data feature space. These decision rules are usually combined by simple majority voting in the final decision rule. See, for example, Ho, “The Random subspace method for constructing decision forests,” IEEE Trans Pattern Analysis and Machine Intelligence, 1998; 20(8): 832-844, which is hereby incorporated by reference in its entirety.

5.5.4 Multiple Additive Regression Trees

Multiple additive regression trees (MART) represents another way to construct a decision rule that can be used in the present invention. A generic algorithm for MART is:

$1. Initialize {f}_{0} ( x ) = arg {min}_{γ} {\sum }_{i = 1}^{N} L ( {y}_{i} , γ ) .$
2. For m = 1 to M:

(a) For I = 1,2, . . . , N compute

${r}_{im} = - {[ \frac{\partial L ( {y}_{i} , f ( {x}_{i} ) )}{\partial f ( {x}_{i} )} ]}_{f = {f}_{m - 1}}$

(b) Fit a regression tree to the targets rᵢₘ giving terminal regions Rⱼₘ, j

= 1,2, . . . , Jₘ.

(c) For j = 1, 2, . . . , Jₘ compute

${γ}_{jm} = arg {min}_{γ} {\sum }_{{x}_{i} ∈ {R}_{jm}} L ( {y}_{i} , {f}_{m - 1} ( {x}_{i} ) + γ ) .$

$( d ) Update {f}_{m} ( x ) = {f}_{m - 1} ( x ) + {\sum }_{j = 1}^{{J}_{m}} {γ}_{jm} I ( x ∈ {R}_{jm} )$

3. Output \hat{f}(x) = f_{M} (x).

Specific algorithms are obtained by inserting different loss criteria L(y,f(x)). The first line of the algorithm initializes to the optimal constant model, which is just a single terminal node tree. The components of the negative gradient computed in line 2(a) are referred to as generalized pseudo residuals, r. Gradients for commonly used loss functions are summarized in Table 10.2, of Hastie et al., 2001, The Elements of Statistical Learning, Springer-Verlag, New York, p. 321, which is hereby incorporated by reference. The algorithm for classification is similar and is described in Hastie et al., Chapter 10, which is hereby incorporated by reference in its entirety. Tuning parameters associated with the MART procedure are the number of iterations M and the sizes of each of the constituent trees Jₘ, m=1, 2, . . . , M.

5.5.5 Decision Rules Derived by Regression

In some embodiments, a decision rule used to classify subjects is built using regression. In such embodiments, the decision rule can be characterized as a regression classifier, preferably a logistic regression classifier. Such a regression classifier includes a coefficient for each of the biomarkers (e.g., a feature for each such biomarker) used to construct the classifier. In such embodiments, the coefficients for the regression classifier are computed using, for example, a maximum likelihood approach. In such a computation, the features for the biomarkers (e.g., RT-PCR, microarray data) is used. In particular embodiments, molecular marker data from only two trait subgroups is used (e.g., trait subgroup a: will acquire sepsis in a defined time period and trait subgroup b: will not acquire sepsis in a defined time period) and the dependent variable is absence or presence of a particular trait in the subjects for which biomarker data is available.

In another specific embodiment, the training population comprises a plurality of trait subgroups (e.g., three or more trait subgroups, four or more specific trait subgroups, etc.). These multiple trait subgroups can correspond to discrete stages in the phenotypic progression from healthy, to SIRS, to sepsis, to more advanced stages of sepsis in a training population. In this specific embodiment, a generalization of the logistic regression model that handles multicategory responses can be used to develop a decision that discriminates between the various trait subgroups found in the training population. For example, measured data for selected molecular markers can be applied to any of the multi-category logit models described in Agresti, An Introduction to Categorical Data Analysis, 1996, John Wiley & Sons, Inc., New York, Chapter 8, hereby incorporated by reference in its entirety, in order to develop a classifier capable of discriminating between any of a plurality of trait subgroups represented in a training population.

5.5.6 Neural Networks

In some embodiments, the feature data measured for select biomarkers of the present invention (e.g., RT-PCR data, mass spectrometry data, microarray data) can be used to train a neural network. A neural network is a two-stage regression or classification decision rule. A neural network has a layered structure that includes a layer of input units (and the bias) connected by a layer of weights to a layer of output units. For regression, the layer of output units typically includes just one output unit. However, neural networks can handle multiple quantitative responses in a seamless fashion.

In multilayer neural networks, there are input units (input layer), hidden units (hidden layer), and output units (output layer). There is, furthermore, a single bias unit that is connected to each unit other than the input units. Neural networks are described in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Neural networks are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, N.Y., each of which is hereby incorporated by reference in its entirety. What is disclosed below is some exemplary forms of neural networks.

The basic approach to the use of neural networks is to start with an untrained network, present a training pattern to the input layer, and to pass signals through the net and determine the output at the output layer. These outputs are then compared to the target values; any difference corresponds to an error. This error or criterion function is some scalar function of the weights and is minimized when the network outputs match the desired outputs. Thus, the weights are adjusted to reduce this measure of error. For regression, this error can be sum-of-squared errors. For classification, this error can be either squared error or cross-entropy (deviation). See, e.g., Hastie et al., 2001, The Elements of Statistical Learning, Springer-Verlag, New York, which is hereby incorporated by reference in its entirety.

Three commonly used training protocols are stochastic, batch, and on-line. In stochastic training, patterns are chosen randomly from the training set and the network weights are updated for each pattern presentation. Multilayer nonlinear networks trained by gradient descent methods such as stochastic back-propagation perform a maximum-likelihood estimation of the weight values in the classifier defined by the network topology. In batch training, all patterns are presented to the network before learning takes place. Typically, in batch training, several passes are made through the training data. In online training, each pattern is presented once and only once to the net.

In some embodiments, consideration is given to starting values for weights. If the weights are near zero, then the operative part of the sigmoid commonly used in the hidden layer of a neural network (see, e.g., Hastie et al., 2001, The Elements of Statistical Learning, Springer-Verlag, New York, hereby incorporated by reference) is roughly linear, and hence the neural network collapses into an approximately linear classifier. In some embodiments, starting values for weights are chosen to be random values near zero. Hence the classifier starts out nearly linear, and becomes nonlinear as the weights increase. Individual units localize to directions and introduce nonlinearities where needed. Use of exact zero weights leads to zero derivatives and perfect symmetry, and the algorithm never moves. Alternatively, starting with large weights often leads to poor solutions.

Since the scaling of inputs determines the effective scaling of weights in the bottom layer, it can have a large effect on the quality of the final solution. Thus, in some embodiments, at the outset all expression values are standardized to have mean zero and a standard deviation of one. This ensures all inputs are treated equally in the regularization process, and allows one to choose a meaningful range for the random starting weights. With standardization inputs, it is typical to take random uniform weights over the range [−0.7, +0.7].

A recurrent problem in the use of three-layer networks is the optimal number of hidden units to use in the network. The number of inputs and outputs of a three-layer network are determined by the problem to be solved. In the present invention, the number of inputs for a given neural network will equal the number of biomarkers selected from the training population. The number of output for the neural network will typically be just one. However, in some embodiments more than one output is used so that more than just two states can be defined by the network. For example, a multi-output neural network can be used to discriminate between, healthy phenotypes, various stages of SIRS, and/or various stages of sepsis. If too many hidden units are used in a neural network, the network will have too many degrees of freedom and is trained too long, there is a danger that the network will overfit the data. If there are too few hidden units, the training set cannot be learned. Generally speaking, however, it is better to have too many hidden units than too few. With too few hidden units, the classifier might not have enough flexibility to capture the nonlinearities in the date; with too many hidden units, the extra weight can be shrunk towards zero if appropriate regularization or pruning, as described below, is used. In typical embodiments, the number of hidden units is somewhere in the range of 5 to 100, with the number increasing with the number of inputs and number of training cases.

One general approach to determining the number of hidden units to use is to apply a regularization approach. In the regularization approach, a new criterion function is constructed that depends not only on the classical training error, but also on classifier complexity. Specifically, the new criterion function penalizes highly complex classifiers; searching for the minimum in this criterion is to balance error on the training set with error on the training set plus a regularization term, which expresses constraints or desirable properties of solutions:

J=Jₚₐₜ+λJ_{reg}.

The parameter λ is adjusted to impose the regularization more or less strongly. In other words, larger values for λ will tend to shrink weights towards zero: typically cross-validation with a validation set is used to estimate λ. This validation set can be obtained by setting aside a random subset of the training population. Other forms of penalty have been proposed, for example the weight elimination penalty (see, e.g., Hastie et al., 2001, The Elements of Statistical Learning, Springer-Verlag, New York, hereby incorporated by reference).

Another approach to determine the number of hidden units to use is to eliminate—prune—weights that are least needed. In one approach, the weights with the smallest magnitude are eliminated (set to zero). Such magnitude-based pruning can work, but is nonoptimal; sometimes weights with small magnitudes are important for learning and training data. In some embodiments, rather than using a magnitude-based pruning approach, Wald statistics are computed. The fundamental idea in Wald Statistics is that they can be used to estimate the importance of a hidden unit (weight) in a classifier. Then, hidden units having the least importance are eliminated (by setting their input and output weights to zero). Two algorithms in this regard are the Optimal Brain Damage (OBD) and the Optimal Brain Surgeon (OBS) algorithms that use second-order approximation to predict how the training error depends upon a weight, and eliminate the weight that leads to the smallest increase in training error.

Optimal Brain Damage and Optimal Brain Surgeon share the same basic approach of training a network to local minimum error at weight w, and then pruning a weight that leads to the smallest increase in the training error. The predicted functional increase in the error for a change in full weight vector δw is:

$$δ J = {( \frac{\partial J}{\partial w} )}^{t} \cdot δ w + \frac{1}{2} δ {w}^{t} \cdot \frac{{\partial }^{2} J}{\partial {w}^{2}} \cdot δ w + O ( \vert \vert δ w {\vert \vert}^{3} )$$

where

$$\frac{{\partial }^{2} J}{\partial {w}^{2}}$$

is the Hessian matrix. The first term vanishes at a local minimum in error; third and higher order terms are ignored. The general solution for minimizing this function given the constraint of deleting one weight is:

$$δ w = - \frac{{w}_{q}}{[ {H}^{- 1} ]} {H}^{- 1} \cdot {u}_{q} and {L}_{q} = \frac{1}{2} - \frac{{w}_{q}^{2}}{{[ {H}^{- 1} ]}_{qq}}$$

Here, u_{q} is the unit vector along the qth direction in weight space and L_{q} is approximation to the saliency of the weight q—the increase in training error if weight q is pruned and the other weights updated δw. These equations require the inverse of H. One method to calculate this inverse matrix is to start with a small value, H₀⁻¹=α⁻¹I, where α is a small parameter—effectively a weight constant. Next the matrix is updated with each pattern according to

$${H}_{m + 1}^{- 1} = {H}_{m}^{- 1} - \frac{{H}_{m}^{- 1} {X}_{m + 1} {X}_{m + 1}^{T} {H}_{m}^{- 1}}{\frac{n}{{a}_{m}} + {X}_{m + 1}^{T} {H}_{m}^{- 1} {X}_{m + 1}} \qquad Eqn . 1$$

where the subscripts correspond to the pattern being presented and aₘ decreases with m. After the full training set has been presented, the inverse Hessian matrix is given by H⁻¹=Hₙ⁻¹. In algorithmic form, the Optimal Brain Surgeon method is:

begin initialize n_{H}, w, θ

train a reasonably large network to minimum error

do compute H⁻¹ by Eqn. 1

${q}^{*} ← arg {min}_{q} {w}_{q}^{2} / ( {2 [ {H}^{- 1} ]}_{qq} ) ( saliency {L}_{q} )$ $w ← w - \frac{{w}_{{q}^{*}}}{{[ {H}^{- 1} ]}_{{q}^{*} {q}^{*}}} {H}^{- 1} {e}_{{q}^{*}} ( saliency {L}_{q} )$

until J(w) > θ

return w

end

The Optimal Brain Damage method is computationally simpler because the calculation of the inverse Hessian matrix in line 3 is particularly simple for a diagonal matrix. The above algorithm terminates when the error is greater than a criterion initialized to be θ. Another approach is to change line 6 to terminate when the change in J(w) due to elimination of a weight is greater than some criterion value. In some embodiments, the back-propagation neural network See, for example Abdi, 1994, “A neural network primer,” J. Biol System. 2, 247-283, hereby incorporated by reference in its entirety.

5.5.7 Clustering

In some embodiments, features for select biomarkers of the present invention are used to cluster a training set. For example, consider the case in which ten features (corresponding to ten biomarkers) described in the present invention is used. Each member m of the training population will have feature values (e.g. expression values) for each of the ten biomarkers. Such values from a member m in the training population define the vector:

X₁ₘ X₂ₘ X₃ₘ X₄ₘ X₅ₘ X₆ₘ X₇ₘ X₈ₘ X₉ₘ X₁₀ₘ

where Xᵢₘ is the expression level of the i^{th} biomarker in organism m. If there are m organisms in the training set, selection of i biomarkers will define m vectors. Note that the methods of the present invention do not require that each the expression value of every single biomarker used in the vectors be represented in every single vector m. In other words, data from a subject in which one of the i^{th} biomarkers is not found can still be used for clustering. In such instances, the missing expression value is assigned either a “zero” or some other normalized value. In some embodiments, prior to clustering, the feature values are normalized to have a mean value of zero and unit variance.

Those members of the training population that exhibit similar expression patterns across the training group will tend to cluster together. A particular combination of genes of the present invention is considered to be a good classifier in this aspect of the invention when the vectors cluster into the trait groups found in the training population. For instance, if the training population includes class a: subjects that do not develop sepsis, and class b: subjects that develop sepsis, an ideal clustering classifier will cluster the population into two groups, with one cluster group uniquely representing class a and the other cluster group uniquely representing class b.

Clustering is described on pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda 1973, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar”. An example of a nonmetric similarity function s(x, x′) is provided on page 216 of Duda 1973.

Once a method for measuring “similarity” or “dissimilarity” between points in a dataset has been selected, clustering requires a criterion function that measures the clustering quality of any partition of the data. Partitions of the data set that extrernize the criterion function are used to cluster the data. See page 217 of Duda 1973. Criterion functions are discussed in Section 6.8 of Duda 1973.

More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

5.5.8 Principal Component Analysis

Principal component analysis (PCA) has been proposed to analyze gene expression data. More generally, PCA can be used to analyze feature value data of biomarkers of the present invention in order to construct a decision rule that discriminates converters from nonconverters. Principal component analysis is a classical technique to reduce the dimensionality of a data set by transforming the data to a new set of variable (principal components) that summarize the features of the data. See, for example, Jolliffe, 1986, Principal Component Analysis, Springer, New York, which is hereby incorporated by reference. Principal component analysis is also described in Draghici, 2003, Data Analysis Tools for DNA Microarrays, Chapman & Hall/CRC, which is hereby incorporated by reference. What follows is non-limiting examples of principal components analysis.

Principal components (PCs) are uncorrelated and are ordered such that the k^{th} PC has the kth largest variance among PCs. The k^{th} PC can be interpreted as the direction that maximizes the variation of the projections of the data points such that it is orthogonal to the first k−1 PCs. The first few PCs capture most of the variation in the data set. In contrast, the last few PCs are often assumed to capture only the residual ‘noise’ in the data.

PCA can also be used to create a classifier in accordance with the present invention. In such an approach, vectors for the select biomarkers of the present invention can be constructed in the same manner described for clustering above. In fact, the set of vectors, where each vector represents the feature values (e.g., abundance values) for the select genes from a particular member of the training population, can be viewed as a matrix. In some embodiments, this matrix is represented in a Free-Wilson method of qualitative binary description of monomers (Kubinyi, 1990, 3D QSAR in drug design theory methods and applications, Pergamon Press, Oxford, pp 589-638), and distributed in a maximally compressed space using PCA so that the first principal component (PC) captures the largest amount of variance information possible, the second principal component (PC) captures the second largest amount of all variance information, and so forth until all variance information in the matrix has been considered.

Then, each of the vectors (where each vector represents a member of the training population) is plotted. Many different types of plots are possible. In some embodiments, a one-dimensional plot is made. In this one-dimensional plot, the value for the first principal component from each of the members of the training population is plotted. In this form of plot, the expectation is that members of a first subgroup (e.g. those subjects that do not develop sepsis in a determined time period) will cluster in one range of first principal component values and members of a second subgroup (e.g., those subjects that develop sepsis in a determined time period) will cluster in a second range of first principal component values.

In one ideal example, the training population comprises two subgroups: “sepsis” and “SIRS.” The first principal component is computed using the molecular marker expression values for the select biomarkers of the present invention across the entire training population data set. Then, each member of the training set is plotted as a function of the value for the first principal component. In this ideal example, those members of the training population in which the first principal component is positive are the “responders” and those members of the training population in which the first principal component is negative are “subjects with sepsis.”

In some embodiments, the members of the training population are plotted against more than one principal component. For example, in some embodiments, the members of the training population are plotted on a two-dimensional plot in which the first dimension is the first principal component and the second dimension is the second principal component. In such a two-dimensional plot, the expectation is that members of each subgroup represented in the training population will cluster into discrete groups. For example, a first cluster of members in the two-dimensional plot will represent subjects that develop sepsis in a given time period and a second cluster of members in the two-dimensional plot will represent subjects that do not develop sepsis in a given time period.

5.5.9 Nearest Neighbor Analysis

Nearest neighbor classifiers are memory-based and require no classifier to be fit. Given a query point x₀, the k training points x₍ᵣ₎, r, . . . , k closest in distance to x₀ are identified and then the point x₀ is classified using the k nearest neighbors. Ties can be broken at random. In some embodiments, Euclidean distance in feature space is used to determine distance as:

d₍i)=∥x₍i)−xₒ∥.

Typically, when the nearest neighbor algorithm is used, the expression data used to compute the linear discriminant is standardized to have mean zero and variance 1. In the present invention, the members of the training population are randomly divided into a training set and a test set. For example, in one embodiment, two thirds of the members of the training population are placed in the training set and one third of the members of the training population are placed in the test set. A select combination of biomarkers of the present invention represents the feature space into which members of the test set are plotted. Next, the ability of the training set to correctly characterize the members of the test set is computed. In some embodiments, nearest neighbor computation is performed several times for a given combination of biomarkers of the present invention. In each iteration of the computation, the members of the training population are randomly assigned to the training set and the test set. Then, the quality of the combination of biomarkers is taken as the average of each such iteration of the nearest neighbor computation.

The nearest neighbor rule can be refined to deal with issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference in its entirety.

5.5.10 Linear Discriminant Analysis

Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. In the present invention, the feature values for the select combinations of biomarkers of the present invention across a subset of the training population serve as the requisite continuous independent variables. The trait subgroup classification of each of the members of the training population serves as the dichotomous categorical dependent variable.

LDA seeks the linear combination of variables that maximizes the ratio of between-group variance and within-group variance by using the grouping information. Implicitly, the linear weights used by LDA depend on how the feature values of a molecular marker across the training set separates in the two groups (e.g., a group a that develops sepsis during a defined time period and a group b that does not develop sepsis during a defined time period) and how these feature values correlate with the feature values of other biomarkers. In some embodiments, LDA is applied to the data matrix of the N members in the training sample by K biomarkers in a combination of biomarkers described in the present invention. Then, the linear discriminant of each member of the training population is plotted. Ideally, those members of the training population representing a first subgroup (e.g. those subjects that develop sepsis in a defined time period) will cluster into one range of linear discriminant values (e.g., negative) and those member of the training population representing a second subgroup (e.g. those subjects that will not develop sepsis in a defined time period) will cluster into a second range of linear discriminant values (e.g., positive). The LDA is considered more successful when the separation between the clusters of discriminant values is larger. For more information on linear discriminant analysis, see Duda, Pattern Classification, Second Edition, 2001, John Wiley & Sons, Inc; and Hastie, 2001, The Elements of Statistical Learning, Springer, New York; and Venables & Ripley, 1997, Modern Applied Statistics with s-plus, Springer, New York, each of which is hereby incorporated by reference in its entirety.

5.5.11 Quadratic Discriminant Analysis

Quadratic discriminant analysis (QDA) takes the same input parameters and returns the same results as LDA. QDA uses quadratic equations, rather than linear equations, to produce results. LDA and QDA are interchangeable, and which to use is a matter of preference and/or availability of software to support the analysis. Logistic regression takes the same input parameters and returns the same results as LDA and QDA.

5.5.12 Support Vector Machines

In some embodiments of the present invention, support vector machines (SVMs) are used to classify subjects using feature values of the genes described in the present invention. SVMs are a relatively new type of learning algorithm. See, for example, Cristianini and Shawe-Taylor, 2000, An Introduction to Support Vector Machines, Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y., Duda, Pattern Classification, Second Edition, 2001, John Wiley & Sons, Inc.; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data training data with a hyper-plane that is maximally distance from them. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

In one approach, when a SVM is used, the feature data is standardized to have mean zero and unit variance and the members of a training population are randomly divided into a training set and a test set. For example, in one embodiment, two thirds of the members of the training population are placed in the training set and one third of the members of the training population are placed in the test set. The expression values for a combination of genes described in the present invention is used to train the SVM. Then the ability for the trained SVM to correctly classify members in the test set is determined. In some embodiments, this computation is performed several times for a given combination of molecular markers. In each iteration of the computation, the members of the training population are randomly assigned to the training set and the test set. Then, the quality of the combination of biomarkers is taken as the average of each such iteration of the SVM computation.

5.5.13 Evolutionary Methods

Inspired by the process of biological evolution, evolutionary methods of decision rule design employ a stochastic search for an decision rule. In broad overview, such methods create several decision rules—a population—from a combination of biomarkers described in the present invention. Each decision rule varies somewhat from the other. Next, the decision rules are scored on feature data across the training population. In keeping with the analogy with biological evolution, the resulting (scalar) score is sometimes called the fitness. The decision rules are ranked according to their score and the best decision rules are retained (some portion of the total population of decision rules). Again, in keeping with biological terminology, this is called survival of the fittest. The decision rules are stochastically altered in the next generation—the children or offspring. Some offspring decision rules will have higher scores than their parent in the previous generation, some will have lower scores. The overall process is then repeated for the subsequent generation: the decision rules are scored and the best ones are retained, randomly altered to give yet another generation, and so on. In part, because of the ranking, each generation has, on average, a slightly higher score than the previous one. The process is halted when the single best decision rule in a generation has a score that exceeds a desired criterion value. More information on evolutionary methods is found in, for example, Duda, Pattern Classification, Second Edition, 2001, John Wiley & Sons, Inc.

5.5.14 Other Data Analysis Algorithms

The data analysis algorithms described above are merely examples of the types of methods that can be used to construct a decision rule for discriminating converters from nonconverters. Moreover, combinations of the techniques described above can be used. Some combinations, such as the use of the combination of decision trees and boosting, have been described. However, many other combinations are possible. In addition, in other techniques in the art such as Projection Pursuit and Weighted Voting can be used to construct decision rules.

5.6 Biomarkers

In specific embodiments, the present invention provides biomarkers that are useful in diagnosing or predicting sepsis and/or its stages of progression in a subject. While the methods of the present invention may use an unbiased approach to identifying predictive biomarkers, it will be clear to the artisan that specific groups of biomarkers associated with physiological responses or with various signaling pathways may be the subject of particular attention. This is particularly the case where biomarkers from a biological sample are contacted with an array that can be used to measure the amount of various biomarkers through direct and specific interaction with the biomarkers (e.g., an antibody array or a nucleic acid array). In this case, the choice of the components of the array may be based on a suggestion that a particular pathway is relevant to the determination of the status of sepsis or SIRS in a subject. The indication that a particular biomarker has a feature that is predictive or diagnostic of sepsis or SIRS may give rise to an expectation that other biomarkers that are physiologically regulated in a concerted fashion likewise may provide a predictive or diagnostic feature. The artisan will appreciate, however, that such an expectation may not be realized because of the complexity of biological systems. For example, if the amount of a specific mRNA biomarker were a predictive feature, a concerted change in mRNA expression of another biomarker might not be measurable, if the expression of the other biomarker was regulated at a post-translational level. Further, the mRNA expression level of a biomarker may be affected by multiple converging pathways that may or may not be involved in a physiological response to sepsis.

Biomarkers can be obtained from any biological sample, which can be, by way of example and not of limitation, whole blood, plasma, saliva, serum, red blood cells, platelets, neutrophils, eosinophils, basophils, lymphocytes, monocytes, urine, cerebral spinal fluid, sputum, stool, cells and cellular extracts, or other biological fluid sample, tissue sample or tissue biopsy from a host or subject. The precise biological sample that is taken from the subject may vary, but the sampling preferably is minimally invasive and is easily performed by conventional techniques.

Measurement of a phenotypic change may be carried out by any conventional technique. Measurement of body temperature, respiration rate, pulse, blood pressure, or other physiological parameters can be achieved via clinical observation and measurement. Measurements of biomarker molecules may include, for example, measurements that indicate the presence, concentration, expression level, or any other value associated with a biomarker molecule. The form of detection of biomarker molecules typically depends on the method used to form a profile of these biomarkers from a biological sample. See Section 5.4, above, and Tables 1, 4, 5, 6, 7, 12, 13, 14, 15, 16, 17, 18, 19 20 and/or 21 below.

In a particular embodiment, the biomarker profile comprises at least 2, 3, 4, 5, 6, 7, 8, 9, 10, 11, 12, 13, 14, 15, 16, 17, 18, 19, or 20 different biomarkers listed in anyone of Tables 1, 4, 5, 6, 7, 12, 13, 14, 15, 16, 17, 18, 19 20, and 21 below. In another particular embodiment, the biomarker profile comprises at least 2, 3, 4, 5, 6, 7, 8, 9, 10, 11, 12, 13, 14, 15, 16, 17, 18, 19, or 20 different biomarkers listed in any combination of Tables 1, 4, 5, 6, 7, 12, 13, 14, 15, 16, 17, 18, 19 20, and 21, below. In still another particular embodiment, the biomarker profile comprises at least 2, 3, 4, 5, 6, 7, 8, 9, or 10 different biomarkers listed in 4 below. In still another particular embodiment, the biomarker profile comprises at least CRP, APOA2, and SERPINC1. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19 20, and 21. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, or more additional biomarkers from any one of Tables 1, 4, 5, 6, 7, 12, 13, 14, 15, 16, 17, 18, 19 20, and 21. The biomarker profile further comprises a respective corresponding feature for each of the biomarkers in the profile. Such biomarkers can be, for example, mRNA transcripts, cDNA or some other nucleic acid, for example amplified nucleic acid, or proteins. Generally, the biomarkers in a biomarker profile are derived from at least two different genes. In the case where a biomarker in the biomarker profile is listed in Tables 1, 4, 5, 6, 7, 12, 13, 14, 15, 16, 17, 18, 19 20 and/or 21, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. Further still, the biomarker can be, for example, a protein of a gene listed in Tables 1, 4, 5, 6 7, 12, 13, 14, 15, 16, 17, 18, 19 20 and/or 21 or a discriminating fragment of the protein, or an indication of any of the above. Here, a discriminating molecule or fragment is a molecule or fragment that, when detected, indicates presence or abundance of the above-identified transcript, cDNA, amplified nucleic acid, or protein. In accordance with this embodiment, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in Tables 1, 4, 5, 6, 7, 12, 13, 14, 15, 16, 17, 18, 19 20 and/or 21). In one embodiment, such an assay utilizes a nucleic acid micro array.

In some embodiments the biomarker profile has between 2 and 100 biomarkers listed in Table 1. In some embodiments, the biomarker profile has between 3 and 50 biomarkers listed in Table 1. In some embodiments, the biomarker profile has between 4 and 25 biomarkers listed in Table 1. In some embodiments, the biomarker profile has at least 3 biomarkers listed in Table 1. In some embodiments, the biomarker profile has at least 4 biomarkers listed in Table 1. In some embodiments, the biomarker profile has at least 6, 7, 8, 9, 10, 11, 12, 13, 14, 15, 16, 17, 18, 19, 20, 25, 30, 35, 40, 45, 50, 54, 5, 60, 65, 70, 75, 80, 85, 90, 95, 96, or 100 biomarkers listed in Table 1. In some embodiments, each such biomarker is a nucleic acid. In some embodiments, each such biomarker is a protein. In some embodiments, some of the biomarkers in the biomarker profile are nucleic acids and some of the biomarkers in the biomarker profile are proteins.

In some embodiments the biomarker profile has between 2 and 10 biomarkers listed in Table 4. In some embodiments, the biomarker profile has between 3 and 8 biomarkers listed in Table 4. In some embodiments, the biomarker profile has at least 2, 3, 4, 5, 6, 7, 8, 9, or 10 biomarkers listed in Table 4. In some embodiments, each such biomarker is a nucleic acid. In some embodiments, each such biomarker is a protein. In some embodiments, some of the biomarkers in the biomarker profile are nucleic acids and some of the biomarkers in the biomarker profile are proteins. In some embodiments, the biomarker profile comprises at least CRP, APOA2, and SERPINC1. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any combination of Tables 1, 4, 5, 6, 7, 12, 13, 14, 15, 16, 17, 18, 19 20, and 21. In some embodiments, the biomarker profile comprises at least one of SERPINC1, APOA2, and CRP, and, additionally, at least 1, 2, 3, 4, 5, 6, 7, 8, 9, 10, 11, 12, 13, 14, 15, 16, 17, 18, 19, 20 or more additional biomarkers from any one of Tables 1, 4, 5, 6, 7, 12, 13, 14, 15, 16, 17, 18, 19 20, and 21.

In some embodiments, some of the biomarkers in the biomarker profile are nucleic acids and some of the biomarkers in the biomarker profile are proteins. In some embodiments the biomarker profile has between 2 and 100 biomarkers listed in Table 5. In some embodiments, the biomarker profile has between 3 and 50 biomarkers listed in Table 5. In some embodiments, the biomarker profile has between 4 and 25 biomarkers listed in Table 5. In some embodiments, the biomarker profile has at least 3 biomarkers listed in Table 5. In some embodiments, the biomarker profile has at least 4 biomarkers listed in Table 5. In some embodiments, the biomarker profile has at least 6, 7, 8, 9, 10, 11, 12, 13, 14, 15, 16, 17, 18, 19, 20, 25, 30, 35, 40, 45, 50, 54, 5, 60, 65, 70, 75, 80, 85, 90 biomarkers listed in Table 5. In some embodiments, each such biomarker is a nucleic acid. In some embodiments, each such biomarker is a protein. In some embodiments, some of the biomarkers in the biomarker profile are nucleic acids and some of the biomarkers in the biomarker profile are proteins.

In some embodiments the biomarker profile has between 2 and 30 biomarkers listed in Table 6. In some embodiments, the biomarker profile has between 3 and 50 biomarkers listed in Table 6. In some embodiments, the biomarker profile has between 4 and 25 biomarkers listed in Table 6. In some embodiments, the biomarker profile has at least 3 biomarkers listed in Table 6. In some embodiments, the biomarker profile has at least 4 biomarkers listed in Table 6. In some embodiments, the biomarker profile has at least 6, 7, 8, 9, 10, 11, 12, 13, 14, 15, 16, 17, 18, 19, 20, 25, 30, 35, 40, 45, 50, 54, 5, 60, 65, 70, 75, 80, 85, 90 biomarkers listed in Table 6. In some embodiments, each such biomarker is a nucleic acid. In some embodiments, each such biomarker is a protein. In some embodiments, some of the biomarkers in the biomarker profile are nucleic acids and some of the biomarkers in the biomarker profile are proteins.

In some embodiments the biomarker profile has between 2 20, and 21 biomarkers listed in Table 7. In some embodiments, the biomarker profile has between 3 and 25 biomarkers listed in Table 7. In some embodiments, the biomarker profile has between 4 and 25 biomarkers listed in Table 7. In some embodiments, the biomarker profile has at least 3 biomarkers listed in Table 7. In some embodiments, the biomarker profile has at least 4 biomarkers listed in Table 7. In some embodiments, the biomarker profile has at least 6, 7, 8, 9, 10, 11, 12, 13, 14, 15, 16, 17, 18, 19 or 20 biomarkers listed in Table 7. In some embodiments, each such biomarker is a nucleic acid. In some embodiments, each such biomarker is a protein. In some embodiments, some of the biomarkers in the biomarker profile are nucleic acids and some of the biomarkers in the biomarker profile are proteins.

In some embodiments the biomarker profile has between 2 20, and 21 biomarkers listed in Table 15. In some embodiments, the biomarker profile has between 3 and 25 biomarkers listed in Table 15. In some embodiments, the biomarker profile has between 4 and 25 biomarkers listed in Table 15. In some embodiments, the biomarker profile has at least 3 biomarkers listed in Table 15. In some embodiments, the biomarker profile has at least 4 biomarkers listed in Table 15. In some embodiments, the biomarker profile has at least 6, 7, 8, 9, 10, 11, 12, 13, 14, 15, 16, 17, 18, 19 or 20 biomarkers listed in Table 15. In some embodiments, each such biomarker is a nucleic acid. In some embodiments, each such biomarker is a protein. In some embodiments, some of the biomarkers in the biomarker profile are nucleic acids and some of the biomarkers in the biomarker profile are proteins.

In some embodiments the biomarker profile has between 2 20, and 21 biomarkers listed in Table 17. In some embodiments, the biomarker profile has between 3 and 25 biomarkers listed in Table 17. In some embodiments, the biomarker profile has between 4 and 25 biomarkers listed in Table 17. In some embodiments, the biomarker profile has at least 3 biomarkers listed in Table 17. In some embodiments, the biomarker profile has at least 4 biomarkers listed in Table 17. In some embodiments, the biomarker profile has at least 6, 7, 8, 9, 10, 11, 12, 13, 14, 15, 16, 17, 18, 19 or 20 biomarkers listed in Table 17. In some embodiments, each such biomarker is a nucleic acid. In some embodiments, each such biomarker is a protein. In some embodiments, some of the biomarkers in the biomarker profile are nucleic acids and some of the biomarkers in the biomarker profile are proteins.

Biomarkers are listed in Tables 1, 4, 5, 6, and 7 by their gene symbol and gene name for reference purposes. However, the present invention encompasses, inter alia, both the nucleic acid product from and protein product form, as well as discriminatory fragments thereof, of such genes. A more detailed description of the biomarkers listed in Tables 1, 4, 5, 6, 7, 12, 13, 14, 15, 16, 17, 18, 19, 20, and 21 is provided in Section 5.6.1, below.

5.6.1 Isolation of Useful Biomarkers

The accession numbers in this section refer to National Center for Biotechnology Information (NCBI) accession numbers, through the NCBI portal Entrez, to the NCBI nucleotide database and the NCBI protein sequence database. The NCBI nucleotide database is a collection of sequences from several sources, including GenBank®, the EST database, the GSS database, HomoloGene, the HTG database, the SNPs database, RefSeq (Release 17), UniSTS, UniGene, and the PDB. GenBank® is the NIH genetic sequence database, an annotated collection of all publicly available DNA sequences (Nucleic Acids Research 2005 Jan. 13; 33 (Database Issue):D34-D36). There are approximately 59,750,386,305 bases in 54,584,635 sequence records in the traditional GenBank divisions and 63,183,065,091 bases in 12,465,546 sequence records in the WGS division as of February 2006. The EST database is a collection of expressed sequence tags, or short, single-pass sequence reads from mRNA (cDNA). The GSS database is a database of genome survey sequences, or short, single-pass genomic sequences. HomoloGene is a gene homology tool that compares nucleotide sequences between pairs of organisms in order to identify putative orthologs. The HTG database is a collection of high-throughput genome sequences from large-scale genome sequencing centers, including unfinished and finished sequences. The SNPs database is a central repository for both single-base nucleotide substitutions and short deletion and insertion polymorphisms. The RefSeq database is a database of non-redundant reference sequences standards, including genomic DNA contigs, mRNAs, and proteins for known genes. For more information on RefSeq, see NCBI Reference Sequence (RefSeq): a curated non-redundant sequence database of genomes, transcripts and proteins Pruitt et al., 2005, Nucleic Acids Res 33: D501-D504. The STS database is a database of sequence tagged sites, or short sequences that are operationally unique in the genome. The UniSTS database is a unified, non-redundant view of sequence tagged sites (STSs). The UniGene database is a collection of ESTs and full-length mRNA sequences organized into clusters, each representing a unique known or putative human gene annotated with mapping and expression information and cross-references to other sources. The NCBI protein database has been compiled from a variety of sources, including SwissProt, Protein Information Resource (PIR), PRF, Protein Data Bank (PDB) (sequences from solved structures), and translations from annotated coding regions in GenBank and RefSeq.

The nucleotide sequence of C4B, (identified by accession no. K02403) is disclosed in, e.g., Belt et al., 1984, “The structural basis of the multiple forms of human complement component C4,” published in Cell 36, 907-914, and the amino acid sequence of C4B (identified by accession no. AAB67980) is disclosed in, e.g., Xie et al., 2003, “Analysis of the gene-dense major histocompatibility complex class III region and its comparison to mouse” published in Genome Res. 13, 2621-2635, each of which is incorporated by reference herein in its entirety.

The nucleotide sequence of SERPINA3 (identified by accession no. NM_{—}001085) is disclosed in, e.g., Furiya, Y. et al., 2005, “Alpha-1-antichymotrypsin gene polymorphism and susceptibility to multiple system atrophy (MSA),” published in Brain Res. Mol. Brain. Res. 138 (2), 178-181, and the amino acid sequence of SERPINA3 (identified by accession no. NP_{—}001076) is disclosed in, e.g., Furiya, Y. et al., 2005, “Alpha-1-antichymotrypsin gene polymorphism and susceptibility to multiple system atrophy (MSA),” published in Brain Res. Mol. Brain. Res. 138 (2), 178-181, each of which is incorporated by reference herein in its entirety.

The nucleotide sequence of ACTB (identified by accession no. NM_{—}001101) is disclosed in, e.g., Dahlen, A. et al., 2004, “Molecular genetic characterization of the genomic ACTB-GLI fusion in pericytoma with t(7;12),” published in Biochem. Biophys. Res. Commun. 325 (4), 1318-1323, and the amino acid sequence of ACTB (identified by accession no. AAS79319) is disclosed in Livingston, R. J et al., and is unpublished and each of which is incorporated by reference herein in its entirety.

The nucleotide sequence of AFM (identified by accession no. NM_{—}001133) is disclosed in, e.g., Jerkovic, L. et al., 2005, “Afamin is a novel human vitamin E-binding glycoprotein characterization and in vitro expression,” published in J. Proteome Res. 4 (3), 889-899, and the amino acid sequence of AFM (identified by accession no. AAA21612) is disclosed in, e.g., Lichenstein, H. S. et al., 1994, “Afamin is a new member of the albumin, alpha-fetoprotein, and vitamin D-binding protein gene family,” published in J. Biol. Chem. 269 (27), 18149-18154, each of which is incorporated by reference herein in its entirety.

The nucleotide sequence of AGT (identified by accession no. NM_{—}000029) is disclosed in, e.g., Rasmussen-Torvik, L. J. et al, 2005, “A population association study of angiotensinogen polymorphisms and haplotypes with left ventricular phenotypes,” published in Hypertension 46 (6), 1294-1299, and the amino acid sequence of AGT (identified by accession no. AAR03501) is disclosed in, e.g., Crawford, D. C. et al., 2004, “Haplotype diversity across 100 candidate genes for inflammation, lipid metabolism, and blood pressure regulation in two populations,” published in Am. J. Hum. Genet. 74 (4), 610-622, each of which is incorporated by reference herein in its entirety.

The nucleotide sequence of AHSG (identified by accession no. NM_{—}001622) is disclosed in, e.g., Matsushima, K. et al., 1982, “Purification and physicochemical characterization of human alpha2-HS-glycoprotein,” published in Biochim. Biophys. Acta 701 (2), 200-205; Keeley, F. W. et al., 1985, “Identification and quantitation of alpha 2-HS-glycoprotein in the mineralized matrix of calcified plaques of atherosclerotic human aorta” published in Atherosclerosis 55 (1), 63-69; Yoshioka, Y. et al., 1986, “The complete amino acid sequence of the A-chain of human plasma alpha 2HS-glycoprotein” published in J. Biol. Chem. 261 (4), 1665-1676 and the amino acid sequence of AHSG (identified by accession no. NP_{—}001613) is disclosed in, e.g., Matsushima, K. et al., 1982, “Purification and physicochemical characterization of human alpha 2-HS-glycoprotein,” published in Biochim. Biophys. Acta 701 (2), 200-205 (1982); Keeley, F. W. et al., 1985, “Identification and quantitation of alpha 2-HS-glycoprotein in the mineralized matrix of calcified plaques of atherosclerotic human aorta” published in Atherosclerosis 55 (1), 63-69; Yoshioka, Y. et al., 1986, “The complete amino acid sequence of the A-chain of human plasma alpha 2HS-glycoprotein” published in J. Biol. Chem. 261 (4), 1665-1676, each of which is incorporated by reference herein in its entirety.

The nucleotide sequence of AMBP (identified by accession no. NM_{—}001633) is disclosed in, e.g., Ekstrom, B. et al., 1977, “Human alpha1-microglobulin. Purification procedure, chemical and physiochemical properties,” published in J. Biol. Chem. 252 (22), 8048-8057; Grubb, A. O. et al., 1983, “Isolation of human complex-forming glycoprotein, heterogeneous in charge (protein HC), and its IgA complex from plasma. Physiochemical and immunochemical properties, normal plasma concentration” published in J. Biol. Chem. 258 (23), 14698-14707; Bourguignon, J. et al., 1985, “Human inter-alpha-trypsin-inhibitor: characterization and partial nucleotide sequencing of a light chain-encoding cDNA” published in Biochem. Biophys. Res. Commun. 131 (3), 1146-1153 and the amino acid sequence of AMBP (identified by accession no. NP_{—}001624) is disclosed in, e.g., Ekstrom, B. et al., 1977, “Human alpha1-microglobulin. Purification procedure, chemical and physiochemical properties,” published in J. Biol. Chem. 252 (22), 8048-8057; Grubb, A. O. et al., 1983, “Isolation of human complex-forming glycoprotein, heterogeneous in charge (protein HC), and its IgA complex from plasma. Physiochemical and immunochemical properties, normal plasma concentration” published in J. Biol. Chem. 258 (23), 14698-14707; Bourguignon, J. et al., 1985, “Human inter-alpha-trypsin-inhibitor: characterization and partial nucleotide sequencing of a light chain-encoding cDNA” published in Biochem. Biophys. Res. Commun. 131 (3), 1146-1153, each of which is incorporated by reference herein in its entirety.

The nucleotide sequence of APOF (identified by accession no. NM_{—}001638) is disclosed in, e.g., Olofsson, S. O. et al., 1978, “Isolation and partial characterization of a new acidic apolipoprotein (apolipoprotein F) from high density lipoproteins of human plasma,” published in Biochemistry 17 (6), 1032-1036; Koren, E. et al., “Isolation and characterization of simple and complex lipoproteins containing apolipoprotein F from human plasma,” published in Biochemistry 21 (21), 5347-5351; Day, J. R. et al., 1994, “Purification and molecular cloning of human apolipoprotein F,” published in Biochem. Biophys. Res. Commun. 203 (2), 1146-1151, and the amino acid sequence of APOF (identified by accession no. AAA65642) is disclosed in, e.g., Day, J. R. et al, 1994, “Purification and molecular cloning of human apolipoprotein F,” published in Biochem. Biophys. Res. Commun. 203 (2), 1146-1151, each of which is incorporated by reference herein in its entirety.

The nucleotide sequence of APOA1 (identified by accession no. NM_{—}000039) is disclosed in, e.g., Breslow et al, 1987, “Isolation and characterization of cDNA clones for human apolipoprotein A-I,” published in Proc. Natl. Acad. Sci. U.S.A. 79 (22), 6861-6865; Karathanasis et al, 1983, “An inherited polymorphism in the human apolipoprotein A-I gene locus related to the development of atherosclerosis,” published in Nature 301 (5902), 718-720; Law et al., 1983, “cDNA cloning of human apoA-I: amino acid sequence of preproapoA-I,” published in Biochem. Biophys. Res. Commun. 112 (1), and the amino acid sequence of APOA1 (identified by accession no. AAD34604) is disclosed in, e.g., Hamidi et al., 1999, “A novel apolipoprotein A-1 variant, Arg173Pro, associated with cardiac and cutaneous amyloidosis,” Biochem. Biophys. Res. Commun. 257, 584-588, each of which is incorporated by reference herein in its entirety.

The nucleotide sequence of APOA1 precursor (identified by accession no. NM_{—}000039) is disclosed in, e.g., Breslow, J. L. et al., 1987, “Isolation and characterization of cDNA clones for human apolipoprotein A-I,” published in Proc. Natl. Acad. Sci. U.S.A. 79 (22), 6861-6865; Karathanasis, S. K. et al., 1983, “An inherited polymorphism in the human apolipoprotein A-I gene locus related to the development of atherosclerosis,” published in Nature 301 (5902), 718-720; Law, S. W. et al., 1983, “cDNA cloning of human apoA-I: amino acid sequence of preproapoA-I,” published in Biochem. Biophys. Res. Commun. 112 (1), and the amino acid sequence of APOA1 precursor (identified by accession no. P02647) is disclosed in, e.g., Shoulders, 1983, “Gene structure of human apolipoprotein A1,” Nucleic Acids Research 1, 1983, 2827-2837, each of which is incorporated by reference herein in its entirety.

The nucleotide sequence of APOA2 (identified by accession no. NM_{—}001643) is disclosed in, e.g., Brewer, H. B. Jr. et al., 1972, “Amino acid sequence of human apoLp-Gln-II (apoA-II), an apolipoprotein isolated from the high-density lipoprotein complex,” published in Proc. Natl. Acad. Sci. U.S.A. 69 (5), 1304-1308; Servillo, L. et al., 1981, “Evaluation of the mixed interaction between apolipoproteins A-II and C-I equilibrium sedimentation,” published in Biophys. Chem. 13 (1), 29-38; Koren, E. et al., 1982, “Isolation and characterization of simple and complex lipoproteins containing apolipoprotein F from human plasma,” published in Biochemistry 21 (21), 5347-5351), and the amino acid sequence of APOA2 (identified by accession no. AAA51701) is disclosed in, e.g., Chan, L. et al., 1987, “Molecular cloning and sequence analysis of human apolipoprotein A-II cDNA,” published in Meth. Enzymol. 128, 745-752, each of which is incorporated by reference herein in its entirety.

The nucleotide sequence of apoliprotein All precursor (identified by accession no. X00955) is disclosed in, e.g., Brewer et al., 1972, “Amino acid sequence of human apoLp-Gln-II (ApoA-II), an apoliprotein isolated from the high-density lipoprotein complex,” Proc. Natl. Acad. Sci. U.S.A. 69, 1304-1308, and the amino acid sequence of apolipoprotein All precursor (identified by accession no. P02652) is disclosed in, e.g., Knott et al., 1985, “The human apolipoprotein All gene structural organization and sites of expression,” Nucleic Acids Research 13, 6387-6398, each of which is incorporated by reference herein in its entirety.

The nucleotide sequence of APOA4 (identified by accession no. NM_{—}000482) is disclosed in, e.g., Karathanasis, S. K., 1985, “Apolipoprotein multigene family: tandem organization of human apolipoprotein AI, CIII, and AIV genes,” published in Proc. Natl. Acad. Sci. U.S.A. 82 (19), 6374-6378; Elshourbagy, N. A. et al., 1986, “The nucleotide and derived amino acid sequence of human apolipoprotein A-IV mRNA and the close linkage of its gene to the genes of apolipoproteins A-I and C-III,” published in J. Biol. Chem. 261 (5), 1998-2002; Karathanasis, S. K. et al., 1986, “Structure, evolution, and tissue-specific synthesis of human apolipoprotein AIV,” published in Biochemistry 25 (13), 3962-3970, and the amino acid sequence of APOA4 (identified by accession no. AAS68228) is disclosed in, e.g., Fullerton et al., 2004, “The effects of scale: variation in the APOA1/C3/A4/A5 gene cluster,” Hum. Genet. 115 (1), 36-56 (2004) each of which is incorporated by reference herein in its entirety.

The nucleotide sequence of APOB (identified by accession no. NM_{—}000384) is disclosed in, e.g., Mahley, R. W. et al., 1984, “Plasma lipoproteins: apolipoprotein structure and function,” published in J. Lipid Res. 25 (12), 1277-1294; Lusis, A. J. et al., 1985, “Cloning and expression of apolipoprotein B, the major protein of low and very low density lipoproteins,” published in Proc. Natl. Acad. Sci. U.S.A. 82 (14), 4597-4601; Deeb, S. S. et al., 1985, “A partial cDNA clone for human apolipoprotein B,” published in Proc. Natl. Acad. Sci. U.S.A. 82 (15), 4983-4986, and the amino acid sequence of APOB (identified by accession no. AAP72970) is disclosed in, Yang et al., 1986, “The complete cDNA and amino acid sequence of human apolipoprotein B-100,” J. Biol. Chem. 261, 12918-12921, each of which is incorporated by reference herein in its entirety.

The nucleotide sequence of APOC1 (identified by accession no. NM_{—}001645) is disclosed in, e.g., Servillo, L. et al., 1981, “Evaluation of the mixed interaction between apolipoproteins A-II and C-I equilibrium sedimentation,” published in Biophys. Chem. 13 (1), 29-38; Curry, M. D. et al., 1981, “Quantitative determination of apolipoproteins C-I and C-II in human plasma by separate electroimmunoassays,” published in Clin. Chem. 27 (4), 543-548; Knott, T. J. et al., 1984, “Characterisation of mRNAs encoding the precursor for human apolipoprotein CT,” published in Nucleic Acids Res. 12 (9), 3909-3915, and the amino acid sequence of APOC1 (identified by accession no. AAQ91813) is disclosed in, Jackson et al., 1974, “The primary structure of apolipoprotein-serine,” J. Biol. Chem. 249:5308-5313, each of which is incorporated by reference herein in its entirety.

The nucleotide sequence of APOC3 (identified by accession no. BC027977) is disclosed in, e.g., Strausberg, R. L. et al., 2002, “Generation and initial analysis of more than 15,000 full-length human and mouse cDNA sequences,” published in Proc. Natl. Acad. Sci. U.S.A. 99 (26), 16899-16903, and the amino acid sequence of APOC3 (identified by accession no. AAB59372) is disclosed in, e.g., Maeda, H. et al., 1987, “Molecular cloning of a human apoC-III variant: Thr 74----Ala 74 mutation prevents O-glycosylation,” published in J. Lipid Res. 28 (12), 1405-1409, each of which is incorporated by reference herein in its entirety.

The nucleotide sequence of APOE (identified by accession no. NM_{—}000041) is disclosed in, e.g., Utermann, G. et al., 1979, “Polymorphism of apolipoprotein E. III. Effect of a single polymorphic gene locus on plasma lipid levels in man,” published in Clin. Genet. (1), 63-72; Rall, S. C. Jr. et al., 1982, “Structural basis for receptor binding heterogeneity of apolipoprotein E from type III hyperlipoproteinemic subjects,” published in Proc. Natl. Acad. Sci. U.S.A. 79 (15), 4696-4700; Breslow, J. L. et al., 1982, “Identification and DNA sequence of a human apolipoprotein E cDNA clone,” published in J. Biol. Chem. 257 (24), 14639-14641, and the amino acid sequence of APOE (identified by accession no. AAB59397) is disclosed in, e.g., Emi, M. et al., 1988, “Genotyping and sequence analysis of apolipoprotein E isoforms,” published in Genomics 3 (4), 373-379; Das, H. K. et al., 1985, “Isolation, characterization, and mapping to chromosome 19 of the human apolipoprotein E gene,” published in J. Biol. Chem. 260 (10), 6240-6247, each of which is incorporated by reference herein in its entirety.

The nucleotide sequence of APOH (identified by accession no. NM_{—}000042) is disclosed in, e.g., Lee, N. S. et al., 1983, “beta 2-Glycoprotein I. Molecular properties of an unusual apolipoprotein, apolipoprotein H,” published in J. Biol. Chem. 258 (8), 4765-4770; Lozier et al., 1984, “Complete amino acid sequence of human plasma beta 2-glycoprotein I,” published in Proc. Natl. Acad. Sci. U.S.A. 81 (12), 3640-3644; Henry et al, 1988, “Inhibition of the activation of Hageman factor (factor XII) by beta 2-glycoprotein I,” published in J. Lab. Clin. Med. 111 (5), 519-523, and the amino acid sequence of APOH (identified by accession no. CAA40977) is disclosed in, e.g. Mehdi et al., 1991, “Nucleotide sequence and expression of the human gene encoding apolipoprotein H (beta 2-glycoprotein I),” published in Gene 108 (2), 293-298, each of which is incorporated by reference herein in its entirety.

The nucleotide sequence of SERPINC1 (identified by accession no. NM_{—}000488) is disclosed in, e.g., Bjork et al., 1981, “The site in human antithrombin for functional proteolytic cleavage by human thrombin,” FEBS Lett. 126 (2), 257-260; Lijnen, H. R. et al., 1983, “Heparin binding properties of human histidine-rich glycoprotein. Mechanism and role in the neutralization of heparin in plasma,” published in J. Biol. Chem. 258, 3803-3808; Chandra et al., 1983, “Isolation and sequence characterization of a cDNA clone of human antithrombin III,” published in Proc. Natl. Acad. Sci. U.S.A. 80, 1845-1848, and the amino acid sequence of SERPINC1 (identified by accession no. CAI14923) is disclosed in, e.g., Sehra, 2005, Direct Submission, Wellcome Trust Sanger Institute, Hinxton, Cambridgeshire, each of which is incorporated by reference herein in its entirety.

The nucleotide sequence of antithrombin-III precursor (ATIII) (identified by accession no. X68793) is disclosed in Bock et al. 1988, “Antithrombin III Utah: proline-407 to leucine mutation in a highly conserved region near the inhibitor reactive site,” Biochemistry 27, 6171-6178 and the amino acid sequence of antithrombin-III precursor is disclosed in Bock, 1982, “Cloning and expression of the cDNA for human antithrombin III,” Nucleic Acids Res 10, 8113-8125 each of which is incorporated by reference herein in its entirety.

The nucleotide sequence of AZGP1 (identified by accession no. NM_{—}001185) is disclosed in, e.g., Burgi et al., 1981, “Preparation and properties of Zn-alpha 2-glycoprotein of normal human plasma,” published in J. Biol. Chem. 236, 1066-1074; Shibata et al., 1982, “Nephritogenic glycoprotein. IX. Plasma Zn-alpha2-glycoprotein as a second source of nephritogenic glycoprotein in urine,” published in Nephron 31 (2), 170-176; Ueyama, H. et al., 1991, “Cloning and nucleotide sequence of a human Zn-alpha 2-glycoprotein cDNA and chromosomal assignment of its gene,” published in Biochem. Biophys. Res. Commun. 177 (2), 696-703, and the amino acid sequence of AZGP1 (identified by accession no. NP_{—}001176) is disclosed in, e.g., Burgi, W. et al., 1981, “Preparation and properties of Zn-alpha 2-glycoprotein of normal human plasma,” published in J. Biol. Chem. 236, 1066-1074; Shibata et al., 1982, “Nephritogenic glycoprotein. IX. Plasma Zn-alpha2-glycoprotein as a second source of nephritogenic glycoprotein in urine,” published in Nephron 31 (2), 170-176; Ueyama, H. et al., 1991, “Cloning and nucleotide sequence of a human Zn-alpha 2-glycoprotein cDNA and chromosomal assignment of its gene,” published in Biochem. Biophys. Res. Commun. 177, 696-703, each of which is incorporated by reference herein in its entirety.

The nucleotide sequence of BF (identified by accession no. NM_{—}001710) is disclosed in, e.g., Amason, A. et al., 1977, “Very close linkage between HLA-B and BF inferred from allelic association” published in Nature 268 (5620), 527-528; Woods, et al., 1982, “Isolation of cDNA clones for the human complement protein factor B, a class III major histocompatibility complex gene product” published in Proc. Natl. Acad. Sci. U.S.A. 79 (18), 5661-5665; Campbell, R. D et al., 1983, “Molecular cloning and characterization of the gene coding for human complement protein factor B,” published in Proc. Natl. Acad. Sci. U.S.A. 80 (14), 4464-4468.

The nucleotide sequence of SERPING1 (identified by accession no. NM_{—}000062) is disclosed in, e.g., Chesne, S. et al, 1982, “Fluid-phase interaction of C1 inhibitor (C1 Inh) and the subcomponents C1r and C1s of the first component of complement, C1” published in Biochem. J. 201 (1), 61-70; Brower, M. S. et al., 1982, “Proteolytic cleavage and inactivation of alpha 2-plasmin inhibitor and C1 inactivator by human polymorphonuclear leukocyte elastase” published in J. Biol. Chem. 257 (16), 9849-9854; Nilsson, T. et al, 1983, “Structural and circular-dichroism studies on the interaction between human C1-esterase inhibitor and C1s” published in Biochem. J. 213 (3), 617-624 (1983), and the amino acid sequence of SERPING1 (identified by accession no. AAW69393) is disclosed in, e.g., Stoppa-Lyonnet, 1990, “Clusters of intragenic Alu repeats predispose the human C1 inhibitor locus to deleterious rearrangements,” Proc. Natl. Acad. Sci. USA 87:1551-1555, each of which is incorporated by reference herein in its entirety.

The nucleotide sequence of plasma protease C1 inhibitor precursor (identified by accession no. AB209826) is disclosed in, e.g., Carter, 1988, “Genomic and cDNA cloning of the human C1 inhibitor. Intron-exon junctions and comparison to other serpins,” Eur. J. Biochem. 173: 163-169, and the amino acid sequence of plasma protease C1 inhibitor precursor (identified by accession no. P05155) is disclosed in, e.g., Dunbar and Fothergill, 1988, Eur. J. Biochem 173, 163-169, each of which is incorporated by reference herein in its entirety.

The nucleotide sequence of C1QB (identified by accession no. NM_{—}000491) is disclosed in, e.g., Reid, K. B. et al, 1978, “Amino acid sequence of the N-terminal 108 amino acid residues of the B chain of subcomponent C1q of the first component of human complement” published in Biochem. J. 173 (3), 863-868; Reid, K. B., 1979, “Complete amino acid sequences of the three collagen-like regions present in subcomponent C1q of the first component of human complement” published in Biochem. J. 179 (2), 367-371; Reid, K. B. et al, 1982, “Completion of the amino acid sequences of the A and B chains of subcomponent C1q of the first component of human complement” published in Biochem. J. 203 (3), 559-569, and the amino acid sequence of C1QB (identified by accession no. NP_{—}000482) is disclosed in, Reid, K. B. et al, 1978, “Amino acid sequence of the N-terminal 108 amino acid residues of the B chain of subcomponent C1q of the first component of human complement” published in Biochem. J. 173 (3), 863-868; Reid, K. B., 1979, “Complete amino acid sequences of the three collagen-like regions present in subcomponent C1q of the first component of human complement” published in Biochem. J. 179 (2), 367-371; Reid, K. B. et al., 1982, “Completion of the amino acid sequences of the A and B chains of subcomponent C1q of the first component of human complement” published in Biochem. J. 203 (3), 559-569, each of which is incorporated by reference herein in its entirety.

The nucleotide sequence of C1S (identified by accession no. NM_{—}201442) is disclosed in, e.g., Nilsson, T. et al., 1983, “Structural and circular-dichroism studies on the interaction between human C1-esterase inhibitor and C1s” published in Biochem. J. 213(3), 617-624; Bock, S. C. et al., 1986, “Human C1 inhibitor: primary structure, cDNA cloning, and chromosomal localization” published in Biochemistry 25 (15), 4292-4301 (1986); Mackinnon, C. M., 1987, “Molecular cloning of cDNA for human complement component C1s. The complete amino acid sequence” published in Eur. J. Biochem. 169 (3), 547-553, and the amino acid sequence of C1S (identified by accession no. AAW69393) is disclosed in, e.g., Nilsson, T. et al., 1983, “Structural and circular-dichroism studies on the interaction between human C1-esterase inhibitor and C1s” published in Biochem. J. 213(3), 617-624; Bock, S. C. et al., 1986, “Human C1 inhibitor: primary structure, cDNA cloning, and chromosomal localization” published in Biochemistry 25 (15), 4292-4301; Mackinnon, C. M. 1987, “Molecular cloning of cDNA for human complement component C1s. The complete amino acid sequence” published in Eur. J. Biochem. 169 (3), 547-553, each of which is incorporated by reference herein in its entirety.

The nucleotide sequence of C2 (identified by accession no. NM_{—}000063) is disclosed in, e.g., Lutsenko, S. M. et al., 1976, “Circulating blood volume and regional hemodynamics in acute gastrointestinal hemorrhage” published in J. Biol. Chem. 2, 38-41; Bentley, D. R. et al., 1984, “Isolation of cDNA clones for human complement component C2” published in Proc. Natl. Acad. Sci. U.S.A. 81 (4), 1212-1215; Bentley, D. R., 1985, “DNA polymorphism of the C2 locus” published in Immunogenetics 22 (4), 377-390.

The nucleotide sequence of C3 (identified by accession no. NM_{—}000064) is disclosed in, e.g., Alper, C. A. et al., 1970, “Studies in vivo and in vitro on an abnormality in the metabolism of C3 in a patient with increased susceptibility to infection” published in J. Clin. Invest. 49 (11), 1975-1985; Renwick, A. G. et al., 1978, “The fate of saccharin impurities: the excretion and metabolism of [3-14C]Benz[d]-isothiazoline-1,1-dioxide (BIT) in man and rat” published in Xenobiotica 8 (8), 475-486; Bischof, P. et al., 1984, “Pregnancy-associated plasma protein A (PAPP-A) specifically inhibits the third component of human complement (C3)” published in Placenta 5 (1), 1-7, and the amino acid sequence of C3 (identified by accession no. AAR89906) is disclosed in, e.g., Hugli, 1975, “Human anaphylatoxin (C3a) from the third component of complment,” J. Biol. Chem. 250: 8293-8301; Oxvig et al., 1995, “Idnentification of angiotensinogen and complement C3dg as novel proteins binding the proform of eosinophil major basic protein in human pregnancy serum and plasma,” J. Biol. Chem. 270: 13645013651; and Thomas et al., 1982, “Third compoment of human complement: localization of the internal thiolester bond,” Proc. Natl. Acad. Sci. USA 79: 1054-1058, each of which is incorporated by reference herein in its entirety.

The nucleotide sequence of C4BPA (identified by accession no. NM_{—}001017367) is disclosed in, e.g., Hillarp, A. et al., 1988, “Novel subunit in C4b-binding protein required for protein S binding” published in J. Biol. Chem. 263 (25), 12759-12764 (1988); Hillarp. A., 1990, “T Cloning of cDNA coding for the beta chain of human complement component C4b-binding protein: sequence homology with the alpha chain” published in Proc. Natl. Acad. Sci. U.S.A. 87 (3), 1183-1187; Andersson, A. et al., 1990, “Genes for C4b-binding protein alpha- and beta-chains (C4BPA and C4BPB) are located on chromosome 1, band 1q32, in humans and on chromosome 13 in rats” published in Somat. Cell Mol. Genet. 16 (5), 493-500 which is incorporated by reference herein in its entirety.

The nucleotide sequence of C5 (identified by accession no. NM_{—}001736) is disclosed in, e.g., Gerard, N. P. et al, 1991, “The chemotactic receptor for human C5a anaphylatoxin” published in Nature 349 (6310), 614-617; Boulay, F., et al., 1991, “T Expression cloning of a receptor for C5a anaphylatoxin on differentiated HL-60 cells” published in Biochemistry 30 (12), 2993-2999; Bao, L., et al, 1992, “Mapping of genes for the human C5a receptor (C5AR), human FMLP receptor (FPR), and two FMLP receptor homologue orphan receptors (FPRH1, FPRH2) to chromosome 19” published in Genomics 13 (2), 437-440, and the amino acid sequence of C5 (identified by accession no. NP_{—}001726) is disclosed in, e.g., Tack, B. F. et al., 1979, “Fifth component of human complement: purification from plasma and polypeptide chain structure” published in Biochemistry 18 (8), 1490-1497; Lundwall, A. B. et al., 1985, “Isolation and sequence analysis of a cDNA clone encoding the fifth complement component” published in J. Biol. Chem. 260 (4), 2108-2112; Wetsel, R. A. et al, 1988, “Molecular analysis of human complement component C5: localization of the structural gene to chromosome 9” published in Biochemistry 27 (5), 1474-1482, each of which is incorporated by reference herein in its entirety.

The nucleotide sequence of C8A (identified by accession no. NM_{—}000562) is disclosed in, e.g., Matthews, 1980, “Recurrent meningococcal infections associated with a functional deficiency of the C8 component of human complement” published in Clin. Exp. Immunol. 39 (1), 53-59; Stewart, J. L. et al., 1985, “Analysis of the specific association of the eighth and ninth components of human complement: identification of a direct role for the alpha subunit of C8” Biochemistry 24 (17), 4598-4602; Rao, A. G., 1987, “Complementary DNA and derived amino acid sequence of the alpha subunit of human complement protein C8: evidence for the existence of a separate alpha subunit messenger RNA” published in Biochemistry 26 (12), 3556-3564, and the amino acid sequence of C8A (identified by accession no. CAI19172) is disclosed in, e.g., Steckel, 1980, “The eight component of human complment,” J. Biol. Chem. 255:11997-12005, each of which is incorporated by reference herein in its entirety.

The nucleotide sequence of C8G (identified by accession no. NM_{—}000606) is disclosed in, e.g., Ng, S. C. et al., 1987, “The eighth component of human complement: evidence that it is an oligomeric serum protein assembled from products of three different genes,” published in Biochemistry 26 (17), 5229-5233; Haefliger, J. A. et al., 1987, “Structural homology of human complement component C8 gamma and plasma protein HC: identity of the cysteine bond pattern,” published in Biochem. Biophys. Res. Commun. 149 (2), 750-754; Kaufman, K. M. et al., 1994, “Genomic structure of the human complement protein C8 gamma: homology to the lipocalin gene family,” published in Biochemistry 33 (17), 5162-5166, and the amino acid sequence of C8G (identified by accession no. CAI19172) is disclosed in, e.g., Schreck et al., 2000, “Human complement protein C8 gamma,” Biochim. Biophys. Acta 1482: 199-208; Ortlund et al., “Crystal structure of human complement protein C8 gamma,” Biochemistry 41:7030-7037, each of which is incorporated by reference herein in its entirety.

The nucleotide sequence of C9 (identified by accession no. NM_{—}001737) is disclosed in, e.g., DiScipio, R. G. et al, 1984, “Nucleotide sequence of cDNA and derived amino acid sequence of human complement component C9,” published in Proc. Natl. Acad. Sci. U.S.A. 81 (23), 7298-7302; Stanley, K. K. et al., 1985, “The sequence and topology of human complement component C9,” published in EMBO J. 4 (2), 375-382; Stewart, J. L. et al., 1985, “Analysis of the specific association of the eighth and ninth components of human complement: identification of a direct role for the alpha subunit of C8,” published in Biochemistry 24 (17), 4598-4602, and the amino acid sequence of C9 (identified by accession no. NP_{—}001728) is disclosed in, e.g., DiScipio, R. G. et al., 1984, “Nucleotide sequence of cDNA and derived amino acid sequence of human complement component C9,” published in Proc. Natl. Acad. Sci. U.S.A. 81 (23), 7298-7302; Stanley, K. K. et al., 1985, “The sequence and topology of human complement component C9,” published in EMBO J. 4 (2), 375-382; Stewart, J. L. et al., 1985, “Analysis of the specific association of the eighth and ninth components of human complement: identification of a direct role for the alpha subunit of C8,” published in Biochemistry 24 (17), 4598-4602, each of which is incorporated by reference herein in its entirety.

The nucleotide sequence of SERPINA6 (identified by accession no. NM_{—}001756) is disclosed in, e.g., Rosner, W. et al., 1976, “Identification of corticosteroid-binding globulin in human milk: measurement with a filter disk assay,” published in J. Clin. Endocrinol. Metab. 42 (6), 1064-1073; Agrimonti, F. et al., 1982, “Circadian and circaseptan rhythmicities in corticosteroid-binding globulin (CBG) binding activity of human milk,” published in J. Chromatogr. 9 (3), 281-290; Hammond, G. L. et al., 1986, “Identification and measurement of sex hormone binding globulin (SHBG) and corticosteroid binding globulin (CBG) in human saliva,” published in Acta Endocrinol. 112 (4), 603-608, and the amino acid sequence of SERPINA6 (identified by accession nos. NP 001002236, NP_{—}000286) is disclosed in, e.g., Kurachi, K. et al, 1981, “Cloning and sequence of cDNA coding for alpha 1-antitrypsin,” published in Proc. Natl. Acad. Sci. U.S.A. 78 (11), 6826-6830; Lobermann, H. et al., 1982, “Interaction of human alpha 1-proteinase inhibitor with chymotrypsinogen A and crystallization of a proteolytically modified alpha 1-proteinase inhibitor,” published in Hoppe-Seyler's Z. Physiol. Chem. 363 (11), 1377-1388; Bollen, A. et al., 1983, “Cloning and expression in Escherichia coli of full-length complementary DNA coding for human alpha 1-antitrypsin,” published in DNA 2 (4), 255-264, each of which is incorporated by reference herein in its entirety.

The nucleotide sequence of CD14 (identified by accession no. NM_{—}000591) is disclosed in, e.g., Goyert, S. M. et al., 1988, “The CD14 monocyte differentiation antigen maps to a region encoding growth factors and receptors,” published in Science 239 (4839), 497-500; Ferrero, E. et al., 1988, “Nucleotide sequence of the gene encoding the monocyte differentiation antigen, CD14,” published in Nucleic Acids Res. 16 (9), 4173; Simmons, D. L. et al., 1989, “Monocyte antigen CD14 is a phospholipid anchored membrane protein,” published in Blood 73 (1), 284-289 which is incorporated by reference herein in its entirety.

The nucleotide sequence of CLU (identified by accession no. NM_{—}203339) is disclosed in, e.g., Murphy, B. F. et al., 1988, “SP-40,40, a newly identified normal human serum protein found in the SC5b-9 complex of complement and in the immune deposits in glomerulonephritis,” published in J. Clin. Invest. 81 (6), 1858-1864; Yokoyama, M. et al., 1988, “Isolation and characterization of sulfated glycoprotein from human pancreatic juice,” published in Biochim. Biophys. Acta 967 (1), 34-42; Kirszbaum, L. et al, 1989, “Molecular cloning and characterization of the novel, human complement-associated protein, SP-40,40: a link between the complement and reproductive systems,” published in EMBO J. 8 (3), 711-718, and the amino acid sequence of CLU (identified by accession no. AAP88927) is disclosed in, e.g., James et al., 1991, “Characterization of a human high density lipoprotein-associated protein,” Arterioscler. Thromb. 11:645-652; de Silva et al., 1990, “Purification and characterization of apolipoprotein J,” J. Biol. Chem. 265: 14292-14297, each of which is incorporated by reference herein in its entirety.

The nucleotide sequence of CP (identified by accession no. NM_{—}000096) is disclosed in, e.g., Kingston, I. B. et al., 1977, “Chemical evidence that proteolytic cleavage causes the heterogeneity present in human ceruloplasmin preparations,” published in Proc. Natl. Acad. Sci. U.S.A. 74 (12), 5377-5381; Polosatov, M. V. et al., 1979, “Interaction of synthetic human big gastrin with blood proteins of man and animals,” published in Proc. Natl. Acad. Sci. U.S.A. 26 (2), 154-159; Rask, L. et al., 1983, “Subcellular localization in normal and vitamin A-deficient rat liver of vitamin A serum transport proteins, albumin, ceruloplasmin and class I major histocompatibility antigens,” published in Exp. Cell Res. 143 (1), 91-102, and the amino acid sequence of CP (identified by accession no. NP_{—}000087) is disclosed in, e.g., Kingston, I. B. et al., 1977, “Chemical evidence that proteolytic cleavage causes the heterogeneity present in human ceruloplasmin preparations,” published in Proc. Natl. Acad. Sci. U.S.A. 74 (12), 5377-5381; Polosatov, M. V. et al., 1979, “Interaction of synthetic human big gastrin with blood proteins of man and animals,” published in Proc. Natl. Acad. Sci. U.S.A. 26 (2), 154-159; Rask, L. et al., 1983, “Subcellular localization in normal and vitamin A-deficient rat liver of vitamin A serum transport proteins, albumin, ceruloplasmin and class I major histocompatibility antigens,” published in Exp. Cell Res. 143 (1), 91-102, each of which is incorporated by reference herein in its entirety.

The nucleotide sequence of CRP (identified by accession no. NM_{—}000567) is disclosed in, e.g., Osmand, A. P. et al., 1977, “Characterization of C-reactive protein and the complement subcomponent Clt as homologous proteins displaying cyclic pentameric symmetry (pentraxins),” published in Proc. Natl. Acad. Sci. U.S.A. 74 (2), 739-743; Oliveira, E. B. et al, 1979, “Primary structure of human C-reactive protein,” published in J. Biol. Chem. 254 (2), 489-502; Whitehead, A. S. et al., 1983, “Isolation of human C-reactive protein complementary DNA and localization of the gene to chromosome 1,” published in Science 221 (4605), 69-71, and the amino acid sequence of CRP (identified by accession no. NP_{—}000558) is disclosed in, e.g., Osmand, A. P. et al, 1977, “Characterization of C-reactive protein and the complement subcomponent Clt as homologous proteins displaying cyclic pentameric symmetry (pentraxins),” published in Proc. Natl. Acad. Sci. U.S.A. 74 (2), 739-743; Oliveira, E. B. et al., 1979, “Primary structure of human C-reactive protein,” published in J. Biol. Chem. 254 (2), 489-502; Whitehead, A. S. et al., 1983, “Isolation of human C-reactive protein complementary DNA and localization of the gene to chromosome 1,” published in Science 221 (4605), 69-71, each of which is incorporated by reference herein in its entirety.

The nucleotide sequence and the amino acid sequence of C-reactive protein precursor (respectively identified by accession nos. M11880 and AAB59526) is disclosed in, e.g., Who et al, 1985, “Characterization of genomic and complementary DNA sequence of human C-reactive poretin, comparison with the complementary DNA sequence of serum amyloid P component,” J. Biol. Chem. 260, 13384-13388 which is incorporated by reference herein in its entirety.

The nucleotide sequence of F2 (identified by accession no. NM_{—}000506) is disclosed in, e.g., Bergmann et al., 1982, “Receptor-bound thrombin is not internalized through coated pits in mouse embryo cells,” published in J. Cell. Biochem. 20 (3), 247-258; Degen et al., 1983, “Characterization of the complementary deoxyribonucleic acid and gene coding for human prothrombin,” published in Biochemistry 22 (9), 2087-2097; Wicki, A. N. et al., 1985, “Structure and function of platelet membrane glycoproteins Ib and V. Effects of leukocyte elastase and other proteases on platelets response to von Willebrand factor and thrombin,” published in Eur. J. Biochem. 153 (1), 1-11, and the amino acid sequence of F2 (identified by accession no. AAL77436) is disclosed in Walz et al., 1972, “Amino Acid Sequence of human prothrombin fragments 1 and 2,” Proc. Natl. Acad. Sci. U.S.A. 74:1969-1972; Butkowski et al., 1977, “Primary structure of human prethrombin 2 and alpha-thrombin,” J. Biol. Chem. 252: 4942-4957, each of which is incorporated by reference herein in its entirety.

The nucleotide sequence of F9 (identified by accession no. NM_{—}000133) is disclosed in, e.g., Davie et al., 1975, “Basic mechanisms in blood coagulation,” published in Annu. Rev. Biochem. 44, 799-829; Gentry, P. A., 1977, “Interaction of heparin with canine coagulation proteins: in vivo and in vitro studies,” published in Can. J. Comp. Med. 41 (4), 396-403; Choo, K. H. et al., 1982, “Molecular cloning of the gene for human anti-haemophilic factor IX,” published in Nature 299 (5879), 178-180, and the amino acid sequence of F9 (identified by accession no. NP_{—}000124) is disclosed in, e.g., Scherer Davie, E. W. et al., 1975, “Basic mechanisms in blood coagulation,” published in Annu. Rev. Biochem. 44, 799-829; Gentry, P. A., 1977, “Interaction of heparin with canine coagulation proteins: in vivo and in vitro studies,” published in Can. J. Comp. Med. 41 (4), 396-403; Choo, K. H. et al., 1982, “Molecular cloning of the gene for human anti-haemophilic factor IX,” published in Nature 299 (5879), 178-180, each of which is incorporated by reference herein in its entirety.

The nucleotide sequence of FGA (identified by accession no. BC070246) is disclosed in, e.g., Strausberg et al., 2002, “Generation and initial analysis of more than 15,000 full-length human and mouse cDNA sequences,” published in Proc. Natl. Acad. Sci. U.S.A. 99 (26), 16899-16903, and the amino acid sequence of FGA (identified by accession no. BAC55116) is disclosed in, e.g., Hamasaki, N., 2002, Direct Submission, Naotaka Hamasaki, Kyushu University Hospital, Department of clinical chemistry and laboratory; 3-1-1 maidasi, Higasi-ku Fukuokasi, Fukuoka 812-8582, Japan, Watanabe, K. et al, unpublished, “Identification of simultaneous mutation of fibrinogen alpha; chain and protain C genes in a Japanese kindred,” each of which is incorporated by reference herein in its entirety.

The nucleotide sequence of FGB (identified by accession no. NM_{—}005141) is disclosed in, e.g., Tarakhovskii et al., 1979, “Temperature-dependent changes in the profile of the sarcoplasmic reticulum membrane hydrophobic zones,” published in Biokhimiia 44 (5), 897-902; Weinger, R. S. et al., 1980, “Fibrinogen Houston: a dysfibrinogen exhibiting defective fibrin monomer aggregation and alpha-chain cross-linkages,” published in Am. J. Hematol. 9 (3), 237-248; Chung, D. W. et al., 1983, “Characterization of complementary deoxyribonucleic acid and genomic deoxyribonucleic acid for the beta chain of human fibrinogen,” published in Biochemistry 22 (13), 3244-3250, and the amino acid sequence of FGB (identified by accession no. AAA18024) is disclosed in, e.g., Chung, D. W. et al., 1991, “Nucleotide sequences of the three genes coding for human fibrinogen”, Fibrinogen, Thrombosis, Coagulation and Fibrinolysis: 39-48; Plenum Press, New York, each of which is incorporated by reference herein in its entirety.

The nucleotide sequence of FGG (identified by accession no. NM_{—}000509) is disclosed in, e.g., Olaisen, B. et al, 1982, “Fibrinogen gamma chain locus is on chromosome 4 in man,” published in Hum. Genet. 61 (1), 24-26, Hawiger, J. et al., 1982, “gamma and alpha chains of human fibrinogen possess sites reactive with human platelet receptors,” published in Proc. Natl. Acad. Sci. U.S.A. 79 (6), 2068-2071; Chung, D. W. et al., 1983, “Characterization of a complementary deoxyribonucleic acid coding for the gamma chain of human fibrinogen,” published in Biochemistry 22 (13), 3250-3256, and the amino acid sequence of FGG (identified by accession no. AAB59531) is disclosed in, e.g., Rixon, M. W. et al., 1985, “Nucleotide sequence of the gene for the gamma chain of human fibrinogen,” published in Biochemistry 24 (8), 2077-2086, each of which is incorporated by reference herein in its entirety.

The nucleotide sequence of FLNA (identified by accession no. NM_{—}001456) is disclosed in, e.g., Wallach, D. et al., 1978, “Cyclic AMP-dependent phosphorylation of the actin-binding protein filamin,” published in Proc. Natl. Acad. Sci. U.S.A. 9, 371-379; Gorlin, J. B. et al, 1990, “Human endothelial actin-binding protein (ABP-280, nonmuscle filamin): a molecular leaf spring,” published in J. Cell Biol. 111 (3), 1089-1105; Maestrini, E. et al., 1990, “Probes for CpG islands on the distal long arm of the human X chromosome are clustered in Xq24 and Xq28,” published in Genomics 8 (4), 664-670, and the amino acid sequence of FLNA (identified by accession no. CA143227) is disclosed in, e.g., Heath, P., 2002, Direct Submission, Wellcome Trust Sanger Institute, Hinxton, Cambridgeshire, CB10 1SA, UK, each of which is incorporated by reference herein in its entirety.

The nucleotide sequence of FN1 (identified by accession no. BT006856) is disclosed in, e.g., Kalnine et al., 2003, Direct Submission, BD Biosciences Clontech, 1020 East Meadow Circle, Palo Alto, Calif. 94303, USA; Kalnine, N. et al., unpublished, “Cloning of human full-length CDSs in BD Creator™ System Donor vector,” and the amino acid sequence of FN1 (identified by accession no. BAD52437) is disclosed in, e.g., Kato, 2004, Direct Submission, Seishi Kato, National Rehabilitation Center for Persons with Disabilities, Research Institute, Department of Rehabilitation Engineering; 4-1 Namiki, Tokorozawa, Saitama 359-8555, Japan; Kato, 2004, “Human full-length cDNA starting with the capped site sequence,” Published only in database, each of which is incorporated by reference herein in its entirety.

The nucleotide sequence of GC (identified by accession no. NM_{—}000583) is disclosed in, e.g., Mikkelsen, M. et al, 1977, “Possible localization of Gc-System on chromosome 4. Loss of long arm 4 material associated with father-child incompatibility within the Gc-System,” published in Hum. Hered. 27 (2), 105-107; Constans, J. et al., 1981, “Binding of the apo and holo forms of the serum vitamin D-binding protein to human lymphocyte cytoplasm and membrane by indirect immunofluorescence,” published in Immunol. Lett. 3 (3), 159-162; Wooten, M. W. et al., 1985, “Identification of a major endogenous substrate for phospholipid/Ca2+-dependent kinase in pancreatic acini as Gc (vitamin D-binding protein),” published in FEBS Lett. 191 (1), 97-101, and the amino acid sequence of GC (identified by accession no. NP_{—}000574) is disclosed in, e.g., Mikkelsen, M. et al., 1977, “Possible localization of Gc-System on chromosome 4. Loss of long arm 4 material associated with father-child incompatibility within the Gc-System,” published in Hum. Hered. 27 (2), 105-107; Constans, J. et al., 1981, “Binding of the apo and holo forms of the serum vitamin D-binding protein to human lymphocyte cytoplasm and membrane by indirect immunofluorescence,” published in Immunol. Lett. 3 (3), 159-162; Wooten, M. W. et al, 1985, “Identification of a major endogenous substrate for phospholipid/Ca2+-dependent kinase in pancreatic acini as Gc (vitamin D-binding protein),” published in FEBS Lett. 191 (1), 97-101, each of which is incorporated by reference herein in its entirety.

The nucleotide sequence of GSN (identified by accession no. BC026033) is disclosed in, e.g., Strausberg et al., 2002, “Generation and initial analysis of more than 15,000 full-length human and mouse cDNA sequences,” published in Proc. Natl. Acad. Sci. U.S.A. 99 (26), 16899-16903 which is incorporated by reference herein in its entirety.

The nucleotide sequence of HBB (identified by accession no. NM_{—}000518) is disclosed in, e.g., Marotta, C. A. et al, 1976, “Nucleotide sequence analysis of coding and noncoding regions of human beta-globin mRNA,” published in Prog. Nucleic Acid Res. Mol. Biol. 19, 165-175; Proudfoot, N.J., 1977, “Complete 3′ noncoding region sequences of rabbit and human beta-globin messenger RNAs, published in Cell 10 (4), 559-570; Marotta, C. A. et al, 1977, “Human beta-globin messenger RNA. III. Nucleotide sequences derived from complementary DNA,” published in J. Biol. Chem. 252 (14), 5040-5053, and the amino acid sequence of HBB (identified by accession no. AAD19696) is disclosed in, e.g., Braunitzer et al., 1961, “The constitution of normal adult human haemoglobin,” Hoppe-Seyler's Z. Physiol. Chem. 325:283-286, each of which is incorporated by reference herein in its entirety.

The nucleotide sequence of SERPIND1 (identified by accession no. NM_{—}000185) is disclosed in, e.g., Ragg, H., 1986, “A new member of the plasma protease inhibitor gene family,” published in Nucleic Acids Res. 14 (2), 1073-1088; Inhorn, R. C. et al., 1986, “Isolation and characterization of a partial cDNA clone for heparin cofactor III,” published in Biochem. Biophys. Res. Commun. 137 (1), 431-436; Hortin, G. et al., 1986, “Identification of two sites of sulfation of human heparin cofactor II,” published in J. Biol. Chem. 261 (34), 15827-15830, and the amino acid sequence of SERPIND1 (identified by accession no. CAG30459) is disclosed in, e.g., Collins et al., 2004, “A genome annotation-driven approach to cloning the human ORFeome,” published in Genome Biol. 5 (10), R84, each of which is incorporated by reference herein in its entirety.

The nucleotide sequence of HP (identified by accession no. BC107587) is disclosed in, e.g. Strausberg et al., 2002, “Generation and initial analysis of more than 15,000 full-length human and mouse cDNA sequences,” published in Proc. Natl. Acad. Sci. U.S.A. 99 (26), 16899-16903, and the amino acid sequence of HP (identified by accession no. NP_{—}005134) is disclosed in, e.g., Kazim, A. L. et al, 1980, “Haemoglobin binding with haptoglobin. Unequivocal demonstration that the beta-chains of human haemoglobin bind to haptoglobin, published in Biochem. J. 185 (1), 285-287; Eaton et al., 1982, “Haptoglobin: a natural bacteriostat,” published in Science 215 (4533), 691-693; Costanzo et al., Sequence of human haptoglobin cDNA: evidence that the alpha and beta subunits are coded by the same mRNA,” published in Nucleic Acids Res. 11 (17), 5811-5819, each of which is incorporated by reference herein in its entirety.

The nucleotide sequence of HPX (identified by accession no. NM_{—}000613) is disclosed in, e.g., Morgan, W. T. et al., 1978, “Interaction of rabbit hemopexin with bilirubin,” published in Biochim. Biophys. Acta 532 (1), 57-64; Takahashi, N. et al., 1984, “Structure of human hemopexin: O-glycosyl and N-glycosyl sites and unusual clustering of tryptophan residues,” published in Proc. Natl. Acad. Sci. U.S.A. 81 (7), 2021-2025; Frantikova, V. et al., Amino acid sequence of the N-terminal region of human hemopexin,” published in FEBS Lett. 178 (2), 213-216, and the amino acid sequence of HPX (identified by accession no. NP_{—}000604) is disclosed in, e.g., Morgan, W. T. et al, 1978, “Interaction of rabbit hemopexin with bilirubin,” published in Biochim. Biophys. Acta 532 (1), 57-64; Takahashi, N. et al., 1984, “Structure of human hemopexin: O-glycosyl and N-glycosyl sites and unusual clustering of tryptophan residues,” published in Proc. Natl. Acad. Sci. U.S.A. 81 (7), 2021-2025; Frantikova, V. et al., Amino acid sequence of the N-terminal region of human hemopexin,” published in FEBS Lett. 178 (2), 213-216, each of which is incorporated by reference herein in its entirety.

The nucleotide sequence of HRG (identified by accession no. NM_{—}000412) is disclosed in, e.g., Heimburger, N. et al., 1972, “Human serum proteins with high affinity to carboxymethylcellulose. II. Physico-chemical and immunological characterization of a histidine-rich 3,8S-2-glycoportein (CM-protein I),” published in Hoppe-Seyler's Z. Physiol. Chem. 353 (7), 1133-1140; Silverstein, R. L. et al., 1984, “Complex formation of platelet thrombospondin with plasminogen. Modulation of activation by tissue activator,” published in J. Clin. Invest. 74 (5), 1625-1633; Leung, L. L., 1986, “Interaction of histidine-rich glycoprotein with fibrinogen and fibrin,” published in J. Clin. Invest. 77 (4), 1305-1311, and the amino acid sequence of HRG (identified by accession no. NP_{—}000403) is disclosed in, e.g., Heimburger, N. et al., 1972, “Human serum proteins with high affinity to carboxymethylcellulose. II. Physico-chemical and immunological characterization of a histidine-rich 3,8S-2-glycoportein (CM-protein I),” published in Hoppe-Seyler's Z. Physiol. Chem. 353 (7), 1133-1140; Silverstein, R. L. et al., 1984, “Complex formation of platelet thrombospondin with plasminogen. Modulation of activation by tissue activator,” published in J. Clin. Invest. 74 (5), 1625-1633; Leung, L. L., 1986, “Interaction of histidine-rich glycoprotein with fibrinogen and fibrin,” published in J. Clin. Invest. 77 (4), 1305-1311, each of which is incorporated by reference herein in its entirety.

The nucleotide sequence of IF (identified by accession no. NM_{—}000204) is disclosed in, e.g., Catterall, C. F. et al., 1987, “Characterization of primary amino acid sequence of human complement control protein factor I from an analysis of cDNA clones,” published in Biochem. J. 242 (3), 849-856; Goldberger, G. et al., 1987, “Human complement factor I: analysis of cDNA-derived primary structure and assignment of its gene to chromosome 4” published in J. Biol. Chem. 262 (21), 10065-10071; Shiang, R. et al., 1989, “Mapping of the human complement factor I gene to 4q25,” published in Genomics 4 (1), 82-86, and the amino acid sequence of IF (identified by accession no. NP_{—}000195) is disclosed in, e.g., Catterall, C. F. et al, 1987, “Characterization of primary amino acid sequence of human complement control protein factor I from an analysis of cDNA clones,” published in Biochem. J. 242 (3), 849-856; Goldberger, G. et al., 1987, “Human complement factor I: analysis of cDNA-derived primary structure and assignment of its gene to chromosome 4” published in J. Biol. Chem. 262 (21), 10065 10071; Shiang, R. et al., 1989, “Mapping of the human complement factor I gene to 4q25,” published in Genomics 4 (1), 82-86, each of which is incorporated by reference herein in its entirety.

The nucleotide sequence of IGFALS (identified by accession no. NM_{—}004970) is disclosed in, e.g., Baxter, R. C. et al., 1989, “High molecular weight insulin-like growth factor binding protein complex. Purification and properties of the acid-labile subunit from human serum,” published I J. Biol. Chem. 264 (20), 11843-11848; Leong, S. R. et al., 1992, “Structure and functional expression of the acid-labile subunit of the insulin-like growth factor-binding protein complex,” published in Mol. Endocrinol. 6 (6), 870-876; Dai, J. et al., 1992, “Molecular cloning of the acid-labile subunit of the rat insulin-like growth factor binding protein complex,” published in Biochem. Biophys. Res. Commun. 188 (1), 304-309, and the amino acid sequence of IGFALS (identified by accession no. NP_{—}004691) is disclosed in, e.g., Kubisch, C. et al., 1999, “KCNQ4, a novel potassium channel expressed in sensory outer hair cells, is mutated in dominant deafness,” published in Cell 96 (3), 437-446; Selyanko, A. A. et al., 2000, “Inhibition of KCNQ1-4 potassium channels expressed in mammalian cells via MI muscarinic acetylcholine receptors,” published in J. Physiol. (Lond.) 522 PT 3, 349-355; Sogaard, R. et al., 2001, “KCNQ4 channels expressed in mammalian cells: functional characteristics and pharmacology,” published in Am. J. Physiol., Cell Physiol. 280 (4), C859-C866, each of which is incorporated by reference herein in its entirety.

The nucleotide sequence of ITGA1 (identified by accession no. NM_{—}181501) is disclosed in, e.g., Takada et al., 1987, “The very late antigen family of heterodimers is part of a superfamily of molecules involved in adhesion and embryogenesis,” published in Proc. Natl. Acad. Sci. U.S.A. 84 (10), 3239-3243; MacDonald, T. T. et al., 1990, “Increased expression of laminin/collagen receptor (VLA-1) on epithelium of inflamed human intestine,” published in J. Clin. Pathol. 43 (4), 313-315; Tawil, N. J. et al., 1990, “Alpha 1 beta 1 integrin heterodimer functions as a dual laminin/collagen receptor in neural cells,” published in Biochemistry 29 (27), 6540-6544, and the amino acid sequence of ITGA1 (identified by accession no. NP_{—}852478) is disclosed in, e.g., Scherer Takada, Y. et al., 1987, “The very late antigen family of heterodimers is part of a superfamily of molecules involved in adhesion and embryogenesis,” published in Proc. Natl. Acad. Sci. U.S.A. 84 (10), 3239-3243; MacDonald, T. T. et al., 1990, “Increased expression of laminin/collagen receptor (VLA-1) on epithelium of inflamed human intestine,” published in J. Clin. Pathol. 43 (4), 313-315; Tawil, N. J. et al., 1990, “Alpha 1 beta 1 integrin heterodimer functions as a dual laminin/collagen receptor in neural cells,” published in Biochemistry 29 (27), 6540-6544, each of which is incorporated by reference herein in its entirety.

The nucleotide sequence of ITIH1 (identified by accession no. BC109115) is disclosed in, e.g., NIH MGC Project, 2005, Direct Submission, National Institutes of Health, Mammalian Gene Collection (MGC), Bethesda, Md. 20892-2590, USA, and the amino acid sequence of ITIH1 (identified by accession nos. NP_{—}002206, NP_{—}032432) is disclosed in, e.g., Salier, J. P. et al., 1987, “Isolation and characterization of cDNAs encoding the heavy chain of human inter-alpha-trypsin inhibitor (1 alpha TI): unambiguous evidence for multipolypeptide chain structure of 1 alpha TI,” published in Proc. Natl. Acad. Sci. U.S.A. 84 (23), 8272-8276; Diarra-Mehrpour, M. et al., 1989, “Human plasma inter-alpha-trypsin inhibitor is encoded by four genes on three chromosomes,” published in Eur. J. Biochem. 179 (1), 147-154; Gebhard, W. et al, 1989, “Two out of the three kinds of subunits of inter-alpha-trypsin inhibitor are structurally related,” published in Eur. J. Biochem. 181 (3), 571-576, each of which is incorporated by reference herein in its entirety.

The nucleotide sequence of ITIH2 (identified by accession no. NM_{—}002216) is disclosed in, e.g., Salier, J. P. et al., 1987, “Isolation and characterization of cDNAs encoding the heavy chain of human inter-alpha-trypsin inhibitor (1 alpha TI): unambiguous evidence for multipolypeptide chain structure of 1 alpha TI,” published in Proc. Natl. Acad. Sci. U.S.A. 84 (23), 8272-8276; Gebhard, W. et al, 1988, “Complementary DNA and derived amino acid sequence of the precursor of one of the three protein components of the inter-alpha-trypsin inhibitor complex,” published in FEBS Lett. 229 (1), 63-67; Salier, J. P. et al., 1988, “Human inter-alpha-trypsin inhibitor. Isolation and characterization of heavy (H) chain cDNA clones coding for a 383 amino-acid sequence of the H chain,” published in Biol. Chem. Hoppe-Seyler 369 SUPPL, 15-18, and the amino acid sequence of ITIH2 (identified by accession no. NP_{—}002207) is disclosed in, e.g., Salier, J. P. et al., 1987, “Isolation and characterization of cDNAs encoding the heavy chain of human inter-alpha-trypsin inhibitor (1 alpha TI): unambiguous evidence for multipolypeptide chain structure of I alpha TI,” published in Proc. Natl. Acad. Sci. U.S.A. 84 (23), 8272-8276; Gebhard, W. et al., 1988, “Complementary DNA and derived amino acid sequence of the precursor of one of the three protein components of the inter-alpha-trypsin inhibitor complex,” published in FEBS Lett. 229 (1), 63-67; Salier, J. P. et al, 1988, “Human inter-alpha-trypsin inhibitor. Isolation and characterization of heavy (H) chain cDNA clones coding for a 383 amino-acid sequence of the H chain,” published in Biol. Chem. Hoppe-Seyler 369 SUPPL, 15-18, each of which is incorporated by reference herein in its entirety.

The nucleotide sequence of ITIH4 (identified by accession no. NM_{—}002218) is disclosed in, e.g., To be, T. et al., 1995, “Mapping of human inter-alpha-trypsin inhibitor family heavy chain-related protein gene (ITIHL1) to human chromosome 3p21->p14,” published in Cytogenet. Cell Genet. 71 (3), 296-298; Saguchi, K. et al., 1995, “Cloning and characterization of cDNA for inter-alpha-trypsin inhibitor family heavy chain-related protein (1HRP), a novel human plasma glycoprotein,” published in J. Biochem. 117 (1), 14-18; Nishimura, H. et al., 1995, “cDNA and deduced amino acid sequence of human PK-120, a plasma kallikrein-sensitive glycoprotein,” published in FEBS Lett. 357 (2), 207-211, and the amino acid sequence of ITIH4 (identified by accession no. NP_{—}002209) is disclosed in, e.g., To be, T. et al., 1995, “Mapping of human inter-alpha-trypsin inhibitor family heavy chain-related protein gene (ITIHL1) to human chromosome 3p21->p14,” published in Cytogenet. Cell Genet. 71 (3), 296-298; Saguchi et al., 1995, “Cloning and characterization of cDNA for inter-alpha-trypsin inhibitor family heavy chain-related protein (1HRP), a novel human plasma glycoprotein,” published in J. Biochem. 117 (1), 14-18; Nishimura, H. et al., 1995, “cDNA and deduced amino acid sequence of human PK-120, a plasma kallikrein-sensitive glycoprotein,” published in FEBS Lett. 357 (2), 207-211, each of which is incorporated by reference herein in its entirety.

The nucleotide sequence of KLKB1 (identified by accession no. NM_{—}000892) is disclosed in, e.g., Aznar, J. A. et al, 1978, “Fletcher factor deficiency: report of a new family,” published in J. Biol. Chem. 21 (2), 94-98; Thompson, R. E. et al., “Studies of binding of prekallikrein and Factor XI to high molecular weight kininogen and its light chain,” published in Proc. Natl. Acad. Sci. U.S.A. 76 (10), 4862-4866; Chung, D. W., et al., “Human plasma prekallikrein, a zymogen to a serine protease that contains four tandem repeats,” published in Biochemistry 25 (9), 2410-2417, and the amino acid sequence of KLKB1 (identified by accession no. NP_{—}000883) is disclosed in, e.g., Aznar, J. A. et al., 1978, “Fletcher factor deficiency: report of a new family,” published in J. Biol. Chem. 21 (2), 94-98; Thompson, R. E. et al., “Studies of binding of prekallikrein and Factor XI to high molecular weight kininogen and its light chain,” published in Proc. Natl. Acad. Sci. U.S.A. 76 (10), 4862-4866; Chung, D. W., et al., “Human plasma prekallikrein, a zymogen to a serine protease that contains four tandem repeats,” published in Biochemistry 25 (9), 2410-2417, each of which is incorporated by reference herein in its entirety.

The nucleotide sequence of KNG1 (identified by accession no. NM_{—}000893) is disclosed in, e.g., Colman, R. W. et al, 1975, “Williams trait. Human kininogen deficiency with diminished levels of plasminogen proactivator and prekallikrein associated with abnormalities of the Hageman factor-dependent pathways,” published in J. Clin. Invest. 56 (6), 1650-1662; Thompson, R. E. et al., 1979, “Studies of binding of prekallikrein and Factor XI to high molecular weight kininogen and its light chain,” published in Proc. Natl. Acad. Sci. U.S.A. 76 (10), 4862-4866; Kerbiriou, D. M. et al., 1979, “Human high molecular weight kininogen. Studies of structure-function relationships and of proteolysis of the molecule occurring during contact activation of plasma,” published in J. Biol. Chem. 254 (23), 12020-12027, and the amino acid sequence of KNG1 (identified by accession no. NP_{—}000884) is disclosed in, e.g., Colman, R. W. et al., 1975, “Williams trait. Human kininogen deficiency with diminished levels of plasminogen proactivator and prekallikrein associated with abnormalities of the Hageman factor-dependent pathways,” published in J. Clin. Invest. 56 (6), 1650-1662; Thompson, R. E. et al., 1979, “Studies of binding of prekallikrein and Factor XI to high molecular weight kininogen and its light chain,” published in Proc. Natl. Acad. Sci. U.S.A. 76 (10), 4862-4866; Kerbiriou, D. M. et al., 1979, “Human high molecular weight kininogen. Studies of structure-function relationships and of proteolysis of the molecule occurring during contact activation of plasma,” published in J. Biol. Chem. 254 (23), 12020-12027, each of which is incorporated by reference herein in its entirety.

The nucleotide sequence of KRT1 (identified by accession no. BC063697) is disclosed in, e.g., Strausberg et al., 2002, “Generation and initial analysis of more than 15,000 full-length human and mouse cDNA sequences,” published in Proc. Natl. Acad. Sci. U.S.A. 99 (26), 16899-16903, and the amino acid sequence of KRT1 (identified by accession no. NP_{—}000412) is disclosed in, e.g., Darmon, M. Y. et al., 1987, “Sequence of a cDNA encoding human keratin No 10 selected according to structural homologies of keratins and their tissue-specific expression,” published in Mol. Biol. Rep. 12 (4), 277-283; Zhou, X. M. et al., 1988, “The complete sequence of the human intermediate filament chain keratin 10. Subdomainal divisions and model for folding of end domain sequences,” published in J. Biol. Chem. 263 (30), 15584-15589, each of which is incorporated by reference herein in its entirety.

The nucleotide sequence of LGALS3BP (identified by accession nos. NM_{—}005567, BC015761, BC002403, BC002998) is disclosed in, e.g., Rosenberg, I. et al., 1991, “Mac-2-binding glycoproteins. Putative ligands for a cytosolic beta-galactoside lectin,” published in J. Biol. Chem. 266 (28), 18731-18736; Koths, K. et al., 1993, “Cloning and characterization of a human Mac-2-binding protein, a new member of the superfamily defined by the macrophage scavenger receptor cysteine-rich domain,” published in J. Biol. Chem. 268 (19), 14245-14249; Ullrich, A. et al., 1994, “The secreted tumor-associated antigen 90K is a potent immune stimulator,” published in J. Biol. Chem. 269 (28), 18401-18407; Strausberg et al., 2002, “Generation and initial analysis of more than 15,000 full-length human and mouse cDNA sequences,” published in Proc. Natl. Acad. Sci. U.S.A. 99 (26), 16899-16903; and the amino acid sequence of LGALS3BP (identified by accession no. NP_{—}005558) is disclosed in, e.g., Rosenberg, I. et al., 1991, “Mac-2-binding glycoproteins. Putative ligands for a cytosolic beta-galactoside lectin,” published in J. Biol. Chem. 266 (28), 18731-18736; Koths, K. et al., 1993, “Cloning and characterization of a human Mac-2-binding protein, a new member of the superfamily defined by the macrophage scavenger receptor cysteine-rich domain,” published in J. Biol. Chem. 268 (19), 14245-14249; Ullrich, A. et al., 1994, “The secreted tumor-associated antigen 90K is a potent immune stimulator,” published in J. Biol. Chem. 269 (28), 18401-18407, each of which is incorporated by reference herein in its entirety.

The nucleotide sequence of LPA (identified by accession no. NM_{—}005577) is disclosed in, e.g., McLean, J. W. et al., 1987, “cDNA sequence of human apolipoprotein (a) is homologous to plasminogen,” published in Nature 330 (6144), 132-137; Frank, S. L. et al., 1998, “The apolipoprotein (a) gene resides on human chromosome 6q26-27, in close proximity to the homologous gene for plasminogen,” published in Hum. Genet. 79 (4), 352-356; Salonen, E. M. et al., 1989, “Lipoprotein (a) binds to fibronectin and has serine proteinase activity capable of cleaving it,” published in EMBO J. 8 (13), 4035-4040, and the amino acid sequence of LPA (identified by accession no. NP_{—}005568) is disclosed in, e.g., McLean, J. W. et al., 1987, “cDNA sequence of human apolipoprotein (a) is homologous to plasminogen,” published in Nature 330 (6144), 132-137; Frank, S. L. et al., 1998, “The apolipoprotein (a) gene resides on human chromosome 6q26-27, in close proximity to the homologous gene for plasminogen,” published in Hum. Genet. 79 (4), 352-356; Salonen, E. M. et al., 1989, “Lipoprotein (a) binds to fibronectin and has serine proteinase activity capable of cleaving it,” published in EMBO J. 8 (13), 4035-4040, each of which is incorporated by reference herein in its entirety.

The nucleotide sequence of MLL (identified by accession no. NM_{—}005934) is disclosed in, e.g., Tkachuk, D. C. et al., 1992, “Involvement of a homolog of Drosophila trithorax by 11q23 chromosomal translocations in acute leukemias,” published in Cell 71 (4), 691-700; Yamamoto, K. et al., 1993, “Two distinct portions of LTG19/ENL at 19p13 are involved in t(11;19) leukemia,” published in Oncogene 8 (10), 2617-2625; Rubnitz, J. E. et al., 1994, “ENL, the gene fused with HRX in t(11;19) leukemias, encodes a nuclear protein with transcriptional activation potential in lymphoid and myeloid cells,” published in Blood 84 (6), 1747-1752, and the amino acid sequence of MLL (identified by accession no. NP_{—}005924) is disclosed in, e.g., Ziemin-van der Poel, S. et al., 1991, “Identification of a gene, MLL, that spans the breakpoint in 11q23 translocations associated with human leukemias,” published in Proc. Natl. Acad. Sci. U.S.A. 88 (23), 10735-10739; Djabali, M. et al., 1992, “A trithorax-like gene is interrupted by chromosome 11q23 translocations in acute leukaemias,” published in Nat. Genet. 2 (2), 113-118; Tkachuk, D. C. et al., 1992, “Involvement of a homolog of Drosophila trithorax by 11q23 chromosomal translocations in acute leukemias,” published in Cell 71 (4), 691 700, each of which is incorporated by reference herein in its entirety.

The nucleotide sequence of MRC1 (identified by accession no. NM_{—}002438) is disclosed in, e.g., Taylor, M. E. et al., 1990, “Primary structure of the mannose receptor contains multiple motifs resembling carbohydrate-recognition domains,” published in J. Biol. Chem. 265 (21), 12156-12162; Ezekowitz, R. A. et al., 1990, Molecular characterization of the human macrophage mannose receptor: demonstration of multiple carbohydrate recognition-like domains and phagocytosis of yeasts in Cos-1 cells,” published in J. Exp. Med. 172 (6), 1785-1794; Taylor, M. E. et al., 1992, “Contribution to ligand binding by multiple carbohydrate-recognition domains in the macrophage mannose receptor,” published in J. Biol. Chem. 267 (3), 1719-1726, and the amino acid sequence of MRC1 (identified by accession no. NP_{—}002429) is disclosed in, e.g., Taylor, M. E. et al., 1990, “Primary structure of the mannose receptor contains multiple motifs resembling carbohydrate-recognition domains,” published in J. Biol. Chem. 265 (21), 12156-12162; Ezekowitz, R. A. et al, 1990, “Molecular characterization of the human macrophage mannose receptor: demonstration of multiple carbohydrate recognition-like domains and phagocytosis of yeasts in Cos-1 cells,” published in J. Exp. Med. 172 (6), 1785-1794; Taylor, M. E. et al, 1992, “Contribution to ligand binding by multiple carbohydrate-recognition domains in the macrophage mannose receptor,” published in J. Biol. Chem. 267 (3), 1719-1726, each of which is incorporated by reference herein in its entirety.

The nucleotide sequence of MYL2 (identified by accession no. NM_{—}000432) is disclosed in, e.g., Dalla Libera, L. et al., 1989, “Isolation and nucleotide sequence of the cDNA encoding human ventricular myosin light chain 2,” published in Nucleic Acids Res. 17 (6), 2360; Macera, M. J. et al., “Localization of the gene coding for ventricular myosin regulatory light chain (MYL2) to human chromosome 12q23-q24.3,” published in Genomics 13 (3), 829-831; Wadgaonkar, R. et al., 1993, “Interaction of a conserved peptide domain in recombinant human ventricular myosin light chain-2 with myosin heavy chain,” published in Cell. Mol. Biol. Res. 39 (1), 13-26, and the amino acid sequence of MYL2 (identified by accession no. AAH31006) is disclosed in, e.g., Strausberg, R. L., “Generation and initial analysis of more than 15,000 full-length human and mouse cDNA sequences,” Proc. Natl. Acad. Sci. U.S.A. 99 (26), 16899-16903 (2002), each of which is incorporated by reference herein in its entirety.

The nucleotide sequence of MYO6 (identified by accession no. NM_{—}004999) is disclosed in, e.g., Bement, W. M. et al., 1994, “Identification and overlapping expression of multiple unconventional myosin genes in vertebrate cell types,” published in Proc. Natl. Acad. Sci. U.S.A. 91 (14), 6549-6553; Avraham, K. B. et al., 1995, “The mouse Snell's waltzer deafness gene encodes an unconventional myosin required for structural integrity of inner ear hair cells,” published in Nat. Genet. 11 (4), 369-375; Avraham, K. B. et al., 1997, “Characterization of unconventional MYO6, the human homologue of the gene responsible for deafness in Snell's waltzer mice,” published in Hum. Mol. Genet. 6 (8), 1225-1231, and the amino acid sequence of KCTD7 (identified by accession no. NP_{—}004990) is disclosed in, e.g., Bement, W. M. et al., 1994, “Identification and overlapping expression of multiple unconventional myosin genes in vertebrate cell types,” published in Proc. Natl. Acad. Sci. U.S.A. 91 (14), 6549-6553; Avraham, K. B. et al., 1995, “The mouse Snell's waltzer deafness gene encodes an unconventional myosin required for structural integrity of inner ear hair cells,” published in Nat. Genet. 11 (4), 369-375; Avraham, K. B. et al., 1997, “Characterization of unconventional MYO6, the human homologue of the gene responsible for deafness in Snell's waltzer mice,” published in Hum. Mol. Genet. 6 (8), 1225-1231, each of which is incorporated by reference herein in its entirety.

The nucleotide sequence of ORM1 (identified by accession no. NM_{—}000607) is disclosed in, e.g., Schmid, K. et al., 1974, “The disulfide bonds of alpha1-acid glycoprotein,” published in Biochemistry 13 (13), 2694-2697; Mbuyi, J. M. et al., 1982, “Plasma proteins in human cortical bone: enrichment of alpha 2 HS-glycoprotein, alpha 1 acid-glycoprotein, and IgE,” published in Calcif. Tissue Int. 34 (3), 229-231; Dente, L. et al., 1985, “Structure of the human alpha 1-acid glycoprotein gene: sequence homology with other human acute phase protein genes,” published in Nucleic Acids Res. 13 (11), 3941-3952, and the amino acid sequence of ORM1 (identified by accession no. CA116859) is disclosed in, e.g., Schmid et al., 1973, “Structure of alpha 1-acid glycoprotein” Biochemistry 12:2711-2724, each of which is incorporated by reference herein in its entirety.

The nucleotide sequence of SERPINF1 (identified by accession nos. NM_{—}002615, BC013984) is disclosed in, e.g., Steele, F. R. et al., 1993, “Pigment epithelium-derived factor: neurotrophic activity and identification as a member of the serine protease inhibitor gene family,” published in Proc. Natl. Acad. Sci. U.S.A. 90 (4), 1526-1530; Pignolo, R. J. et al., 1993, “Senescent WI-38 cells fail to express EPC-1, a gene induced in young cells upon entry into the GO state,” published in J. Biol. Chem. 268 (12), 8949-8957; Becerra, S. P. et al., 1993, “Overexpression of fetal human pigment epithelium-derived factor in Escherichia coli. A functionally active neurotrophic factor,” published in J. Biol. Chem. 268 (31), 23148-23156, and the amino acid sequence of SERPINF1 (identified by accession no. AAH13984) is disclosed in, e.g., Petersen et al., 2003, “Pigment-epithelium-derived factor occurs at a physiologically relevant concentration in human blood: purification and characterization,” Biochem J. 374: 199-206, each of which is incorporated by reference herein in its entirety.

The nucleotide sequence of SERPINA1 (identified by accession nos. BC015642, NM_{—}000295) is disclosed in, e.g., NIH MGC Project, 2001, Direct Submission, National Institutes of Health, Mammalian Gene Collection (MGC), Bethesda, Md. 20892-2590, USA; Strausberg, R. L. et al., 2002, “Generation and initial analysis of more than 15,000 full-length human and mouse cDNA sequences,” published in Proc. Natl. Acad. Sci. U.S.A. 99 (26), 16899-16903; Kurachi, K. et al., 1981, “Cloning and sequence of cDNA coding for alpha 1-antitrypsin,” published in Proc. Natl. Acad. Sci. U.S.A. 78 (11), 6826-6830; Lobermann, H. et al., 1982, “Interaction of human alpha 1-proteinase inhibitor with chymotrypsinogen A and crystallization of a proteolytically modified alpha 1-proteinase inhibitor,” published in Hoppe-Seyler's Z. Physiol. Chem. 363 (11), 1377-1388; Bollen, A. et al., 1983, “Cloning and expression in Escherichia coli of full-length complementary DNA coding for human alpha 1-antitrypsin,” published in DNA 2 (4), 255-264, and the amino acid sequence of SERPINA1 (identified by accession nos. NP 001002235, NP_{—}000286) is disclosed in, e.g., Kurachi, K. et al, 1981, “Cloning and sequence of cDNA coding for alpha 1-antitrypsin,” published in Proc. Natl. Acad. Sci. U.S.A. 78 (11), 6826-6830; Lobermann, H. et al., 1982, “Interaction of human alpha 1-proteinase inhibitor with chymotrypsinogen A and crystallization of a proteolytically modified alpha 1-proteinase inhibitor,” published in Hoppe-Seyler's Z. Physiol. Chem. 363 (11), 1377-1388; Bollen, A. et al., 1983, “Cloning and expression in Escherichia coli of full-length complementary DNA coding for human alpha 1-antitrypsin,” published in DNA 2 (4), 255-264, each of which is incorporated by reference herein in its entirety.

The nucleotide sequence of SERPINA4 (identified by accession no. NM_{—}006215) is disclosed in, e.g., Wang, M. Y. et al., 1989, “Human kallistatin, a new tissue kallikrein-binding protein: purification and characterization,” published in Adv. Exp. Med. Biol. 247B, 1-8; Zhou, G. X. et al, 1992, “Kallistatin: a novel human tissue kallikrein inhibitor. Purification, characterization, and reactive center sequence,” published in J. Biol. Chem. 267 (36), 25873-25880; Chai, K. X. et al, 1993, “Kallistatin: a novel human serine proteinase inhibitor. Molecular cloning, tissue distribution, and expression in Escherichia coli,” published in J. Biol. Chem. 268 (32), 24498-24505, and the amino acid sequence of SERPINA4 (identified by accession no. NP_{—}006206) is disclosed in, e.g., Wang, M. Y. et al., 1989, “Human kallistatin, a new tissue kallikrein-binding protein: purification and characterization,” published in Adv. Exp. Med. Biol. 247B, 1-8; Zhou, G. X. et al., 1992, “Kallistatin: a novel human tissue kallikrein inhibitor. Purification, characterization, and reactive center sequence,” published in J. Biol. Chem. 267 (36), 25873-25880; Chai, K. X. et al., 1993, “Kallistatin: a novel human serine proteinase inhibitor. Molecular cloning, tissue distribution, and expression in Escherichia coli,” published in J. Biol. Chem. 268 (32), 24498-24505, each of which is incorporated by reference herein in its entirety.

The nucleotide sequence of SERPINF2 (identified by accession no. BC031592) is disclosed in, e.g., NIH MGC Project, 2002, Direct Submission, National Institutes of Health, Mammalian Gene Collection (MGC), Bethesda, Md. 20892-2590, USA; Strausberg, R. L. et al., 2002, “Generation and initial analysis of more than 15,000 full-length human and mouse cDNA sequences,” published in Proc. Natl. Acad. Sci. U.S.A. 99 (26), 16899-16903, and the amino acid sequence of SERPINF2 (identified by accession no. NP_{—}000925) is disclosed in, e.g., Wiman, B. et al., 1979, “On the mechanism of the reaction between human alpha 2-antiplasmin and plasmin,” published in J. Biol. Chem. 254 (18), 9291-9297; Yoshioka, A. et al, 1982, “Congenital deficiency of alpha 2-plasmin inhibitor in three sisters,” published in Haemostasis 11 (3), 176-184; Brower, M. S. et al., 1982, “Proteolytic cleavage and inactivation of alpha 2-plasmin inhibitor and C1 inactivator by human polymorphonuclear leukocyte elastase,” published in J. Biol. Chem. 257 (16), 9849-9854, each of which is incorporated by reference herein in its entirety.

The nucleotide sequence of PROS1 (identified by accession no. NM_{—}000313) is disclosed in, e.g., Dahlback, B. et al., 1981, “High molecular weight complex in human plasma between vitamin K-dependent protein S and complement component C4b-binding protein,” published in Proc. Natl. Acad. Sci. U.S.A. 78 (4), 2512-2516; Comp, P. C. et al, 1984, “Recurrent venous thromboembolism in patients with a partial deficiency of protein S,” published in N. Engl. J. Med. 311 (24), 1525-1528; Lundwall, A. et al., 1986, “Isolation and sequence of the cDNA for human protein S, a regulator of blood coagulation,” published in Proc. Natl. Acad. Sci. U.S.A. 83 (18), 6716-6720, and the amino acid sequence of PROS1 (identified by accession no. NP_{—}000304) is disclosed in, e.g., Dahlback, B. et al, 1981, “High molecular weight complex in human plasma between vitamin K-dependent protein S and complement component C4b-binding protein,” published in Proc. Natl. Acad. Sci. U.S.A. 78 (4), 2512-2516; Comp, P. C. et al., 1984, “Recurrent venous thromboembolism in patients with a partial deficiency of protein S,” published in N. Engl. J. Med. 311 (24), 1525-1528; Lundwall, A. et al., 1986, “Isolation and sequence of the cDNA for human protein S, a regulator of blood coagulation,” published in Proc. Natl. Acad. Sci. U.S.A. 83 (18), 6716-6720, each of which is incorporated by reference herein in its entirety.

The nucleotide sequence of QSCN6 (identified by accession no. NM_{—}002826) is disclosed in, e.g. Coppock, D. L. et al, 1993, “Preferential gene expression in quiescent human lung fibroblasts” published in Cell Growth Differ. 4 (6), 483-493 (1993); Hoober, K. L., et al., 1999, “Homology between egg white sulfhydryl oxidase and quiescin Q6 defines a new class of flavin-linked sulfhydryl oxidases” published in “J. Biol. Chem. 274 (45), 31759-31762 (1999); Coppock, D. et al, 2000, “Regulation of the quiescence-induced genes: quiescin Q6, decorin, and ribosomal protein S29” published in Biochem. Biophys. Res. Commun. 269 (2), 604-610 (2000) and the amino acid sequence of QSCN6 (identified by accession no. AAQ89300) is disclosed in, e.g., Clark, H. F. et al., 2003, “The Secreted Protein Discovery Initiative (SPDI), a Large-ScaleEffort to Identify Novel Human Secreted and Transmembrane Proteins: A Bioinformatics Assessment” published in Genome Res. 13 (10), 2265-2270 (2003), each of which is incorporated by reference herein in its entirety.

The nucleotide sequence of RGS4 (identified by accession no. NM_{—}005613) is disclosed in, e.g., Druey, K. M. et al, 1996, “Inhibition of G-protein-mediated MAP kinase activation by a new mammalian gene family,” published in Nature 379 (6567), 742-746; Berman, D. M. et al., 1996, “GAIP and RGS4 are GTPase-activating proteins for the Gi subfamily of G protein alpha subunits,” published in Cell 86 (3), 445-452; Heximer, S. P. et al., 1997, “RGS2/GOS8 is a selective inhibitor of Gqalpha function,” published in Proc. Natl. Acad. Sci. U.S.A. 94 (26), 14389-14393, and the amino acid sequence of RGS4 (identified by accession no. NP_{—}005604) is disclosed in, e.g. Druey, K. M. et al., 1996, “Inhibition of G-protein-mediated MAP kinase activation by a new mammalian gene family,” published in Nature 379 (6567), 742-746; Berman, D. M. et al., 1996, “GAIP and RGS4 are GTPase-activating proteins for the Gi subfamily of G protein alpha subunits,” published in Cell 86 (3), 445-452; Heximer, S. P. et al, 1997, “RGS2/GOS8 is a selective inhibitor of Gqalpha function,” published in Proc. Natl. Acad. Sci. U.S.A. 94 (26), 14389-14393, each of which is incorporated by reference herein in its entirety.

The nucleotide sequence of SAA1 (identified by accession no. BC105796) is disclosed in, e.g., NIH MGC Project, 2005, Direct Submission, National Institutes of Health, Mammalian Gene Collection (MGC), Bethesda, Md.; Strausberg et al., 2002, “Generation and initial analysis of more than 15,000 full-length human and mouse cDNA sequences,” published in Proc. Natl. Acad. Sci. U.S.A. 99, 16899-16903, and the amino acid sequence of SAA1 (identified by accession nos. AAA64799, AAA30968) is disclosed in, e.g., Kluve-Beckerman, B. et al., 1998, “Human serum amyloid A. Three hepatic mRNAs and the corresponding proteins in one person,” published in J. Clin. Invest. 82 (5), 1670-1675; Marhaug, G. et al., 1990, “Mink serum amyloid A protein. Expression and primary structure based on cDNA sequences,” published in J. Biol. Chem. 265, 10049-10054, each of which is hereby incorporated by reference herein in its entirety.

The nucleotide sequence of SAA4 (identified by accession no. NM_{—}006512) is disclosed in, e.g., Bausserman, L. L. et al., 1983, “Interaction of the serum amyloid A proteins with phospholipid,” published in J. Biol. Chem. 258 (17), 10681-10688; Whitehead, A. S. et al, 1992, “Identification of novel members of the serum amyloid A protein superfamily as constitutive apolipoproteins of high density lipoprotein,” published in J. Biol. Chem. 267 (6), 3862-3867; Watson, G. et al, 1992, “Analysis of the genomic and derived protein structure of a novel human serum amyloid A gene, SAA4,” published in Scand. J. Immunol. 36 (5), 703-712, and the amino acid sequence of SAA4 (identified by accession no. NP_{—}006503) is disclosed in, e.g., Bausserman, L. L. et al., 1983, “Interaction of the serum amyloid A proteins with phospholipid,” published in J. Biol. Chem. 258 (17), 10681-10688; Whitehead, A. S. et al., 1992, “Identification of novel members of the serum amyloid A protein superfamily as constitutive apolipoproteins of high density lipoprotein,” published in J. Biol. Chem. 267 (6), 3862-3867; Watson, G. et al, 1992, “Analysis of the genomic and derived protein structure of a novel human serum amyloid A gene, SAA4,” published in Scand. J. Immunol. 36 (5), 703-712; and Kang et al., 1987, “The precursor of Alzheimer's disease amyloid A4 protein resembles a cell-surface receptor, Nature 325, 733-736, each of which is incorporated by reference herein in its entirety.

The nucleotide sequence of serum amyloid A-4 protein precursor (identified by accession no. M81349) is disclosed in, e.g., Whitehead et al., 1992, “Identification of novel members of the serum amyloid A protein superfamily as constitutive apolipoproteins of high density lipoprotein and the amino acid sequence of SAA4,” and the amino acid sequence of serum amyloid A-4 protein precursor (identified by accession no. P02375) is disclosed in Sipe, 1985, “Human serum amyloid A (SAA): biosynthesis and postsynthetic processing of preSAA and structural variants defined by complementary DNA,” Biochemistry 24, 2931-2936, each of which is incorporated by reference herein in its entirety.

The nucleotide sequence of SERPINA7 (identified by accession no. NM_{—}000354) is disclosed in, e.g., Flink, I. L. et al., 1986, “Complete amino acid sequence of human thyroxine-binding globulin deduced from cloned DNA: close homology to the serine antiproteases,” published in Proc. Natl. Acad. Sci. U.S.A. 83 (20), 7708-7712; Takeda, K. et al., 1989, “Sequence of the variant thyroxine-binding globulin of Australian aborigines. Only one of two amino acid replacements is responsible for its altered properties,” published in J. Clin. Invest. 83 (4), 1344-1348; Mori, Y. et al, 1990, “Replacement of Leu227 by Pro in thyroxine-binding globulin (TBG) is associated with complete TBG deficiency in three of eight families with this inherited defect,” published in J. Clin. Endocrinol. Metab. 70 (3), 804-809, and the amino acid sequence of SERPINA7 (identified by accession no. CAB06092) is disclosed in, e.g., Cheng, “Partial amino acid sequence of human thyroxine binding globulin,” Biochem. Biophys. Res. Commun. 79: 1212-1218, each of which is incorporated by reference herein in its entirety.

The nucleotide sequence of TF (identified by accession no. NM_{—}001063) is disclosed in, e.g., Enns, C. A. et al., 1981, “Physical characterization of the transferrin receptor in human placentae,” published in J. Biol. Chem. 256 (19), 9820-9823; Sass-Kuhn, S. P. et al., 1984, “Human granulocyte/pollen-binding protein. Recognition and identification as transferrin,” published in J. Clin. Invest. 73 (1), 202-210; Uzan, G. et al., 1984, “Molecular cloning and sequence analysis of cDNA for human transferrin,” published in Biochem. Biophys. Res. Commun. 119 (1), 273-281, and the amino acid sequence of TF (identified by accession no. NP_{—}001054) is disclosed in, e.g., Enns, C. A. et al., 1981, “Physical characterization of the transferrin receptor in human placentae,” published in J. Biol. Chem. 256 (19), 9820-9823; Sass-Kuhn, S. P. et al., 1984, “Human granulocyte/pollen-binding protein. Recognition and identification as transferrin,” published in J. Clin. Invest. 73 (1), 202-210; Uzan, G. et al., 1984, “Molecular cloning and sequence analysis of cDNA for human transferrin,” published in Biochem. Biophys. Res. Commun. 119 (1), 273-281, each of which is incorporated by reference herein in its entirety.

The nucleotide sequence of TFRC (identified by accession no. NM_{—}003234) is disclosed in, e.g., Enns, C. A. et al., 1981, “Physical characterization of the transferrin receptor in human placentae,” published in J. Biol. Chem. 256 (19), 9820-9823; Omary, M. B. et al., 1981, “Biosynthesis of the human transferrin receptor in cultured cells,” published in J. Biol. Chem. 256 (24), 12888-12892; Miller, Y. E. et al., 1983, “Chromosome 3q (22-ter) encodes the human transferrin receptor,” published in Am. J. Hum. Genet. 35 (4), 573-583, and the amino acid sequence of TFRC (identified by accession no. NP_{—}003225) is disclosed in, e.g., Enns, C. A. et al., 1981, “Physical characterization of the transferrin receptor in human placentae,” published in J. Biol. Chem. 256 (19), 9820-9823; Omary, M. B. et al., 1981, “Biosynthesis of the human transferrin receptor in cultured cells,” published in J. Biol. Chem. 256 (24), 12888-12892; Miller, Y. E. et al, 1983, “Chromosome 3q (22-ter) encodes the human transferrin receptor,” published in Am. J. Hum. Genet. 35 (4), 573-583, each of which is incorporated by reference herein in its entirety.

The nucleotide sequence of TTN (identified by accession no. BC013396) is disclosed in, e.g., Strausberg et al., 2002, “Generation and initial analysis of more than 15,000 full-length human and mouse cDNA sequences,” published in Proc. Natl. Acad. Sci. U.S.A. 99 (26), 16899-16903, and the amino acid sequence of TTN (identified by accession no. CAD12456) is disclosed in, e.g., Bang et al., 2001, “The complete gene sequence of titin, expression of an unusual approximately 700-kDa titin isoform, and its interaction with obscurin identify a novel Z-line to I-band linking system,” published in Circ. Res. 89 (11), 1065-1072, each of which is incorporated by reference herein in its entirety.

The nucleotide sequence of TTR (identified by accession no. NM_{—}000371) is disclosed in, e.g., Fex et al., 1979, “Interaction between prealbumin and retinol-binding protein studied by affinity chromatography, gel filtration and two-phase partition,” published in Eur. J. Biochem. 99 (2), 353-360; Mita et al., 1984, “Cloning and sequence analysis of cDNA for human prealbumin,” published in Biochem. Biophys. Res. Commun. 124 (2), 558-564, and the amino acid sequence of TTR (identified by accession nos. AAH05310, AAP35853) is disclosed in, e.g., Kanda et al., “The amino acid sequence of human plasma prealbumin,” J. Biol. Chem. 249: 6796-6805, each of which is incorporated by reference herein in its entirety.

The nucleotide sequence of transthyretin precursor (prealbumin) (TBPA) (TTR) (ATTR) is disclosed in Gu et al, 1991, “Transthyretin (prealbumin) gene in human primary hepatic cancer,” Chem. Life Scie Earth Sci. 34, 1312-1318 and the amino acid sequence is disclosed in Mita et al., 1984, “Cloning and sequence analysis of cDNA for human prealbumin,” Biophys. Res. Commun. 124, 558-564 each of which is hereby incorporated by reference in its entirety.

The nucleotide sequence of UBC (identified by accession no. NM_{—}021009) is disclosed in, e.g., Wiborg, O. et al., 1985, “The human ubiquitin multigene family: some genes contain multiple directly repeated ubiquitin coding sequences,” published in EMBO J. 4 (3), 755-759; Einspanier, R. et al., 1987, “Cloning and sequence analysis of a cDNA encoding poly-ubiquitin in human ovarian granulosa cells,” published in Biochem. Biophys. Res. Commun. 147 (2), 581-587; Baker, R. T. et al., 1989, “Unequal crossover generates variation in ubiquitin coding unit number at the human UbC polyubiquitin locus,” published in Am. J. Hum. Genet. 44 (4), 534-542, and the amino acid sequence of UBC (identified by accession no. NP_{—}066289) is disclosed in, e.g., Wiborg, O. et al., 1985, “The human ubiquitin multigene family: some genes contain multiple directly repeated ubiquitin coding sequences,” published in EMBO J. 4 (3), 755-759; Einspanier, R. et al., 1987, “Cloning and sequence analysis of a cDNA encoding poly-ubiquitin in human ovarian granulosa cells,” published in Biochem. Biophys. Res. Commun. 147 (2), 581-587; Baker, R. T. et al, 1989, “Unequal crossover generates variation in ubiquitin coding unit number at the human UbC polyubiquitin locus,” published in Am. J. Hum. Genet. 44 (4), 534-542, each of which is incorporated by reference herein in its entirety.

The nucleotide sequence of VTN (identified by accession no. NM_{—}000638) is disclosed in, e.g., Suzuki, S. et al., 1984, “Domain structure of vitronectin. Alignment of active sites,” published in J. Biol. Chem. 259 (24), 15307-15314; Suzuki, S. et al, 1985, “Complete amino acid sequence of human vitronectin deduced from cDNA. Similarity of cell attachment sites in vitronectin and fibronectin,” published in EMBO J. 4 (10), 2519-2524; Jenne, D. et al., 1985, “Molecular cloning of S-protein, a link between complement, coagulation and cell-substrate adhesion,” published in EMBO J. 4 (12), 3153-3157, and the amino acid sequence of VTN (identified by accession no. P04004) is disclosed in, e.g., Zhou, A. et al., 2003, “How vitronectin binds PAI-1 to modulate fibrinolysis and cell migration,” published in Nat. Struct. Biol. 10 (7), 541-544; Kamikubo, Y. et al., 2002, “Identification of the disulfide bonds in the recombinant somatomedin B domain of human vitronectin,” published in J. Biol. Chem. 277 (30), 27109-27119; Seger, D. et al, 1998, “Phosphorylation of vitronectin by casein kinase II. Identification of the sites and their promotion of cell adhesion and spreading,” published in J. Biol. Chem. 273 (38), 24805-24813, each of which is incorporated by reference herein in its entirety The nucleotide sequence of VWF (identified by accession no. NM_{—}000552) is disclosed in, e.g., Coller, B. S. et al., 1983, “Studies with a murine monoclonal antibody that abolishes ristocetin-induced binding of von Willebrand factor to platelets: additional evidence in support of GPIb as a platelet receptor for von Willebrand factor,” published in Blood 61 (1), 99-110; Lynch, D. C. et al., 1985, “Molecular cloning of cDNA for human von Willebrand factor: authentication by a new method,” published in Cell 41 (1), 49-56; Ginsburg, D. et al, 1985, “Human von Willebrand factor (vWF): isolation of complementary DNA (cDNA) clones and chromosomal localization,” published in Science 228 (4706), 1401-1406, and the amino acid sequence of VWF (identified by accession no. AAB59458) is disclosed in, e.g., Mancuso, D. J. et al, 1989, “Structure of the gene for human von Willebrand factor,” published in J. Biol. Chem. 264 (33), 19514-19527, each of which is incorporated by reference herein in its entirety.

The nucleotide sequence of ALMS1 (identified by accession no. NM_{—}015120) is disclosed in, e.g., Collin, G. B. et al., 1999, “Alstrom syndrome: further evidence for linkage to human chromosome 2p13,” published in Hum. Genet. 105 (5), 474-479; Collin, G. B. et al., 2002, “Mutations in ALMS1 cause obesity, type 2 diabetes and neurosensory degeneration in Alstrom syndrome,” published in Nat. Genet. 31 (1), 74-78; Hearn, T. et al., Mutation of ALMS1, a large gene with a tandem repeat encoding 47 amino acids, causes Alstrom syndrome,” published in Nat. Genet. 31 (1), 79-83, and the amino acid sequence of ALMS1 (identified by accession no. NP_{—}055935) is disclosed in, e.g., Collin, G. B. et al., 1999, “Alstrom syndrome: further evidence for linkage to human chromosome 2p13,” published in Hum. Genet. 105 (5), 474-479; Collin, G. B. et al., 2002, “Mutations in ALMS1 cause obesity, type 2 diabetes and neurosensory degeneration in Alstrom syndrome,” published in Nat. Genet. 31 (1), 74-78; Hearn, T. et al., Mutation of ALMS1, a large gene with a tandem repeat encoding 47 amino acids, causes Alstrom syndrome,” published in Nat. Genet. 31 (1), 79-83, each of which is incorporated by reference herein in its entirety.

The nucleotide sequence of ATRN (identified by accession nos. BC101705, NM_{—}139321) is disclosed in, e.g., Strausberg, R. L. et al., 2002, “Generation and initial analysis of more than 15,000 full-length human and mouse cDNA sequences,” published in Proc. Natl. Acad. Sci. U.S.A. 99 (26), 16899-16903; Mori, M. et al., 1992, “Topical timolol and blood-aqueous barrier permeability to protein in human eyes,” published in Nippon Ganka Gakkai Zasshi 96 (11), 1418-1422; Duke-Cohan, J. S. et al, 1996, “Serum high molecular weight dipeptidyl peptidase IV (CD26) is similar to a novel antigen DPPT-L released from activated T cells,” published in J. Immunol. 156 (5), 1714-1721; Duke-Cohan, J. S. et al., 1998, “Attractin (DPPT-L), a member of the CUB family of cell adhesion and guidance proteins, is secreted by activated human T lymphocytes and modulates immune cell interactions,” published in Proc. Natl. Acad. Sci. U.S.A. 95 (19), 11336-11341, and the amino acid sequence of ATRN (identified by accession no. CA122615) is disclosed in, e.g., Sehra, H., 2005, Direct Submission, Wellcome Trust Sanger Institute, Hinxton, Cambridgeshire, CB10 1SA, UK, each of which is incorporated by reference herein in its entirety.

The nucleotide sequence of APOL1 (identified by accession nos. BC017331, NM_{—}003661) is disclosed in, e.g., Strausberg et al., 2002, “Generation and initial analysis of more than 15,000 full-length human and mouse cDNA sequences,” published in Proc. Natl. Acad. Sci. U.S.A. 99 (26), 16899-16903; Duchateau, P. N. et al., 1997, “Apolipoprotein L, a new human high density lipoprotein apolipoprotein expressed by the pancreas. Identification, cloning, characterization, and plasma distribution of apolipoprotein L,” published in Biol. Chem. 272 (41), 25576-25582; Duchateau, P. N. et al., 2000, “Plasma apolipoprotein L concentrations correlate with plasma triglycerides and cholesterol levels in normolipidemic, hyperlipidemic, and diabetic subjects,” published in J. Lipid Res. 41 (8), 1231-1236; Duchateau, P. N. et al., 2001, “Apolipoprotein L gene family: tissue specific expression, splicing, promoter regions; discovery of a new gene,” published in J. Lipid Res. 42 (4), 620-630, and the amino acid sequence of APOL1 (identified by accession no. AAK20210) is disclosed in, e.g., Page et al, 2001, “The human apolipoprotein L gene cluster: identification, classification, and sites of distribution,” published in Genomics 74 (1), 71-78, each of which is incorporated by reference herein in its entirety.

The nucleotide sequence of TRIP11 (identified by accession no. NM_{—}004239) is disclosed in, e.g., Lee, J. W. et al., 1995, “Two classes of proteins dependent on either the presence or absence of thyroid hormone for interaction with the thyroid hormone receptor,” published in Mol. Endocrinol. 9 (2), 243-254; Chang, K. H. et al., 1997, “A thyroid hormone receptor coactivator negatively regulated by the retinoblastoma protein,” published in Proc. Natl. Acad. Sci. U.S.A. 94 (17), 9040-9045; Abe, A. et al., 1997, “Fusion of the platelet-derived growth factor receptor beta to a novel gene CEV14 in acute myelogenous leukemia after clonal evolution,” published in Blood 90 (11), 4271 4277, and the amino acid sequence of TRIP11 (identified by accession no. NP_{—}004230) is disclosed in, e.g., Lee, J. W. et al, 1995, “Two classes of proteins dependent on either the presence or absence of thyroid hormone for interaction with the thyroid hormone receptor,” published in Mol. Endocrinol. 9 (2), 243-254; Chang, K. H. et al., 1997, “A thyroid hormone receptor coactivator negatively regulated by the retinoblastoma protein,” published in Proc. Natl. Acad. Sci. U.S.A. 94 (17), 9040-9045; Abe, A. et al., 1997, “Fusion of the platelet-derived growth factor receptor beta to a novel gene CEV14 in acute myelogenous leukemia after clonal evolution,” published in Blood 90 (11), 4271-4277, each of which is incorporated by reference herein in its entirety.

The nucleotide sequence of PDCD11 (identified by accession no. NM_{—}014976) is disclosed in, e.g., Lacana, E. et al., 1999, “Regulation of Fas ligand expression and cell death by apoptosis-linked gene 4,” published in Nat. Med. 5 (5), 542-547; Sweet, T. et al., 2003, “Identification of a novel protein from glial cells based on its ability to interact with NF-kappaB subunits,” published in J. Cell. Biochem. 90 (5), 884-891, and the amino acid sequence of PDCD11 (identified by accession no. NP_{—}055791) is disclosed in, e.g., Lacana, E. et al., 1999, “Regulation of Fas ligand expression and cell death by apoptosis-linked gene 4,” published in Nat. Med. 5 (5), 542-547; Sweet, T. et al, 2003, “Identification of a novel protein from glial cells based on its ability to interact with NF-kappaB subunits,” published in J. Cell. Biochem. 90 (5), 884-891, each of which is incorporated by reference herein in its entirety.

The nucleotide sequence of KIAA0433 (identified by accession no. AB007893) is disclosed in, e.g., Ishikawa et al., 1997, “Prediction of the coding sequences of unidentified human genes. VIII. 78 new cDNA clones from brain which code for large proteins in vitro,” DNA Res. 4 (5), 307-313, and the amino acid sequence of KIAA0433 (identified by accession no. BAA24863) is disclosed in, e.g., Kisarazu et al., 1997, “Prediction of the coding sequences of unidentified human genes. VIII. 78 new cDNA clones from brain which code for large proteins in vitro,” published in DNA Res. 4 (5), 307-313, each of which is incorporated by reference herein in its entirety.

The nucleotide sequence of SERPINA10 (identified by accession no. NM_{—}016186) is disclosed in, e.g., Han, X. et al., 1998, “Isolation of a protein Z-dependent plasma protease inhibitor,” published in Proc. Natl. Acad. Sci. U.S.A. 95 (16), 9250-9255; Han, X. et al., 1999, “The protein Z-dependent protease inhibitor is a serpin,” published in Biochemistry 38 (34), 11073-11078; Yin, Z. F. et al., 2000, “Prothrombotic phenotype of protein Z deficiency,” published in Proc. Natl. Acad. Sci. U.S.A. 97 (12), 6734-6738, and the amino acid sequence of SERPINA10 (identified by accession no. NP_{—}057270) is disclosed in, e.g., Han, X. et al., 1998, “Isolation of a protein Z-dependent plasma protease inhibitor,” published in Proc. Natl. Acad. Sci. U.S.A. 95 (16), 9250-9255; Han, X. et al., 1999, “The protein Z-dependent protease inhibitor is a serpin,” published in Biochemistry 38 (34), 11073-11078; Yin, Z. F. et al., 2000, “Prothrombotic phenotype of protein Z deficiency,” published in Proc. Natl. Acad. Sci. U.S.A. 97 (12), 6734-6738, each of which is incorporated by reference herein in its entirety.

The nucleotide sequence of BCOR (identified by accession no. BC063536) is disclosed in, e.g., Strausberg et al., 2002, “Generation and initial analysis of more than 15,000 full-length human and mouse cDNA sequences,” Proc. Natl. Acad. Sci. U.S.A. 99 (26), 16899-16903, and the amino acid sequence of BCOR (identified by accession no. AAG41429) is disclosed in, e.g., Huynh et al, 2000, “BCOR, a novel corepressor involved in BCL-6 repression,” Genes Dev. 14 (14), 1810-1823, each of which is incorporated by reference herein in its entirety.

The nucleotide sequence of C10orf18 (identified by accession no. BC001759) is disclosed in, e.g., Strausberg et al., 2002, “Generation and initial analysis of more than 15,000 full-length human and mouse cDNA sequences,” Proc. Natl. Acad. Sci. U.S.A. 99 (26), 16899-16903, and the amino acid sequence of C10orf18 (identified by accession no. CA113368) is disclosed in, e.g., Wray, P., 2005, Direct Submission, Wellcome Trust Sanger Institute, Hinxton, Cambridgeshire, CB10 1SA, UK, each of which is incorporated by reference herein in its entirety.

The nucleotide sequence of YY1AP1 (identified by accession nos. BC044887, BC014906) is disclosed in, e.g., Strausberg et al., 2002, “Generation and initial analysis of more than 15,000 full-length human and mouse cDNA sequences,” published in Proc. Natl. Acad. Sci. U.S.A. 99 (26), 16899-16903 and the amino acid sequence of YY1AP1 (identified by accession nos. AAL75971, CAH71646) is disclosed in, e.g., Liang et al., “Cloning and characterization of a novel YY1 associated protein,” unpublished, Almeida, J., 2005, Direct Submission, Wellcome Trust Sanger Institute, Hinxton, Cambridgeshire, CB10 1SA, UK each of which is incorporated by reference herein in its entirety.

The nucleotide sequence of FLJ10006 (identified by accession nos. BC110537, BC110536) is disclosed in, e.g., Strausberg et al., 2002, “Generation and initial analysis of more than 15,000 full-length human and mouse cDNA sequences,” Proc. Natl. Acad. Sci. U.S.A. 99 (26), 16899-16903, and the amino acid sequence of FLJ10006 (identified by accession no. AAH17012) is disclosed in, e.g., Director MGC Project, 2005, Direct Submission, National Institutes of Health, Mammalian Gene Collection (MGC), Cancer Genomics Office, National Cancer Institute, 31 Center Drive, Room 11A03, Bethesda, Md. 20892-2590, USA; Strausberg et al., 2002, “Generation and initial analysis of more than 15,000 full-length human and mouse cDNA sequences,” Proc. Natl. Acad. Sci. U.S.A. 99 (26), 16899-16903, each of which is incorporated by reference herein in its entirety.

The nucleotide sequence of BDP1 (identified by accession no. NM_{—}018429) is disclosed in, e.g., Schramm, L. et al., 2000, “Different human TFIIIB activities direct RNA polymerase III transcription from TATA-containing and TATA-less promoters,” published in Genes Dev. 14 (20), 2650-2663; Kelter, A. R. et al., 2000, “The transcription factor-like nuclear regulator (TFNR) contains a novel 55-amino-acid motif repeated nine times and maps closely to SMN1,” published in Genomics 70 (3), 315-326; Weser, S. et al., Transcription factor (TF)-like nuclear regulator, the 250-kDa form of Homo sapiens TFIIIB′, is an essential component of human TFIIIC1 activity,” published in J. Biol. Chem. 279 (26), 27022-27029, and the amino acid sequence of BDP1 (identified by accession no. AAH32146) is disclosed in, e.g., Strausberg, R. L et al, “Generation and initial analysis of more than 15,000 full-length human and mouse cDNA sequences” published in Proc. Natl. Acad. Sci. U.S.A. 99 (26), 16899-16903 (2002), each of which is incorporated by reference herein in its entirety.

The nucleotide sequence of SMARCAD1 (identified by accession no. NM_{—}020159) is disclosed in, e.g., Soininen, R. et al., 1992, “The mouse Enhancer trap locus 1 (Etl-1): a novel mammalian gene related to Drosophila and yeast transcriptional regulator genes,” published in Mech. Dev. 39 (1-2), 111-123; Adra, C. N. et al, 2000, “SMARCAD1, a novel human helicase family-defining member associated with genetic instability: cloning, expression, and mapping to 4q22-q23, a band rich in breakpoints and deletion mutants involved in several human diseases,” published in Genomics 69 (2), 162-173, and the amino acid sequence of SMARCAD1 (identified by accession no. NP_{—}064544) is disclosed in, e.g., Soininen, R. et al., 1992, “The mouse Enhancer trap locus 1 (Etl-1): a novel mammalian gene related to Drosophila and yeast transcriptional regulator genes,” published in Mech. Dev. 39 (1-2), 111-123; Adra, C. N. et al., 2000, “SMARCAD1, a novel human helicase family-defining member associated with genetic instability: cloning, expression, and mapping to 4q22-q23, a band rich in breakpoints and deletion mutants involved in several human diseases,” published in Genomics 69 (2), 162-173, each of which is incorporated by reference herein in its entirety.

The nucleotide sequence of MKL2 (identified by accession no. NM_{—}014048) is disclosed in, e.g., Cen, B. et al., 2003, “Megakaryoblastic leukemia 1, a potent transcriptional coactivator for serum response factor (SRF), is required for serum induction of SRF target genes,” published in Mol. Cell. Biol. 23 (18), 6597-6608; Selvaraj, A. et al., 2003, “Megakaryoblastic leukemia-1/2, a transcriptional co-activator of serum response factor, is required for skeletal myogenic differentiation,” published in J. Biol. Chem. 278 (43), 41977-41987, and the amino acid sequence of MKL2 (identified by accession no. AAH47761) is disclosed in, e.g., Strausberg et al., 2002, “Generation and initial analysis of more than 15,000 full-length human and mouse cDNA sequences,” Proc. Natl. Acad. Sci. U.S.A. 99 (26), 16899-16903, each of which is incorporated by reference herein in its entirety.

The nucleotide sequence of CHST8 (identified by accession nos. NM_{—}022467, BC018723) is disclosed in, e.g., Xia et al., 2000, “Molecular cloning and expression of the pituitary glycoprotein hormone N-acetylgalactosamine-4-O-sulfotransferase,” published in J. Biol. Chem. 275 (49), 38402-38409; Okuda, T. et al, 2000, “Molecular cloning and characterization of GalNAc 4-sulfotransferase expressed in human pituitary gland,” published in J. Biol. Chem. 275 (51), 40605-40613; Hiraoka, N et al, 2001, “Molecular cloning and expression of two distinct human N-acetylgalactosamine 4-O-sulfotransferases that transfer sulfate to GalNAc beta 1->4GlcNAc beta 1->R in both N- and O-glycans,” Glycobiology 11 (6), 495-504; Strausberg et al., 2002, “Generation and initial analysis of more than 15,000 full-length human and mouse cDNA sequences,” Proc. Natl. Acad. Sci. U.S.A. 99 (26), 16899-16903, and the amino acid sequence of CHST8 (identified by accession no. NP_{—}071912) is disclosed in, e.g., Xia et al., 2000, “Molecular cloning and expression of the pituitary glycoprotein hormone N acetylgalactosamine-4-O-sulfotransferase,” J. Biol. Chem. 275 (49), 38402-38409; Okuda et al., 2000, “Molecular cloning and characterization of GalNAc 4-sulfotransferase expressed in human pituitary gland,” published in J. Biol. Chem. 275 (51), 40605-40613; Hiraoka, N et al., 2001, “Molecular cloning and expression of two distinct human N-acetylgalactosamine 4-O-sulfotransferases that transfer sulfate to GalNAc beta 1->4GlcNAc beta 1->R in both N- and O-glycans,” published in Glycobiology 11 (6), 495-504, each of which is incorporated by reference herein in its entirety.

The nucleotide sequence of MCPH1 (identified by accession nos. NM_{—}024596, BC030702) is disclosed in, e.g., Jackson, A. P. et al., 1998, “Primary autosomal recessive microcephaly (MCPH1) maps to chromosome 8p22-pter,” published in Am. J. Hum. Genet. 63 (2), 541-546; Jackson, A. P. et al., 2002, “Identification of microcephalin, a protein implicated in determining the size of the human brain,” published in Am. J. Hum. Genet. 71 (1), 136-142; Kumar, A. et al., 2002, “Primary microcephaly: microcephalin and ASPM determine the size of the human brain,” published in J. Biosci. 27 (7), 629 632, and the amino acid sequence of MCPH1 (identified by accession no. AAH30702) is disclosed in, e.g., Strausberg, R. L. et al., 2002, “Generation and initial analysis of more than 15,000 full-length human and mouse cDNA sequences” published in Proc. Natl. Acad. Sci. U.S.A. 99 (26), 16899-16903 (2002), each of which is incorporated by reference herein in its entirety.

The nucleotide sequence of MYO18B (identified by accession no. NM_{—}032608) is disclosed in, e.g., Nishioka, M. et al., 2002, “MYO18B, a candidate tumor suppressor gene at chromosome 22q12.1, deleted, mutated, and methylated in human lung cancer,” published in Proc. Natl. Acad. Sci. U.S.A. 99 (19), 12269-12274; Salamon, M. et al., 2003, “Human MYO18B, a novel unconventional myosin heavy chain expressed in striated muscles moves into the myonuclei upon differentiation,” J. Mol. Biol. 326 (1), 137-149; Yanaihara, N. et al., 2004, “Reduced expression of MYO18B, a candidate tumor-suppressor gene on chromosome arm 22q, in ovarian cancer,” published in Int. J. Cancer 112 (1), 150-154, and the amino acid sequence of MYO18B (identified by accession no. NP_{—}115997) is disclosed in, e.g., Nishioka, M. et al., 2002, “MYO18B, a candidate tumor suppressor gene at chromosome 22q12.1, deleted, mutated, and methylated in human lung cancer,” published in Proc. Natl. Acad. Sci. U.S.A. 99 (19), 12269-12274; Salamon, M. et al., 2003, “Human MYO18B, a novel unconventional myosin heavy chain expressed in striated muscles moves into the myonuclei upon differentiation,” J. Mol. Biol. 326 (1), 137-149; Yanaihara, N. et al., 2004, “Reduced expression of MYO18B, a candidate tumor-suppressor gene on chromosome arm 22q, in ovarian cancer,” published in Int. J. Cancer 112 (1), 150-154, each of which is incorporated by reference herein in its entirety.

The nucleotide sequence of MICAL-L1 (identified by accession no. NM_{—}033386) is disclosed in, e.g., Marzesco, A. M. et al., 2002, “The small GTPase Rab13 regulates assembly of functional tight junctions in epithelial cells,” published in Mol. Biol. Cell 13 (6), 1819-1831; Terman, J. R. et al., 2002, “MICALs, a family of conserved flavoprotein oxidoreductases, function in plexin-mediated axonal repulsion,” published in Cell 109 (7), 887-900; Collins, J. E. et al., 2004, “A genome annotation driven approach to cloning the human ORFeome,” published in Genome Biol. 5 (10), R84, and the amino acid sequence of MICAL-L1 (identified by accession nos. AAH82243, AAH01090) is disclosed in, e.g., Strausberg et al., 2002, “Generation and initial analysis of more than 15,000 full-length human and mouse cDNA sequences,” published in Proc. Natl. Acad. Sci. U.S.A. 99 (26), 16899-16903, each of which is incorporated by reference herein in its entirety.

The nucleotide sequence of PGLYRP2 (identified by accession no. NM_{—}052890) is disclosed in, e.g., Liu, C. et al., 2002, “Peptidoglycan recognition proteins: a novel family of four human innate immunity pattern recognition molecules,” published in J. Biol. Chem. 276 (37), 34686-34694; Xu, X. R. et al., 2001, “Insight into hepatocellular carcinogenesis at transcriptome level by comparing gene expression profiles of hepatocellular carcinoma with those of corresponding noncancerous liver,” published in Proc. Natl. Acad. Sci. U.S.A. 98 (26), 15089-15094; Kibardin, A. V. et al., 2003, “Expression analysis of proteins encoded by genes of the tag7/tagL(PGRP-S,L) family in human peripheral blood cells,” Genetika 39 (2), 244-249, and the amino acid sequence of PGLYRP2 (identified by accession no. Q96PD5) is disclosed in, e.g., Zhang, H. et al., 2003, “Identification and quantification of N-linked glycoproteins using hydrazide chemistry, stable isotope labeling and mass spectrometry,” published in Nat. Biotechnol. 21 (6), 660-666; Wang, Z. M. et al., 2003, “Human peptidoglycan recognition protein-L is an N-acetylmuramoyl-L-alanine amidase,” published in J. Biol. Chem. 278 (49), 49044-49052; Zhang, Z. et al., 2004, “Signal peptide prediction based on analysis of experimentally verified cleavage sites,” published in Protein Sci. 13 (10), 2819-2824, each of which is incorporated by reference herein in its entirety.

The nucleotide sequence of LRG1 (identified by accession no. NM_{—}052972) is disclosed in, e.g., Takahashi, N. et al, 1985, “Periodicity of leucine and tandem repetition of a 24-amino acid segment in the primary structure of leucine-rich alpha 2 glycoprotein of human serum,” published in Proc. Natl. Acad. Sci. U.S.A. 82 (7), 1906 1910; O'Donnell, L. C. et al., 2002, “Molecular characterization and expression analysis of leucine-rich alpha2-glycoprotein, a novel marker of granulocytic differentiation,” published in J. Leukoc. Biol. 72 (3), 478-485; Bunkenborg, J. et al., 2004, “Screening for N-glycosylated proteins by liquid chromatography mass spectrometry,” published in Proteomics 4 (2), 454-465, and the amino acid sequence of LRG1 (identified by accession no. AAH70198) is disclosed in, e.g., Strausberg et al., 2002, “Generation and initial analysis of more than 15,000 full-length human and mouse cDNA sequences,” Proc. Natl. Acad. Sci. U.S.A. 99 (26), each of which is incorporated by reference herein in its entirety.

The nucleotide sequence of KCTD7 (identified by accession no. NM_{—}153033) is disclosed in, e.g., Scherer, S. W. et al, 2003, “Human chromosome 7: DNA sequence and biology,” published in Science 300 (5620), 767-772, and the amino acid sequence of KCTD7 (identified by accession no. NP_{—}694578) is disclosed in, e.g., Scherer, S. W. et al., 2003, “Human chromosome 7: DNA sequence and biology,” published in Science 300 (5620), 767-772, each of which is incorporated by reference herein in its entirety.

The nucleotide sequence of MGC27165 (identified by accession nos. BC087841 and BC005951) is disclosed in, e.g., Strausberg et al., 2002, “Generation and initial analysis of more than 15,000 full-length human and mouse cDNA sequences,” Proc. Natl. Acad. Sci. U.S.A. 99 (26), 16899-16903 and the amino acid sequence of MGC27165 (identified by accession no. AAH87841) is disclosed in, e.g., Strausberg et al., 2002, “Generation and initial analysis of more than 15,000 full-length human and mouse cDNA sequences,” published in Proc. Natl. Acad. Sci. U.S.A. 99 (26), 16899-16903, each of which is incorporated by reference herein in its entirety.

The nucleotide sequence of A1BG (identified by accession no. NM_{—}130786) is disclosed in, e.g., Ishioka, N. et al., 1986, “Amino acid sequence of human plasma alpha 1B-glycoprotein: homology to the immunoglobulin supergene family,” published in Proc. Natl. Acad. Sci. U.S.A. 83 (8), 2363-2367; Gahne, B. et al., 1987, “Genetic polymorphism of human plasma alpha 1B-glycoprotein: phenotyping by immunoblotting or by a simple method of 2-D electrophoresis,” published in Hum. Genet. 76 (2), 111 115; Eiberg, H. et al., 1989, “Linkage between alpha 1B-glycoprotein (A1BG) and Lutheran (LU) red blood group system: assignment to chromosome 19: new genetic variants of A1BG,” published in Clin. Genet. 36 (6), 415-418, and the amino acid sequence of A1BG (identified by accession no. NP_{—}570602) is disclosed in, e.g., Ishioka, N. et al., 1986, “Amino acid sequence of human plasma alpha 1B-glycoprotein: homology to the immunoglobulin supergene family,” published in Proc. Natl. Acad. Sci. U.S.A. 83 (8), 2363-2367; Gahne, B. et al., 1987, “Genetic polymorphism of human plasma alpha 1B-glycoprotein: phenotyping by immunoblotting or by a simple method of 2-D electrophoresis,” published in Hum. Genet. 76 (2), 111-115; Eiberg, H. et al., 1989, “Linkage between alpha 1B-glycoprotein (A1BG) and Lutheran (LU) red blood group system: assignment to chromosome 19: new genetic variants of A1BG,” published in Clin. Genet. 36 (6), 415-418, each of which is incorporated by reference herein in its entirety.

The nucleotide sequence of A2M (identified by accession no. NM_{—}000014) is disclosed in, e.g., Nartikova et al., 1979, “Uniform method for determining the alpha 1-antitrypsin and alpha 2-macroglobulin activity in human blood serum (plasma),” published in Vopr. Med. Khim. 25 (4), 494-499; Gustavsson et al., 1980, “Interaction between human pancreatic elastase and plasma protease inhibitors,” Hoppe-Seyler's Z. Physiol. Chem. 361 (2), 169-176; Murata et al., 1983, “Radioimmunoassay of human pancreatic elastase 1. In vitro interaction of human pancreatic elastase 1 with serum protease inhibitors,” Enzyme 30 (1), 29-37, and the amino acid sequence of A2M (identified by accession no. AAT02228) is disclosed in, e.g., Sottrup-Jensen et al., 1984, “Primary structure of human alpha 2-macroglobulin,” J. Biol. Chem. 259:8318-8327, each of which is incorporated by reference herein in its entirety.

The nucleotide sequence of ABLIM1 (identified by accession no. NM_{—}002313) is disclosed in, e.g., Adams et al, 1995, “Initial assessment of human gene diversity and expression patterns based upon 83 million nucleotides of cDNA sequence,” Nature 377 (6547 SUPPL), 3-174; Roof et al., 1997, “Molecular characterization of abLIM, a novel actin-binding and double zinc finger protein,” J. Cell Biol. 138 (3), 575-588; Kim et al., 1997, “Limatin (LIMAB1), an actin-binding LIM protein, maps to mouse chromosome 19 and human chromosome 10q25, a region frequently deleted in human cancers,” published in Genomics 46 (2), 291-293, and the amino acid sequence of ABLIM1 (identified by accession no. CAI10910) is disclosed in, e.g., Tracey, A., 2005, Direct Submission, Wellcome Trust Sanger Institute, Hinxton, Cambridgeshire, CB10 1SA, UK, each of which is incorporated by reference herein in its entirety.

The nucleotide sequence of ACTA1 (identified by accession no. NM_{—}001100) is disclosed in, e.g., Gunning, P. et al., 1983, “Isolation and characterization of full-length cDNA clones for human alpha-, beta-, and gamma-actin mRNAs: skeletal but not cytoplasmic actins have an amino-terminal cysteine that is subsequently removed,” published in Mol. Cell. Biol. 3 (5), 787-795; Hanauer, A. et al., 1983, “Isolation and characterization of cDNA clones for human skeletal muscle alpha actin,” published in Nucleic Acids Res. 11 (11), 3503-3516; Kedes, L. et al., 1985, “The human beta-actin multigene family,” published in Trans. Assoc. Am. Physicians 98, 42-46, and the amino acid sequence of ACTA1 (identified by accession no. CAI19052) is disclosed in, e.g., Matthews, N., 2005, Direct Submission, Wellcome Trust Sanger Institute, Hinxton, Cambridgeshire, CB10 1SA, UK, each of which is incorporated by reference herein in its entirety.

The nucleotide sequence of ANK3 (identified by accession no. NM_{—}020987) is disclosed in, e.g., Kordeli, E. et al., 1995, “AnkyrinG. A new ankyrin gene with neural-specific isoforms localized at the axonal initial segment and node of Ranvier,” published in J. Biol. Chem. 270 (5), 2352-2359; Kapfhamer, D. et al., Chromosomal localization of the ankyrinG gene (ANK3/Ank3) to human 10q21 and mouse 10,” published in Genomics 27 (1), 189-191; Devarajan, P. et al., 1996, “Identification of a small cytoplasmic ankyrin (AnkG119) in the kidney and muscle that binds beta I sigma spectrin and associates with the Golgi apparatus,” published in J. Cell Biol. 133 (4), 819-830, and the amino acid sequence of ANK3 (identified by accession no. CA140519) is disclosed in, e.g., Chapman, J., 2005, Direct Submission, Wellcome Trust Sanger Institute, Hinxton, Cambridgeshire, CB10 1SA, UK, each of which is incorporated by reference herein in its entirety.

The nucleotide sequence of APCS (identified by accession no. BT006750) is disclosed in, e.g., Mantzouranis et al., 1985, “Human serum amyloid P component. cDNA isolation, complete sequence of pre-serum amyloid P component, and localization of the gene to chromosome 1,” J. Biol. Chem. 260:7752-7756, and the amino acid sequence of APCS (identified by accession no. CAH73651) is disclosed in, e.g., Cobley, V., 2005, Direct Submission, Wellcome Trust Sanger Institute, Hinxton, Cambridgeshire, CB10 1SA, UK, each of which is incorporated by reference herein in its entirety.

The nucleotide sequence of serum amyloid P component precursor (identified by accession no. BC007058) is disclosed in, Strausberg, 2002, “Generation and Initial analysis of more than 15,000 full-length human and mouse cDNA sequences,” Proc. Natl. Acad. Sci. USA 99, 16899-16802 and the amino acid sequence of serum amyloid P component precursor (identified by accession no. NP001630) is disclosed in, e.g., Veerhuis et al., 2005, “Activation of human microglia by fibrillar prion protein-related peptides is enhanced by amyloid-associated factors SAP and C1q,” Neurobiol. Dis. 19, 273-282, each of which is incorporated by reference herein in its entirety.

The nucleotide sequence of B2M (identified by accession no. NM_{—}004048) is disclosed in, e.g., Krangel, M. S. et al., 1979, “Assembly and maturation of HLA-A and HLA-B antigens in vivo,” published in Cell 18 (4), 979-991; Suggs, S. V. et al., 1981, “Use of synthetic oligonucleotides as hybridization probes: isolation of cloned cDNA sequences for human beta 2-microglobulin,” published in Proc. Natl. Acad. Sci. U.S.A. 78 (11), 6613-6617; Rosa, F. et al., 1983, “The beta2-microglobulin mRNA in human Daudi cells has a mutated initiation codon but is still inducible by interferon,” published in EMBO J. 2 (2), 239-243, and the amino acid sequence of B2M (identified by accession no. AAA51811) is disclosed in, e.g., Gussow, D. et al, 1987, “The human beta 2-microglobulin gene. Primary structure and definition of the transcriptional unit,” published in J. Immunol. 139 (9), 3132-3138, each of which is incorporated by reference herein in its entirety.

The nucleotide sequence of C1R (identified by accession no. NM_{—}001733) is disclosed in, e.g., Lee, S. L. et al., 1978, “Familial deficiency of two subunits of the first component of complement. C1r and C1s associated with a lupus erythematosus-like disease,” published in Arthritis Rheum. 21 (8), 958-967; Leytus, S. P. et al., 1986, “Nucleotide sequence of the cDNA coding for human complement C1r,” published in Biochemistry 25 (17), 4855-4863; Journet, A. et al., 1986, “Cloning and sequencing of full-length cDNA encoding the precursor of human complement component C1r,” published in Biochem. J. 240 (3), 783-787, and the amino acid sequence of CIR (identified by accession no. NP_{—}001724) is disclosed in, e.g., Lee, S. L. et al., 1978, “Familial deficiency of two subunits of the first component of complement. C1r and C1s associated with a lupus erythematosus-like disease,” published in Arthritis Rheum. 21 (8), 958-967; Leytus, S. P. et al., 1986, “Nucleotide sequence of the cDNA coding for human complement C1r,” published in Biochemistry 25 (17), 4855-4863; Journet, A. et al., 1986, “Cloning and sequencing of full-length cDNA encoding the precursor of human complement component C1r,” published in Biochem. J. 240 (3), 783-787, each of which is incorporated by reference herein in its entirety.

The nucleotide sequence of C4B (identified by accession no. NM_{—}000592) is disclosed in, e.g., Teisberg, P. et al., 1976, “Genetic polymorphism of C4 in man and localisation of a structural C4 locus to the HLA gene complex of chromosome 6,” published in Nature 264 (5583), 253-254; Moon, K. E. et al., 1981, “Complete primary structure of human C4a anaphylatoxin,” published in J. Biol. Chem. 256 (16), 8685-8692; Mascart-Lemone, F. et al., 1983, “Genetic deficiency of C4 presenting with recurrent infections and a SLE-like disease. Genetic and immunologic studies,” published in Am. J. Med. 75 (2), 295-304, and the amino acid sequence of C4B (identified by accession no. AAR89095) is disclosed in, e.g., Sayer, D. et al., 2003, Direct Submission, Dept of Clinical Immunology, Royal Perth Hospital, Wellington Street, Perth, Western Australia, Australia; Sayer, D. et al., unpublished, “Molecular genetics of complement C4: implications for MHC evolution and disease susceptibility gene mapping,” each of which is incorporated by reference herein in its entirety.

The nucleotide sequence of C6 (identified by accession no. NM_{—}000065) is disclosed in, e.g., Hetland, G. et al., 1986, “Synthesis of complement components C5, C6, C7, C8 and C9 in vitro by human monocytes and assembly of the terminal complement complex,” published in Scand. J. Immunol. 24 (4), 421-428; Chakravarti, D. N. et al., 1988, “Biochemical characterization of the human complement protein C6. Association with alpha-thrombin-like enzyme and absence of serine protease activity in cytolytically active C6,” published in J. Biol. Chem. 263 (34), 18306-18312; Chakravarti, D. N. et al., 1989, “Structural homology of complement protein C6 with other channel-forming proteins of complement,” published in Proc. Natl. Acad. Sci. U.S.A. 86 (8), 2799-2803, and the amino acid sequence of C6 (identified by accession no. BAD02322) is disclosed in, e.g., Soejima et al., 2005, “Nucleotide sequence analyses of human complement 6 (C6) gene suggest balancing selection,” Ann. Hum. Genet. 69 (PT 3), 239-252, each of which is incorporated by reference herein in its entirety.

The nucleotide sequence of C7 (identified by accession no. NM_{—}000587) is disclosed in, e.g., DiScipio, R. G. et al., 1988, “The structure of human complement component C7 and the C5b-7 complex,” published in J. Biol. Chem. 263 (1), 549-560; Nurnberger, W. et al., 1989, “Familial deficiency of the seventh component of complement associated with recurrent meningococcal infections,” published in Eur. J. Pediatr. 148 (8), 758-760; Coto, E. et al., 1991, “DNA polymorphisms and linkage relationship of the human complement component C6, C7, and C9 genes,” published in Immunogenetics 33 (3), 184-187, and the amino acid sequence of C7 (identified by accession no. CAA72407) is disclosed in, e.g., Gonzalez, S., 1997, Direct Submission, S. Gonzalez, Servicio de Immunologia, Hospital Central Asturias, Julian Clayeria s.n., 33006 Oviedo, Asturias, SPAIN; Gonzalez, S. et al., 2002, “Cloning and characterization of human complement component C7 promoter,” each of which is incorporated by reference herein in its entirety.

The nucleotide sequence of C8B (identified by accession no. NM_{—}000066) is disclosed in, e.g., Howard, O. M. et al., 1987, “Complementary DNA and derived amino acid sequence of the beta subunit of human complement protein C8: identification of a close structural and ancestral relationship to the alpha subunit and C9,” published in Biochemistry 26 (12), 3565-3570; Haefliger, J. A. et al., 1987, “Complementary DNA cloning of complement C8 beta and its sequence homology to C9,” published in Biochemistry 26 (12), 3551-3556; Stewart, J. L. et al., 1987, “Evidence that C5b recognizes and mediates C8 incorporation into the cytolytic complex of complement,” published in J. Immunol. 139 (6), 1960-1964, and the amino acid sequence of C8B (identified by accession no. CAC18532) is disclosed in, e.g., Howden, P., 2005, Direct Submission, Wellcome Trust Sanger Institute, Hinxton, Cambridgeshire, CB10 1SA, UK, each of which is incorporated by reference herein in its entirety.

The nucleotide sequence of CDK5RAP2 (identified by accession no. NM_{—}018249) is disclosed in, e.g., Ching, Y. P. et al., 2000, “Cloning of three novel neuronal Cdk5 activator binding proteins,” published in Gene 242 (1-2), 285-294; Wang, X. et al., 2000, “Identification of a common protein association region in the neuronal Cdk5 activator,” published in J. Biol. Chem. 275 (41), 31763-31769; Andersen, J. S. et al., 2003, “Proteomic characterization of the human centrosome by protein correlation profiling,” published in Nature 426 (6966), 570-574, and the amino acid sequence of CDK5RAP2 (identified by accession no. CA140927) is disclosed in, e.g., Beasley, H., 2005, Direct Submission, Wellcome Trust Sanger Institute, Hinxton, Cambridgeshire, CB10 1SA, UK, each of which is incorporated by reference herein in its entirety.

The nucleotide sequence of CHGB (identified by accession no. NM_{—}001819) is disclosed in, e.g., Benedum, U. M. et al., 1987, “The primary structure of human secretogranin I (chromogranin B): comparison with chromogranin A reveals homologous terminal domains and a large intervening variable region,” published in EMBO J. 6 (5), 1203-1211; Gill, B. M. et al., 1991, “Chromogranin B: isolation from pheochromocytoma, N-terminal sequence, tissue distribution and secretory vesicle processing,” published in Regul. Pept. 33 (2), 223-235; Levine, M. A. et al., 1991, “Mapping of the gene encoding the alpha subunit of the stimulatory G protein of adenylyl cyclase (GNAS1) to 20q13.2----q13.3 in human by in situ hybridization,” published in Genomics 11 (2), 478-479, and the amino acid sequence of CHGB (identified by accession no. CAB55272) is disclosed in, e.g., Pelan, S., 2005, Direct Submission, Wellcome Trust Sanger Institute, Hinxton, Cambridgeshire, CB10 1SA, UK, each of which is incorporated by reference herein in its entirety.

The nucleotide sequence of COMP (identified by accession no. NM_{—}000095) is disclosed in, e.g., Briggs, M. D. et al., 1993, “Genetic linkage of mild pseudoachondroplasia (PSACH) to markers in the pericentromeric region of chromosome 19,” published in Genomics 18 (3), 656-660; Oehlmann, R. et al, 1994, “Genetic linkage mapping of multiple epiphyseal dysplasia to the pericentromeric region of chromosome 19,” published in Am. J. Hum. Genet. 54 (1), 3-10; Newton, G. et al., 1994, “Characterization of human and mouse cartilage oligomeric matrix protein,” published in Genomics 24 (3), 435-439, and the amino acid sequence of COMP (identified by accession no. AAC83643) is disclosed in, e.g., Deere et al., 2001, “Analysis of the promoter region of human cartilage oligomeric matrix protein (COMP),” Matrix Biol. 19 (8), 783-792, each of which is incorporated by reference herein in its entirety.

The nucleotide sequence of CORO1A (identified by accession no. NM_{—}007074) is disclosed in, e.g., Suzuki, K. et al., 1995, “Molecular cloning of a novel actin-binding protein, p57, with a WD repeat and a leucine zipper motif,” published in FEBS Lett. 364 (3), 283-288; Okumura, M., et al, 1998, “Definition of family of coronin-related proteins conserved between humans and mice: close genetic linkage between coronin-2 and CD45-associated protein,” published in DNA Cell Biol. 17 (9), 779-787; Ferrari, G. et al., 1999, “A coat protein on phagosomes involved in the intracellular survival of mycobacteria,” published in Cell 97 (4), 435-447, and the amino acid sequence of CORO1A (identified by accession no. NP_{—}009005) is disclosed in, e.g., Suzuki, K. et al., 1995, “Molecular cloning of a novel actin-binding protein, p57, with a WD repeat and a leucine zipper motif,” published in FEBS Lett. 364 (3), 283-288; Okumura, M. et al., 1998, “Definition of family of coronin-related proteins conserved between humans and mice: close genetic linkage between coronin-2 and CD45-associated protein,” published in DNA Cell Biol. 17 (9), 779-787; Ferrari, G. et al., 1999, “A coat protein on phagosomes involved in the intracellular survival of mycobacteria,” published in Cell 97 (4), 435-447, each of which is incorporated by reference herein in its entirety.

The nucleotide sequence of CPN1 (identified by accession no. NM_{—}001308) is disclosed in, e.g., Skidgel, R. A. et al., 1988, “Amino acid sequence of the N-terminus and selected tryptic peptides of the active subunit of human plasma carboxypeptidase N: comparison with other carboxypeptidases,” published in Biochem. Biophys. Res. Commun. 154 (3), 1323-1329; Gebhard, W. et al, 1989, “cDNA cloning of kinase 1,” published in Adv. Exp. Med. Biol. 247B, 261-264; Gebhard, W. et al., 1989, “cDNA cloning and complete primary structure of the small, active subunit of human carboxypeptidase N (kinase 1),” published in Eur. J. Biochem. 178 (3), 603-607, and the amino acid sequence of CPN1 (identified by accession no. NP_{—}001299) is disclosed in, e.g., Skidgel, R. A. et al., 1988, “Amino acid sequence of the N-terminus and selected tryptic peptides of the active subunit of human plasma carboxypeptidase N: comparison with other carboxypeptidases,” published in Biochem. Biophys. Res. Commun. 154 (3), 1323-1329; Gebhard, W. et al., 1989, “cDNA cloning of kinase 1,” published in Adv. Exp. Med. Biol. 247B, 261-264; Gebhard, W. et al., 1989, “cDNA cloning and complete primary structure of the small, active subunit of human carboxypeptidase N (kinase 1),” published in Eur. J. Biochem. 178 (3), 603-607, each of which is incorporated by reference herein in its entirety.

The nucleotide sequence of CUL1 (identified by accession no. NM_{—}003592) is disclosed in, e.g., Kipreos, E. T. et al., 1996, “cul-1 is required for cell cycle exit in C. elegans and identifies a novel gene family,” published in Cell 85 (6), 829-839; Lisztwan, J. et al., 1998, “Association of human CUL-1 and ubiquitin-conjugating enzyme CDC34 with the F-box protein p45 (SKP2): evidence for evolutionary conservation in the subunit composition of the CDC34-SCF pathway,” published in EMBO J. 17 (2), 368-383; Michel, J. J. et al., 1998, “Human CUL-1, but not other cullin family members, selectively interacts with SKP1 to form a complex with SKP2 and cyclin A,” published in Cell Growth Differ. 9 (6), 435-449, and the amino acid sequence of CUL1 (identified by accession no. NP_{—}003583) is disclosed in, e.g., Kipreos, E. T. et al., 1996, “cul-1 is required for cell cycle exit in C. elegans and identifies a novel gene family,” published in Cell 85 (6), 829-839; Lisztwan, J. et al, 1998, “Association of human CUL-1 and ubiquitin-conjugating enzyme CDC34 with the F-box protein p45 (SKP2): evidence for evolutionary conservation in the subunit composition of the CDC34-SCF pathway,” published in EMBO J. 17 (2), 368-383; Michel, J. J. et al., 1998, “Human CUL-1, but not other cullin family members, selectively interacts with SKP1 to form a complex with SKP2 and cyclin A,” published in Cell Growth Differ. 9 (6), 435-449, each of which is incorporated by reference herein in its entirety.

The nucleotide sequence of DET1 (identified by accession no. NM_{—}017966) is disclosed in, e.g., Eastman, S. W. et al., 2005, “Identification of human VPS37C, a component of endosomal sorting complex required for transport-I important for viral budding,” published in J. Biol. Chem. 280 (1), 628-636, and the amino acid sequence of DET1 (identified by accession no. NP_{—}060466) is disclosed in, e.g., Wertz, I. E. et al., 2004, “Human De-etiolated-1 regulates c-Jun by assembling a CUL4A ubiquitin ligase,” published in Science 303 (5662), 1371-1374, each of which is incorporated by reference herein in its entirety.

The nucleotide sequence of DSC1 (identified by accession no. BC109161) is disclosed in, e.g., Strausberg, R. L. et al., 2002, “Generation and initial analysis of more than 15,000 full-length human and mouse cDNA sequences,” published in Proc. Natl. Acad. Sci. U.S.A. 99 (26), 16899-16903, and the amino acid sequence of DET1 (identified by accession no. NP_{—}060466) is disclosed in, e.g., Wertz, I. E. et al., 2004, “Human De-etiolated-1 regulates c-Jun by assembling a CUL4A ubiquitin ligase,” published in Science 303 (5662), 1371-1374, each of which is incorporated by reference herein in its entirety.

The nucleotide sequence of F13A1 (identified by accession no. NM_{—}000129) is disclosed in, e.g., Takahashi, N. et al, 1986, “Primary structure of blood coagulation factor XIIIa (fibrinoligase, transglutaminase) from human placenta,” published in Proc. Natl. Acad. Sci. U.S.A. 83 (21), 8019-8023; Grundmann, U. et al., 1986, “Characterization of cDNA coding for human factor XIIIa,” published in Proc. Natl. Acad. Sci. U.S.A. 83 (21), 8024-8028; Ichinose, A. et al., 1986, “Amino acid sequence of the a subunit of human factor XIII,” published in Biochemistry 25 (22), 6900-6906, and the amino acid sequence of F13A1 (identified by accession no. CAC36886) is disclosed in, e.g., Sehra, H., 2005, Direct Submission, Wellcome Trust Sanger Institute, Hinxton, Cambridgeshire, CB10 1SA, UK, each of which is incorporated by reference herein in its entirety.

The nucleotide sequence of F5 (identified by accession no. NM_{—}000130) is disclosed in, e.g., Suzuki, K. et al., 1982, “Thrombin-catalyzed activation of human coagulation factor V,” published in J. Biol. Chem. 257 (11), 6556-6564; Kane, W. H. et al., 1986, “Cloning of a cDNA coding for human factor V, a blood coagulation factor homologous to factor VIII and ceruloplasmin,” published in Proc. Natl. Acad. Sci. U.S.A. 83 (18), 6800-6804; Jenny, R. J. et al., 1987, “Complete cDNA and derived amino acid sequence of human factor V,” published in Proc. Natl. Acad. Sci. U.S.A. 84 (14), 4846 4850, and the amino acid sequence of F5 (identified by accession nos. CA123065, CAB16748) is disclosed in, e.g., Bird, C., 2005, Direct Submission, Wellcome Trust Sanger Institute, Hinxton, Cambridgeshire, CB10 1SA, UK, each of which is incorporated by reference herein in its entirety.

The nucleotide sequence of GOLGA1 (identified by accession no. NM_{—}002077) is disclosed in, e.g., Griffith, K. J. et al., 1997, “Molecular cloning of a novel 97-kd Golgi complex autoantigen associated with Sjogren's syndrome,” published in Arthritis Rheum. 40 (9), 1693-1702; Barr, F. A., 1999, “A novel Rab6-interacting domain defines a family of Golgi-targeted coiled-coil proteins,” published in Curr. Biol. 9 (7), 381-384; Lu, L. et al., 2003, “Interaction of Arl1-GTP with GRIP domains recruits autoantigens Golgin-97 and Golgin-245/p230 onto the Golgi,” published in Mol. Biol. Cell 14 (9), 3767-3781, and the amino acid sequence of GOLGA1 (identified by accession no. CA139632) is disclosed in, e.g., Tracey, A., 2005, Direct Submission, Wellcome Trust Sanger Institute, Hinxton, Cambridgeshire, CB10 1SA, UK, each of which is incorporated by reference herein in its entirety.

The nucleotide sequence of HBA1 (identified by accession no. NM_{—}000558) is disclosed in, e.g., Kleihauer, E. F. et al., 1968, “Hemoglobin-Bibba or alpha-2-136Pro-beta 2, an unstable alpha chain abnormal hemoglobin,” published in Biochim. Biophys. Acta 154 (1), 220-222; Boyer, S. H. et al., 1968, “A survey of hemoglobins in the Republic of Chad and characterization of hemoglobin Chad:alpha-2-23Glu--Lys-beta-2,” published in Am. J. Hum. Genet. 20 (6), 570-578; Fujiwara, N. et al., 1971, “Hemoglobin Atago (alpha2-85Tyr beta-2) a new abnormal human hemoglobin found in Nagasaki. Biochemical studies on hemoglobins and myoglobins. VI,” published in Int. J. Protein Res. 3 (1), 35-39, and the amino acid sequence of HBA1 (identified by accession no. AAO22464) is disclosed in, e.g., Elam, D. et al., 2002, Direct Submission, Medicine/Hematology-Oncology/Hemoglobin DNA Laboratory, Medical College of Georgia, 15 th Street, AC-1000, Augusta, Ga. 30912, USA, each of which is incorporated by reference herein in its entirety.

The nucleotide sequence of HSPA5 (identified by accession no. NM_{—}005347) is disclosed in, e.g., Munro, S. et al., 1986, “An Hsp70-like protein in the ER: identity with the 78 kd glucose-regulated protein and immunoglobulin heavy chain binding protein,” published in Cell 46 (2), 291-300; Pollok, B. A. et al., 1987, “Molecular basis of the cell-surface expression of immunoglobulin mu chain without light chain in human B lymphocytes,” published in Proc. Natl. Acad. Sci. U.S.A. 84 (24), 9199-9203; Ting, J. et al., 1988, “Human gene encoding the 78,000-dalton glucose-regulated protein and its pseudogene: structure, conservation, and regulation,” published in DNA 7 (4), 275-286, and the amino acid sequence of HSPA5 (identified by accession no. NP_{—}005338) is disclosed in, e.g., Munro, S. et al., 1986, “An Hsp70-like protein in the ER: identity with the 78 kd glucose-regulated protein and immunoglobulin heavy chain binding protein,” published in Cell 46 (2), 291-300; Pollok, B. A. et al., 1987, “Molecular basis of the cell-surface expression of immunoglobulin mu chain without light chain in human B lymphocytes,” published in Proc. Natl. Acad. Sci. U.S.A. 84 (24), 9199-9203; Ting, J. et al., 1988, “Human gene encoding the 78,000-dalton glucose-regulated protein and its pseudogene: structure, conservation, and regulation,” published in DNA 7 (4), 275-286, each of which is incorporated by reference herein in its entirety.

The nucleotide sequence of HUNK (identified by accession no. NM_{—}014586) is disclosed in, e.g., Gardner, H. P. et al., 2002, “Cloning and characterization of Hunk, a novel mammalian,” published in Genomics 63 (1), 46-59; Korobko, I. V. et al., 2000, “The MAK-V protein kinase regulates endocytosis in mouse,” published in Mol. Gen. Genet. 264 (4), 411-418; Korobko, I. V. et al., 2004, “Proteinkinase MAK-V/Hunk as a possible diagnostic and prognostic marker of human breast carcinoma,” published in Arkh. Patol. 66 (5), 6-9, and the amino acid sequence of HUNK (identified by accession no. NP_{—}055401) is disclosed in, e.g., Gardner, H. P. et al., 2002, “Cloning and characterization of Hunk, a novel mammalian,” published in Genomics 63 (1), 46-59; Korobko, I. V. et al, 2000, “The MAK-V protein kinase regulates endocytosis in mouse,” published in Mol. Gen. Genet. 264 (4), 411-418; Korobko, I. V. et al., 2004, “Proteinkinase MAK-V/Hunk as a possible diagnostic and prognostic marker of human breast carcinoma,” published in Arkh. Patol. 66 (5), 6-9, each of which is incorporated by reference herein in its entirety.

The nucleotide sequence of IGFBP5 (identified by accession no. NM_{—}000599) is disclosed in, e.g., Kiefer, M. C. et al., 1991, “Molecular cloning of a new human insulin-like growth factor binding protein,” published in Biochem. Biophys. Res. Commun. 176 (1), 219-225; Ehrenborg, E. et al., 1991, “Structure and localization of the human insulin-like growth factor-binding protein 2 gene,” published in Biochem. Biophys. Res. Commun. 176 (3), 1250-1255; Shimasaki, S. et al., 1991, “Identification of five different insulin-like growth factor binding proteins (IGFBPs) from adult rat serum and molecular cloning of a novel IGFBP-5 in rat and human,” published in J. Biol. Chem. 266 (16), 10646-10653, and the amino acid sequence of IGFBP5 (identified by accession no. NP_{—}000590) is disclosed in, e.g., Kiefer, M. C. et al., 1991, “Molecular cloning of a new human insulin-like growth factor binding protein,” published in Biochem. Biophys. Res. Commun. 176 (1), 219-225; Ehrenborg, E. et al., 1991, “Structure and localization of the human insulin-like growth factor-binding protein 2 gene,” published in Biochem. Biophys. Res. Commun. 176 (3), 1250-1255; Shimasaki, S. et al., 1991, “Identification of five different insulin-like growth factor binding proteins (IGFBPs) from adult rat serum and molecular cloning of a novel IGFBP-5 in rat and human,” published I J. Biol. Chem. 266 (16), 10646-10653, each of which is incorporated by reference herein in its entirety.

The nucleotide sequence of IGHG1 (identified by accession no. BC092518) is disclosed in, e.g., Strausberg, R. L. et al., 2002, “Generation and initial analysis of more than 15,000 full-length human and mouse cDNA sequences,” published in Proc. Natl. Acad. Sci. U.S.A. 99 (26), 16899-16903, and the amino acid sequence of IGHG1 (identified by accession no. CAC20454) is disclosed in, e.g., McLean et al., 2000, “Human and murine immunoglobulin expression vector cassettes,” Mol. Immunol. 37 (14), 837-845, each of which is incorporated by reference herein in its entirety.

The nucleotide sequence of IGLV4-3 (identified by accession no. BC020236) is disclosed in, e.g., Strausberg, R. L. et al., 2002, “Generation and initial analysis of more than 15,000 full-length human and mouse cDNA sequences,” published in Proc. Natl. Acad. Sci. U.S.A. 99 (26), 16899-16903, and the amino acid sequence of IGLV4-3 (identified by accession no. AAH20236) is disclosed in, e.g., Strausberg, R. L. et al., 2002, “Generation and initial analysis of more than 15,000 full-length human and mouse cDNA sequences,” published in Proc. Natl. Acad. Sci. U.S.A. 99 (26), 16899-16903, each of which is incorporated by reference herein in its entirety.

The nucleotide sequence of KIF5C (identified by accession no. NM_{—}004984) is disclosed in, e.g., Niclas, J. et al., “Cloning and localization of a conventional kinesin motor expressed exclusively in neurons,” Neuron 12 (5), 1059-1072, 1994; and the amino acid sequence of KIF5C (identified by accession no. AAH17298) is disclosed in, e.g., Strausberg, R. L. et al., “Generation and initial analysis of more than 15,000 full-length human and mouse cDNA sequences,” Proc. Natl. Acad. Sci. U.S.A. 99 (26), 16899-16903 (2002) each of which is incorporated by reference herein in its entirety.

The nucleotide sequence of KRT10 (identified by accession no. NM_{—}000421) is disclosed in, e.g., Darmon, M. Y. et al., 1987, “Sequence of a cDNA encoding human keratin No. 10 selected according to structural homologies of keratins and their tissue-specific expression,” published in Mol. Biol. Rep. 12 (4), 277-283; Zhou, X. M. et al., 1988, “The complete sequence of the human intermediate filament chain keratin 10. Subdomainal divisions and model for folding of end domain sequences,” published in J. Biol. Chem. 263 (30), 15584-15589; Lessin, S. R. et al., 1988, “Chromosomal mapping of human keratin genes: evidence of non-linkage,” published in J. Invest. Dermatol. 91 (6), 572-578, and the amino acid sequence of KRT10 (identified by accession no. NP_{—}000412) is disclosed in, e.g., Darmon, M. Y. et al., 1987, “Sequence of a cDNA encoding human keratin No 10 selected according to structural homologies of keratins and their tissue-specific expression,” published in Mol. Biol. Rep. 12 (4), 277-283; Zhou, X. M. et al., 1988, “The complete sequence of the human intermediate filament chain keratin 10. Subdomainal divisions and model for folding of end domain sequences,” published in J. Biol. Chem. 263 (30), 15584-15589; Lessin, S. R. et al., 1988, “Chromosomal mapping of human keratin genes: evidence of non-linkage,” published in J. Invest. Dermatol. 91 (6), 572-578, each of which is incorporated by reference herein in its entirety.

The nucleotide sequence of KRT9 (identified by accession no. NM_{—}000226) is disclosed in, e.g., Reis, A. et al., 1992, “Mapping of a gene for epidermolytic palmoplantar keratoderma to the region of the acidic keratin gene cluster at 17q12-q21,” published in Hum. Genet. 90 (1-2), 113-116; Rogaev, E. I. et al., 1993, “Identification of the genetic locus for keratosis palmaris et plantaris on chromosome 17 near the RARA and keratin type I genes,” published in Nat. Genet. 5 (2), 158-162; Langbein, L. et al., 1993, “Molecular characterization of the body site-specific human epidermal cytokeratin 9: cDNA cloning, amino acid sequence, and tissue specificity of gene expression,” published in Differentiation 55 (1), 57-71, and the amino acid sequence of KRT9 (identified by accession no. NP_{—}000217) is disclosed in, e.g., Reis, A. et al., 1992, “Mapping of a gene for epidermolytic palmoplantar keratoderma to the region of the acidic keratin gene cluster at 17q12-q21,” published in Hum. Genet. 90 (1-2), 113-116; Rogaev, E. I. et al., 1993, “Identification of the genetic locus for keratosis palmaris et plantaris on chromosome 17 near the RARA and keratin type I genes,” published in Nat. Genet. 5 (2), 158-162; Langbein, L. et al., 1993, “Molecular characterization of the body site-specific human epidermal cytokeratin 9: cDNA cloning, amino acid sequence, and tissue specificity of gene expression,” published in Differentiation 55 (1), 57-71, each of which is incorporated by reference herein in its entirety.

The nucleotide sequence of LBP (identified by accession no. AF105067) is disclosed in, e.g., Long et al, 1998, “Cloning and sequencing of human lipopolysaccharide-binding protein gene,” Shengwu Huaxue Yu Shengwu Wuli Jinzhan 25, 469-471, and the amino acid sequence of LBP (identified by accession no. AAC39547) is disclosed in, e.g., Kirschning et al, 1997, “Similar organization of the lipopolysaccharide-binding protein (LBP) and phospholipid transfer protein (PLTP) genes suggests a common gene family of lipid-binding proteins,” Genomics 46 (3), 416-425, each of which is incorporated by reference herein in its entirety.

The nucleotide sequence of LUM (identified by accession no. BC035997) is disclosed in, e.g., Strausberg et al., 2002, “Generation and initial analysis of more than 15,000 full-length human and mouse cDNA sequences,” Proc. Natl. Acad. Sci. U.S.A. 99 (26), 16899-16903, and the amino acid sequence of LUM (identified by accession no. AAP35353) is disclosed in, e.g., Kalnine, N. et al., 2003, Direct Submission, BD Biosciences Clontech, 1020 East Meadow Circle, Palo Alto, Calif. 94303, USA, each of which is incorporated by reference herein in its entirety.

The nucleotide sequence of MMP14 (identified by accession no. NM_{—}004995) is disclosed in, e.g., Sato, H. et al., 1994, “A matrix metalloproteinase expressed on the surface of invasive tumour cells,” published in Nature 370 (6484), 61-65; Okada, A. et al., 1995, “Membrane-type matrix metalloproteinase (MT-MMP) gene is expressed in stromal cells of human colon, breast, and head and neck carcinomas,” published in Proc. Natl. Acad. Sci. U.S.A. 92 (7), 2730-2734; Takino, T. et al., 1995, “Cloning of a human gene potentially encoding a novel matrix metalloproteinase having a C-terminal transmembrane domain,” published in Gene 155 (2), 293-298, and the amino acid sequence of MMP14 (identified by accession no. AAV40837) is disclosed in, e.g., Livingston, R. J. et al., 2004, Direct Submission, Genome Sciences, University of Washington, 1705 NE Pacific, Seattle, Wash. 98195, USA, each of which is incorporated by reference herein in its entirety.

The nucleotide sequence of MYH4 (identified by accession no. NM_{—}017533) is disclosed in, e.g., Soussi-Yanicostas et al, 1993, “Five skeletal myosin heavy chain genes are organized as a multigene complex in the human genome,” Hum. Mol. Genet. 2 (5), 563-569; Sant'ana Pereira et al., 1995, “New method for the accurate characterization of single human skeletal muscle fibres demonstrates a relation between mATPase and MyHC expression in pure and hybrid fibre types,” J. Muscle Res. Cell. Motil. 16 (1), 21-34, and the amino acid sequence of MYH4 (identified by accession no. NP_{—}060003) is disclosed in, e.g., Soussi-Yanicostas et al., 1993, “Five skeletal myosin heavy chain genes are organized as a multigene complex in the human genome,” Hum. Mol. Genet. 2 (5), 563-569; Sant'ana Pereira et al., 1995, “New method for the accurate characterization of single human skeletal muscle fibres demonstrates a relation between mATPase and MyHC expression in pure and hybrid fibre types,” J. Muscle Res. Cell. Motil. 16 (1), 21-34; and Weiss et al., 1999, “Organization of human and mouse skeletal myosin heavy chain gene clusters is highly conserved,” Proc. Natl. Acad. Sci. U.S.A. 96 (6), 2958-2963, each of which is incorporated by reference herein in its entirety.

The nucleotide sequence of NEB (identified by accession no. NM_{—}004543) is disclosed in, e.g., Stedman, H. et al, 1988, “Nebulin cDNAs detect a 25-kilobase transcript in skeletal muscle and localize to human chromosome 2,” published in Genomics 2 (1), 1-7; Zeviani, M. et al., 1988, “Cloning and expression of human nebulin cDNAs and assignment of the gene to chromosome 2q31-q32,” published in Genomics 2 (3), 249-256; Labeit, S. et al., 1991, “Evidence that nebulin is a protein-ruler in muscle thin filaments,” published in FEBS Lett. 282 (2), 313-316, and the amino acid sequence of NEB (identified by accession no. NP_{—}004534) is disclosed in, e.g., Stedman, H. et al, 1988, “Nebulin cDNAs detect a 25-kilobase transcript in skeletal muscle and localize to human chromosome 2,” published in Genomics 2 (1), 1-7; Zeviani, M. et al., 1988, “Cloning and expression of human nebulin cDNAs and assignment of the gene to chromosome 2q31-q32,” published in Genomics 2 (3), 249-256; Labeit, S. et al., 1991, “Evidence that nebulin is a protein-ruler in muscle thin filaments,” published in FEBS Lett. 282 (2), 313-316, each of which is incorporated by reference herein in its entirety.

The nucleotide sequence of NUCB2 (identified by accession no. NM_{—}005013) is disclosed in, e.g., Barnikol-Watanabe, S. et al., 1994, “Human protein NEFA, a novel DNA binding/EF-hand/leucine zipper protein. Molecular cloning and sequence analysis of the cDNA, isolation and characterization of the protein,” published in Biol. Chem. Hoppe-Seyler 375 (8), 497-512; Kroll, K. A. et al., 1999, “Heterologous overexpression of human NEFA and studies on the two EF-hand calcium-binding sites,” published in Biochem. Biophys. Res. Commun. 260 (1), 1-8; Taniguchi, N. et al., 2000, “The post mitotic growth suppressor necdin interacts with a calcium-binding protein (NEFA) in neuronal cytoplasm,” published in J. Biol. Chem. 275 (41), 31674-31681, and the amino acid sequence of NUCB2 (identified by accession no. NP_{—}005004) is disclosed in, e.g., Barnikol-Watanabe, S. et al, 1994, “Human protein NEFA, a novel DNA binding/EF-hand/leucine zipper protein. Molecular cloning and sequence analysis of the cDNA, isolation and characterization of the protein,” published in Biol. Chem. Hoppe-Seyler 375 (8), 497-512; Kroll, K. A. et al., 1999, “Heterologous overexpression of human NEFA and studies on the two EF-hand calcium-binding sites,” published in Biochem. Biophys. Res. Commun. 260 (1), 1-8; Taniguchi, N. et al., 2000, “The postmitotic growth suppressor necdin interacts with a calcium-binding protein (NEFA) in neuronal cytoplasm,” published in J. Biol. Chem. 275 (41), 31674-31681, each of which is incorporated by reference herein in its entirety.

The nucleotide sequence of ORM2 (identified by accession no. NM_{—}000608) is disclosed in, e.g., Schmid, K. et al., 1973, “Structure of 1-acid glycoprotein. The complete amino acid sequence, multiple amino acid substitutions, and homology with the immunoglobulins,” published in Biochemistry 12 (14), 2711-2724; Schmid, K. et al., 1974, “The disulfide bonds of alpha1-acid glycoprotein,” published in Biochemistry 13 (13), 2694-2697; Dente, L. et al, 1987, “Structure and expression of the genes coding for human alpha 1-acid glycoprotein,” published in EMBO J. 6 (8), 2289-2296, and the amino acid sequence of ORM2 (identified by accession no. NP_{—}000599) is disclosed in, e.g., Schmid, K. et al., 1973, “Structure of 1-acid glycoprotein. The complete amino acid sequence, multiple amino acid substitutions, and homology with the immunoglobulins,” published in Biochemistry 12 (14), 2711-2724; Schmid, K. et al., 1974, “The disulfide bonds of alpha1-acid glycoprotein,” published in Biochemistry 13 (13), 2694-2697; Dente, L. et al., 1987, “Structure and expression of the genes coding for human alpha 1-acid glycoprotein,” published in EMBO J. 6 (8), 2289-2296, each of which is incorporated by reference herein in its entirety.

The nucleotide sequence of PF4V1 (identified by accession no. NM_{—}002620) is disclosed in, e.g., Green, C. J. et al., 1989, “Identification and characterization of PF4varl, a human gene variant of platelet factor 4,” published in Mol. Cell. Biol. 9 (4), 1445-1451; Eisman, R. et al., 1990, “Structural and functional comparison of the genes for human platelet factor 4 and PF4alt,” published in Blood 76 (2), 336-344, and the amino acid sequence of PF4V1 (identified by accession no. NP_{—}002611) is disclosed in, e.g., Green, C. J. et al., 1989, “Identification and characterization of PF4varl, a human gene variant of platelet factor 4,” published in Mol. Cell. Biol. 9 (4), 1445-1451; Eisman, R. et al., 1990, “Structural and functional comparison of the genes for human platelet factor 4 and PF4alt,” published in Blood 76 (2), 336-344, each of which is incorporated by reference herein in its entirety.

The nucleotide sequence of PIGR (identified by accession no. NM_{—}002644) is disclosed in, e.g., Mizoguchi, A. et al., 1982, “Structures of the carbohydrate moieties of secretory component purified from human milk,” published in J. Biol. Chem. 257 (16), 9612-9621; Hood, L. et al., 1985, “T cell antigen receptors and the immunoglobulin supergene family,” published in Cell 40 (2), 225-229; Davidson, M. K. et al., 1988, “Genetic mapping of the human polymeric immunoglobulin receptor gene to chromosome region 1q31----q41,” published in Cytogenet. Cell Genet. 48 (2), 107-111, and the amino acid sequence of PIGR (identified by accession no. CAC10060) is disclosed in, e.g., Schjerven, 2000, “Mechanism of IL-4-mediated up-regulation of the polymeric Ig receptor: role of STAT6 in cell type-specific delayed transcriptional response,” J. Immunol. 165 (7), 3898-3906, each of which is incorporated by reference herein in its entirety.

The nucleotide sequence of PLG (identified by accession no. NM_{—}000301) is disclosed in, e.g., Robbins, K. C. et al., 1967, “The peptide chains of human plasmin. Mechanism of activation of human plasminogen to plasmin,” published in J. Biol. Chem. 242 (10), 2333-2342; Deutsch, D. G. et al. 1970, “Plasminogen: purification from human plasma by affinity chromatography,” published in Science 170 (962), 1095-1096; Wiman, B. et al., 1979, “On the mechanism of the reaction between human alpha 2-antiplasmin and plasmin,” published in J. Biol. Chem. 254 (18), 9291-9297, and the amino acid sequence of PLG (identified by accession no. AAH60513) is disclosed in, e.g., Strausberg et al., 2002, “Generation and initial analysis of more than 15,000 full-length human and mouse cDNA sequences,” Proc. Natl. Acad. Sci. U.S.A. 99 (26), 16899-16903, each of which is incorporated by reference herein in its entirety.

The nucleic acid sequence for plasminogen precursor is disclosed in, e.g., Forsgren et al., 1987, FEBS Lett 213, 254-260, and amino acid sequence of plasminogen precursor (identified by accession no. P00747) is disclosed in, e.g., Petersen et al., “Characterization of the gene for human plasminogen, a key proenzyme in the fibrinolytic system, 1990, J. Biol. Chem. 265 (11), 6104-6111; and Forsgren et al., 1987, “Molecular cloning and characterization of a full-length cDNA clone for human plasminogen, FEBS Lett. 213 (2), 254-260, each of which is incorporated by reference herein in its entirety.

The nucleotide sequence of PON1 (identified by accession no. NM_{—}000446) is disclosed in, e.g., Ortigoza-Ferado, J. et al., 1984, “Paraoxon hydrolysis in human serum mediated by a genetically variable arylesterase and albumin,” published in Am. J. Hum. Genet. 36 (2), 295-305; Gan, K. N. et al., 1991, “Purification of human serum paraoxonase/arylesterase. Evidence for one esterase catalyzing both activities,” published in Drug Metab. Dispos. 19 (1), 100-106; Hassett, C. et al., 1991, “Characterization of cDNA clones encoding rabbit and human serum paraoxonase: the mature protein retains its signal sequence,” published in Biochemistry 30 (42), 10141-10149, and the amino acid sequence of PON1 (identified by accession no. NP_{—}000437) is disclosed in, e.g., Ortigoza-Ferado, J. et al., 1984, “Paraoxon hydrolysis in human serum mediated by a genetically variable arylesterase and albumin,” published in Am. J. Hum. Genet. 36 (2), 295-305; Gan, K. N. et al., 1991, “Purification of human serum paraoxonase/arylesterase. Evidence for one esterase catalyzing both activities,” published in Drug Metab. Dispos. 19 (1), 100-106; Hassett, C. et al., 1991, “Characterization of cDNA clones encoding rabbit and human serum paraoxonase: the mature protein retains its signal sequence,” published in Biochemistry 30 (42), 10141-10149, each of which is incorporated by reference herein in its entirety.

The nucleotide sequence of PPBP (identified by accession no. NM_{—}002704) is disclosed in, e.g., Begg, G. S. et al., 1978, “Complete covalent structure of human beta-thromboglobulin,” published in Biochemistry 17 (9), 1739-1744; Kaplan, K. L., 1979, “Platelet alpha-granule proteins: studies on release and subcellular localization,” published in Blood 53 (4), 604-618; McLaren, K. M. et al., 1982, “Immunological localisation of beta-thromboglobulin and platelet factor 4 in human megakaryocytes and platelets,” published in J. Clin. Pathol. 35 (11), 1227-1231, and the amino acid sequence of PPBP (identified by accession no. CAG33086) is disclosed in, e.g., Ebert, L. et al., 2004, Direct Submission, RZPD Deutsches Ressourcenzentrum fuer Genomforschung GmbH, Im Neuenheimer Feld 580, D-69120 Heidelberg, Germany; Ebert, L. et al., unpublished, “Cloning of human full open reading frames in Gateway™ system entry vector (pDONR201),” each of which is incorporated by reference herein in its entirety.

The nucleotide sequence of RBP4 (identified by accession no. NM_{—}006744) is disclosed in, e.g., Rask, L. et al., 1971, “Studies on two physiological forms of the human retinol-binding protein differing in vitamin A and arginine content,” published in J. Biol. Chem. 246 (21), 6638-6646; Fex, G. et al., 1979, “Retinol-binding protein from human urine and its interaction with retinol and prealbumin,” published in Eur. J. Biochem. 94 (1), 307-313; Fex, G. et al., 1979, “Interaction between prealbumin and retinol-binding protein studied by affinity chromatography, gel filtration and two-phase partition,” published in Eur. J. Biochem. 99 (2), 353-360, and the amino acid sequence of RBP4 (identified by accession no. CAH72328) is disclosed in, e.g., Brown, A., 2005, Direct Submission, Wellcome Trust Sanger Institute, Hinxton, Cambridgeshire, CB10 1SA, UK, each of which is incorporated by reference herein in its entirety.

The nucleotide sequence of RIMS1 (identified by accession no. NM_{—}014989) is disclosed in, e.g., Kelsell, R. E. et al., 1998, “Localization of a gene (CORD7) for a dominant cone-rod dystrophy to chromosome 6q,” published in Am. J. Hum. Genet. 63 (1), 274-279; Betz, A. et al, 2001, “Functional interaction of the active zone proteins Munc13-1 and RIM1 in synaptic vesicle priming,” published in Neuron 30 (1), 183-196; Coppola, T. et al., 2001, “Direct interaction of the Rab3 effector RIM with Ca2+ channels, SNAP-25, and synaptotagmin,” published in J. Biol. Chem. 276 (35), 32756-32762, and the amino acid sequence of RIMS1 (identified by accession no. NP_{—}055804) is disclosed in, e.g., Kelsell, R. E. et al., 1998, “Localization of a gene (CORD7) for a dominant cone-rod dystrophy to chromosome 6q,” published in Am. J. Hum. Genet. 63 (1), 274-279; Betz, A. et al., 2001, “Functional interaction of the active zone proteins Munc13-1 and RIM1 in synaptic vesicle priming,” published in Neuron 30 (1), 183-196; Coppola, T. et al., 2001, “Direct interaction of the Rab3 effector RIM with Ca2+ channels, SNAP-25, and synaptotagmin,” published in J. Biol. Chem. 276 (35), 32756-32762, each of which is incorporated by reference herein in its entirety.

The nucleotide sequence of RNF6 (identified by accession no. NM_{—}005977) is disclosed in, e.g., Macdonald, D. H. et al., 1999, “Cloning and characterization of RNF6, a novel RING finger gene mapping to 13q12” published in Genomics 58 (1), 94-97; Lopez, P., et al., 2002, “Gene control in germinal differentiation: RNF6, a transcription regulatory protein in the mouse sertoli cell” published in Mol. Cell. Biol. 22 (10), 3488-3496; Lo, H. S. et al., 2002, “Identification of somatic mutations of the RNF6 gene in human esophageal squamous cell carcinoma” published in Cancer Res. 62 (15), 4191-4193, and the amino acid sequence of RNF6 (identified by accession no. CAH73183) is disclosed in, e.g., Tromans, A. et al., 2004, Direct Submission, Wellcome Trust Sanger Institute, Hinxton, Cambridgeshire, CB10 1SA, UK, each of which is incorporated by reference herein in its entirety.

The nucleotide sequence of SEMA3D (identified by accession no. NM_{—}152754) is disclosed in, e.g., Scherer, S. W. et al, 2003, “Human chromosome 7: DNA sequence and biology” published in Science 300 (5620), 767-772; Clark, H. F., et al., 2003, “The secreted protein discovery initiative (SPDI), a large-scale effort to identify novel human secreted and transmembrane proteins: a bioinformatics assessment” published in Genome Res. 13 (10), 2265-2270; and the amino acid sequence of SEMA3D (identified by accession no. EAL24184) is disclosed in, e.g., Scherer et al., 2003, “Human chromosome 7: DNA sequence and biology,” Science 300 (5620), 767-772, each of which is incorporated by reference herein in its entirety.

The nucleotide sequence of SF3B1 (identified by accession no. NM_{—}012433) is disclosed in, e.g., Wang, C. et al., 1998, “Phosphorylation of spliceosomal protein SAP 155 coupled with splicing catalysis,” published in Genes Dev. 12 (10), 1409-1414; Pauling, M. H. et al. 2000, “Functional Cus1p is found with Hsh155p in a multiprotein splicing factor associated with U2 snRNA” published in Mol. Cell. Biol. 20 (6), 2176-2185; Will, C. L. et al., 2001, “A novel U2 and U11/U12 snRNP protein that associates with the pre-mRNA branch site” published in EMBO J. 20 (16), 4536-4546, and the amino acid sequence of SF3B1 (identified by accession no. NP_{—}006833) is disclosed in, e.g., Gozani, O. et al, 1996, “Evidence that sequence-independent binding of highly conserved U2 snRNP proteins upstream of the branch site is required for assembly of spliceosomal complex A” published in Genes Dev. 10 (2), 233-243; Agell, N., et al., 1998, “New nuclear functions for calmodulin” published in Cell Calcium 23 (2-3), 115-121; Das, B. K., et al, 1999, “Characterization of a protein complex containing spliceosomal proteins SAPs 49, 130, 145, and 155” published in Mol. Cell. Biol. 19 (10), 6796-6802, each of which is incorporated by reference herein in its entirety.

The nucleotide sequence of SPINK1 (identified by accession no. NM_{—}003122) is disclosed in, e.g., Huhtala, M. L. et al., 1982, inhibitor from the urine of a patient with ovarian cancer,” published in J. Biol. Chem. 257 (22), 13713-13716; Yamamoto, T. et al., 1985, “Molecular cloning and nucleotide sequence of human pancreatic secretory trypsin inhibitor (PSTI) cDNA” published in Biochem. Biophys. Res. Commun. 132 (2), 605-612; Horii, A., et al., 1987, “Primary structure of human pancreatic secretory trypsin inhibitor (PSTI) gene” published in Biochem. Biophys. Res. Commun. 149 (2), 635-641, and the amino acid sequence of SPINK1 (identified by accession no. NP_{—}003113) is disclosed in, e.g., Huhtala, M. L. et al., 1982, inhibitor from the urine of a patient with ovarian cancer,” published in J. Biol. Chem. 257 (22), 13713-13716; Yamamoto, T. et al., 1985, “Molecular cloning and nucleotide sequence of human pancreatic secretory trypsin inhibitor (PSTI) cDNA” published in Biochem. Biophys. Res. Commun. 132 (2), 605-612; Horii, A. et al., 1987, “Primary structure of human pancreatic secretory trypsin inhibitor (PSTI) gene” published in Biochem. Biophys. Res. Commun. 149 (2), 635-641, each of which is incorporated by reference herein in its entirety.

The nucleotide sequence of SPP1 (identified by accession no. NM_{—}000582) is disclosed in, e.g., Kiefer, M. C. et al., 1989, “The cDNA and derived amino acid sequence for human osteopontin” published in Nucleic Acids Res. 17 (8), 3306; Nemir, M., et al., 1989, “Normal rat kidney cells secrete both phosphorylated and nonphosphorylated forms of osteopontin showing different physiological properties” published in J. Biol. Chem. 264 (30), 18202-18208; Fisher, L. W. et al., “Human bone sialoprotein. Deduced protein sequence and chromosomal localization” published in J. Biol. Chem. 265 (4), 2347-2351, and the amino acid sequence of SPP1 (identified by accession no. AAH17387) is disclosed in, e.g., Strausberg, R. L. et al., 2002, “T Generation and initial analysis of more than 15,000 full-length human and mouse cDNA sequences published in Proc. Natl. Acad. Sci. U.S.A. 99 (26), 16899-16903, each of which is incorporated by reference herein in its entirety.

The nucleotide sequence of SPTB (identified by accession no. NM_{—}001024858) is disclosed in, e.g., Carlier, M. F. et al., 1984, “Interaction between microtubule-associated protein tau and spectrin” published in Biochimie 66 (4), 305-311; Prchal, J. T., et al., 1987, “Isolation and characterization of cDNA clones for human erythrocyte beta-spectrin” published in Proc. Natl. Acad. Sci. U.S.A. 84 (21), 7468-7472; Winkelmann, J. C. et al., 1988, “Molecular cloning of the cDNA for human erythrocyte beta-spectrin” published in Blood 72 (1), 328-334, and the amino acid sequence of SPTB (identified by accession no. BAD92652) is disclosed in, e.g., Totoki, Y. et al, 2000, Direct Submission, Osamu Ohara, Kazusa DNA Research Institute, Department of Human Gene Research; 2-6-7 Kazusa-kamatari, Kisarazu, Chiba, 292-0818, Japan, each of which is incorporated by reference herein in its entirety.

The nucleotide sequence of SYNE1 (identified by accession no. NM_{—}182961) is disclosed in, e.g., Zhang, Q. et al., “Nesprins: a novel family of spectrin-repeat-containing proteins that localize to the nuclear membrane in multiple tissues,” J. Cell. Sci. 114 (PT 24), 4485-4498 (2001); Apel, E. D. et al., “Syne-1, a dystrophin- and Klarsicht-related protein associated with synaptic nuclei at the neuromuscular junction,” J. Biol. Chem. 275 (41), 31986-31995 (2000); Nedivi, E., et al., “A set of genes expressed in response to light in the adult cerebral cortex and regulated during development,” Proc. Natl. Acad. Sci. U.S.A. 93 (5), 2048-2053 (1996), and the amino acid sequence of SYNE1 (identified by accession no. AAH39121) is disclosed in, e.g., Strausberg, R. L. et al., “Generation and initial analysis of more than 15,000 full-length human and mouse cDNA sequences,” Proc. Natl. Acad. Sci. U.S.A. 99 (26), 16899-16903 (2002), each of which is incorporated by reference herein in its entirety.

The nucleotide sequence of TAF4B (identified by accession no. NM_{—}003187) is disclosed in, e.g., Parada, C. A., et al., “A novel LBP-1-mediated restriction of HIV-1 transcription at the level of elongation in vitro,” J. Biol. Chem. 270 (5), 2274-2283 (1995); Zhou and Sharp, “Novel mechanism and factor for regulation by HIV-1 Tat,” EMBO J. 14 (2), 321-328 (1995); Ou et al, “Role of flanking E box motifs in human immunodeficiency virus type 1 TATA element function,” J. Virol. 68 (11), 7188-7199 (1994); Kashanchi, F. et al. “Direct interaction of human TFIID with the HIV-1 transactivator tat,” Nature 367 (6460), 295-299 (1994), each of which is hereby incorporated herein by reference in its entirety and the amino acid sequence of TAF4B (identified by accession no. XP_{—}290809) is predicted by automated computational analysis of the annotated genomic sequence (NT_{—}010966) using gene prediction method: GNOMON.

The nucleotide sequence of TBC1D1 (identified by accession no. NM_{—}015173) is disclosed in, e.g., White, R. A. et al., 2000, “The gene encoding TBC1D1 with homology to the tre-2/USP6 oncogene, BUB2, and cdc 16 maps to mouse chromosome 5 and human chromosome 4” published in Cytogenet. Cell Genet. 89 (3-4), 272-275; and the amino acid sequence of TBC1D1 (identified by accession no. NP_{—}055988) is disclosed in, e.g., White, R. A. et al, 2000, “The gene encoding TBC1D1 with homology to the tre-2/USP6 oncogene, BUB2, and cdc16 maps to mouse chromosome 5 and human chromosome 4,” published in Cytogenet. Cell Genet. 89 (3-4), 272-275, each of which is incorporated by reference herein in its entirety.

The nucleotide sequence of TLN1 (identified by accession no. NM_{—}006289) is disclosed in, e.g., Kupfer et al, 1990, “The PMA-induced specific association of LFA-1 and talin in intact cloned T helper cells” published in J. Mol. Cell. Immunol. 4 (6), 317-325; Luna, E. J. et al, 1992, “Cytoskeleton—plasma membrane interactions” published in Science 258 (5084), 955-964; Salgia, R. et al., 1995, “Molecular cloning of human paxillin, a focal adhesion protein phosphorylated by P21 OBCR/ABL” published in J. Biol. Chem. 270 (10), 5039-5047, and the amino acid sequence of TLN1 (identified by accession no. NP_{—}006280) is disclosed in, e.g., Kupfer, A. et al., 1990, “The PMA-induced specific association of LFA-1 and talin in intact cloned T helper cells” published in J. Mol. Cell. Immunol. 4 (6), 317-325; Luna, E. J. et al., 1992, “Cytoskeleton—plasma membrane interactions” published in Science 258 (5084), 955-964; Salgia, R. et al., 1995, “Molecular cloning of human paxillin, a focal adhesion protein phosphorylated by P21 OBCR/ABL” published in J. Biol. Chem. 270 (10), 5039-5047, each of which is incorporated by reference herein in its entirety.

The nucleotide sequence of TMSB4X (identified by accession no. NM_{—}021109) is disclosed in, e.g., Erickson-Viitanen, S. et al., 1983, “Distribution of thymosin beta 4 in vertebrate classes” published in Arch. Biochem. Biophys. 221 (2), 570-576; Friedman, R. L. et al., 1984, “Transcriptional and posttranscriptional regulation of interferon-induced gene expression in human cells” published in Cell 38 (3), 745-755; Soma, G. et al., 1985, “Detection of a countertranscript in promyelocytic leukemia cells HL60 during early differentiation by TPA” published in Biochem. Biophys. Res. Commun. 132 (1), 100-109, and the amino acid sequence of TMSB4X (identified by accession no. NP_{—}066932) is disclosed in, e.g., Erickson-Viitanen, S. et al., 1983, “Distribution of thymosin beta 4 in vertebrate classes” published in Arch. Biochem. Biophys. 221 (2), 570-576; Friedman, R. L. et al., 1984, “Transcriptional and posttranscriptional regulation of interferon-induced gene expression in human cells” published in Cell 38 (3), 745-755; Soma, G. et al., 1985, “Detection of a countertranscript in promyelocytic leukemia cells HL60 during early differentiation by TPA” published in Biochem. Biophys. Res. Commun. 132 (1), 100-109, each of which is incorporated by reference herein in its entirety.

The nucleotide sequence of UROC1 (identified by accession no. NM_{—}144639) is disclosed in, e.g., Yamada, S. et al., 2004, “Expression profiling and differential screening between hepatoblastomas and the corresponding normal livers: identification of high expression of the PLK1 oncogene as a poor-prognostic indicator of hepatoblastomas” published in Oncogene 23 (35), 5901-5911, and the amino acid sequence of UROC1 (identified by accession no. NP_{—}653240) is disclosed in, e.g., Yamada, S. et al., 2004, “Expression profiling and differential screening between hepatoblastomas and the corresponding normal livers: identification of high expression of the PLK1 oncogene as a poor-prognostic indicator of hepatoblastomas” published in Oncogene 23 (35), 5901-5911, each of which is incorporated by reference herein in its entirety.

The nucleotide sequence of ZFHX2 (identified by accession no. NM_{—}033400) is disclosed in, e.g., Nagase, T. et al., 2000, “Prediction of the coding sequences of unidentified human genes. XIX. The complete sequences of 100 new cDNA clones from brain which code for large proteins in vitro” published in DNA Res. 7 (6), 347-355; and the amino acid sequence of ZFHX2 (identified by accession no. NP_{—}207646) is disclosed in, e.g., Nagase, T. et al, 2000, “Prediction of the coding sequences of unidentified human genes. XIX. The complete sequences of 100 new cDNA clones from brain which code for large proteins in vitro” published in DNA Res. 7 (6), 347-355, each of which is incorporated by reference herein in its entirety.

The nucleotide sequence of ZYX (identified by accession no. NM_{—}003461) is disclosed in, e.g., Wang, L. F. et al, 1994, “Gene encoding a mammalian epididymal protein” published in Biochem. Mol. Biol. Int. 34 (6), 1131-1136; Reinhard, M. et al, 1995, “Identification, purification, and characterization of a zyxin-related protein that binds the focal adhesion and microfilament protein VASP (vasodilator-stimulated phosphoprotein),” Proc. Natl. Acad. Sci. U.S.A. 92 (17), 7956-7960; Hobert, O. et al, 1996, “SH3 domain-dependent interaction of the proto-oncogene product Vav with the focal contact protein zyxin” published in Oncogene 12 (7), 1577-1581, and the amino acid sequence of ZYX (identified by accession no. NP_{—}001010972) is disclosed in, e.g., Wang, L. F. et al., 1994, “Gene encoding a mammalian epididymal protein” published in Biochem. Mol. Biol. Int. 34 (6), 1131-1136; Reinhard, M. et al., 1995, “Identification, purification, and characterization of a zyxin-related protein that binds the focal adhesion and microfilament protein VASP (vasodilator-stimulated phosphoprotein)” Proc. Natl. Acad. Sci. U.S.A. 92 (17), 7956-7960; Hobert, O. et al., 1996, “SH3 domain-dependent interaction of the proto-oncogene product Vav with the focal contact protein zyxin” published in Oncogene 12 (7), 1577-1581, each of which is incorporated by reference herein in its entirety.

5.6.2 Isolation of Useful Biomarkers

The biomarkers of the present invention may, for example, be used to raise antibodies that bind the biomarker if it is a protein (using methods described herein, or any method well known to those of skill in the art), or they may be used to develop a specific oligonucleotide probe, if it is a nucleic acid, for example, using a method described herein, or any method well known to those of skill in the art. The skilled artisan will readily appreciate that useful features can be further characterized to determine the molecular structure of the biomarker. Methods for characterizing biomarkers in this fashion are well-known in the art and include X-ray crystallography, high-resolution mass spectrometry, infrared spectrometry, ultraviolet spectrometry and nuclear magnetic resonance. Methods for determining the nucleotide sequence of nucleic acid biomarkers, the amino acid sequence of polypeptide biomarkers, and the composition and sequence of carbohydrate biomarkers also are well-known in the art.

### 5.7 Application of the Present Invention to Sirs Subjects

In one embodiment, the presently described methods are used to screen SIRS subjects who are at risk for developing sepsis. A one or more biological samples are taken from a SIRS-positive subject at one or more different time points and used to construct a biomarker profile. The biomarker profile is then evaluated to determine whether the feature values of the biomarker profile satisfy a first value set associated with a particular decision rule. This evaluation classifies the subject as a converter or a nonconverter. A treatment regimen may then be initiated to forestall or prevent the progression of sepsis when the subject is classified as a converter.

### 5.8 Application of the Present Invention to Stages of Sepsis

In one embodiment, the presently described methods are used to screen subjects who are particularly at risk for developing a certain stage of sepsis. A biological sample is taken from a subject and used to construct a biomarker profile. The biomarker profile is then evaluated to determine whether the feature values of the biomarker profile satisfy a first value set associated with a particular decision rule. This evaluation classifies the subject as having or not having a particular stage of sepsis. A treatment regimen may then be initiated to treat the specific stage of sepsis. In some embodiments, the stage of sepsis is for example, onset of sepsis, severe sepsis, septic shock, or multiple organ dysfunction.

### 5.9 Exemplary Embodiments

In some embodiments of the present invention, a biomarker profile is obtained using a biological sample from a test subject, particularly a subject at risk of developing sepsis, having sepsis, or suspected of having sepsis. The biomarker profile in such embodiments is evaluated. This evaluation can be made, for example, by applying a decision rule to the test subject. The decision rule can, for example, be or have been constructed based upon the biomarker profiles obtained from subjects in the training population. The training population, in one embodiment, includes (a) subjects that had SIRS and were then diagnosed as septic during an observation time period as well as (b) subjects that had SIRS and were not diagnosed as septic during an observation time period. If the biomarker profile from the test subject contains appropriately characteristic features, then the test subject is diagnosed as having a more likely chance of becoming septic, as being afflicted with sepsis or as being at the particular stage in the progression of sepsis. Various populations of subjects including those who are suffering from SIRS (e.g., SIRS-positive subjects) or those who are suffering from an infection but who are not suffering from SIRS (e.g., SIRS-negative subjects) can serve as training populations. Accordingly, the present invention allows the clinician to distinguish, inter alia, between those subjects who do not have SIRS, those who have SIRS but are not likely to develop sepsis within a given time frame, those who have SIRS and who are at risk of eventually becoming septic, and those who are suffering from a particular stage in the progression of sepsis.

### 5.10 Use of Annotation Data to Identify Discriminating Biomarkers

In some embodiments, data analysis algorithms identify a large set of biomarkers whose features discriminate between converters and nonconverters. For example, in some embodiments, application of a data analysis algorithm to a training population results in the selection of more than 500 biomarkers, more than 1000 biomarkers, or more than 10,000 biomarkers. In some embodiments, further reduction in the number of biomarkers that are deemed to be discriminating is desired. Accordingly, in some embodiments, filtering rules that are complementary to data analysis algorithms (e.g., the data analysis algorithms of Section 5.5) are used to further reduce the list of discriminating biomarkers identified by the data analysis algorithms. Specifically, the list of biomarkers identified by application of one or more data analysis algorithms to the biomarker profile data measured in a training population is further refined by application of annotation data based filtering rules to the list. In such embodiments, those biomarkers in the set of biomarkers identified by the one or more data analysis algorithms that satisfy the one or more applied annotation data based filtering rules remain in the set of discriminating biomarkers. In some instances, those biomarkers in the set of biomarkers identified by the one or more data analysis algorithms that do not satisfy the one or more applied annotation data based filtering rules are removed from the set. In other instances, those biomarkers in the set of biomarkers identified by the one or more data analysis algorithms that do not satisfy the one or more applied annotation data based filtering rules stay in the set and those that satisfy the one or more applied annotation data based filtering rules are removed from the set. In this way, annotation data can be used to reduce the number of biomarkers in the set of discriminating biomarkers identified by the data analysis algorithms.

Annotation data based filtering rules are rules based upon annotation data. Annotation data refers to any type of data that describes a property of a biomarker. An example of annotation data is the identification of biological pathways to which a given biomarker belongs. Another example of annotation data is enzymatic class (e.g., phosphodiesterases, kinases, metalloproteinases, etc.). Still other examples of annotation data include, but are not limited to, protein domain information, enzymatic substrate information, enzymatic reaction information, and protein interaction data. Yet another example of annotation data is disease association, in other words, which disease process a given biomarker has been linked to or otherwise affects. Another form of annotation data is any type of data that associates biomarker expression, other forms of biomarker abundance, and/or biomarker activity, with cellular localization, tissue type localization, and/or cell type localization.

As the name implies, annotation data is used to construct an annotation data based filtering rule. An example of an annotation data based filtering rule is:

Annotation Rule 1:

remove all transcription factors from the training set.

Application of this filtering rule to a set of biomarkers will remove all transcription factors from the set.

Another type of annotation data based filtering rule is:

Annotation Rule 2:

keep all biomarkers that are enriched for annotation X in a biomarker list.

Application of this filtering rule will only keep those biomarkers in a given list that are enriched (overrepresented) for annotation X in the list. To more fully appreciate this filtering rule, consider an exemplary biomarker set that has been identified by application of a data analysis algorithm (Section 5.5) to biomarker profiles measured using training population data measured in accordance with a technique disclosed in Section 5.4. This exemplary biomarker set has 500 biomarkers. Assume, for in this illustrative example, that the full set of biomarkers in a human consists of 25,000 biomarkers. Here, the 25,000 biomarkers is a population and the 500 biomarker set is the sample. As used here, the term “population” consists of all possible observable biomarkers. The term “sample” is the data that is actually considered. Now, for this example, let X=kinases. Suppose there are 800 known human kinases and further suppose that the set of 500 biomarkers was randomly selected with respect to kinases. Under these circumstances, the list of 500 biomarkers identified by the data analysis algorithms should select about (500/25,000)*800=16 kinases. Since there are, in fact, 50 kinases in the sample, a conclusion can be reached that kinases are indeed enriched in the sample relative to the population.

More formally, in this example, a determination can be made as to whether kinases are enriched in the set of biomarkers identified by the data analysis algorithm (the sample) relative to the population by analysis of the two-way contingency table that describes the observed sample and population:

Kinase

| Group | Yes | No | Total |
|---|---|---|---|
| Population | 800 | 24,200 | 25,000 |
| Sample | 50 | 450 | 500 |

Following Agresti, 1996, An Introduction to Categorical Data Analysis, John Wiley & Sons, New York, which is hereby incorporated by reference in its entirety, this two-way contingency table can be analyzed by treating each row as an independent bionomial variable. In such instances, the true difference in proportions, termed π₁-π₂, compares the probabilities in the two rows. This difference falls between −1 and +1. It equals zero when π₁=π₂; that is, when the selection of kinases in the sample from the population is independent of the kinase annotation. Of the N₁=25,000 biomarkers in the population, 800 are kinases, a proportion of p₁= 800/25,000=0.032. Of the N₂=500 biomarkers in the sample identified using a data analysis algorithm, 50 are kinases, a proportion p₂ of 50/500=0.10. The sample difference of proportions is 0.032−0.10=−0.068. In accordance with Agresti, when the counts in the two rows are independent binomial samples, the estimated standard error of p₁−p₂ is:

$${σ}^{)} ( {p}_{1} - {p}_{2} ) = \sqrt{\frac{{p}_{1} ( 1 - {p}_{1} )}{{N}_{1}} + \frac{{p}_{2} ( 1 - {p}_{2} )}{{N}_{2}}}$$

where N₁ and N₂ are the samples sizes for the population and the sample selected by data analysis algorithm, respectively. The standard error decreases, and hence the estimate of π₁−π₂ improves, as the sample sizes increase. A large-sample (100(1−α)) % confidence interval for π₁−π₂ is

(p₁+p₂)±z_{a/2}=z_{0.025}=1.96

Thus, for this example, the estimated error is

$$\sqrt{\frac{0.032 ( 1 - 0.032 )}{\text{25,000}} + \frac{0.10 ( 1 - 0.10 )}{500} =} 0.013$$

and a 95% confidence interval for the true difference π₁−π₂ is −0.068±1.96(0.013), or −0.068±0.025. Since the 95% confidence interval contains only negative values, the conclusion can be reached that kinases are enriched in the sample (the biomarker set produced by the data analysis algorithm) relative to the population of 25,000 biomarkers.

The two-way contingency table in the example above can be analysed using methods known in the art other than the one disclosed above. For example, the chi-square test for independence and/or Fisher's exact test can be used to test the null hypothesis that the row and column classification variables of the two-way contingency table are independent.

The term “X” in annotation rule 2 can be any form of annotation data. In one embodiment, “X” is any biological pathway. As such the annotation data based filtering rule has the following form.

Annotation Rule 3:

Select all biomarkers that are in any biological pathway that is enriched in the biomarker list.

To determine whether a particular biological pathway is enriched, the number of biomarkers in a particular biological pathway in the sample is compared with the number of biomarkers that are in the particular biological pathway in the population using, for example, the two-way contingency table analysis described above, or other techniques known in the art. If the biological pathway is enriched in the sample, then all biomarkers in the sample that are also in the biological pathway are retained for further analysis, in accordance with the annotation data based filtering rule.

An example of enrichment, in which it was shown that the proportion of kinases in the sample was greater than the proportion of kinases in the population across its entire 95% confidence interval has been given. In one embodiment, biomarkers having a given annotation are considered enriched in the sample relative to the population when the proportion of biomarkers having the annotation in the sample is greater than the proportion of biomarkers having the annotation in the population across its entire 95% degree confidence interval as determined by two-way contingency table analysis. In another embodiment, biomarkers having a given annotation are considered enriched in the sample relative to the population if a p value as determined by the Fisher exact test, Chi-square test, or relative algorithms is 0.05 or less, 0.005 or less or 0.0005 or less.

Another form of annotation data based filtering rule has the following form:

Annotation Rule 4:

Select all biomarkers that are in biological pathway X.

In an embodiment, a set of biomarkers is determined using a data analysis algorithm. Exemplary data analysis algorithms are disclosed in Section 5.5. In addition, Section 6 describes certain tests that can also serve as data analysis algorithms. These tests include, but are not limited to a Wilcoxon test and the like with a statistically significant p value (e.g., 0.05 or less, 0.04, or less, etc.), and/or a requirement that a biomarker exhibit a mean differential abundance between biological samples obtained from converters and biological samples obtained from nonconverters in a training population. Upon application of the data analysis algorithm, a set of biomarkers that discriminates between converters and nonconverters is determined. Next, an annotation rule, for example annotation rule 4, is applied to the set of discriminating biomarkers in order to further reduce the set of biomarkers. Those of skill in the art will appreciate that the order in which these rules are applied is generally not important. For example, annotation rule 4 can be applied first and then certain data analysis algorithms can be applied, or vice versa. In some embodiments, biomarkers ultimately deemed as discriminating between converters and nonconverters satisfy each of the following criteria: (i) a p value of 0.05 or less (p<0.05) as determined from a Wilcoxon adjusted test using static (single time point) data; (ii) a mean-fold change of 1.2 or greater between converters and nonconverters across the training set using static (single time point data), and (iii) present in a specific biological pathway. See also, Section 6.7, infra, for a detailed example. In this example, there is no requirement that members of the pathway are enriched in the set of biomarkers identified by the data analysis algorithms. Furthermore, it is noted that criteria (i) and (ii) are forms of data analysis algorithms and criterion (iii) is a annotation data based filtering rule.

In another embodiment, once a list of discriminating biomarkers is identified, the biomarkers can then be used to determine the identity of the particular biological pathways from which the discriminating biomarkers are implicated. In certain embodiments, annotation data-based filtering rules are applied to the list of discriminating biomarkers identified by the methods of the present invention (e.g., the methods described in Sections 5.4, 5.5 and 6). Such annotation data-based filtering rules identify the particular biological pathway or pathways that are enriched in the discriminating list of biomarkers identified by the data analysis algorithms. In an exemplary embodiment of the invention, DAVID 2.0 software is used to identify and apply such annotation data-based filtering rules to the set of biomarkers identified by the data analysis algorithms in order to identify pathways that are enriched in the set. In some embodiments, those biomarkers that are in an enriched biological pathway are selected for use as discriminating biomarkers in the kits of the present invention.

In some embodiments of the present invention, biomarkers that are in biological pathways that are enriched in the biomarker set determined by application of a data analysis algorithm to a training population that includes converters and nonconverters can be used as filtering step to reduce the number of biomarkers in the set. In one such approach, biological samples from subjects in a training population are obtained using, e.g., any of one or more of the methods described in Section 5.4, supra, and in Section 6, infra. In accordance with this embodiment, a nucleic acid array, such as a cDNA microarray, may be employed to generate features of biomarkers in a biomarker profile by detecting the expression of any one or more of the genes known to be or suspected to be involved in the selected biological pathways. Data derived from the cDNA microarray analysis may then be analyzed using any one or more of the analysis algorithms described in Section 5.5, supra, to identify biomarkers whose features discriminate between converters and nonconverters. Biomarkers whose corresponding feature values are capable of discriminating, for example, between converters (i.e., SIRS patients who subsequently develop sepsis) and non-converters (i.e., SIRS patients who do not subsequently develop sepsis) can thus be identified and classified as discriminating biomarkers. Biomarkers that are in enriched biological pathways can be selected from this set by applying Annotation rule 3, from above. Representative biological pathways that could be found include, for example, genes involved in the Th1/Th2 cell differentiation pathway). In one embodiments, biomarkers ultimately deemed as discriminating between converters and nonconverters satisfy each of the following criteria: (i) a p value of 0.05 or less (p<0.05) as determined from a Wilcoxon adjusted test; (ii) a mean-fold change of 1.2 or greater between converters and nonconverters across the training set, and (iii) present in a biological pathway that is enriched in the set of biomarkers derived by application of criteria (i) and (ii).

In some embodiments of the present invention, annotation data based filtering rules are used to identify biological pathways that are enriched in a given biomarker set. This biomarker set can be, for example, a set of biomarkers that is identified by application of a data analysis algorithm to training data comprising converters and nonconverters. Then, biomarkers in these enriched biological pathways are analyzed using any of the data analysis algorithms disclosed herein in order to identify biomarkers that discriminate between converters and nonconverters. In some instances, some of the biomarkers analyzed in the enriched biological pathways were not among the biomarkers in the original given biomarker set. In some instances, some of the biomarkers in the enriched biological pathways are among the biomarkers in the original given biomarker set. In some embodiments, a secondary assay is used to collect feature data for biomarkers that are in enriched pathways and it is this data that is used to determine whether the biomarkers in the enriched biological pathways discriminate between converters and nonconverters.

In some embodiments, biomarkers in biological pathways of interest are identified. In one example, genes involved in the Th1/Th2 cell differentiation pathway are identified. Then, these biomarkers are evaluated using the data analysis algorithms disclosed herein to determine whether they discriminate between converters and nonconverters.

### 6. EXAMPLES

### Example 1

SIRS positive subjects admitted to an ICU were recruited for the study. Subjects were eighteen years of age or older and gave informed consent to comply with the study protocol. Subjects were excluded from the study if they were (i) pregnant, (ii) taking antibiotics to treat a suspected infection, (iii) were taking systemic corticosteroids (total dosage greater than 100 mg hydrocortisone or equivalent in the past 48 hours prior to study entry), (iv) had a spinal cord injury or other illness requiring high-dose corticosteroid therapy, (v) pharmacologically immunosuppressed (e.g., azathioprine, methotrexate, cyclosporin, tacrolimus, cyclophosphamide, etanercept, anakinra, infliximab, leuflonamide, mycophenolic acid, OKT3, pentoxyphylin, etc.), (vi) were an organ transplant recipient, (vii) had active or metastatic cancer, (viii) had received chemotherapy or radiation therapy within eight weeks prior to enrollment, and/or (ix) had taken investigational use drugs within thirty days prior to enrollment.

In the study SIRS criteria were evaluated daily. APACHE II and SOFA scoring was performed following ICU admission. APACHE II is a system for rating the severity of medical illness. APACHE stands for “Acute Physiology and Chronic Health Evaluation,” and frequently used to predict in-hospital death for patients in an intensive care unit. See, for example, Gupta et al., 2004, Indian Journal of Medical Research 119, 273-282, which is hereby incorporated herein by reference in its entirety. SOFA is a test to measure the severity of sepsis. See, for example, Vincent et al., 1996, Intensive Care Med. 22, 707-710, which is hereby incorporated herein by reference in its entirety. Patients were monitored daily for up to two weeks for clinical suspicion of sepsis including, but not limited to, any of the following signs and symptoms:
pneumonia: temperature>38.3° C. or <36° C.+white blood cell count (WBC)>12,000/mm³ or <4,000/mm³+new-onset of purulent sputum+new or progressive infiltrate on chest radiograph (3 out of 4 findings);
wound infection: temperature>38.3° C. or <36° C.+pain+erythema+purulent discharge (3 out of 4 findings);
urinary tract infection: temperature>38.3° C. or WBC>12,000/mm³ or <4,000/mm³+bacteruria and pyuria (>10 WBC/hpf or positive leukocyte esterase) (all findings);
line sepsis: temperature>38.3° C. or <36° C.+erythema/pain/purulence at catheter exit site (3 out of 4 findings, including fever);
intra-abdominal abscess: temperature>38.3° C. or <36° C.+WBC>12,000/mm³ or <4,000/mm³+radiographic evidence of fluid collection (2 out of 3 criteria);
CNS Infection: temperature>38.3° C. or <36° C.+WBC>12,000/mm³ or <4,000/mm³+CSF pleocytosis via LP or Ventricular drainage.

Blood was drawn daily for a minimum of four consecutive days beginning within 24 hours following study entry. Patients were followed and blood samples were drawn daily for a maximum of fourteen consecutive days unless clinical suspicion of infection occurred. The maximum volume of blood drawn from any one subject did not exceed 210 mL over the course of a fourteen day study maximum. Blood draws for the study were discontinued if the loss of blood posed risk to the patient as defined by physician's judgment. Each patient had two Paxgene (RNA) tubes drawn on each day.

Blood samples were collected in PreAnalytiX PAXgene™ blood collection tubes from male and female patients over 18 years of age admitted to a trauma center or ICU. All patients were at risk of sepsis based on meeting two of four SIRS criteria. Blood samples were drawn each day for a minimum of four and maximum of fourteen days. Thus, plasma samples were collected prospectively upon admission to an ICU and divided into septic versus SIRS patients retrospectively based on whether they developed infection. Sepsis samples represent time points prior to the clinical diagnosis of sepsis and were compared to time-matched uninfected SIRS patients.

The protein profiling experiment was divided into two separate parts. Part I examined plasma for differentially expressed proteins between Sepsis and SIRS pooled samples using three-dimensional LC fractionation with electrospray ion trap mass spectrometry (3D LC-MSAMS). Two rounds (batches) of plasma samples were pooled at study time points “DOE (Day of Entry)”, T₋₆₀ and T₋₁₂. An additional round (batch) also included a pool from the T₋₃₆ time point for a total of three batches each for Sepsis and SIRS. Part II of the experiment also examined plasma for differentially expressed proteins between Sepsis and SIRS pooled samples. For part II, a single round of LCMS/MS was run on a pool from time point T₋₁₂.

Part I. Plasma samples from 25 SIRS and 25 septic patients (150 μL) were used in this part of the study. Equal volumes of plasma (50 μL) were taken randomly from individual patient samples from the same disease state (either sepsis or SIRS) for the creation of six pools (three sepsis batches and three SIRS batches), each containing a total of 20 individual samples. The goal of these studies was to identify proteins common to each pooled dataset that were either up-regulated or down-regulated in the early stages of sepsis, ultimately allowing for the identification of protein biomarkers.

Immunodepletion. Plasma samples first were immunodepleted to remove abundant proteins. The pooled plasma samples were immunodepleted using the Agilent Multiple Affinity Removal System (5185-5985, 4.6 mm×50 mm, Agilent Technologies). This immunodepletion column is based upon affinity purified polyclonal antibody technology (Maccarone et al., 2004, Electrophoresis 25, 2402-2412; Björhall et al., 2005, Proteomics 5, 307-317; and Echan et al., 2005, Proteomics 5, 3292-3303, each of which is hereby incorporated herein by reference in its entirety), containing six types of antibodies to specifically remove six target proteins: albumin, transferrin, haptoglobin, anti-trypsin, IgG, and IgA. The six antibodies are oriented on the surface of solid beads and chemically cross linked via the FC (Fragment Crystallizable) region resulting in a stable, non-leaching product, with the material packed in an HPLC column format.

A 25 μL aliquot from each plasma sample was diluted five times with column loading buffer A (Agilent Technologies, #5185-5987) and followed by centrifugation at 12,000 rpm for ten minutes. The column was attached to a capillary HPLC pump and equilibrated with buffer A at a flow rate of 0.5 mL/min for 10 min. The diluted plasma sample was then injected onto the column and the column was washed with buffer A at a flow rate of 0.25 mL/min. The first 1.5 mL flow-through containing low abundance plasma proteins was collected. The retained proteins were then removed by elution using buffer B (Agilent Technologies # 5185-5988) at a flow rate of 0.5 mL/min for seven minutes. Retained proteins were checked by 2D-Gel and no other proteins except these six binding proteins have been found (Maccarone et al, 2004, Electrophoresis 25, 2402-2412; and Echan et al., 2005, Proteomics 5, 3292-3303). The column was either stored or re-used. The protein concentrations of samples before and after immunodepletion were measured by Coomassie protein assay reagent kit (Pierce #23200) in order to validate the protein removal approach. It was determined that 85% of total protein was removed from the original plasma sample after immunodepletion.

Protein Digestion. A 2 mg protein alliquot from the immunodepletion column was collected for each pooled plasma sample. The proteins were concentrated by Vacufuge (eppendorf) to 0.25 mL (4 mg/mL protein) and denatured in 8M urea (ACROS) for 10 minutes. The proteins were reduced and alkylated using 5 mM DL-dithiothreitol (Sigma) at 37° C. for 30 minutes and 10 mM iodoacetamide (Sigma) at 37° C. for 30 minutes, respectively. The samples were then diluted four times to a final concentration of 2M urea using 100 mM ammonium hydrogen carbonate (Fluka). The proteins were digested with trypsin (Promega) at the ratio of 1:50 (w/w) overnight and followed by the second trypsin digestion under the same conditions. The protein digestion efficiency was checked with a Coomassie protein assay reagent kit.

Three dimensional chromatography and sample loading. Using this approach, the peptide mixture was pre-fractionated by a first reverse phase column (RP1) based on hydrophobicity and then each fraction was then further fractionated by a SCX column based on the peptide ion strengths. The final high resolution separation was performed on a second reverse phase (RP2) column by a shallow reverse phase gradient which was determined by the first reverse phase (RP1) fractionation gradient.

Approximately 2 mg digested protein was harvested from each pooled sample and 0.5 mg was then subjected to 3-D LC-MS/MS analysis using an Agilent 1100 LC/MSD Trap system coupled directly to an Agilent 1100 nano-pump and a micro-autosampler. Using an in-house constructed pressure cell, 5 μm Zorbax SB-C18 packing material (Agilent Technologies) was packed into a 500 μm ID 1/32″ OD PEEK tubing (Upchurch Scientific). A 10 cm section was cut off to form the first dimension RP column (RP1). A similar column (500 μm ID, 4 cm length) packed with 5 μm PolySulfoethyl (Western Analytic Production) packing material was used as the SCX column. A second C18 column 4 cm in length and 250 μm ID was used as the trap column. A zero dead volume 1 μm filter (Upchurch, M548) was attached to the exit of each column for column packing and connecting. A fused silica capillary (100 μm ID, 360 um OD, 20 cm length) packed with 5 μm Zorbax SB-C18 packing material (Agilent Technologies) was used as the analytical column (RP2). One end of the fused silica tubing was pulled to a sharp tip with the ID smaller than 5 μm using a Sutter P-2000 laser puller (Sutter Instrument Company, Novato, Calif., USA). The peptide mixtures were loaded onto the first C18 column (RP1) using the same in-house pressure cell. To avoid sample carry-over, a new set of the four columns was used for each sample. In order to maintain good reproducibility for quantitation, each of the above four columns was packed to the exact same length for every 3D experiment. The 3-dimensional LC separation consisted of two HPLC pumps, four micro- and nano-flow LC columns constructed In-house, together with a switch valve. A 1 mg alliquot of each digested protein sample was loaded onto the first dimension reverse phase (RP) column for every analysis. Up to five RP fractions and up to eight strong cation exchange (SCX) fractions were eluted sequentially from the loading column to the analytical column for high resolution peptide separation. All fractionation and separation methods were identical for the samples within the same batch. The runtime for each fraction was about 2.5 hours and total runtime for each sample was about 3 days. A scan range of 200-2000 m/z was employed in the positive mode.

Spectra were analyzed using Spectrum Mill MS Proteomics Workbench (version 2.7 software, Agilent Technologies). Over ten million spectra were generated and peaks identified using Agilent Technologies SpectrumMill Xtractor. The total spectra numbers were normalized across all rounds and entries were removed if they had a sequence tag length of 1 or less. Remaining MS/MS spectra were searched against the National Center for Biotechnology Information (NCBI) non-redundant protein database (NCBI-nr human Nov. 6, 2003, 97027 sequences) limited to human taxonomy. The enzyme parameter was limited to full tryptic peptides with a maximum miscleavage of 2. All other search parameters were set to SpectrumMill's default settings (carbamidomethylation of cysteines, +/−2.5 Dalton tolerance for precursor ions, +/−0.7 Dalton tolerance for fragment ions, and a minimum matched peak intensity of 50%).

The false positive rate was estimated by searching one 3D dataset against a combined forward-reverse database (NCBI-nr human Nov. 6, 2003, 97027 sequences, Peng, 2003 J. Proteome Res. 2, 43-50). A total of 4294 spectra and 107 proteins were auto-validated. Among them, 16 spectra and 12 proteins were from the reverse database. Thus the false positive rate of the filtering criteria was 0.75% spectra, and 22% protein. The false positive rate for proteins with a minimum unique peptide of 2 was 0.19% spectra, and 2.8% protein. Only proteins with at least 2 unique peptides were selected for relative quantitative analysis.

SpectrumMill grouped the proteins with the same set or subsets of unique peptides together in order to minimize protein redundancy. The number of identified proteins reported in SpectrumMill is the number of identified “protein groups” rather than the number of identified protein sequences in the database. Spectrum mounting was used for relative protein quantitation. The number of valid MS/MS spectra from each protein was normalized to the total MS/MS spectra number of each dataset. Samples were divided into two patient groups, SIRS vs. sepsis. The Z-test was used for statistical analysis. The Z-scores (Δ/stdev) of each protein were calculated between those 2 groups and proteins with Z-scores above 2 were considered to be biomarker candidates. The candidate list was further filtered by relative standard deviation (<100%), absolute MS/MS spectrum number (>=10), unique peptide number (>=2) and manual inspection to remove obvious false hits such as keratin.

The Spectrum Mill Workbench output produced 2,810 protein entries across the three pools. The total spectra numbers were normalized across all pools and entries with a distinct sum tag score less than 13. The Genbank Accession numbers for each hit were cross referenced with their corresponding Entrez Gene ID using the gene2accession table from the National Institute of Health. Only proteins with a gene ID were included for further analysis (484 remaining entries). Sepsis to SIRS ratios were calculated using the normalized total spectra numbers. Where SIRS>Sepsis, the ratio was calculated using 1/(Sepsis/SIRS). If either number was zero, so a ratio cannot be calculated, the value was tagged SEPSIS+ or SIRS+ as appropriate. The range of values across time points was calculated for each round and protein entries were included if they had a range>1.5 or contained SEPSIS+ or SIRS+ at any time point. This left 151 remaining entries constituting 103 unique gene IDs identified in Table 1 below.

TABLE 1
Biomarkers that discriminate between Sepsis and Sirs in part 1 of example one.

| | | Gene Accession | Protein Accession |
|---|---|---|---|
| Gene Symbol | Gene Name | Number | Number |
| Column 1 | Column 1 | Column 3 | Column 4 |
| SERPINA3 | serine (or cysteine) | NM_001085 | NP_001076 |
| | proteinase inhibitor, clade | | |
| | A (alpha-1 antiproteinase, | | |
| | antitrypsin), member 3 | | |
| ACTB | actin, beta | NM_001101 | AAS79319 |
| AFM | Afamin | NM_001133 | AAA21612 |
| AGT | angiotensinogen (serine | NM_000029 | AAR03501 |
| | (or cysteine) proteinase | | |
| | inhibitor, clade A (alpha-1 | | |
| | antiproteinase, | | |
| | antitrypsin), member 8) | | |
| AHSG | alpha-2-HS-glycoprotein | NM_001622 | NP_001613 |
| AMBP | alpha-1-microglobulin/bikunin | NM_001633 | NP_001624 |
| | precursor | | |
| APOF | apolipoprotein F | NM_001638 | AAA65642 |
| APOA1 | apolipoprotein A-I | NM_000039 | AAD34604 |
| APOA2 | apolipoprotein A-II | NM_001643 | AAA51701 |
| APOA4 | apolipoprotein A-IV | NM_000482 | AAS68228 |
| APOB | apolipoprotein B | NM_000384 | AAP72970 |
| | (including Ag(x) antigen) | | |
| APOC1 | apolipoprotein C-I | NM_001645 | AAQ91813 |
| APOC3 | apolipoprotein C-III | BC027977 | AAB59372 |
| APOE | apolipoprotein E | NM_000041 | AAB59397 |
| APOH | apolipoprotein H (beta-2- | NM_000042 | CAA40977 |
| | glycoprotein I) | | |
| SERPINC1 | serine (or cysteine) | NM_000488 | CAI19423 |
| | proteinase inhibitor, clade | X68793 | P01008 |
| | C (antithrombin), member | | |
| | 1; Antithrombin-III | | |
| | precursor (ATIII) | | |
| AZGP1 | alpha-2-glycoprotein 1, | NM_001185 | NP_001176 |
| | zinc | | |
| BF | B-factor, properdin | NM_001710 | CAI17456 |
| SERPING1 | serine (or cysteine) | NM_000062; | AAW69393 |
| | proteinase inhibitor, clade | BC011171 | |
| | G (C1 inhibitor), member | | |
| | 1, (angioedema, | | |
| | hereditary) | | |
| C1QB | complement component 1, | NM_000491 | NP_000482 |
| | q subcomponent, beta | | |
| | polypeptide | | |
| C1S | complement component 1, | NM_201442; | NP_958850 |
| | s subcomponent | NM_001734 | |
| C2 | complement component 2 | NM_000063 | CAI17451 |
| C3 | complement component 3 | NM_000064 | AAR89906 |
| C4BPA | complement component 4 | NM_001017367 | CAH70782 |
| | binding protein, alpha | | |
| C5 | complement component 5 | NM_001736 | NP_001726 |
| C8A | complement component 8, | NM_000562 | CAI19172 |
| | alpha polypeptide | | |
| C8G | complement component 8, | NM_000606 | NP_000597 |
| | gamma polypeptide | | |
| C9 | complement component 9 | NM_001737 | NP_001728 |
| SERPINA6 | serine (or cysteine) | NM_001756 | NP_001002236 |
| | proteinase inhibitor, clade | | NP_000286 |
| | A (alpha-1 antiproteinase, | | |
| | antitrypsin), member 6 | | |
| CD14 | CD14 antigen | NM_000591 | AAP35995 |
| CLU | clusterin (complement | NM_001831 | AAP88927 |
| | lysis inhibitor, SP-40,40, | | |
| | sulfated glycoprotein 2, | | |
| | testosterone-repressed | | |
| | prostate message 2, | | |
| | apolipoprotein J) | | |
| CP | ceruloplasmin | NM_000096 | NP_000087 |
| | (ferroxidase) | | |
| CRP | C-reactive protein | NM_000567 | NP_000558 |
| CSK | c-src tyrosine kinase | NM_004383 | NP_004374 |
| F2 | coagulation factor II | NM_000506 | AAL77436 |
| | (thrombin) | | |
| F9 | coagulation factor IX | NM_000133 | NP_000124 |
| | (plasma thromboplastic | | |
| | component, Christmas | | |
| | disease, hemophilia B) | | |
| FGA | fibrinogen alpha chain | BC070246 | BAC55116 |
| FGB | fibrinogen beta chain | NM_005141 | AAA18024 |
| FGG | fibrinogen gamma chain | NM_000509 | AAB59531 |
| FLNA | filamin A, alpha (actin | NM_001456 | CAI43227 |
| | binding protein 280) | | |
| FN1 | fibronectin 1 | BT006856 | BAD52437 |
| GC | group-specific component | NM_000583 | NP_000574 |
| | (vitamin D binding | | |
| | protein) | | |
| GSN | gelsolin (amyloidosis, | BC026033 | CAI14413 |
| | Finnish type) | | |
| HBB | hemoglobin, beta | NM_000518 | AAD19696 |
| SERPIND1 | serine (or cysteine) | NM_000185 | CAG30459 |
| | proteinase inhibitor, clade | | |
| | D (heparin cofactor), | | |
| | member 1 | | |
| HP | Haptoglobin | BC107587 | NP_005134 |
| HPX | Hemopexin | NM_000613 | NP_000604 |
| HRG | histidine-rich glycoprotein | NM_000412 | NP_000403 |
| IF | I factor (complement) | NM_000204 | NP_000195 |
| IGFALS | insulin-like growth factor | NM_004970 | NP_004961 |
| | binding protein, acid labile | | |
| | subunit | | |
| ITGA1 | integrin, alpha 1 | NM_181501 | NP_852478 |
| ITIH1 | inter-alpha (globulin) | BC109115 | NP_002206 |
| | inhibitor H1 | | NP_032432 |
| ITIH2 | inter-alpha (globulin) | NM_002216 | NP_002207 |
| | inhibitor H2 | | |
| ITIH4 | inter-alpha (globulin) | NM_002218 | NP_002209 |
| | inhibitor H4 (plasma | | |
| | Kallikrein-sensitive | | |
| | glycoprotein) | | |
| KLKB1 | kallikrein B, plasma | NM_000892 | NP_000883 |
| | (Fletcher factor) 1 | | |
| KNG1 | kininogen 1 | NM_000893 | NP_000884 |
| KRT1 | keratin 1 (epidermolytic | BC063697 | NP_000412 |
| | hyperkeratosis) | | |
| LGALS3BP | lectin, galactoside- | NM_005567 | NP_005558 |
| | binding, soluble, 3 binding | BC015761 | |
| | protein | BC002403 | |
| | | BC002998 | |
| LPA | lipoprotein, Lp(a) | NM_005577 | NP_005568 |
| MLL | myeloid/lymphoid or | NM_005934 | NP_005924 |
| | mixed-lineage leukemia | | |
| | (trithorax homolog, | | |
| | Drosophila) | | |
| MRC1 | mannose receptor, C type 1 | NM_002438 | NP_002429 |
| MYL2 | myosin, light polypeptide | NM_000432 | AAH31006 |
| | 2, regulatory, cardiac, | | |
| | slow | | |
| MYO6 | Myosin VI | NM_004999 | NP_004990 |
| ORM1 | orosomucoid 1 | NM_000607 | CAI16859 |
| SERPINF1 | serine (or cysteine) | NM_002615 | AAH13984 |
| | proteinase inhibitor, clade | BC013984 | |
| | F (alpha-2 antiplasmin, | | |
| | pigment epithelium | | |
| | derived factor), member 1 | | |
| SERPINA1 | serine (or cysteine) | BC015642 | NP_001002235 |
| | proteinase inhibitor, clade | NM_000295 | NP_000286 |
| | A (alpha-1 antiproteinase, | | |
| | antitrypsin), member 1 | | |
| SERPINA4 | serine (or cysteine) | NM_006215 | NP_006206 |
| | proteinase inhibitor, clade | | |
| | A (alpha-1 antiproteinase, | | |
| | antitrypsin), member 4 | | |
| SERPINF2 | serine (or cysteine) | BC031592 | NP_000925 |
| | proteinase inhibitor, clade | | |
| | F (alpha-2 antiplasmin, | | |
| | pigment epithelium | | |
| | derived factor), member 2 | | |
| PROS1 | Protein S (alpha) | NM_000313 | NP_000304 |
| QSCN6 | quiescin Q6 | NM_002826 | AAQ89300 |
| RGS4 | regulator of G-protein | NM_005613 | NP_005604 |
| | signalling 4 | | |
| SAA1 | serum amyloid A1 | BC105796 | AAA64799 |
| | | | AAA30968 |
| SAA4 | serum amyloid A4, | NM_006512 | NP_006503 |
| | constitutive/Serum | P05067 | |
| | Amyloid A-4 protein | | |
| | precursor (constitutively | | |
| | expressed serum amyloid A) | | |
| | (C-SAA) | | |
| SERPINA7 | serine (or cysteine) | NM_000354 | CAB06092 |
| | proteinase inhibitor, clade | | |
| | A (alpha-1 antiproteinase, | | |
| | antitrypsin), member 7 | | |
| TF | transferrin | NM_001063 | NP_001054 |
| TFRC | transferrin receptor (p90, | NM_003234 | NP_003225 |
| | CD71) | | |
| TTN | titin | BC013396 | CAD12456 |
| TTR | transthyretin (prealbumin, | NM_000371 | AAH05310 |
| | amyloidosis type I) | | AAP35853 |
| UBC | ubiquitin C | NM_021009 | NP_066289 |
| VTN | vitronectin (serum | NM_000638 | P04004 |
| | spreading factor, | | |
| | somatomedin B, | | |
| | complement S-protein) | | |
| VWF | von Willebrand factor | NM_000552 | AAB59458 |
| ALMS1 | Alstrom syndrome 1 | NM_015120 | NP_055935 |
| ATRN | attractin | BC101705 | CAI22615 |
| | | NM_139321 | |
| APOL1 | apolipoprotein L, 1 | BC017331 | AAK20210 |
| | | NM_003661 | |
| TRIP11 | thyroid hormone receptor | NM_004239 | NP_004230 |
| | interactor 11 | | |
| PDCD11 | programmed cell death 11 | NM_014976 | NP_055791 |
| KIAA0433 | — | AB007893 | BAA24863 |
| SERPINA10 | serine (or cysteine) | NM_016186 | NP_057270 |
| | proteinase inhibitor, clade | | |
| | A (alpha-1 antiproteinase, | | |
| | antitrypsin), member 10 | | |
| BCOR | BCL6 co-repressor | BC063536 | AAG41429 |
| C10orf18 | chromosome 10 open | BC001759 | CAI13368 |
| | reading frame 18 | | |
| YY1AP1 | YY1 associated protein 1 | BC044887 | AAL75971 |
| | | BC014906 | CAH71646 |
| FLJ10006 | — | BC110537 | AAH17012 |
| | | BC110536 | |
| BDP1 | B double prime 1, subunit | NM_018429 | AAH32146 |
| | of RNA polymerase III | | |
| | transcription initiation factor IIIB | | |
| SMARCAD1 | SWI/SNF-related, matrix- | NM_020159 | NP_064544 |
| | associated actin-dependent | | |
| | regulator of chromatin, | | |
| | subfamily a, containing | | |
| | DEAD/H box 1 | | |
| MKL2 | MKL/myocardin-like 2 | NM_014048 | AAH47761 |
| CHST8 | carbohydrate (N- | NM_022467 | NP_071912 |
| | acetylgalactosamine 4-0) | BC018723 | |
| | sulfotransferase 8 | | |
| MCPH1 | microcephaly, primary | NM_024596 | AAH30702 |
| | autosomal recessive 1 | BC030702 | |
| MYO18B | myosin XVIIIB | NM_032608 | NP_115997 |
| MICAL-L1 | — | NM_033386 | AAH82243 |
| | | | AAH01090 |
| PGLYRP2 | peptidoglycan recognition | NM_052890 | Q96PD5 |
| | protein 2 | | |
| LRG1 | leucine-rich alpha-2- | NM_052972 | AAH70198 |
| | glycoprotein 1 | | |
| KCTD7 | potassium channel | NM_153033 | NP_694578 |
| | tetramerisation domain | | |
| | containing 7 | | |
| MGC27165 | — | BC087841 | AAH87841 |
| | | BC005951 | |

The 103 proteins (Entrez Gene ID's) were uploaded into DAVID 2.1 (Database for Annotation, Visualization and Integrated Discovery, Dennis et al., 2003, Genome Biol. 4:P3). All 103 genes were recognized by DAVID. The canonical pathways contained in the data were examined by selecting output from Biocarta and KEGG pathways. As set forth in Table 2, below, any pathways containing at least two genes from the list of 103 and having a probability score (p-value)≦0.1 were included. In Table 2, the “count” is the number of proteins from that particular pathway that are present in Table 1 and the “Percent” is the above-described count divided by the total protein number of proteins in the given database that are in the pathway. The data indicates participation of the Complement and Coagulation systems.

TABLE 2
Pathways associated with Sepsis and Sirs in part 1 of example one.

| Categorgy System | Term | Count | Percent | P Value |
|---|---|---|---|---|
| KEGG_PATHWAY | Complement and | 25 | 24 | 2.25E−33 |
| | Coagulation | | | |
| | Cascades | | | |
| BIOCARTA | Intrinsic | 8 | 7 | 5.81E−09 |
| | Prothrombin | | | |
| | Activation | | | |
| | Pathway | | | |
| BIOCARTA | Complement | 7 | 6 | 1.04E−06 |
| | Pathway | | | |
| BIOCARTA | Classical | 6 | 5 | 2.70E−06 |
| | Complement | | | |
| | Pathway | | | |
| BIOCARTA | Alternative | 5 | 4 | 2.86E−05 |
| | Complement | | | |
| | Pathway | | | |
| BIOCARTA | Lectin Induced | 5 | 4 | 8.36E−05 |
| | Complement | | | |
| | Pathway | | | |
| BIOCARTA | Extrinsic | 4 | 3 | 8.54E−04 |
| | Prothrombin | | | |
| | Activation | | | |
| | Pathway | | | |
| BIOCARTA | Acute Myocardial | 4 | 3 | 1.44E−03 |
| | Infarction | | | |
| KEGG_PATHWAY | Regulation of | 8 | 7 | 8.59E−03 |
| | Actin | | | |
| | Cytoskeleton | | | |
| KEGG_PATHWAY | Focal Adhesion | 7 | 6 | 4.48E−02 |
| KEGG_PATHWAY | ECM-Receptor | 4 | 3 | 7.31E−02 |
| | Interaction | | | |

Additionally the molecular functions and biological process inherent in the data set set forth in Table 1 were examined by outputting any “over-represented” gene ontology categories. Gene ontology categories over-represented in the data are set forth in Table 3 below. As in the case of Table 2, “count” is the number of proteins present in Table 1 from that particular pathway and “Percent” is the count (as defined here)/total protein number from that pathway in the database. Similar to the pathway output, Complement and Coagulation activity is highly represented in this data set. The major theme of the data present in Table 3 is immune system activity. Additionally, lipid transport (apolipoproteins) is a functional process that may prove to be important in distinguishing Sepsis from SIRS.

TABLE 3
Gene ontology categories over-represented in table 1.

| Term | Count | Percent | P Value |
|---|---|---|---|
| Acute-Phase Response | 18 | 17 | 5.07E−28 |
| Response to Pest, Pathogen or Parasite | 34 | 33 | 5.70E−26 |
| Complement Activation | 14 | 13 | 9.01E−20 |
| Blood Coagulation | 16 | 15 | 1.13E−17 |
| Serine-Type Endopeptidase Inhibitor | 16 | 15 | 1.52E−17 |
| Activity | | | |
| Wound Healing | 16 | 15 | 2.96E−17 |
| Lipid Transport | 12 | 11 | 1.76E−13 |
| Humoral Immune Response | 14 | 13 | 2.56E−11 |
| Immune Response | 39 | 37 | 4.73E−11 |
| Humoral Defense Mechanism | 12 | 11 | 1.61E−10 |
| (Sensu Vertebrata) | | | |
| Inflammatory Response | 12 | 11 | 3.87E−08 |

The ontologies were filtered to include only those with a level 5 distinction (most specific gene ontologies) and greater than 10 percent of the input gene list (Table 1). Only ontologies from the Molecular Function or Biological Processes were incorporated. See the website at geneontology.org. The canonical pathways identified by DAVID are shown in Table 2. Each pathway was over-represented in this data set, implying that they contained more proteins from the data than would be expected by chance. The results indicate a significant focus on both the complement and coagulation cascades, known to play a major part in sepsis.

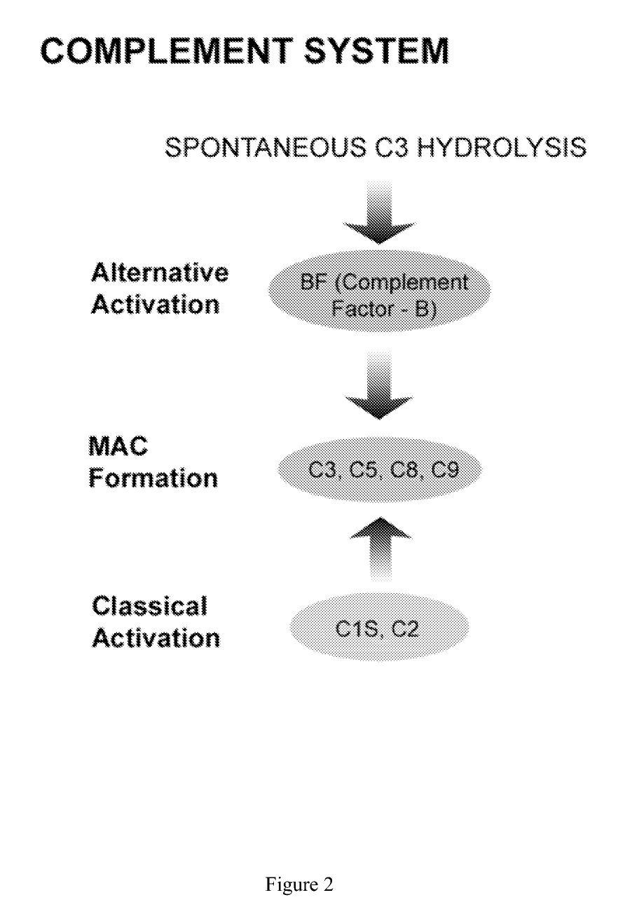

The complement pathway consists of a complex series of over thirty plasma proteins which are part of the immune response, providing a critical defense against infection. FIG. 2 shows the identified proteins from this study in the complement cascade. Activation of the complement system lyses bacterial cells, forms chemotactic peptides (C3a and C5a) that attract immune cells, and increases phagocytotic clearance of infecting cells. Additionally, the complement pathway can result in increased permeability of vascular walls and inflammation. Most complement proteins exist in plasma as inactive precursors that cleave and activate each other in a proteolytic cascade leading ultimately to the formation of the membrane attack complex (MAC), which causes lysis of cells. MAC formation may be activated by three pathways distinct in the initiation of the proteolytic cascade but share most of their components; the classical pathway, alternative pathway and membrane attack pathway. Here, the classical and alternative pathways are discussed. The classical pathway is activated by the recognition of foreign cells by antibodies bound to the surface of the cells. In this data, the proteins C1S and C2 were unique to this pathway. Proteolysis is triggered in the alternative pathway by the spontaneous activation of C3 convertase from C3. Complement Factor-B (Protein BF, properdin) was found in the data presented here and is unique to the alternative activation pathway. Additionally the proteins C3, C5, C8 and C9, discovered in the plasma samples in this study, are common to all methods of complement activation.

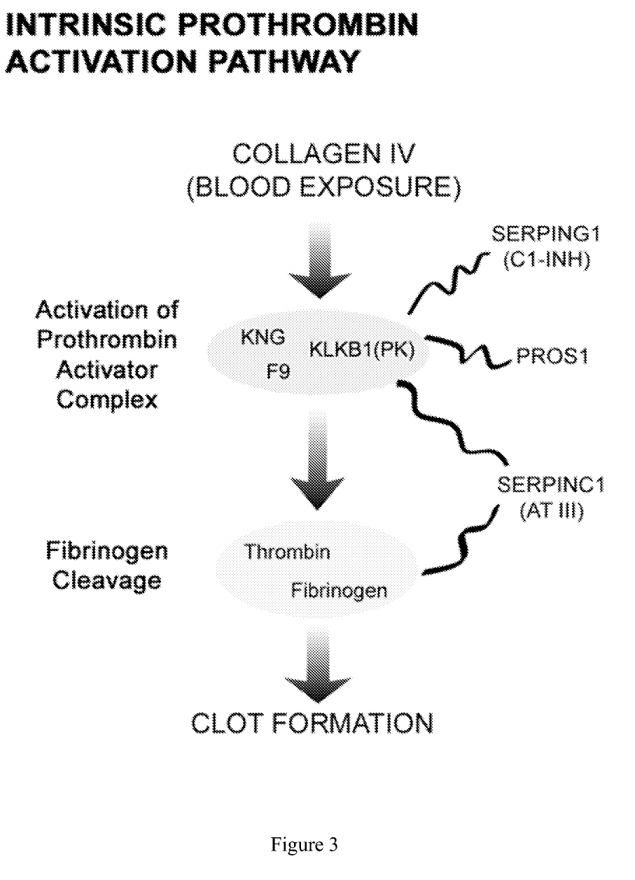

Activation of coagulation is a normal component of the acute inflammatory response and disorders of coagulation are common in sepsis. Tissue factor production is increased and leads to the activation of both the intrinsic and extrinsic prothrombin activation pathways. In this study, the data strongly indicated participation of the intrinsic prothrombin activation pathway (FIG. 3). Briefly, blood coagulation or clotting takes place in three essential phases. First is the activation of a prothrombin activator complex, followed by the second stage of prothrombin activation. The third stage is clot formation as a result of fibrinogen cleavage by activated thrombin. The prothrombin activation complex is formed by two pathways, each of which results in a different form of the prothrombin activator. The intrinsic mechanism of prothrombin activator formation begins with trauma to the blood or exposure of blood to collagen in a traumatized vessel wall. While the extrinsic pathway was identified by DAVID, it appeared to be included because of the overlapping proteins PROS, SERPINC1, Thrombin and Fibrinogen. The data also contained SERPING1, KNG, KLKB1 and F9 which are all uniquely involved in the formation of the prothrombin activator complex specific to the intrinsic prothrombin activation pathway. The inclusion of gene ontologies in Table 3 covering both complement and coagulation also further support the role of these pathways in distinguishing sepsis from SIRS samples. Utilizing these criteria, seven proteins showed a common increase in the plasma from sepsis patients in all three batches, while three proteins (where both the precursor and the final product are counted as a single biomarker) showed a common decrease as illustrated in Table 4.

TABLE 4
Up-regulated and down-regulated proteins in the plasma of sepsis patients
compared to SIRS patients in example one.

| | | | |
|---|---|---|---|
| | | Gene Accession | Protein Accession |
| Gene Symbol | Gene Name | Number | Number |
| Column 1 | Column 2 | Column 3 | Column 4 |

UPREGULATED

| | | | |
|---|---|---|---|
| C4B | Complement component | K02403 | AAB67980 |
| | C4 | | |
| CRP | C-reactive protein | NM_000567 | NP_000558 |
| CRP | C-reactive protein | M11880 | AAB59526 |
| | precursor | | |
| PLG | plasminogen | NM_000301 | AAH60513 |
| PLG | plasminogen precursor | X05199 | P00747 |
| APOA2 | apolipoprotein A-II | NM_001643 | AAA51701 |
| APOA2 | apolipoprotein A-II | X00955 | P02652 |
| | precursor | | |
| SERPING1 | serine (or cysteine) | NM_000062; | AAW69393 |
| | proteinase inhibitor, clade | BC011171 | |
| | G (C1 inhibitor), member | | |
| | 1, (angioedema, | | |
| | hereditary) | | |
| SERPING1 | plasma protease C1 | AB209826 | P05155 |
| | inhibitor precursor | | |
| TTR | transthyretin (prealbumin, | NM_000371 | AAH05310 |
| | amyloidosis type I) | | AAP35853 |
| TTR, TBPA, ATTR | transthyretin precursor | U19780 | P02766 |
| | (prealbumin) (TBPA) | | |
| | (TTR)(ATTR) | | |
| APCS | amyloid P component, | BT006750 | CAH73651 |
| | serum | | |
| APCS | serum amyloid | BC007058 | NP001630 |
| | P-component precursor | | |

DOWNREGULATED

| | | | |
|---|---|---|---|
| APOA1 | apolipoprotein A-I | NM_000039 | AAD34604 |
| APOA1 | apolipoprotein A-I | NM_000039 | P02647 |
| | precursor | | |
| SERPINC1 | serine (or cysteine) | NM_000488 | CAI19423 |
| | proteinase inhibitor, clade | X68793 | P01008 |
| | C (antithrombin), member | | |
| | 1; Antithrombin-III | | |
| | precursor (ATIII) | | |
| SAA4 | serum amyloid A4, | NM_006512 | NP_006503 |
| | constitutive/Serum | | |

UPREGULATED

| | | | |
|---|---|---|---|
| | Amyloid A-4 protein | P05067 | |
| | precursor (constitutively | | |
| | expressed serum amyloid | | |
| | A) (C-SAA) | | |
| SAA4 | Serum amyloid A-4 | M81349 | P02375 |
| | protein precursor | | |

The possibility of non-specific protein binding to the immunodepletion column, which could cause losses of the lower abundance proteins, was investigated by randomly analyzing the samples twice without immunodepletion. Even in the absence of immunodepletion, these ten proteins were still identified as strong biomarker candidates.

Interrogation of the data using DAVID had shown the complement and coagulation pathways to be over-represented, suggesting that they could play an important role in distinguishing sepsis from SIRS. These findings were supported by some of the proteins identified here. Many of the proteins in Table 4 are known to be acute phase proteins (C-reactive protein, plasminogen and serum amyloid P), involved in the complement pathway (complement component C4), the coagulation pathway (antithrombin) or both (plasma protease C1 inhibitor) or lipid transport (apolipoproteins). Altered levels of several of these proteins have been reported to correlate with SIRS and sepsis (Mesters, 1996, Mannucci et al., Blood 88, 881-886 (antithrombin-III); Nakae et al., 1996, Surg Today 26, 225-229 (complement component C3 and complement component C4); Chenaud et al., 2004, Crit. Care Med. 32, 632-637 (ApoA1); Roemish et al., 2002, Blood Coagul. Fibrinolysis 13, 657-670 (antithrombin III); and Sierra et al., 2004, Intensive Care Med. 30, 2038-2045 (C-reactive protein), each of which is hereby incorporated by reference in its entirety), as both the complement and coagulation pathways are known to be activated (Mesters et al., 1996, Blood 88, 881-886; Haeney 1998, J. Antimicrob Chemother. 41, Suppl A:41-6; Wheeler et al., 1999, N. Engl J. Med. 340, 207-214; and Aird, 2005, Crit. Care Clin 21, 417-431, each of which is hereby incorporated by reference).

Sepsis is a complex disease, common in the critically ill, that still has no truly effective early diagnosis strategy or treatment. It can strike rapidly, in a matter of days, and is associated with substantial morbidity and mortality. Plasma represents a proven resource in the quest for understanding the complex interactions of the biochemical cascades that lead to disease and, further, in the identification of biomarkers for disease diagnosis. The above experimental data provides a unique combination of immunodepletion, 3D LC separation and MS/MS analysis to offer some important insights into the interactions that surround the onset of sepsis and the potential identification of protein biomarkers in this event. This platform allowed for the removal of the highly abundant proteins and thus the detection of previously suppressed low abundance proteins. Subsequent analysis using an in-house developed high resolution separation and tandem mass analysis enabled the detection of 3000 lower abundance plasma proteins and the ultimate observation of these ten potential sepsis biomarkers (where both the precursor and the final product are counted as a single biomarker), with the down-regulation of seven proteins including those involved in lipid transport, as well as the up-regulation of three proteins observed in plasma from SIRS patients.

Part II. Methods used in part II vary slightly from that given for Table I above, in that only a single set of pooled samples, at the T₋₁₂ hour time point was analyzed. Procedurally, the difference was that samples were analyzed using LC/MS-MS (not LC³) and no immunodepletion of the samples was performed prior to analysis. Thus, part II of the experiment also examined plasma for differentially expressed proteins between Sepsis and SIRS pooled samples. A single round of LCMS/MS was run on a pool from time point T₋₁₂. The data was also analyzed using Spectrum Mill Workbench software. The final output report contained 142 entries all with a distinct sum tag score>13. The data was normalized to the same scale as used in part I. Each entry was identified using a Uniprot ID and was cross references to its appropriate Entrez Gene ID using data from the International Protein Index and annotated using data from NCBI. Entries that could be linked to an Entrez gene ID were included. Ratio data was calculated as in part I. Since only a single time point was examined, it's not possible to calculate a range of ratios over time. Entries were included when the ratio>1.5 or was SEPSIS+ or SIRS+. That left 93 remaining entries representing 93 unique genes identified in Table 5 below.

TABLE 5
Biomarkers that discriminate between Sepsis and Sirs in part II of example one.

| | | Gene Accession | Protein Accession |
|---|---|---|---|
| Gene Symbol | Gene Name | Number | Number |
| Column 1 | Column 2 | Column 3 | Column 4 |
| A1BG | alpha-1-B glycoprotein | NM_130786 | NP_570602 |
| A2M | alpha-2-macroglobulin | NM_000014 | AAT02228 |
| ABLIM1 | Actin binding LIM protein 1 | NM_002313 | CAI10910 |
| ACTA1 | Actin, alpha 1, skeletal muscle | NM_001100 | CAI19052 |
| AGT | angiotensinogen (serine | NM_000029 | AAR03501 |
| | (or cysteine) proteinase | | |
| | inhibitor, clade A (alpha- | | |
| | 1 antiproteinase, | | |
| | antitrypsin), member 8) | | |
| AHSG | alpha-2-HS-glycoprotein | NM_001622 | NP_001613 |
| ANK3 | ankyrin 3, node of | NM_020987 | CAI40519 |
| | Ranvier (ankyrin G) | | |
| APCS | amyloid P component, serum | BT006750 | CAH73651 |
| APOA1 | apolipoprotein A-I | NM_000039 | AAD34604 |
| APOA4 | apolipoprotein A-IV | NM_000482 | AAS68228 |
| APOB | apolipoprotein B | NM_000384 | AAP72970 |
| | (including Ag(x) antigen) | | |
| APOC3 | apolipoprotein C-III | BC027977 | AAB59372 |
| APOL1 | apolipoprotein L, 1 | BC017331 | AAK20210 |
| | | NM_003661 | |
| AZGP1 | alpha-2-glycoprotein 1, | NM_001185 | NP_001176 |
| | zinc | | |
| B2M | beta-2-microglobulin | NM_004048 | AAA51811 |
| BF | B-factor, properdin | NM_001710 | CAI17456 |
| C1R | complement component | NM_001733 | NP_001724 |
| | 1, r subcomponent | | |
| C1S | complement component | NM_201442; | NP_958850 |
| | 1, s subcomponent | NM_001734 | NP_001725 |
| C2 | complement component 2 | NM_000063 | CAI17451 |
| C4B | complement component | NM_000592 | AAR89095 |
| | 4B | | |
| C5 | complement component 5 | NM_001736 | NP_001726 |
| C6 | complement component 6 | NM_000065 | BAD02322 |
| C7 | complement component 7 | NM_000587 | CAA72407 |
| C8A | complement component | NM_000562 | CAI19172 |
| | 8, alpha polypeptide | | |
| C8B | complement component | NM_000066 | CAC18532 |
| | 8, beta polypeptide | | |
| CDK5RAP2/ | CDK5 regulatory subunit | NM_018249 | CAI40927 |
| CDK5RA2 | associated protein 2 | | |
| CHGB | chromogranin B | NM_001819 | CAB55272 |
| | (secretogranin 1) | | |
| CLU | clusterin (complement | NM_001831 | AAP88927 |
| | lysis inhibitor, SP-40,40, | | |
| | sulfated glycoprotein 2, | | |
| | testosterone-repressed | | |
| | prostate message 2, | | |
| | apolipoprotein J) | | |
| COMP | cartilage oligomeric | NM_000095 | AAC83643 |
| | matrix protein | | |
| CORO1A | coronin, actin binding | NM_007074 | NP_009005 |
| | protein, 1A | | |
| CPN1 | carboxypeptidase N, | NM_001308 | NP_001299 |
| | polypeptide 1, 50 kD | | |
| CUL1 | cullin 1 | NM_003592 | NP_003583 |
| DET1 | de-etiolated homolog 1 | NM_017996 | NP_060466 |
| | (Arabidopsis) | | |
| DSC1 | desmocollin 1 | BC109161 | NP_060466 |
| F13A1 | coagulation factor XIII, | NM_000129 | CAC36886 |
| | A1 polypeptide | | |
| F2 | coagulation factor II | NM_000506 | AAL77436 |
| | (thrombin) | | |
| F5 | coagulation factor V | NM_000130 | CAI23065 |
| | (proaccelerin, labile | | CAB16748 |
| | factor) | | |
| FGB | fibrinogen beta chain | NM_005141 | AAA18024 |
| GOLGA1 | golgi autoantigen, golgin | NM_002077 | CAI39632 |
| | subfamily a, 1 | | |
| GSN | gelsolin (amyloidosis, | BC026033 | CAI14413 |
| | Finnish type) | | |
| HBA1 | hemoglobin, alpha 1 | NM_000558 | AAO22464 |
| HBB | hemoglobin, beta | NM_000518 | AAD19696 |
| HP | haptoglobin | BC107587 | NP_005134 |
| HPX | hemopexin | NM_000613 | NP_000604 |
| HSPA5 | heat shock 70 kDa protein | NM_005347 | NP_005338 |
| | 5 (glucose-regulated | | |
| | protein, 78 kDa) | | |
| HUNK | hormonally upregulated | NM_014586 | NP_055401 |
| | Neu-associated kinase | | |
| IGFBP5 | insulin-like growth factor | NM_000599 | NP_000590 |
| | binding protein 5 | | |
| IGHG1 | immunoglobulin heavy | BC092518 | CAC20454 |
| | constant gamma 1 (G1m | | |
| | marker) | | |
| IGLV4-3 | immunoglobulin lambda | BC020236 | AAH20236 |
| | variable 4-3 | | |
| KIF5C | kinesin family member | NM_004984 | AAH17298 |
| | 5C | | |
| KNG1 | kininogen 1 | NM_000893 | NP_000884 |
| KRT1 | keratin 1 (epidermolytic | BC063697 | NP_000412 |
| | hyperkeratosis) | | |
| KRT10 | keratin 10 (epidermolytic | NM_000421 | NP_000412 |
| | hyperkeratosis; keratosis | | |
| | palmaris et plantaris) | | |
| KRT9 | keratin 9 (epidermolytic | NM_000226 | NP_000217 |
| | palmoplantar | | |
| | keratoderma) | | |
| LBP | lipopolysaccharide | AF105067 | AAC39547 |
| | binding protein | | |
| LGALS3BP | lectin, galactoside- | NM_005567 | NP_005558 |
| | binding, soluble, 3 | BC015761 | |
| | binding protein | BC002403 | |
| | | BC002998 | |
| LRG1 | leucine-rich alpha-2- | NM_052972 | AAH70198 |
| | glycoprotein 1 | | |
| LUM | lumican | BC035997 | AAP35353 |
| MMP14 | matrix metalloproteinase | NM_004995 | AAV40837 |
| | 14 (membrane-inserted) | | |
| MYH4 | myosin, heavy | NM_017533 | NP_060003 |
| | polypeptide 4, skeletal | | |
| | muscle | | |
| NEB | nebulin | NM_004543 | NP_004534 |
| NUCB2 | nucleobindin 2 | NM_005013 | NP_005004 |
| ORM2 | orosomucoid 2 | NM_000608 | NP_000599 |
| PF4V1 | platelet factor 4 variant 1 | NM_002620 | NP_002611 |
| PIGR | polymeric | NM_002644 | CAC10060 |
| | immunoglobulin receptor | | |
| PLG | plasminogen | NM_000301 | AAH60513 |
| PON1 | paraoxonase 1 | NM_000446 | NP_000437 |
| PPBP | pro-platelet basic protein | NM_002704 | CAG33086 |
| | (chemokine (C—X—C | | |
| | motif) ligand 7) | | |
| RBP4 | retinol binding protein 4, | NM_006744 | CAH72328 |
| | plasma | | |
| RIMS1 | regulating synaptic | NM_014989 | NP_055804 |
| | membrane exocytosis 1 | | |
| RNF6 | ring finger protein | NM_005977 | CAH73183 |
| | (C3H2C3 type) 6 | | |
| SAA1 | serum amyloid A1 | BC105796 | AAA64799 |
| | | | AAA30968 |
| SEMA3D | sema domain, | NM_152754 | EAL24184 |
| | immunoglobulin domain | | |
| | (Ig), short basic domain, | | |
| | secreted, (semaphorin) | | |
| | 3D | | |
| SERPINA1 | serine (or cysteine) | BC015642 | NP_001002235 |
| | proteinase inhibitor, clade | NM_000295 | NP_000286 |
| | A (alpha-1 antiproteinase, | | |
| | antitrypsin), member 1 | | |
| SERPIND1 | serine (or cysteine) | NM_000185 | CAG30459 |
| | proteinase inhibitor, clade | | |
| | D (heparin cofactor), | | |
| | member 1 | | |
| SERPINF2 | serine (or cysteine) | BC031592 | NP_000925 |
| | proteinase inhibitor, clade | | |
| | F (alpha-2 antiplasmin, | | |
| | pigment epithelium | | |
| | derived factor), member 2 | | |
| SERPING1 | serine (or cysteine) | NM_000062; | AAW69393 |
| | proteinase inhibitor, clade | | |
| | G (C1 inhibitor), member | BC011171 | |
| | 1, (angioedema, | | |
| | hereditary) | | |
| SF3B1 | splicing factor 3b, subunit | NM_012433 | NP_006833 |
| | 1, 155 kDa | | |
| SPINK1 | serine protease inhibitor, | NM_003122 | NP_003113 |
| | Kazal type 1 | | |
| SPP1 | secreted phosphoprotein 1 | NM_000582 | AAH17387 |
| | (osteopontin, bone | | |
| | sialoprotein I, early T- | | |
| | lymphocyte activation 1) | | |
| SPTB | spectrin, beta, | NM_001024858 | BAD92652 |
| | erythrocytic (includes | | |
| | spherocytosis, clinical | | |
| | type I) | | |
| SYNE1 | spectrin repeat | NM_182961 | AAH39121 |
| | containing, nuclear | | |
| | envelope 1 | | |
| TAF4B | TAF4b RNA polymerase | NM_003187 | XP_290809 |
| | II, TATA box binding | | |
| | protein (TBP)-associated | | |
| | factor, 105 kDa | | |
| TBC1D1 | TBC1 (tre-2/USP6, | NM_015173 | NP_055988 |
| | BUB2, cdc 16) domain | | |
| | family, member 1 | | |
| TLN1 | talin 1 | NM_006289 | NP_006280 |
| TMSB4X | thymosin, beta 4, X- | NM_021109 | NP_066932 |
| | linked | | |
| TRIP11 | thyroid hormone receptor | NM_004239 | NP_004230 |
| | interactor 11 | | |
| TTR | transthyretin (prealbumin, | NM_000371 | AAH05310 |
| | amyloidosis type I) | | AAP35853 |
| UROC1 | urocanase domain | NM_144639 | NP_653240 |
| | containing 1 | | |
| VTN | Vitronectin (serum | NM_000638 | P04004 |
| | spreading factor, | | |
| | somatomedin B, | | |
| | complement S-protein) | | |
| VWF | von Willebrand factor | NM_000552 | AAB59458 |
| ZFHX2 | zinc finger homeobox 2 | NM_033400 | NP_207646 |
| ZYX | Zyxin | NM_003461 | NP_001010972 |

An embodiment of the present invention consists of those biomarkers that are present in both Tables 1 and Table 5, which is listed in Table 6 below.

TABLE 6
Biomarkers that are present in both Tables 1 and 5.

| | | | Protein |
|---|---|---|---|
| | | Gene Accession | Accession |
| Gene Symbol | Gene Name | Number | Number |
| Column 1 | Column 2 | Column 3 | Column 4 |
| AGT | angiotensinogen (serine or | NM_000029 | AAR03501 |
| | cysteine) proteinase inhibitor, | | |
| | clade A (alpha-1 antiproteinase, | | |
| | antitrypsin), member 8) | | |
| AHSG | alpha-2-HS-glycoprotein | NM_001622 | NP_001613 |
| APOA1 | apolipoprotein A-I | NM_000039 | AAD34604 |
| APOA4 | apolipoprotein A-IV | NM_000482 | AAS68228 |
| APOB | apolipoprotein B (including | NM_000384 | AAP72970 |
| | Ag(x) antigen) | | |
| APOC3 | apolipoprotein C-III | BC027977 | AAB59372 |
| AZGP1 | alpha-2-glycoprotein 1, zinc | NM_001185 | NP_001176 |
| BF | B-factor, properdin | NM_001710 | CAI17456 |
| SERPING1 | serine (or cysteine) proteinase | NM_000062; | AAW69393 |
| | inhibitor, clade G (C1 inhibitor), | BC011171 | |
| | member 1, (angioedema, | | |
| | hereditary) | | |
| C1S | complement component 1, s | NM_201442; | NP_958850 |
| | subcomponent | NM_001734 | NP_001725 |
| C2 | complement component 2 | NM_000063 | CAI17451 |
| C5 | complement component 5 | NM_001736 | NP_001726 |
| C8A | complement component 8, alpha | NM_000562 | CAI19172 |
| | polypeptide | | |
| CLU | clusterin (complement lysis | NM_001831 | AAP88927 |
| | inhibitor, SP-40,40, sulfated | | |
| | glycoprotein 2, testosterone- | | |
| | repressed prostate message 2, | | |
| | apolipoprotein J) | | |
| F2 | coagulation factor II (thrombin) | NM_000506 | AAL77436 |
| FGB | fibrinogen beta chain | NM_005141 | AAA18024 |
| GSN | gelsolin (amyloidosis, Finnish | BC026033 | CAI14413 |
| | type) | | |
| HBB | hemoglobin, beta | NM_000518 | AAD19696 |
| SERPIND1 | serine (or cysteine) proteinase | NM_000185 | CAG30459 |
| | inhibitor, clade D (heparin | | |
| | cofactor), member 1 | | |
| HP | haptoglobin | BC107587 | NP_005134 |
| HPX | hemopexin | NM_000613 | NP_000604 |
| KNG1 | kininogen 1 | NM_000893 | NP_000884 |
| KRT1 | keratin 1 (epidermolytic | BC063697 | NP_000412 |
| | hyperkeratosis) | | |
| LGALS3BP | lectin, galactoside-binding, | NM_005567 | NP_005558 |
| | soluble, 3 binding protein | BC015761 | |
| | | BC002403 | |
| | | BC002998 | |
| SERPINA1 | serine (or cysteine) proteinase | BC015642 | NP_001002235 |
| | inhibitor, clade A (alpha-1 | NM_000295 | NP_000286 |
| | antiproteinase, antitrypsin), | | |
| | member 1 | | |
| SERPINF2 | serine (or cysteine) proteinase | BC031592 | NP_000925 |
| | inhibitor, clade F (alpha-2 | | |
| | antiplasmin, pigment epithelium | | |
| | derived factor), member 2 | | |
| SAA1 | serum amyloid A1 | BC105796 | AAA64799 |
| | | | AAA30968 |
| TTR | transthyretin (prealbumin, | NM_000371 | AAH05310 |
| | amyloidosis type I) | | AAP35853 |
| VTN | vitronectin (serum spreading | NM_000638 | P04004 |
| | factor, somatomedin B, | | |
| | complement S-protein) | | |
| VWF | von Willebrand factor | NM_000552 | AAB59458 |
| APOL1 | apolipoprotein L, 1 | BC017331 | AAK20210 |
| | | NM_003661 | |
| TRIP11 | thyroid hormone receptor | NM_004239 | NP_004230 |
| | interactor 11 | | |
| LRG1 | leucine-rich alpha-2- | NM_052972 | AAH70198 |
| | glycoprotein 1 | | |
| AGT | angiotensinogen (serine (or | NM_000029 | AAR03501 |
| | cysteine) proteinase inhibitor, | | |
| | clade A (alpha-1 antiproteinase, | | |
| | antitrypsin), member 8) | | |

Biomarkers from Table 1 or 5 known to be associated with coagulation were determined and constitute another embodiment of the present invention as set forth in Table 7.

TABLE 7
Biomarkers from Table 1 or 5 known to be associated with coagulation

| | | | Gene |
|---|---|---|---|
| | | Gene Accession | Protein |
| Gene Symbol | Gene Name | Name | Name |
| Column 1 | Column 2 | Column 3 | Column 4 |
| AGT | angiotensinogen (serine (or | NM_000029 | AAR03501 |
| | cysteine) proteinase inhibitor, | | |
| | clade A (alpha-1 antiproteinase, | | |
| | antitrypsin), member 8) | | |
| APCS | amyloid P component, serum | BT006750 | CAH73651 |
| BF | B-factor, properdin | NM_001710 | CAI17456 |
| SERPING1 | serine (or cysteine) proteinase | NM_000062 | AAW69393 |
| | inhibitor, clade G (C1 inhibitor), | BC011171 | |
| | member 1, (angioedema, | | |
| | hereditary) | | |
| C1QB | complement component 1, q | NM_000491 | NP_000482 |
| | subcomponent, beta polypeptide | | CAI22896 |
| C1R | complement component 1, r | NM_001733 | NP_001724 |
| | subcomponent | | |
| C1S | complement component 1, s | NM_201442; | NP_958850 |
| | subcomponent | NM_001734 | NP_001725 |
| C2 | complement component 2 | NM_000063 | CAI17451 |
| | | | CAI41858 |
| C3 | complement component 3 | NM_000064 | AAR89906 |
| C4BPA | complement component 4 | NM_001017367 | CAH70782 |
| | binding protein, alpha | | |
| C5 | complement component 5 | NM_001736 | NP_001726 |
| C6 | complement component 6 | NM_000065 | BAD02322 |
| C7 | complement component 7 | NM_000587 | CAA72407 |
| C8A | complement component 8, alpha | NM_000562 | CAI19172 |
| | polypeptide | | |
| C8B | complement component 8, beta | NM_000066 | CAC18532 |
| | polypeptide | | |
| C8G | complement component 8, | NM_000606 | NP_000597 |
| | gamma polypeptide | | |
| C9 | complement component 9 | NM_001737 | NP_001728 |
| | | | AAH20721 |
| CLU | clusterin (complement lysis | NM_001831 | AAP88927 |
| | inhibitor, SP-40,40, sulfated | | |
| | glycoprotein 2, testosterone- | | |
| | repressed prostate message 2, | | |
| | apolipoprotein J) | | |
| CPN1 | carboxypeptidase N, polypeptide | NM_001308 | NP_001299 |
| | 1, 50 kD | | |
| CRP | C-reactive protein, pentraxin- | NM_000567 | NP_000558 |
| | related | | |
| IF | I factor (complement) | NM_000204 | LOOK UP |
| | | | LATER |
| IGFBP5 | insulin-like growth factor binding | NM_000599 | NP_000590 |
| | protein 5 | | |
| KRT1 | keratin 1 (epidermolytic | BC063697 | NP_000412 |
| | hyperkeratosis) | | |
| PLG | Plasminogen | NM_000301 | AAH60513 |
| C4B | — | NM_000592 | AAR89095 |

### Example 2

Recent developments in proteomics have allowed for analysis of complex protein fluids in greater detail than previously possible. Mass spectrometry has allowed for biomarker study and differentiation of complex samples in a multitude of diseases. Specifically the diagnosis of renal cell cancer (Tolson et al. 2004, Lab Invest. 84:845-56) breast cancer (Paweletza et al., 2001, Dis Markers. 17:301-307) ovarian cancer (Zhang et al., 2004, Cancer Res. 64: 5882-5890) and even the identification of intra-uterine inflammation (Buhimschi et al., 2005, BJOG. 2005; 112:173-81) have been suggested using mass spectrometry technologies.

The study described in this second example was designed to evaluate differences in protein composition of plasma between critically ill SIRS patients who are becoming septic (converters), as compared to critically ill SIRS patients who remain uninfected (nonconverters). Specifically, it was hypothesized that the plasma protein composition of critically ill SIRS patients with sepsis (converters) would be different than plasma protein composition of phenotypically similar uninfected patients manifesting SIRS (nonconverters). Furthermore, it was hypothesized that these differences would be detectable prior to the clinical diagnosis of sepsis.

As part of an ongoing study to characterize differences between sterile inflammation and sepsis, critically ill uninfected SIRS patients were prospectively evaluated for the development of clinical sepsis. Patients over the age of 18, admitted to a trauma intensive care unit were screened. Trauma patients who met 2 of 4 standard SIRS criteria (Table 8), drawn from Anonymous, 1992, American College of Chest Physician/Society of Critical Care Medicine Consensus Conference: Definitions for sepsis and organ failure and guidelines for the use of innovative therapies in sepsis, Crit. Care Med. 20: 864-874, which is hereby incorporated by reference herein), and were clinically uninfected were enrolled.

TABLE 8
Selection criteria.

| SIRS criteria: | Must meet 2 of 4 for study entry |
|---|---|
| 1) Temperature | >38° or <36° centigrade |
| 2) Respiratory status | RR > 20, pCO2 < 32 or mechanical |
| | Ventilation |
| 3) Heart rate | >90 BPM |
| 4) White Blood Cell Count | >12k/mcl or >10% immature forms |

Exclusion criteria (Table 9) included potential immunocompromising states, administration of antibiotics for treatment, and extended prophylactic antibiotic use.

TABLE 9
Exclusion criteria

| | |
|---|---|
| Known HIV positive at entry | Pregnancy |
| Organ transplant recipients | Spinal cord injuries having |
| | received steroids |
| Pharmacologic immunosuppression | Empiric antibiotic use upon entry |
| Recent chemo- or radiotherapy | Investigational drug use within |
| (within 8 weeks prior to enrollment) | 30 days of enrollment |
| Prophylactic antibiotics longer than | |
| 48 hours duration | |

Patients were divided into 2 groups: 1) Uninfected SIRS: patients who remained uninfected for the course of the study and 2) Pre-septic SIRS: SIRS patients who developed clinical sepsis during the course of the study. Sepsis diagnoses were based on the standard clinical criteria for SIRS and Sepsis. See, Bone et al., 1992, “Definitions for sepsis and organ failure,” Crit. Care Med 20: 724-726; and Levy et al., 2003, 2001 SCCM/ESICM/ACCP/ATS/SIS International Sepsis Definitions Conference. Crit. Care Med. 31:1250-1256, each of which is hereby incorporated by reference herein

Plasma was collected daily until ICU discharge (maximum 14 days) in the uninfected SIRS group. For the pre-septic SIRS group, plasma was collected daily until the clinical diagnosis of sepsis then for a subsequent 3 days (maximum 17 days). Patient plasma was collected predominantly via a previously placed central venous catheter using PPT (plasma preparation tube, Becton Dickenson, Vacutainer, Franklin Lakes, N.J.). Immediately after collection, samples were centrifuged at 1100×g for 20 mins, and plasma was subsequently removed by pipetting, and divided into 0.5 mL aliquots. Samples were stored frozen at −70° C. until analyzed.

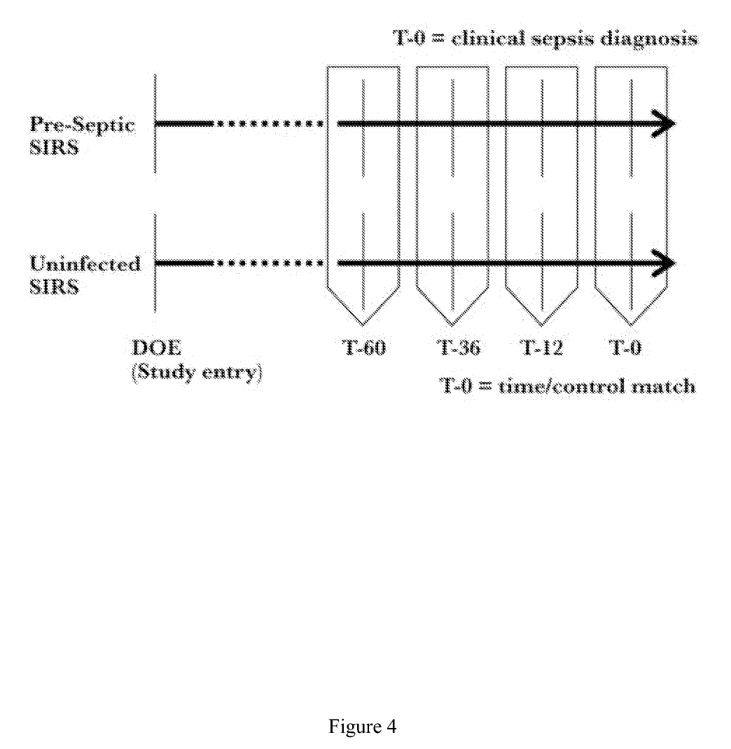

In order to group patients by similar severity of disease, and since pre-septic patients converted to sepsis at varying time points after enrollment, all pre-septic patients were retrospectively normalized, using their clinical conversion to sepsis as the normalization point (T₋₀). See FIG. 4. In this second example, clinical conversion time (T₋₀) was defined as the time: 1) a positive culture was obtained from an otherwise sterile location or direct visualization of perforated or necrotic bowel; and 2) a clinical treatment (antibiotics and/or surgical procedure) was initiated for the infection as determined by majority consensus of an infectious disease attending, surgery attending, and critical care attendance. For the uninfected SIRS group, samples were time matched and T₋₀ normalized to clinically similar pre-septic SIRS samples, based on demographic information, continued presence of SIRS, and elapsed time in the study. For both groups, samples were analyzed at four time points: (i) Date of entry (samples drawn at study entry when both groups were uninfected), (ii) T₋₁₂ (samples collected between 1 to 24 hours prior to the T₋₀ time point), (iii) T₋₃₆ (samples collected 25-48 hours prior to T₋₀) and, (iv) T₋₆₀: samples drawn 49-72 hours prior to T₋₀.

Protein profiling was performed in two experiments. Experiment one evaluated proteins differentially expressed at all time points tested between pre-septic SIRS and uninfected SIRS in pooled plasma samples using a 3-dimensional reverse phase/strong cation exchange/reverse phase liquid chromatography with electrospray ion trap mass spectrometry (LC³ MS²), and spectrum counting for comparative quantitation. See Shen et al., 2006, J Proteome Res. 10: 1021/pr060327k. This mass spectrometry was performed by Mass Consortium Corporation, San Diego, Calif. Briefly, plasma samples from 18 pre-septic patients and 17 SIRS patients were pooled into 6 plasma pools (3 reseptic and 3 uninfected SIRS). Each individual pool was run at each time point. Samples were prepared by immunodepletion of abundant proteins (albumin, transferrin, haptoglobin, anti-trypsin, IgG and IgA) via Agilent Multiple Affinity Removal System (Agilent Technologies, Palo Alto, Calif.). Remaining protein was concentrated, denatured in urea, reduced and alkylated, rediluted then digested twice with trypsin. The 3-dimensional liquid chromatographic (LC³) separation process previously described (see, Wei et al. 2005, J Proteome Res. 4: 801-808) was performed prior to loading. This process was necessary since traditional two-dimensional liquid chromatography (LC²) is insufficient for these complex mixtures. Instead, the digest underwent a reverse-phase (RP) separation based on hydrophobicity, followed by strong-cation exchange (SCX) separation based on ion strength and then a third RP column was used to perform high resolution separation of the sample. Spectra peaks were identified and semi-quantitated using Agilent Technologies Spectrum Mill MS Proteomics Workbench software (version 2.7, Agilent Technologies, Palo-Alto, Calif.). MS/MS (MS2) spectra were searched against the National Center for Biotechnology Information (NCBI) non-redundant protein database. The false positive rate was estimated by auto-validating 4294 spectra and 107 proteins by searching against a combined forward-reverse database. For proteins with at least two unique peptides, the false positive rate was 2.8%. Spectrum counting was used for relative protein quantification. The total spectra numbers were normalized across all rounds and entries were removed if they had a distinct sum tag score less than 13. Sepsis to SIRS ratios were calculated using the normalized total spectra numbers. Where SIRS>Sepsis, the ratio was calculated using 1/(Sepsis/SIRS). If either number was zero, the entry was tagged SEPSIS⁺ or SIRS⁺ as appropriate. Discovered proteins were matched to Entrez Gene identifiers.

In experiment two, a slightly different procedure was performed. Electrospray ionization (ESI) LTQ-FTMS (Thermo Electron, Waltham Mass.) mass spectrometry profiling was run on pooled plasma on both groups collected at the T₋₁₂ time point. Large proteins were removed by centrifugal ultracentrifugation using a 30-kDa cut-off Centriplus ultrafilter (Millipore, Billerica, Mass.). This was followed by passing samples through an SCX and C₁₈ column, prior to a single round of liquid chromatography. Eleven pre-septic patients were compared to 10 uninfected SIRS patients. Peaks were identified using Agilent Technologies Spectrum Mill Workbench software. The data was normalized and ratios calculated identically to the first experiment. Proteins were matched to Entrez-gene identifiers.

To ascertain functional and relevant biologic pathways, the list of proteins identified as differential between the pre-septic and uninfected SIRS groups was uploaded as their corresponding Entrez-gene identifiers to the Database for Annotation, Visualization, and integrated Discovery version 2.1 (DAVID 2.1) software available from the National Institute of Allergy and Infectious Disease (Dennis et al. 2003, DAVID: Database for Annotation, Visualization, and Integrated Discovery. Genome Biol. 4: P3; and Hosack et al., 2003, Identifying Biological Themes within Lists of Genes with EASE. Genome Biol. 4: P4. This allowed for annotation to biologic pathways. Statistical significance of pathways was analyzed by EASE score. The EASE score, a modification of the Fisher-exact test, allows for the ranking of biologic pathways associated with sets of genes and identifies functional categories over-represented in a gene list relative to its representation within the genome of a given species. Significant genes are mapped to known KEGG (Kanehisa et al., 2000 Nucleic Acids Res. 28: 27-30, and Kanehisa et al., 2006, Nucleic Acids Res. 34: D354-357) and Biocarta (available from Biocarta.com) pathways.

For experiment one, the patients were well matched for age and APACHE II scores. While APACHE II scores trended higher in the pre-septic group, this difference was not significant (Table 10).

TABLE 10
Experiment one demographics

| | Uninfected SIRS | Preseptic SIRS | P Value |
|---|---|---|---|
| Age (years) | 45 ± 22 | 45 ± 28 | Ns |
| Gender (M:F) | 72:28 | 70:30 | Ns |
| APACHE II | 12.2 ± 5.1 | 14.6 ± 4.4 | P = .161 |
| ISS | 29.2 ± 9.9 | 29.7 ± 9.6 | Ns |
| TRISS | 0.84 ± .15 | 0.73 ± .30 | P = .19 |
| Mechanism | 100% Blunt | 72% Blunt | |
| Closed Head Injury | 8 | 8 | |
| Solid Organ Injury | 5 | 8 | |
| (liver or spleen) | | | |
| Hollow viscus injury | 0 | 4 | |
| Pulmonary/cardiac | 6 | 10 | |
| injury | | | |
| Major orthopedic | 7 | 10 | |
| injury (proximal | | | |
| long bone/pelvis) | | | |

The pre-septic group did have a higher number of penetrating injuries and intra-abdominal injuries, but despite this, both ISS and TRISS were well matched between groups. Similar demographics were noted in experiment two (Table 11).

TABLE 11
Experiment two demographics

| | Uninfected SIRS | Preseptic SIRS | P Value |
|---|---|---|---|
| Age (years) | 44 ± 18 | 37 ± 16 | Ns |
| Gender (M:F) | 70:30 | 90:10 | |
| APACHE II | 12.2 ± 5.5 | 14.6 ± 4.5 | P = .32 |
| ISS | 27.0 ± 9.1 | 30.1 ± 9.9 | P = .23 |
| TRISS | 0.80 ± .13 | 0.68 ± .35 | P = .48 |
| Mechanism | 100% Blunt | 45% Blunt | |
| Closed Head Injury | 6 | 2 | |
| Solid Organ Injury | 0 | 5 | |
| (liver or spleen) | | | |
| Hollow viscus injury | 0 | 4 | |
| Pulmonary/cardiac | 3 | 4 | |
| injury | | | |
| Major orthopedic | 4 | 2 | |
| injury (proximal | | | |
| long bone/pelvis) | | | |

In experiment one, at date of entry, 55 proteins were differential between groups: 37 were semi-quantitatively greater in the pre-septic group (converters), while 18 were decreased. At T₋₆₀, 54 unique proteins were noted to be differential between groups (Table 12) of which 22 were semi-quantitatively greater in the sepsis group.

TABLE 12
Unique proteins noted to be differential at T₋₆₀ in
experiment one of example two.

| | | | | Directional |
|---|---|---|---|---|
| | | | | change |
| | | Gene Acession | Gene Protein | (Sepsis/ |
| Symbol | Description | Name | Name | SIRS) |
| Column 1 | Column 2 | Column 3 | Column 4 | Column 5 |
| AFM | Afamin | NM_001133 | AAA21612 | increased |
| AHSG | alpha-2-HS- | NM_001622 | NP_001613 | decreased |
| | glycoprotein | | | |
| APOA1 | apolipoprotein A-I | NM_000039 | AAD34604 | decreased* |
| APOA2 | apolipoprotein A-II | NM_001643 | AAA51701 | increased |
| APOA4 | apolipoprotein A-IV | NM_000482 | AAS68228 | increased |
| APOB | apolipoprotein B | NM_000384 | AAP72970 | decreased |
| | (including Ag(x) | | | |
| | antigen) | | | |
| APOC3 | apolipoprotein C-III | BC027977 | AAB59372 | decreased |
| APOH | apolipoprotein H | NM_000042 | CAA40977 | decreased |
| | (beta-2-glycoprotein | | | |
| | I) | | | |
| APOL1 | apolipoprotein L, 1 | BC017331 | AAK20210 | increased |
| | | NM_003661 | | |
| BCOR | BCL6 co-repressor | BC063536 | AAG41429 | decreased |
| BDP1 | B double prime 1, | NM_018429 | AAH32146 | decreased |
| | subunit of RNA | | | |
| | polymerase III | | | |
| | transcription initiation | | | |
| | factor IIIB | | | |
| C1QB | complement | NM_000491 | NP_000482 | increased |
| | component 1, q | | | |
| | subcomponent, beta | | | |
| | polypeptide | | | |
| C1S | complement | NM_201442; | NP_958850 | increased |
| | component 1, s | NM_001734 | | |
| | subcomponent | | | |
| C3 | complement | NM_000064 | AAR89906 | decreased |
| | component 3 | | | |
| C5 | complement | NM_001736 | NP_001726 | decreased |
| | component 5 | | | |
| C8A | complement | NM_000562 | CAI19172 | decreased* |
| | component 8, alpha | | | |
| | polypeptide | | | |
| C9 | complement | NM_001737 | NP_001728 | decreased* |
| | component 9 | | | |
| CD14 | CD14 antigen | NM_000591 | AAP35995 | increased* |
| CP | ceruloplasmin | NM_000096 | NP_000087 | decreased |
| | (ferroxidase) | | | |
| CRP | C-reactive protein, | NM_000567 | NP_000558 | decreased |
| | pentraxin-related | | | |
| FGA | fibrinogen alpha chain | BC070246 | BAC55116 | decreased* |
| FGB | fibrinogen beta chain | NM_005141 | AAA18024 | decreased* |
| FLNA | filamin A, alpha (actin | NM_001456 | CAI43227 | increased |
| | binding protein 280) | | | |
| FN1 | fibronectin 1 | BT006856 | BAD52437 | increased* |
| GC | group-specific | NM_000583 | NP_000574 | decreased* |
| | component (vitamin D | | | |
| | binding protein) | | | |
| HBB | hemoglobin, beta | NM_000518 | AAD19696 | decreased |
| HP | haptoglobin | BC107587 | NP_005134 | decreased |
| HPX | hemopexin | NM_000613 | NP_000604 | decreased |
| HRG | histidine-rich | NM_000412 | NP_000403 | increased |
| | glycoprotein | | | |
| IF | I factor (complement) | NM_000204 | NP_000195 | increased |
| ITIH1 | inter-alpha (globulin) | BC109115 | NP_002206 | increased* |
| | inhibitor H1 | | NP_032432 | |
| ITIH2 | inter-alpha (globulin) | NM_002216 | NP_002207 | increased |
| | inhibitor H2 | | | |
| ITIH4 | inter-alpha (globulin) | NM_002218 | NP_002209 | decreased |
| | inhibitor H4 (plasma | | | |
| | Kallikrein-sensitive | | | |
| | glycoprotein) | | | |
| KLKB1 | kallikrein B, plasma | NM_000892 | NP_000883 | increased |
| | (Fletcher factor) 1 | | | |
| KNG1 | kininogen 1 | NM_000893 | NP_000884 | decreased* |
| KRT1 | keratin 1 | BC063697 | NP_000412 | decreased |
| | (epidermolytic | | | |
| | hyperkeratosis) | | | |
| LGALS3BP | lectin, galactoside- | NM_005567 | NP_005558 | increased |
| | binding, soluble, 3 | BC015761 | | |
| | binding protein | BC002403 | | |
| | | BC002998 | | |
| LPA | lipoprotein, Lp(a) | NM_005577 | NP_005568 | decreased |
| LRG1 | leucine-rich alpha-2- | NM_052972 | AAH70198 | decreased |
| | glycoprotein 1 | | | |
| MGC275 | hypothetical protein | BC087841 | AAH87841 | increased |
| | MGC27165 | BC005951 | | |
| MYO18B | myosin XVIIIB | NM_032608 | NP_115997 | decreased |
| ORM1 | orosomucoid 1 | NM_000607 | CAI16859 | decreased* |
| PGLYRP2 | peptidoglycan | | | decreased |
| | recognition protein 2 | | | |
| QSCN6 | quiescin Q6 | NM_002826 | AAQ89300 | decreased |
| RGS4 | regulator of G-protein | NM_005613 | NP_005604 | decreased |
| | signalling 4 | | | |
| SAA1 | Serum amyloid A1 | BC105796 | AAA64799 | increased* |
| | | | AAA30968 | |
| SERPINA1 | serine (or cysteine) | BC015642 | NP_001002235 | increased* |
| | proteinase inhibitor, | NM_000295 | NP_000286 | |
| | clade A (alpha-1 | | | |
| | antiproteinase, | | | |
| | antitrypsin), member 1 | | | |
| SERPINA3 | serine (or cysteine) | NM_001085 | NP_001076 | decreased |
| | proteinase inhibitor, | | | |
| | clade A (alpha-1 | | | |
| | antiproteinase, | | | |
| | antitrypsin), member 3 | | | |
| SERPINA6 | serine (or cysteine) | NM_001756 | NP_001002236 | increased |
| | proteinase inhibitor, | | NP_000286 | |
| | clade A (alpha-1 | | | |
| | antiproteinase, | | | |
| | antitrypsin), member 6 | | | |
| SERPINC1 | serine (or cysteine) | NM_000488 | CAI19423 | increased |
| | proteinase inhibitor, | X68793 | P01008 | |
| | clade C | | | |
| | (antithrombin), | | | |
| | member 1 | | | |
| SERPIND1 | serine (or cysteine) | NM_000185 | CAG30459 | decreased |
| | proteinase inhibitor, | | | |
| | clade D (heparin | | | |
| | cofactor), member 1 | | | |
| SERPING1 | serine (or cysteine) | NM_000062; | AAW69393 | decreased* |
| | proteinase inhibitor, | BC011171 | | |
| | clade G (C1 | | | |
| | inhibitor), member 1 | | | |
| TRIP11 | thyroid hormone | NM_004239 | NP_004230 | increased |
| | receptor interactor 11 | | | |
| VTN | vitronectin (serum | NM_000638 | P04004 | increased* |
| | spreading factor, | | | |
| | somatomedin B, | | | |
| | complement S- | | | |
| | protein) | | | |

* = discordance between pools, predominant direction noted is listed.

At T₋₃₆, 27 unique proteins were noted to be differential between groups (Table 13) of which 10 were semi-quantitatively greater in the sepsis group.

TABLE 13
Unique proteins noted to be differential at T₋₃₆ in experiment one of example two.

| | | Gene | | Directional |
|---|---|---|---|---|
| | | Acession | Gene Protein | change (Sepsis/ |
| Symbol | Description | Name | Name | SIRS) |
| Column 1 | Column 2 | Column 3 | Column 4 | Column 5 |
| AFM | afamin | NM_001133 | AAA21612 | increased |
| AGT | angiotensinogen | NM_000029 | AAR03501 | decreased |
| | (serine (or cysteine) | | | |
| | proteinase inhibitor | | | |
| AHSG | alpha-2-HS- | NM_001622 | NP_001613 | decreased |
| | glycoprotein | | | |
| ALMS1 | Alstrom syndrome 1 | NM_015120 | NP_055935 | increased |
| APOA1 | apolipoprotein A-I | NM_000039 | AAD34604 | increased |
| APOB | apolipoprotein B | NM_000384 | AAP72970 | decreased |
| | (including Ag(x) | | | |
| | antigen) | | | |
| APOE | apolipoprotein E | NM_000041 | AAB59397 | decreased |
| C2 | complement | NM_000063 | CAI17451 | decreased |
| | component 2 | | | |
| C3 | complement | NM_000064 | AAR89906 | decreased |
| | component 3 | | | |
| CP | ceruloplasmin | NM_000096 | NP_000087 | decreased |
| | (ferroxidase) | | | |
| F2 | coagulation factor II | NM_000506 | AAL77436 | decreased |
| | (thrombin) | | | |
| FGB | fibrinogen beta chain | NM_005141 | AAA18024 | increased |
| FLJ10006 | hypothetical protein | BC110537 | AAH17012 | decreased |
| | FLJ10006 | BC110536 | | |
| GC | group-specific | NM_000583 | NP_000574 | decreased |
| | component (vitamin D | | | |
| | binding protein) | | | |
| IF | I factor (complement) | NM_000204 | NP_000195 | increased |
| IGFALS | insulin-like growth | NM_004970 | NP_004961 | decreased |
| | factor binding protein, | | | |
| | acid labile subunit | | | |
| ITIH1 | inter-alpha (globulin) | BC109115 | NP_002206 | increased |
| | inhibitor H1 | | NP_032432 | |
| KCTD7 | potassium channel | NM_153033 | NP_694578 | decreased |
| | tetramerisation domain | | | |
| | containing 7 | | | |
| KNG1 | kininogen 1 | NM_000893 | NP_000884 | decreased |
| LPA | lipoprotein, Lp(a) | | | increased |
| ORM1 | orosomucoid | NM_000607 | CAI16859 | decreased |
| PDCD11 | programmed cell death | NM_014976 | NP_055791 | increased |
| | 11 | | | |
| SERPINA1 | serine (or cysteine) | BC015642 | NP_001002235 | decreased |
| | proteinase inhibitor, | NM_000295 | NP_000286 | |
| | clade A (alpha-1 | | | |
| | antitrypsin), member 1 | | | |
| SERPINA3 | serine (or cysteine) | NM_001085 | NP_001076 | decreased |
| | proteinase inhibitor, | | | |
| | clade A (alpha-1) | | | |
| SERPINC1 | serine (or cysteine) | NM_000488 | CAI19423 | decreased |
| | proteinase inhibitor, | X68793 | P01008 | |
| | clade C (antithrombin), | | | |
| | member 1 | | | |
| SERPING1 | serine (or cysteine) | NM_000062; | AAW69393 | increased |
| | proteinase inhibitor, | BC011171 | | |
| | clade G (C1 inhibitor), | | | |
| | member 1, | | | |
| VTN | vitronectin (serum | NM_000638 | P04004 | increased |
| | spreading factor, | | | |
| | somatomedin B, | | | |
| | complement S-protein) | | | |

At T₋₁₂, 38 unique proteins (Table 14) were noted to be differential between groups of which 28 were semi-quantitatively greater in the sepsis group.

TABLE 14
Unique proteins noted to be differential at T₋₁₂ in experiment one of example two.

| | | | | Directional |
|---|---|---|---|---|
| | | Gene Acession | Gene Protein | change (Sepsis/ |
| Symbol | Description | Name | Name | SIRS) |
| Column 1 | Column 2 | Column 3 | Column 4 | Column 5 |
| APOA2 | apolipoprotein A-II | NM_001643 | AAA51701 | increased |
| APOA4 | apolipoprotein A-IV | NM_000482 | AAS68228 | increased |
| APOC1 | apolipoprotein C-I | NM_001645 | AAQ91813 | increased |
| APOC3 | apolipoprotein C-III | BC027977 | AAB59372 | decreased |
| APOE | apolipoprotein E | NM_000041 | AAB59397 | decreased |
| APOH | apolipoprotein H | NM_000042 | CAA40977 | increased |
| | (beta-2-glycoprotein I) | | | |
| BF | B-factor, properdin | NM_001710 | CAI17456 | increased |
| C1S | complement | NM_201442; | NP_958850 | decreased |
| | component 1, s | NM_001734 | | |
| | subcomponent | | | |
| C3 | complement | NM_000064 | AAR89906 | increased |
| | component 3 | | | |
| C4BPA | complement | NM_001017367 | CAH70782 | decreased |
| | component 4 binding | | | |
| | protein, alpha | | | |
| C9 | complement | NM_001737 | NP_001728 | increased |
| | component 9 | | | |
| CLU | clusterin (complement | NM_001831 | AAP88927 | increased |
| | lysis inhibitor, SP- | | | |
| | 40,40, sulfated | | | |
| | glycoprotein 2) | | | |
| F9 | coagulation factor IX | NM_000133 | NP_000124 | increased |
| | (plasma | | | |
| | thromboplastic | | | |
| | component) | | | |
| FN1 | fibronectin 1 | BT006856 | BAD52437 | increased |
| GC | group-specific | NM_000583 | NP_000574 | increased* |
| | component (vitamin D | | | |
| | binding protein) | | | |
| HBB | hemoglobin, beta | NM_000518 | AAD19696 | increased |
| HPX | hemopexin | NM_000613 | NP_000604 | decreased |
| IF | I factor (complement) | NM_000204 | NP_000195 | increased |
| ITIH1 | inter-alpha (globulin) | BC109115 | NP_002206 | increased |
| | inhibitor H1 | | NP_032432 | |
| ITIH2 | inter-alpha (globulin) | NM_002216 | NP_002207 | decreased |
| | inhibitor H2 | | | |
| ITIH4 | inter-alpha (globulin) | NM_002218 | NP_002209 | increased |
| | inhibitor H4 (plasma | | | |
| | Kallikrein-sensitive | | | |
| KLKB1 | kallikrein B, plasma | NM_000892 | NP_000883 | decreased |
| | (Fletcher factor) 1 | | | |
| KNG1 | kininogen 1 | NM_000893 | NP_000884 | increased |
| LPA | lipoprotein, Lp(a) | NM_005577 | NP_005568 | decreased |
| LRG1 | leucine-rich alpha-2- | NM_052972 | AAH70198 | increased* |
| | glycoprotein 1 | | | |
| ORM1 | orosomucoid 1 | NM_000607 | CAI16859 | increased* |
| QSCN6 | quiescin Q6 | NM_002826 | AAQ89300 | increased |
| SAA1 | serum amyloid A1 | BC105796 | AAA64799 | increased |
| | | | AAA30968 | |
| SERPINA1 | serine (or cysteine) | BC015642 | NP_001002235 | increased |
| | proteinase inhibitor, | NM_000295 | NP_000286 | |
| | clade A (alpha-1 | | | |
| | antiproteinase, | | | |
| | antitrypsin), member 1 | | | |
| SERPINF2 | serine (or cysteine) | BC031592 | NP_000925 | decreased |
| | proteinase inhibitor, | | | |
| | clade F (alpha-2 | | | |
| | antiplasmin, pigment | | | |
| | epithelium derived | | | |
| | factor), member 2 | | | |
| SERPING1 | serine (or cysteine) | NM_000062; | AAW69393 | increased |
| | proteinase inhibitor, | BC011171 | | |
| | clade G (C1 inhibitor), | | | |
| | member 1, | | | |
| SMARCAD1 | SWI/SNF-related, | NM_020159 | NP_064544 | increased |
| | matrix-associated | | | |
| | actin-dependent | | | |
| | regulator of chromatin, | | | |
| | subfamily a, | | | |
| | containing DEAD/H | | | |
| | box 1 | | | |
| TF | Transferring | NM_001063 | NP_001054 | increased |
| TTN | Titin | BC013396 | CAD12456 | decreased |
| TTR | transthyretin | NM_000371 | AAH05310 | increased |
| | (prealbumin, | | AAP35853 | |
| | amyloidosis type I) | | | |
| VWF | Von Willibrand Factor | NM_000552 | AAB59458 | increased |

In all, accounting for proteins apparent in more than one time point, there were 71 unique proteins in experiment 1 corresponding to unique Entrez gene identifiers demonstrating significant differences between groups at the three time points prior to sepsis diagnosis excluding DOE. These 71 unique proteins are listed in Table 15.

TABLE 15
Unique proteins that are differentially expressed at T₋₆₀, T₋₃₆ and/or T₋₁₂ in
experiment one of example two.

| | | | | Directional |
|---|---|---|---|---|
| | | | | change |
| | | Gene Acession | Gene Protein | (Sepsis/ |
| Symbol | Description | Name | Name | SIRS) |
| Column 1 | Column 2 | Column 3 | Column 4 | Column 5 |
| AFM | afamin | NM_001133 | AAA21612 | increased T₋₆₀ |
| | | | | increased T₋₃₆ |
| AGT | Angiotensinogen | NM_000029 | AAR03501 | decreased T₋₃₆ |
| | (serine or cysteine) | | | |
| | proteinase inhibitor | | | |
| AHSG | alpha-2-HS- | NM_001622 | NP_001613 | decreased T₋₆₀ |
| | glycoprotein | | | decreased T₋₃₆ |
| ALMS1 | Alstrom syndrome 1 | NM_015120 | NP_055935 | increased T₋₃₆ |
| APOA1 | apolipoprotein A-I | NM_000039 | AAD34604 | decreased T₋₆₀ |
| | | | | increased T₋₃₆ |
| APOA2 | apolipoprotein A-II | NM_001643 | AAA51701 | increased T₋₆₀ |
| | | | | increased T₋₁₂ |
| APOA4 | apolipoprotein A-IV | NM_000482 | AAS68228 | increased T₋₆₀ |
| | | | | increased T₋₁₂ |
| APOB | apolipoprotein B | NM_000384 | AAP72970 | decreased T₋₆₀ |
| | (including Ag(x) | | | decreased T₋₃₆ |
| | antigen) | | | |
| APOC1 | apolipoprotein C-I | NM_001645 | AAQ91813 | increased T₋₁₂ |
| APOC3 | apolipoprotein C-III | BC027977 | AAB59372 | decreased T₋₆₀ |
| | | | | decreased T₋₁₂ |
| APOE | Apolipoprotein E | NM_000041 | AAB59397 | decreased T₋₃₆ |
| | | | | decreased T₋₁₂ |
| APOH | apolipoprotein H | NM_000042 | CAA40977 | decreased T₋₆₀ |
| | (beta-2-glycoprotein | | | increased T₋₁₂ |
| | I) | | | |
| APOL1 | apolipoprotein L, 1 | BC017331 | AAK20210 | increased T₋₆₀ |
| | | NM_003661 | | |
| BCOR | BCL6 co-repressor | BC063536 | AAG41429 | decreased T₋₆₀ |
| BDP1 | B double prime 1, | NM_018429 | AAH32146 | decreased T₋₆₀ |
| | subunit of RNA | | | |
| | polymerase III | | | |
| | transcription initiation | | | |
| | factor IIIB | | | |
| BF | B-factor, properdin | NM_001710 | CAI17456 | increased T₋₁₂ |
| C1QB | complement | NM_000491 | NP_000482 | increased T₋₆₀ |
| | component 1, q | | | |
| | subcomponent, beta | | | |
| | polypeptide | | | |
| C1S | complement | NM_201442; | NP_958850 | increased T₋₆₀ |
| | component 1, s | | | |
| | subcomponent | NM_001734 | | decreased T₋₁₂ |
| C2 | complement | NM_000063 | CAI17451 | decreased T₋₃₆ |
| | component 2 | | | |
| C3 | complement | NM_000064 | AAR89906 | decreased T₋₆₀ |
| | component 3 | | | decreased T₋₃₆ |
| | | | | increased T₋₁₂ |
| C4BPA | complement | NM_001017367 | CAH70782 | decreased T₋₁₂ |
| | component 9 | | | |
| C5 | complement | NM_001736 | NP_001726 | decreased T₋₆₀ |
| | component 5 | | | |
| C8A | complement | NM_000562 | CAI19172 | decreased T₋₆₀ |
| | component 8, alpha | | | |
| | polypeptide | | | |
| C9 | complement | NM_001737 | NP_001728 | decreased T₋₆₀ |
| | component 9 | | | increased T₋₁₂ |
| CD14 | CD14 antigen | NM_000591 | AAP35995 | increased T₋₆₀ |
| CLU | clusterin (complement | NM_001831 | AAP88927 | increased T₋₁₂ |
| | lysis inhibitor, SP- | | | |
| | 40,40, sulfated | | | |
| | glycoprotein 2) | | | |
| CP | ceruloplasmin | NM_000096 | NP_000087 | decreased T₋₆₀ |
| | (ferroxidase) | | | decreased T₋₃₆ |
| CRP | C-reactive protein, | NM_000567 | NP_000558 | decreased T₋₆₀ |
| | pentraxin-related | | | |
| F2 | Coagulation factor II | NM_000506 | AAL77436 | decreased T₋₃₆ |
| | (thrombine) | | | |
| F9 | Coagulation factor IX | NM_000133 | NP_000124 | increased T₋₁₂ |
| | (plasma | | | |
| | thromboplastic | | | |
| | component) | | | |
| FGA | fibrinogen alpha chain | BC070246 | BAC55116 | decreased T₋₆₀ |
| FGB | fibrinogen beta chain | NM_005141 | AAA18024 | decreased T₋₆₀ |
| | | | | increased T₋₃₆ |
| FLJ10006 | Hypothetical protein | BC110537 | AAH17012 | decreased T₋₃₆ |
| | FLJ10006 | BC110536 | | |
| FLNA | filamin A, alpha (actin | NM_001456 | CAI43227 | increased T₋₆₀ |
| | binding protein 280) | | | |
| FN1 | fibronectin 1 | BT006856 | BAD52437 | increased T₋₆₀ |
| GC | group-specific | NM_000583 | NP_000574 | decreased T₋₆₀ |
| | component (vitamin D | | | decreased T₋₃₆ |
| | binding protein) | | | increased T₋₁₂ |
| HBB | hemoglobin, beta | NM_000518 | AAD19696 | decreased T₋₆₀ |
| | | | | increased T₋₁₂ |
| HP | Haptoglobin | BC107587 | NP_005134 | decreased T₋₆₀ |
| HPX | Hemopexin | NM_000613 | NP_000604 | decreased T₋₆₀ |
| | | | | decreased T₋₁₂ |
| HRG | histidine-rich | NM_000412 | NP_000403 | increased T₋₆₀ |
| | glycoprotein | | | |
| IF | I factor (complement) | NM_000204 | NP_000195 | increased T₋₆₀ |
| | | | | increased T₋₃₆ |
| | | | | increased T₋₁₂ |
| IGFALS | insulin-like growth | NM_004970 | NP_004961 | decreased T₋₃₆ |
| | factor binding protein, | | | |
| | acid labile subunit | | | |
| ITIH1 | inter-alpha (globulin) | BC109115 | NP_002206 | increased T₋₆₀ |
| | inhibitor H1 | | NP_032432 | increased T₋₃₆ |
| | | | | increased T₋₁₂ |
| ITIH2 | inter-alpha (globulin) | NM_002216 | NP_002207 | increased T₋₆₀ |
| | inhibitor H2 | | | increased T₋₁₂ |
| ITIH4 | inter-alpha (globulin) | NM_002218 | NP_002209 | decreased T₋₆₀ |
| | inhibitor H4 (plasma | | | increased T₋₁₂ |
| | Kallikrein-sensitive | | | |
| | glycoprotein) | | | |
| KCTD7 | Potassium channel | NM_153033 | NP_694578 | decreased T₋₃₆ |
| | tetramerisation | | | |
| | domain containing 7 | | | |
| KLKB1 | kallikrein B, plasma | NM_000892 | NP_000883 | increased T₋₆₀ |
| | (Fletcher factor) 1 | | | decreased T₋₁₂ |
| KNG1 | kininogen 1 | NM_000893 | NP_000884 | decreased T₋₆₀ |
| | | | | decreased T₋₃₆ |
| | | | | increased T₋₁₂ |
| KRT1 | keratin 1 | BC063697 | NP_000412 | decreased T₋₆₀ |
| | (epidermolytic | | | |
| | hyperkeratosis) | | | |
| LGALS3BP | lectin, galactoside- | NM_005567 | NP_005558 | increased T₋₆₀ |
| | binding, soluble, 3 | BC015761 | | |
| | binding protein | BC002403 | | |
| | | BC002998 | | |
| LPA | lipoprotein, Lp(a) | NM_005577 | NP_005568 | decreased T₋₆₀ |
| | | | | increased T₋₃₆ |
| | | | | decreased T₋₁₂ |
| LRG1 | leucine-rich alpha-2- | NM_052972 | AAH70198 | decreased T₋₆₀ |
| | glycoprotein 1 | | | increased T₋₁₂ |
| MGC275 | hypothetical protein | BC087841 | AAH87841 | increased T₋₆₀ |
| | MGC27165 | BC005951 | | |
| MYO18B | myosin XVIIIB | NM_032608 | NP_115997 | decreased T₋₆₀ |
| ORM1 | orosomucoid 1 | NM_000607 | CAI16859 | decreased T₋₆₀ |
| | | | | decreased T₋₃₆ |
| | | | | increased T₋₁₂ |
| PDCD11 | Programmed cell | NM_014976 | NP_055791 | increased T₋₃₆ |
| | death 11 | | | |
| PGLYRP2 | peptidoglycan | NM_052890 | Q96PD5 | decreased T₋₆₀ |
| | recognition protein 2 | | | |
| QSCN6 | quiescin Q6 | NM_002826 | AAQ89300 | decreased T₋₆₀ |
| | | | | increased T₋₁₂ |
| RGS4 | regulator of G-protein | NM_005613 | NP_005604 | decreased T₋₆₀ |
| | signalling 4 | | | |
| SAA1 | serum amyloid A1 | BC105796 | AAA64799 | increased T₋₆₀ |
| | | | AAA30968 | increased T₋₁₂ |
| SERPINA1 | serine (or cysteine) | BC015642 | NP_001002235 | increased T₋₆₀ |
| | proteinase inhibitor, | NM_000295 | NP_000286 | decreased T₋₃₆ |
| | clade A (alpha-1 | | | increased T₋₁₂ |
| | antiproteinase, | | | |
| | antitrypsin), member 1 | | | |
| SERPINA3 | serine (or cysteine) | NM_001085 | NP_001076 | decreased T₋₆₀ |
| | proteinase inhibitor, | | | decreased T₋₃₆ |
| | clade A (alpha-1 | | | |
| | antiproteinase, | | | |
| | antitrypsin), member 3 | | | |
| SERPINA6 | serine (or cysteine) | NM_001756 | NP_001002236 | increased T₋₆₀ |
| | proteinase inhibitor, | | NP_000286 | |
| | clade A (alpha-1 | | | |
| | antiproteinase, | | | |
| | antitrypsin), member 6 | | | |
| SERPINC1 | serine (or cysteine) | NM_000488 | CAI19423 | increased T₋₆₀ |
| | proteinase inhibitor, | X68793 | P01008 | decreased T₋₃₆ |
| | clade C | | | |
| | (antithrombin), | | | |
| | member 1 | | | |
| SERPIND1 | serine (or cysteine) | NM_000185 | CAG30459 | decreased T₋₆₀ |
| | proteinase inhibitor, | | | |
| | clade D (heparin | | | |
| | cofactor), member 1 | | | |
| SERPINF2 | Serine (or cysteine) | BC031592 | NP_000925 | decreased T₋₁₂ |
| | proteinase inhibitor, | | | |
| | clade F (alpha-2 | | | |
| | antiplasmin, pigment | | | |
| | epithelium derived | | | |
| | factor), member 2 | | | |
| SERPING1 | senile (or cysteine) | NM_000062; | AAW69393 | decreased T₋₆₀ |
| | proteinase inhibitor, | BC011171 | | increased T₋₃₆ |
| | clade G (C1 inhibitor), | | | increased T₋₁₂ |
| | member 1 | | | |
| SMARCAD1 | SW1/SNF-related, | NM_020159 | NP_064544 | increased T₋₁₂ |
| | matrix-associated | | | |
| | actin-dependent | | | |
| | regulator of | | | |
| | chromatin, subfamily | | | |
| | a, containing | | | |
| | DEAD/H box 1 | | | |
| TF | transferring | NM_001063 | NP_001054 | increased T₋₁₂ |
| TRIP11 | thyroid hormone | NM_004239 | NP_004230 | increased T₋₆₀ |
| | receptor interactor 11 | | | |
| TTN | Titin | BC013396 | CAD12456 | decreased T₋₁₂ |
| TTR | Transthyretin | NM_000371 | AAH05310 | increased T₋₁₂ |
| | (prealbumin, | | AAP35853 | |
| | amyloidosis type 1) | | | |
| VTN | vitronectin (serum | NM_000638 | P04004 | increased T₋₆₀ |
| | spreading factor, | | | |
| | somatomedin B, | | | |
| | complement S- | | | |
| | protein) | | | |
| VWF | Von Willibrand Factor | NM_000552 | AAB59458 | increased T₋₁₂ |

In experiment two of example two, samples were run at T₋₁₂. A total of 93 proteins corresponding to 93 unique gene identifiers were found to be differentially expressed between groups at the T₋₁₂ time point. The identity of these 93 proteins is given in Table 16. Table 16 is identical to that given as Table 5 in example one above. However, what is not given in Table 5 is an indication of the directional change.

TABLE 16
Proteins that are differentially expressed at T₋₁₂ in experiment two of example
two

| | | | | Directional |
|---|---|---|---|---|
| | | Gene Acession | Gene Protein | change (Sepsis/ |
| Symbol | Description | Name | Name | SIRS) |
| Column 1 | Column 2 | Column 3 | Column 4 | Column 5 |
| A1BG | alpha-1-B glycoprotein | NM_130786 | NP_570602 | decreased |
| A2M | alpha-2-macroglobulin | NM_000014 | AAT02228 | decreased |
| ABLIM1 | actin binding LIM | NM_002313 | CAI10910 | decreased |
| | protein 1 | | | |
| ACTA1 | actin, alpha 1, skeletal | NM_001100 | CAI19052 | increased |
| | muscle | | | |
| AGT | angiotensinogen (serine | NM_000029 | AAR03501 | decreased |
| | (or cysteine) proteinase | | | |
| | inhibitor, clade (alpha- | | | |
| | 1 antiproteinase, | | | |
| | antitrypsin), member 8) | | | |
| AHSG | alpha-2-HS- | NM_001622 | NP_001613 | decreased |
| | glycoprotein | | | |
| ANK3 | ankyrin 3, node of | NM_020987 | CAI40519 | decreased |
| | Ranvier (ankyrin G) | | | |
| APCS | amyloid P component, | BT006750 | CAH73651 | increased |
| | serum | | | |
| APOA1 | apolipoprotein A-I | NM_000039 | AAD34604 | decreased |
| APOA4 | apolipoprotein A-IV | NM_000482 | AAS68228 | decreased |
| APOB | apolipoprotein B | NM_000384 | AAP72970 | decreased |
| | (including Ag(x) | | | |
| | antigen) | | | |
| APOC3 | apolipoprotein C-III | BC027977 | AAB59372 | decreased |
| APOL1 | apolipoprotein L, 1 | BC017331 | AAK20210 | decreased |
| | | NM_003661 | | |
| AZGP1 | alpha-2-glycoprotein 1, | NM_001185 | NP_001176 | decreased |
| | zinc | | | |
| B2M | beta-2-microglobulin | NM_004048 | AAA51811 | increased |
| BF | B-factor, properdin | NM_001710 | CAI17456 | decreased |
| C1R | complement | NM_001733 | NP_001724 | decreased |
| | component 1, r | | | |
| | subcomponent | | | |
| C1S | complement | NM_201442; | NP_958850 | increased |
| | component 1, s | NM_001734 | NP_001725 | |
| | subcomponent | | | |
| C2 | complement | NM_000063 | CAI17451 | decreased |
| | component 2 | | | |
| C4B | complement | NM_000592 | AAR89095 | increased |
| | component 4 beta | | | |
| C5 | complement | NM_001736 | NP_001726 | decreased |
| | component 5 | | | |
| C6 | complement | NM_000065 | BAD02322 | decreased |
| | component 6 | | | |
| C7 | complement | NM_000587 | CAA72407 | decreased |
| | component 7 | | | |
| C8A | complement | NM_000562 | CAI19172 | decreased |
| | component 8, alpha | | | |
| | polypeptide | | | |
| C8B | complement | NM_000066 | CAC18532 | decreased |
| | component 8, beta | | | |
| | polypeptide | | | |
| CDK5RA2 | CDK5 regulatory | NM_018249 | CAI40927 | increased |
| | subunit associated | | | |
| | protein 2 | | | |
| CHGB | chromogranin B | NM_001819 | CAB55272 | increased |
| | (secretogranin 1) | | | |
| CLU | clusterin (complement | NM_001831 | AAP88927 | decreased |
| | lysis inhibitor, SP- | | | |
| | 40,40, sulfated | | | |
| | glycoprotein 2, | | | |
| | testosterone-repressed | | | |
| | prostate message | | | |
| | 2, apolipoprotein J) | | | |
| COMP | cartilage oligomeric | NM_000095 | AAC83643 | increased |
| | matrix protein | | | |
| CORO1A | coronin, actin binding | NM_007074 | NP_009005 | increased |
| | protein, 1A | | | |
| CPN1 | carboxypeptidase N, | NM_001308 | NP_001299 | increased |
| | polypeptide 1, 50 kD | | | |
| CUL1 | cullin 1 | NM_003592 | NP_003583 | decreased |
| DET1 | de-etiolated homolog 1 | NM_017996 | NP_060466 | decreased |
| | (Arabidopsis) | | | |
| DSC1 | desmocollin 1 | BC109161 | NP_060466 | increased |
| F13A1 | coagulation factor XIII, | NM_000129 | CAC36886 | increased |
| | A1 polypeptide | | | |
| F2 | coagulation factor II | NM_000506 | AAL77436 | decreased |
| | (thrombin) | | | |
| F5 | coagulation factor V | NM_000130 | CAI23065 | decreased |
| | (proaccelerin, labile | | CAB16748 | |
| | factor) | | | |
| FGB | fibrinogen beta chain | NM_005141 | AAA18024 | increased |
| GOLGA1 | golgi autoantigen, | NM_002077 | CAI39632 | increased |
| | golgin subfamily a, 1 | | | |
| GSN | gelsolin (amyloidosis, | BC026033 | CAI14413 | decreased |
| | Finnish type) | | | |
| HBA1 | hemoglobin, alpha 1 | NM_000558 | AAO22464 | decreased |
| HBB | hemoglobin, beta | NM_000518 | AAD19696 | decreased |
| HP | haptoglobin | BC107587 | NP_005134 | decreased |
| HPX | hemopexin | NM_000613 | NP_000604 | decreased |
| HSPA5 | heat shock 70 kDa | NM_005347 | NP_005338 | increased |
| | protein 5 (glucose- | | | |
| | regulated protein, | | | |
| | 78 kDa) | | | |
| HUNK | hormonally upregulated | NM_014586 | NP_055401 | decreased |
| | Neu-associated kinase | | | |
| IGFBP5 | insulin-like growth | NM_000599 | NP_000590 | decreased |
| | factor binding protein 5 | | | |
| IGHG1 | immunoglobulin heavy | BC092518 | CAC20454 | decreased |
| | constant gamma 1 | | | |
| | (G1m marker) | | | |
| IGLV4-3 | immunoglobulin | BC020236 | AAH20236 | increased |
| | lambda variable 4-3 | | | |
| KIF5C | kinesin family member | NM_004984 | AAH17298 | decreased |
| | 5C | | | |
| KNG1 | kininogen 1 | NM_000893 | NP_000884 | increased |
| KRT1 | keratin 1 | BC063697 | NP_000412 | increased |
| | (epidermolytic | | | |
| | hyperkeratosis) | | | |
| KRT9 | keratin 9 | NM_000421 | NP_000412 | decreased |
| | (epidermolytic | | | |
| | palmoplantar | | | |
| | keratoderma) | | | |
| KRT10 | keratin 10 | NM_000226 | NP_000217 | decreased |
| | (epidermolytic | | | |
| | hyperkeratosis; | | | |
| | keratosis palmaris et | | | |
| | plantaris) | | | |
| LBP | lipopolysaccharide | AF105067 | AAC39547 | increased |
| | binding protein | | | |
| LGALS3BP | lectin, galactoside- | NM_005567 | NP_005558 | decreased |
| | binding, soluble, 3 | BC015761 | | |
| | binding protein | BC002403 | | |
| | | BC002998 | | |
| LRG1 | leucine-rich alpha-2- | NM_052972 | AAH70198 | decreased |
| | glycoprotein 1 | | | |
| LUM | lumican | BC035997 | AAP35353 | decreased |
| MMP14 | matrix | NM_004995 | AAV40837 | decreased |
| | metalloproteinase 14 | | | |
| | (membrane-inserted) | | | |
| MYH4 | myosin, heavy | NM_017533 | NP_060003 | decreased |
| | polypeptide 4, skeletal | | | |
| | muscle | | | |
| NEB | nebulin | NM_004543 | NP_004534 | increased |
| NUCB2 | nucleobindin 2 | NM_005013 | NP_005004 | increased |
| ORM2 | orosomucoid 2 | NM_000608 | NP_000599 | increased |
| PF4V1 | platelet factor 4 variant 1 | NM_002620 | NP_002611 | decreased |
| PIGR | polymeric | NM_002644 | CAC10060 | increased |
| | immunoglobulin | | | |
| | receptor | | | |
| PLG | plasminogen | NM_000301 | AAH60513 | decreased |
| PON1 | paraoxonase 1 | NM_000446 | NP_000437 | decreased |
| PPBP | pro-platelet basic | NM_002704 | CAG33086 | increased |
| | protein (chemokine | | | |
| | (C—X—C motif) | | | |
| | ligand 7) | | | |
| RBP4 | retinol binding protein | NM_006744 | CAH72328 | decreased |
| | 4, plasma | | | |
| RIMS1 | regulating synaptic | NM_014989 | NP_055804 | decreased |
| | membrane exocytosis 1 | | | |
| RNF6 | ring finger protein | NM_005977 | CAH73183 | increased |
| | (C3H2C3 type) 6 | | | |
| SAA1 | serum amyloid A1 | BC105796 | AAA64799 | decreased |
| | | | AAA30968 | |
| SEMA3D | sema domain, | NM_152754 | EAL24184 | increased |
| | immunoglobulin | | | |
| | domain (Ig), short basic | | | |
| | domain, secreted, | | | |
| | (semaphorin) 3D | | | |
| SERPINA1 | serine (or cysteine) | BC015642 | NP_001002235 | decreased |
| | proteinase inhibitor, | NM_000295 | NP_000286 | |
| | clade A (alpha-1 | | | |
| | antiproteinase, | | | |
| | antitrypsin), member 1 | | | |
| SERPIND1 | serine (or cysteine) | NM_000185 | CAG30459 | decreased |
| | proteinase inhibitor, | | | |
| | clade D (heparin | | | |
| | cofactor), member 1 | | | |
| SERPINF2 | serine (or cysteine) | BC031592 | NP_000925 | decreased |
| | proteinase inhibitor, | | | |
| | clade F (alpha-2 | | | |
| | antiplasmin, pigment | | | |
| | epithelium derived | | | |
| | factor), member 2 | | | |
| SERPING1 | serine (or cysteine) | NM_000062; | AAW69393 | decreased |
| | proteinase inhibitor, | BC011171 | | |
| | clade G (C1 inhibitor), | | | |
| | member 1, | | | |
| | (angioedema, | | | |
| | hereditary) | | | |
| SF3B1 | splicing factor 3b, | NM_012433 | NP_006833 | decreased |
| | subunit 1, 155 kDa | | | |
| SPINK1 | serine protease | NM_003122 | NP_003113 | increased |
| | inhibitor, Kazal type 1 | | | |
| SPP1 | secreted | NM_000582 | AAH17387 | increased |
| | phosphoprotein 1 | | | |
| | (osteopontin, bone | | | |
| | sialoprotein I, early T- | | | |
| | lymphocyte activation | | | |
| | 1) | | | |
| SPTB | spectrin, beta, | NM_001024858 | BAD92652 | increased |
| | erythrocytic (includes | | | |
| | spherocytosis, clinical | | | |
| | type I) | | | |
| SYNE1 | spectrin repeat | NM_182961 | AAH39121 | increased |
| | containing, nuclear | | | |
| | envelope 1 | | | |
| TAF4B | TAF4b RNA | NM_003187 | XP_290809 | decreased |
| | polymerase II, TATA | | | |
| | box binding protein | | | |
| | (TBP)-associated | | | |
| | factor, 105 kDa | | | |
| TBC1D1 | TBC1 (tre-2/USP6, | NM_015173 | NP_055988 | increased |
| | BUB2, cdc16) domain | | | |
| | family, member 1 | | | |
| TLN1 | talin 1 | NM_006289 | NP_006280 | decreased |
| TMSB4X | thymosin, beta 4, X- | NM_021109 | NP_066932 | decreased |
| | linked | | | |
| TRIP11 | thyroid hormone | NM_004239 | NP_004230 | decreased |
| | receptor interactor 11 | | | |
| TTR | transthyretin | NM_000371 | AAH05310 | decreased |
| | (prealbumin, | | AAP35853 | |
| | amyloidosis type I) | | | |
| UROC1 | urocanase domain | NM_144639 | NP_653240 | decreased |
| | containing 1 | | | |
| VTN | vitronectin (serum | NM_000638 | P04004 | increased |
| | spreading factor, | | | |
| | somatomedin B, | | | |
| | complement S-protein) | | | |
| VWF | von Willebrand factor | NM_000552 | AAB59458 | increased |
| ZFHX2 | zinc finger homeobox 2 | NM_033400 | NP_207646 | increased |
| ZYX | zyxin | NM_003461 | NP_001010972 | decreased |

Of the 71 proteins from experiment one, and 93 from experiment two, 30 were identical between experiments (Table 17). Of the 71 proteins from experiment one, and 93 from experiment two, 13 were identical at the T₋₁₂ time point. Seventeen of the proteins found at T₋₁₂ in experiment two were identical to proteins found at other time points in experiment one.

TABLE 17
Proteins found in both experiment two and at least one of time point in experiment
one of example two.

| | | Gene Acession | Gene Protein | |
|---|---|---|---|---|
| Symbol | Description | Name | Name | Direction^{†} |
| Column 1 | Column 2 | Column 3 | Column 4 | Column 5 |
| AGT | angiotensinogen | NM_000029 | AAR03501 | T₋₃₆ one D |
| | (serine (or cysteine) | | | T₋₁₂ two D |
| | proteinase inhibitor, | | | |
| | clade A (alpha-1 | | | |
| | antiproteinase, antitrypsin), | | | |
| | member 8) | | | |
| AHSG | Alpha-2-HS- | NM_001622 | NP_001613 | T₋₃₆ one D |
| | glycoprotein | | | T₋₆₀ one D |
| | | | | T₋₁₂ two D |
| APOA1 | apolipoprotein A-I | NM_000039 | AAD34604 | T₋₃₆ one I |
| | | | | T₋₆₀ one D |
| | | | | T₋₁₂ two D |
| APOA4 | apolipoprotein A-IV | NM_000482 | AAS68228 | T₋₆₀ one I |
| | | | | T₋₁₂ one I |
| | | | | T₋₁₂ two D |
| APOB | apolipoprotein B | NM_000384 | AAP72970 | T₋₆₀ one D |
| | (including Ag(x) | | | T₋₃₆ one D |
| | antigen) | | | T₋₁₂ two D |
| APOC3 | apolipoprotein C-III | BC027977 | AAB59372 | T₋₆₀ one D |
| | | | | T₋₁₂ one D |
| | | | | T₋₁₂ two D |
| APOL1 | apolipoprotein L, 1 | BC017331 | AAK20210 | T₋₆₀ one I |
| | | NM_003661 | | T₋₁₂ two D |
| BF | B-factor, properdin | NM_001710 | CAI17456 | T₋₁₂ one I |
| | (Alternate | | | T₋₁₂ two D |
| | complement pathway) | | | |
| C1s | complement | NM_201442; | NP_958850 | T₋₆₀ one I |
| | component 1, subunits | NM_001734 | | T₋₁₂ one D |
| | | | | T₋₁₂ two I |
| C2 | complement | NM_000063 | CAI17451 | T₋₃₆ one D |
| | component 2 | | | T₋₁₂ two D |
| C5 | complement | NM_001736 | NP_001726 | T₋₆₀ one D |
| | component 5 | | | T₋₁₂ two D |
| C8A | complement | NM_000562 | CAI19172 | T₋₆₀ one D |
| | component 8, alpha | | | T₋₁₂ two D |
| | polypeptide | | | |
| CLU | clusterin (complement | NM_001831 | AAP88927 | T₋₁₂ one I |
| | lysis inhibitor, SP- | | | T₋₁₂ two D |
| | 40,40, sulfated | | | |
| | glycoprotein 2, | | | |
| | testosterone-repressed | | | |
| | prostate message 2, | | | |
| | apolipoprotein J) | | | |
| F2 | coagulation factor II | NM_000506 | AAL77436 | T₋₃₆ one D |
| | (thrombin) | | | T₋₃₆ one D |
| FGB | fibrinogen beta chain | NM_005141 | AAA18024 | T₋₃₆ one I |
| | | | | T₋₆₀ one D |
| | | | | T₋₁₂ two I |
| HBB | hemoglobin, beta | NM_000518 | AAD19696 | T₋₁₂ one I |
| | | | | T₋₆₀ one D |
| | | | | T₋₁₂ two D |
| HP | haptoglobin | BC107587 | NP_005134 | T₋₆₀ one D |
| | | | | T₋₁₂ two D |
| HPX | hemopexin | NM_000613 | NP_000604 | T₋₆₀ one D |
| | | | | T₋₁₂ one D |
| | | | | T₋₁₂ two D |
| KNG1 | kininogen 1 | NM_000893 | NP_000884 | T₋₆₀ one D |
| | | | | T₋₃₆ one D |
| | | | | T₋₁₂ one I |
| | | | | T₋₁₂ two I |
| KRT1 | keratin 1 | BC063697 | NP_000412 | T₋₆₀ one D |
| | (epidermolytic | | | |
| | hyperkeratosis) | | | T₋₁₂ two I |
| LGALS3BP | lectin, galactoside- | NM_005567 | NP_005558 | T₋₆₀ one I |
| | binding, soluble, 3 | BC015761 | | T₋₁₂ two D |
| | binding protein | BC002403 | | |
| | | BC002998 | | |
| LRG1 | leucine-rich alpha-2- | NM_052972 | AAH70198 | T₋₆₀ one D |
| | glycoprotein 1 | | | T₋₁₂ one I |
| | | | | T₋₁₂ two D |
| SERPINA1 | serine (or cysteine) | BC015642 | NP_001002235 | T₋₆₀ one I |
| | proteinase inhibitor, | NM_000295 | NP_000286 | T₋₃₆ one D |
| | clade A (alpha-1 | | | T₋₁₂ one I |
| | antiproteinase, | | | T₋₁₂ two D |
| | antitrypsin), member 1 | | | |
| SERPIND1 | serine (or cysteine) | NM_000185 | CAG30459 | T₋₆₀ one D |
| | proteinase inhibitor, | | | T₋₁₂ one I |
| | clade D (heparin | | | T₋₁₂ two D |
| | cofactor), member 1 | | | |
| SERPINF2 | serine (or cysteine) | BC031592 | NP_000925 | T₋₁₂ one D |
| | proteinase inhibitor, | | | T₋₁₂ two D |
| | clade F (alpha-2 | | | |
| | antiplasmin, pigment | | | |
| | epithelium derived | | | |
| | factor), member 2 | | | |
| SERPING1 | serine (or cysteine) | NM_000062; | AAW69393 | T₋₆₀ one D |
| | proteinase inhibitor, | BC011171 | | T₋₃₆ one I |
| | clade G (C1 | | | T₋₁₂ one I |
| | inhibitor), member 1, | | | T₋₁₂ two D |
| | (angioedema, | | | |
| | hereditary) | | | |
| SAA1 | serum amyloid A1 | BC105796 | AAA64799 | T₋₆₀ one I |
| | | | AAA30968 | T₋₁₂ one I |
| | | | | T₋₁₂ two D |
| TTR | transthyretin | | | T₋₁₂ one I |
| | (prealbumin, | | | T₋₁₂ two D |
| | amyloidosis type I) | | | |
| VTN | vitronectin (serum | NM_000638 | P04004 | T₋₆₀ one I |
| | spreading factor, | | | T₋₃₆ one I |
| | somatomedin B, | | | T₋₁₂ two I |
| | complement S- | | | |
| | protein) | | | |
| VWF | von Willebrand factor | NM_000552 | AAB59458 | T₋₃₆ one I |
| | | | | T₋₁₂ two I |

^{†}Data given as time point, (T₋₆₀, T₋₃₆, or T₋₁₂), experiment one of example two (one) or experiment two of example two (two), and decrease (D) or increase (I).

The union of unique proteins discovered by each experiment generated a total list of 134 unique proteins. In order to obtain an overall picture of the system changes occurring between septic and uninfected inflammation, this list was uploaded into DAVID 2.1 for analysis. Pathway analysis via EASE score demonstrated 32 of the 134 (23.5%, p=2.5×10-42) mapped to the KEGG pathway: Complement and coagulation cascade (Tables 18 & 19).

TABLE 18
Experiment one proteins annotated by DAVID 2.1 to KEGG pathway
Complement and Coagulation Cascade.

| | | Gene Acession | Gene Protein |
|---|---|---|---|
| Symbol | Description | Name | Name |
| Column 1 | Column 2 | Column 3 | Column 4 |
| BF | B-factor, properdin | NM_001710 | CAI17456 |
| F2 | coagulation factor II | NM_000506 | AAL77436 |
| | (thrombin) | | |
| F9 | coagulation factor | NM_000133 | NP_000124 |
| | IX (plasma | | |
| | thromboplastic | | |
| | component, | | |
| | Christmas | | |
| | disease, | | |
| | hemophilia B) | | |
| C1QB | complement | NM_000491 | NP_000482 |
| | component 1, q | | |
| | subcomponent, beta | | |
| | polypeptide | | |
| C1S | complement | NM_201442; | NP_958850 |
| | component 1, s | NM_001734 | |
| | subcomponent | | |
| C2 | complement | NM_000063 | CAI17451 |
| | component 2 | | |
| C3 | complement | NM_000064 | AAR89906 |
| | component 3 | | |
| C4BPA | complement | NM_001017367 | CAH70782 |
| | component 4 | | |
| | binding protein, | | |
| | alpha | | |
| C5 | complement | NM_001736 | NP_001726 |
| | component 5 | | |
| C8A | complement | NM_000562 | CAI19172 |
| | component 8, alpha | | |
| | polypeptide | | |
| C9 | complement | NM_001737 | NP_001728 |
| | component 9 | | |
| FGA | fibrinogen alpha | BC070246 | BAC55116 |
| | chain | | |
| FGB | fibrinogen beta | NM_005141 | AAA18024 |
| | chain | | |
| FGG | fibrinogen gamma | NM_000509 | AAB59531 |
| | chain | | |
| IF | I factor | NM_000204 | NP_000195 |
| | (complement) | | |
| KLKB1 | kallikrein B, plasma | NM_000892 | NP_000883 |
| | (Fletcher factor) 1 | | |
| KNG1 | kininogen 1 | NM_000893 | NP_000884 |
| SERPINA1 | serine (or cysteine) | BC015642 | NP_001002235 |
| | proteinase inhibitor, | NM_000295 | NP_000286 |
| | clade A (alpha-1 | | |
| | antiproteinase, | | |
| | antitrypsin), | | |
| | member 1 | | |
| SERPINC1 | serine (or cysteine) | NM_000488 | CAI19423 |
| | proteinase inhibitor, | X68793 | P01008 |
| | clade C | | |
| | (antithrombin), | | |
| | member 1 | | |
| SERPIND1 | serine (or cysteine) | NM_000185 | CAG30459 |
| | proteinase inhibitor, | | |
| | clade D (heparin co- | | |
| | factor), member 2 | | |
| SERPINF2 | serine (or cysteine) | BC031592 | NP_000925 |
| | proteinase inhibitor, | | |
| | clade F (alpha-2 | | |
| | antiplasmin, | | |
| | pigment | | |
| | epithelium derived | | |
| | factor), member 2 | | |
| SERPING1 | serine (or cysteine) | NM_000062; | AAW69393 |
| | proteinase inhibitor, | BC011171 | |
| | clade G | | |
| | (C1 inhibitor), | | |
| | member 2 | | |

TABLE 19
Experiment two proteins annotated by DAVID 2.1 to KEGG pathway
Complement and Coagulation Cascade.

| | | Gene | |
|---|---|---|---|
| | | Acession | Gene Protein |
| Symbol | Description | Name | Name |
| A2M | alpha-2-macroglobulin | | |
| F2 | coagulation factor II | NM_000506 | AAL77436 |
| | (thrombin) | | |
| F5 | coagulation factor V | NM_000130 | CAI23065 |
| | (proaccelerin, labile | | CAB16748 |
| | factor) | | |
| F13A1 | coagulation factor | NM_000129 | CAC36886 |
| | XIII, A1 polypeptide | | |
| C1R | complement | NM_001733 | NP_001724 |
| | component 1, r | | |
| | subcomponent | | |
| C1S | complement | NM_201442; | NP_958850 |
| | component 1, s | NM_001734 | |
| | subcomponent | | |
| C2 | complement | NM_000063 | CAI17451 |
| | component 2 | | |
| C4B | complement | K02403 | AAB67980 |
| | component 4B | | |
| C5 | complement | NM_001736 | NP_001726 |
| | component 5 | | |
| C6 | complement | NM_000065 | BAD02322 |
| | component 6 | | |
| C7 | complement | NM_000587 | CAA72407 |
| | component 7 | | |
| C8A | complement | NM_000562 | CAI19172 |
| | component 8, alpha | | |
| | polypeptide | | |
| C8B | complement | NM_000066 | CAC18532 |
| | component 8, beta | | |
| | polypeptide | | |
| FGB | fibrinogen beta chain | NM_005141 | AAA18024 |
| KNG1 | kininogen 1 | NM_000893 | NP_000884 |
| PLG | plasminogen | | |
| SERPINA1 | serine (or cysteine) | BC015642 | NP_001002235 |
| | proteinase inhibitor, | NM_000295 | NP_000286 |
| | clade A (alpha-1 | | |
| | antiproteinase, | | |
| | antitrypsin), member 1 | | |

Other major pathways (Biocarta) over-represented by the list include (all p<3×10⁻⁸): classic complement pathway; 10 proteins (7.4%), complement pathway; 11 (8.1%), lectin induced complement pathway 8 (5.9%), intrinsic prothrombin activation pathway; 8 (5.9%), and the alternative complement pathway 7 (5.1%). The fibrinolysis pathway; 4 (2.9%), and extrinsic prothrombin activation pathway 4 (2.9%) both were significant at p<0.003.

There were very few non-complement/coagulation pathways statistically significantly represented within the group. Among the KEGG pathways, cell communication (10 proteins, 7.4%, p<0.0001) and focal adhesion (9 proteins, 6.6%, p=0.012) were significant. In the Biocarta subset, acute myocardial infarction (4 proteins, 2.9%, p<0.004), cells and molecules involved in local inflammatory response (4 proteins, 2.9%, P=0.011), and platelet amyloid precursor protein pathway (3 proteins, 2.2%, p=0.021) were the only other significant pathways. As DAVID v2.1 analysis compares lists to the entire human genome, the significance of the data compared to the known protein composition of plasma was investigated. The coagulation and complement pathway contains 63 proteins as listed by DAVID v2.1. The number of proteins in human plasma has been estimated to be between 1000 to 4000. Using a conservative estimate of 1275 (Anderson et al. 2004, “The human plasma proteome: a nonredundant list developed by a combination of four separate sources,” Mol Cell Proteomics 3: 311-26), the 32 proteins related to complement and coagulation pathway listed above still yield significance via Fisher exact testing of P<0.0001.

Discussion. Using novel mass spectrometry technology, differential proteins in the plasma proteome of critically ill septic patients compared to critically ill uninfected patients manifesting SIRS were identified. Over 20% of the proteins demonstrating differences between these two groups are related to complement and coagulation.

The innate immune system is essential for the early recognition and defense against microbial invasion. Complement activation is considered an integral component of the innate immune system and the involvement of the complement system in this example is consistent with this concept. Activation of other components of innate immunity via gene expression profiling has been demonstrated in similar patient populations. See Lissauer et al., 2005, Crit. Care Med. 33supp:A-16322. Complement allows for elimination of invading cells and activation of the adaptive immune response by stimulating secretion of various cytokines. It has been suggested that the complement system could be a potential therapeutic target for sepsis. See Bhole and Stahl, 2003, Crit. Care Med. 31: S97-104. Complement is activated by three distinct pathways. In the classical pathway, an antibody-antigen complex causes generation of C1q from C1. C1q binds to the Fc portion of the complex and activates C1r and C1s esterases. These cleave C2 and C4 forming C4b2a (C3 convertase). The alternative pathway does not involve antibodies. Instead, yeast zymogen, tissue-type plasminogen activator and other substances such as some biomaterials allow formation of the alternative C3 convertase: C3bBb. Finally, the Lectin pathway (mannose-binding pathway, MBL) is activated by MBL binding to carbohydrate structures on invading pathogens. The serine proteases MASP-1 and MASP-2 then cleave C2 and C4 forming the classic C3 convertase. C3 convertase cleaves C5, and formation of C5b-9 membrane attack complex ensues. The above results show that elements of all three pathways are differential between sepsis and sterile inflammation.

This study emphasizes the close association between sepsis and coagulation. The understanding of coagulation and sepsis at the molecular level has demonstrated the interconnected and intertwined nature of these systems. The profibrinolytic, antithrombotic, and anti-inflammatory drug, human recombinant activated protein C has been shown to reduce mortality from sepsis. See Bernard et al., 2001, N Engl J. Med. 344:699-709. In these same septic patients, markers of coagulation and inflammation were related to disease severity. See Kinasewitz et al., 2004, Crit. Care. 8:R82-R90. Tissue factor is expressed in monocytes and macrophages in response to many inflammatory insults. See, for example, Utter et al., 2002, J of Trauma, 52: 1071-1077; Drake et al., 1989, Am J Pathol. 134: 1087-1097; and Volk and Kox, 2000, Inflamm Res. 49:185-198. Additionally, pro-inflammatory cytokines cause increased expression of plasminogen activator inhibitor-1, and cause a decrease in protein C receptors. While immune activation stimulates coagulation, the reverse is also true, as various coagulation proteins such as thrombin, Factor Xa, TF-VIIa complexes stimulate cytokine production. Activated platelets also secrete chemokines, promote neutrophil adherence and through CD-40 promote adhesion molecule expression on endothelium.

One advantage of this experiment is the patient population and control groups. Instead of comparing sepsis to normal, healthy subjects, the pre-septic group was compared to clinically similar critically ill patients manifesting SIRS. This allows for a better distinction between infected and uninfected SIRS in the ICU. While evidence exists suggesting similar mechanisms for induction of inflammation via both infectious and non-infectious etiologies (see, for example, Fan et al, 2006, Am J Physiol Lung Cell Mol Physiol 290:738-746; Barsness et al., 2004, Am J Physiol Regul Integr Comp Physiol 287:R592-R599; and Tsung et al., 2005, J Immunol. 175:7661-8), this experiment demonstrated coagulation and complement differences in these patient populations. Further, using two separate methods and different pools of plasma, a large group of proteins identical between sets was identified.

Understanding the complex interactions and changes in the plasma proteome of patients becoming septic could allow for better diagnostics and therapeutics. Many of the proteins identified do not have commercially available immuno-assays and therefore new assays are being developed to verify and precisely quantify results obtained with this experiment. Future studies will test these proteins as potential biomarkers for sepsis. Understanding the complex systems events leading to sepsis may yield novel therapeutic targets. Further, since a subset of proteins demonstrated differential quantitation at study entry, it may be possible to stratify critically ill patients into various categories of risk of developing sepsis immediately on admission to the ICU.

One concern of this experiment is the calculated false-positive rate of 2.8% that may have resulted in up to four of the proteins considered false positives. However, even if all four map to coagulation and protein pathways, the list would still include 28 proteins or 20% of differentially expressed proteins as members of this group and the pathway would still be highly significant. Additionally, if a significant number of false positive proteins were present, a larger variance in the categories of pathways identified would be expected. The fact that the vast majority revolve around similar themes of complement and coagulation suggests a low impact of the false positive rate. For instance, no metabolism, endocrine, or cancer pathways were found. Another concern was a small set of discordant findings. As experiment one was run as three pools per time point, there were fifteen proteins at T-₆₀, and 5 at T₋₁₂ that demonstrated differences in directional changes between pools. Despite this, an overall picture of the direction and magnitude of change was still noted for these proteins.

A third concern with this experiment is the differences in mechanism of injury between groups. Among all proteins measured, there were 55 proteins significantly different at date of entry. Forty-seven (85.4%) of these were also noted to be different at later time points preceding sepsis diagnosis. These 47 represent only 35% of the 134 unique proteins that were different prior to sepsis. While these proteins may represent differences in mechanisms of injury, they may also suggest a protein related predisposition to sepsis. This concept would potentially hold prognostic and/or predictive value and further study is indicated to ascertain if they are markers of a predisposition to sepsis. Specific to complement and coagulation proteins, there are 15 annotated proteins that were significant at day of study entry, in addition to the later study periods (Table 20).

TABLE 20
Complement and coagulation proteins significant at DOE
and at least one time point prior to sepsis diagnosis.

| | | Gene | |
|---|---|---|---|
| | | Acession | Gene Protein |
| Symbol | Description | Name | Name |
| Column 1 | Column 2 | Column 3 | Column 4 |
| C1QB | Complement | NM_000491 | NP_000482 |
| | component 1, q | | |
| | subcomponent, beta | | |
| | polypeptide | | |
| C1S | complement | NM_201442; | NP_958850 |
| | component 1, s | NM_001734 | |
| | subcomponent | | |
| C2 | complement | NM_000063 | CAI17451 |
| | component 2 | | |
| C3 | complement | NM_000064 | AAR89906 |
| | component 3 | | |
| C8A | complement | NM_000562 | CAI19172 |
| | component 8, alpha | | |
| | polypeptide | | |
| C8B | complement | NM_000066 | CAC18532 |
| | component 8, beta | | |
| | polypeptide | | |
| C9 | complement | NM_001737 | NP_001728 |
| | component 9 | | |
| FGA | fibrinogen alpha chain | BC070246 | BAC55116 |
| FGB | fibrinogen beta chain | NM_005141 | AAA18024 |
| FGG | Fibrinogen gamma | NM_000509 | AAB59531 |
| | chain | | |
| IF | I factor (complement) | NM_000204 | NP_000195 |
| KLKB1 | kallikrien B, plasma | NM_000892 | NP_000883 |
| | (Fletcher factor) 1 | | |
| KNG1 | kininogen 1 | NM_000893 | NP_000884 |
| SERPIND1 | serine (or cysteine) | NM_000185 | CAG30459 |
| | proteinase inhibitor, | | |
| | clade D (heparin | | |
| | cofactor), member 1 | | |
| SERPINF2 | serine (or cysteine) | BC031592 | NP_000925 |
| | proteinase inhibitor, | | |
| | clade F (alpha-2 | | |
| | antiplasmin, pigment | | |
| | epithelium derived | | |
| | factor), member 2 | | |

This group represented 46.9% of the 32 differentially expressed complement and coagulation proteins noted leading up to T₋₀. However since the average time to T₋₀ was 7 days in both groups, and since APACHE II, ISS and TRISS were well matched, those proteins demonstrating differences at the three time points prior to sepsis diagnosis but not at DOE, represent changes related to development of sepsis rather than differences in mechanism of injury.

This experiment has identified specific plasma proteomic differences between critically ill SIRS patients who subsequently develop sepsis, and clinically similar SIRS patients who remained uninfected. These differences appear as early as three days prior to the clinical diagnosis of sepsis. Complement and coagulation proteins are statistically significantly overrepresented in this set. It is possible that a subset of these proteins may be useful as biomarkers for sepsis. Future study is warranted to evaluate these proteins for their potential predictive or diagnostic role.

In one embodiment of the present invention, a biomarker profile comprises any number of biomarkers selected from Table 21.

TABLE 21
Exemplary embodiment

| | | Gene Acession | Gene Protein | |
|---|---|---|---|---|
| Symbol | Description | Name | Name | Direction^{†} |
| Column 1 | Column 2 | Column 3 | Column 4 | Column 5 |
| AGT | Angiotensinogen | NM_000029 | AAR03501 | T₋₃₆ one D |
| | (serine (or cysteine) | | | T₋₁₂ two D |
| | proteinase inhibitor, | | | |
| | clade A (alpha-1 | | | |
| | antiproteinase, antitrypsin), | | | |
| | member 8) | | | |
| AHSG | Alpha-2-HS- | NM_001622 | NP_001613 | T₋₆₀ one D |
| | glycoprotein | | | T₋₃₆ one D |
| | | | | T₋₁₂ two D |
| APOA1 | apolipoprotein A-I | NM_000039 | AAD34604 | T₋₆₀ one D |
| | | | | T₋₃₆ one I |
| | | | | T₋₁₂ two D |
| APOB | apolipoprotein B | NM_000384 | AAP72970 | T₋₆₀ one D |
| | (including Ag(x) | | | T₋₃₆ one D |
| | antigen) | | | T₋₁₂ two D |
| APOC3 | apolipoprotein C-III | BC027977 | AAB59372 | T₋₆₀ one D |
| | | | | T₋₁₂ one D |
| | | | | T₋₁₂ two D |
| APOL1 | apolipoprotein L, 1 | BC017331 | AAK20210 | T₋₆₀ one I |
| | | NM_003661 | | T₋₁₂ two D |
| C2 | complement | NM_000063 | CAI17451 | T₋₃₆ one D |
| | component 2 | | | T₋₁₂ two D |
| C5 | complement | NM_001736 | NP_001726 | T₋₆₀ one D |
| | component 5 | | | T₋₁₂ two D |
| C8A | complement | NM_000562 | CAI19172 | T₋₆₀ one D |
| | component 8, alpha | | | T₋₁₂ two D |
| | polypeptide | | | |
| F2 | coagulation factor II | NM_000506 | AAL77436 | T₋₃₆ one D |
| | (thrombin) | | | T₋₃₆ one D |
| HP | haptoglobin | BC107587 | NP_005134 | T₋₆₀ one D |
| | | | | T₋₁₂ two D |
| HPX | hemopexin | NM_000613 | NP_000604 | T₋₆₀ one D |
| | | | | T₋₁₂ one D |
| | | | | T₋₁₂ two D |
| KNG1 | kininogen 1 | NM_000893 | NP_000884 | T₋₆₀ one D |
| | | | | T₋₃₆ one D |
| | | | | T₋₁₂ one I |
| | | | | T₋₁₂ two I |
| KRT1 | keratin 1 | BC063697 | NP_000412 | T₋₆₀ one D |
| | (epidermolytic | | | T₋₁₂ two I |
| | hyperkeratosis) | | | |
| LGALS3BP | Lectin, galactoside- | NM_005567 | NP_005558 | T₋₆₀ one I |
| | binding, soluble, 3 | BC015761 | | T₋₁₂ two D |
| | binding protein | BC002403 | | |
| | | BC002998 | | |
| SERPINF2 | serine (or cysteine) | BC031592 | NP_000925 | T₋₁₂ one D |
| | proteinase inhibitor, | | | T₋₁₂ two D |
| | clade F (alpha-2 | | | |
| | antiplasmin, pigment | | | |
| | epithelium derived | | | |
| | factor), member 2 | | | |
| VTN | vitronectin (serum | NM_000638 | P04004 | T₋₆₀ one I |
| | spreading factor, | | | T₋₃₆ one I |
| | somatomedin B, | | | T₋₁₂ two I |
| | complement S- | | | |
| | protein) | | | |
| VWF | von Willebrand factor | NM_000552 | AAB59458 | T₋₃₆ one I |
| | | | | T₋₁₂ two I |

^{†}Data given as time point, (T₋₆₀, T₋₃₆, or T₋₁₂), experiment one of example two (one) or experiment two of example two (two), and decrease (D) or increase (I).
d at least one time point prior to sepsis diagnosis.

### 7. ALTERNATIVE EMBODIMENTS AND REFERENCES CITED

All references cited herein are incorporated by reference herein in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a computer readable storage medium. Further, any of the methods of the present invention that don't involve a measuring step can be implemented in one or more computers or computer systems. Further still, any of the methods of the present invention that don't involve a measuring step can be implemented in one or more computer program products. Some embodiments of the present invention provide a computer system or a computer program product that encodes or has instructions for performing any or all of the methods disclosed herein. Such methods/instructions can be stored on a CD-ROM, DVD, magnetic disk storage product, or any other computer readable data or program storage product. Such methods can also be embedded in permanent storage, such as ROM, one or more programmable chips, or one or more application specific integrated circuits (ASICs). Such permanent storage can be localized in a server, 802.11 access point, 802.11 wireless bridge/station, repeater, router, mobile phone, or other electronic devices. Such methods encoded in the computer program product can also be distributed electronically.

Some embodiments of the present invention provide a computer program product that contains any or all of the program modules shown in FIG. 1. These program modules can be stored on a CD-ROM, DVD, magnetic disk storage product, or any other computer readable data or program storage product. The program modules can also be embedded in permanent storage, such as ROM, one or more programmable chips, or one or more application specific integrated circuits (ASICs). Such permanent storage can be localized in a server, 802.11 access point, 802.11 wireless bridge/station, repeater, router, mobile phone, or other electronic devices. The software modules in the computer program product can also be distributed electronically, via the Internet or otherwise.

Having now fully described the invention with reference to certain representative embodiments and details, it will be apparent to one of ordinary skill in the art that changes and modifications can be made thereto without departing from the spirit or scope of the invention as set forth herein. The specific embodiments described herein are offered by way of example only, and the invention is to be limited only by the terms of the appended claims, along with the full scope of equivalents to which such claims are entitled.

We claim:

1. A method of predicting the development of sepsis in a test subject at risk for developing sepsis, the method comprising:
evaluating whether a plurality of features in a biomarker profile of the test subject satisfies a first value set, wherein satisfying the first value set predicts that the test subject is likely to develop sepsis, and wherein the plurality of features are measurable aspects of a plurality of biomarkers listed in Table 1, 4, 5, 6, 7, 12, 13, 14, 15, 16, 17, 18, 19 20 and/or 21, wherein, when the plurality of biomarkers comprises complement component C3 and complement component C4, the plurality of biomarkers comprises three or more biomarkers.

2. The method of claim 1, the method further comprising:
evaluating whether the plurality of features in the biomarker profile of the test subject satisfies a second value set, wherein satisfying the second value set predicts that the test subject is not likely to develop sepsis.

3. The method of claim 1, wherein said plurality of biomarkers consists of between 3 and 25 biomarkers listed in one of Tables 1, 4, 5, 6, 7, 12, 13, 14, 15, 16, 17, 18, 19 20, and 21.

4. The method of claim 1, wherein said plurality of biomarkers consists of between 4 and 25 biomarkers listed in one of Tables 1, 4, 5, 6, 7, 12, 13, 14, 15, 16, 17, 18, 19 20, and 21.

5. The method of claim 1, wherein said plurality of biomarkers consists of between 5 and 25 biomarkers listed in one of Tables 1, 4, 5, 6, 7, 12, 13, 14, 15, 16, 17, 18, 19 20, and 21.

6. The method of claim 1, wherein said plurality of biomarkers comprises at least four biomarkers listed in one of Tables 1, 4, 5, 6, 7, 12, 13, 14, 15, 16, 17, 18, 19 20, and 21.

7. The method of claim 1, wherein said plurality of biomarkers comprises at least five biomarkers listed in one of Table 1, 4, 5, 6, 7, 12, 13, 14, 15, 16, 17, 18, 19 20, and 21.

8. The method of claim 1, wherein said plurality of biomarkers comprises C-reactive protein, apolipoprotein All, and antithrombin-III.

9. The method of claim 1, wherein said plurality of features consists of between 3 and 100 features corresponding to between 3 and 100 biomarkers in the plurality of biomarkers.

10. The method of claim 1, wherein said plurality of features consists of between 4 and 40 features corresponding to between 4 and 40 biomarkers in the plurality of biomarkers.

11. The method of claim 1, wherein said plurality of features consists of between 5 and 25 features corresponding to between 5 and 25 biomarkers in the plurality of biomarkers.

12. The method of claim 1, wherein said plurality of features comprises at least 4 features corresponding to at least 4 biomarkers in said plurality of biomarkers.

13. The method of claim 1, wherein said plurality of features comprises at least 5 features corresponding to at least 5 biomarkers in said plurality of biomarkers.

14. The method of claim 1, wherein each biomarker in said plurality of biomarkers is a biomarker listed in Table 1.

15. The method of claim 1, wherein each biomarker in said plurality of biomarkers is a biomarker listed in Table 4.

16. The method of claim 1, wherein each biomarker in said plurality of biomarkers is a biomarker listed in Table 5.

17. The method of claim 1, wherein each biomarker in said plurality of biomarkers is a biomarker listed in Table 6.

18. The method of claim 1, wherein each biomarker in said plurality of biomarkers is a biomarker listed in Table 7.

19. The method of claim 1, wherein each biomarker in said plurality of biomarkers is a biomarker listed in Table 12.

20. The method of claim 1, wherein each biomarker in said plurality of biomarkers is a biomarker listed in Table 13.

21. The method of claim 1, wherein each biomarker in said plurality of biomarkers is a biomarker listed in Table 14.

22. The method of claim 1, wherein each biomarker in said plurality of biomarkers is a biomarker listed in Table 15.

23. The method of claim 1, wherein each biomarker in said plurality of biomarkers is a biomarker listed in Table 16.

24. The method of claim 1, wherein each biomarker in said plurality of biomarkers is a biomarker listed in Table 17.

25. The method of claim 1, wherein each biomarker in said plurality of biomarkers is a biomarker listed in Table 18.

26. The method of claim 1, wherein each biomarker in said plurality of biomarkers is a biomarker listed in Table 19.

27. The method of claim 1, wherein each biomarker in said plurality of biomarkers is a biomarker listed in Table 20.

28. The method of claim 1, wherein each biomarker in said plurality of biomarkers is a nucleic acid.

29. The method of claim 1, wherein each biomarker in said plurality of biomarkers is a protein.

30. The method of claim 1, wherein a feature in said plurality of features is a measurable aspect of a biomarker in said plurality of biomarkers and a feature value for said feature is determined using a biological sample taken from said test subject at a single point in time.

31. The method of claim 30, wherein said feature is abundance of said biomarker in said sample.

32. The method of claim 30, wherein said feature is absence or presence of said biomarker in said sample.

33. The method of claim 30, wherein said feature is an identification of a species of said biomarker in said sample.

34. The method of claim 30, wherein said biological sample is whole blood.

35. The method of claim 30, wherein said biological sample is plasma, serum, saliva, sputum, urine, cerebral spinal fluid, cells, a cellular extract, a tissue specimen, a tissue biopsy, or a stool specimen.

36. The method of claim 30, wherein said biological sample is isolated neutrophils, isolated eosinophils, isolated basophils, isolated lymphocytes, or isolated monocytes.

37. The method of claim 1, wherein a feature in said plurality of features is a measurable aspect of a biomarker in said biomarker profile and a feature value for said feature is determined using a plurality of samples taken from said test subject at different points in time.

38. The method of claim 37, wherein said feature indicates whether an abundance of said biomarker is increasing or decreasing over time.

39. The method of claim 37, wherein a first sample in said plurality of samples is taken on a first day before the subject acquires sepsis and a second sample in said plurality of samples is taken on a second day before the subject acquires sepsis.

40. The method of claim 1, wherein a biomarker in said biomarker profile is an indication of a nucleic acid, an indication of a protein, an indication of a metabolite, or an indication of a carbohydrate.

41. The method of claim 1, wherein a biomarker in said biomarker profile is an indication of mRNA molecule or an indication of a cDNA molecule.

42. The method of claim 1, wherein a biomarker in said biomarker profile is an indication of an antibody.

43. The method of claim 1, wherein a first biomarker in said biomarker profile is an indication of a nucleic acid and a second biomarker in said biomarker profile is an indication of a protein.

44. The method of claim 1, the method further comprising constructing, prior to the evaluating step, said biomarker profile.

45. The method of claim 44, wherein said constructing step comprises obtaining said plurality of features from a sample of said test subject.

46. The method of claim 45, wherein said sample is whole blood.

47. The method of claim 45, wherein said sample is plasma, serum, saliva, sputum, urine, cerebral spinal fluid, cells, a cellular extract, a tissue specimen, a tissue biopsy, or a stool specimen.

48. The method of claim 45, wherein said sample is isolated neutrophils, isolated eosinophiles, isolated basophils, isolated lymphocytes, or isolated monocytes.

49. The method of claim 44, wherein the constructing step comprises applying a data analysis algorithm to features corresponding to biomarkers listed in Table 1, 4, 5, 6, 7, 12, 13, 14, 15, 16, 17, 18, 19, 20 and/or 21 that are obtained from members of a population.

50. The method of claim 49, wherein said population comprises subjects that subsequently develop sepsis (sepsis subjects) and subjects that do not subsequently develop sepsis (SIRS subjects).

51. The method of claim 49, wherein the features corresponding to biomarkers listed in Table 1, 4, 5, 6, 7, 12, 13, 14, 15, 16, 17, 18, 19, 20 and/or 21 that are obtained from members of said population are obtained at a time prior to when subjects in the population acquire sepsis.

52. The method of claim 49, wherein said data analysis algorithm is a decision tree, predictive analysis of microarrays, a multiple additive regression tree, a neural network, a clustering algorithm, principal component analysis, a nearest neighbor analysis, a linear discriminant analysis, a quadratic discriminant analysis, a support vector machine, an evolutionary method, a projection pursuit, or weighted voting.

53. The method of claim 1, the method further comprising constructing, prior to the evaluating step, said first value set.

54. The method of claim 53, wherein the constructing step comprises applying a data analysis algorithm to a plurality of features obtained from members of a population.

55. The method of claim 54, wherein said population comprises subjects that develop sepsis during an observation time period and subjects that do not develop sepsis during an observation time period.

56. The method of claim 54, wherein said data analysis algorithm is a decision tree, predictive analysis of microarrays, a multiple additive regression tree, a neural network, a clustering algorithm, principal component analysis, a nearest neighbor analysis, a linear discriminant analysis, a quadratic discriminant analysis, a support vector machine, an evolutionary method, a projection pursuit, or weighted voting.

57. The method of claim 54, wherein the constructing step generates a decision rule and wherein said evaluating step comprises applying said decision rule to the plurality of features in order to determine whether they satisfy the first value set.

58. The method of claim 57, wherein said decision rule classifies subjects in said population as (i) subjects that subsequently develop sepsis and (ii) subjects that do not subsequently develop sepsis with an accuracy of seventy percent or greater.

59. The method of claim 57, wherein said decision rule classifies subjects in said population as (i) subjects that subsequently develop sepsis and (ii) subjects that do not subsequently develop sepsis with an accuracy, specificity, or sensitivity of ninety percent or greater.

60. The method of claim 1, wherein a first biomarker in said biomarker profile is up-regulated in patients likely to develop sepsis.

61. The method of claim 1, wherein at least five biomarkers in said biomarker profile are up-regulated in patients likely to develop sepsis.

62. The method of claim 1, wherein a first biomarker in said biomarker profile is down-regulated in patients likely to develop sepsis.

63. The method of claim 1, wherein at least five biomarkers in said biomarker profile are down-regulated in patients likely to develop sepsis.

64. The method of claim 1, wherein a first biomarker in said biomarker profile is up-regulated at a first time point, and down-regulated at a second time point in a converter population relative to a nonconverter population.

65. The method of claim 1, wherein at least five biomarkers in said biomarker profile are up-regulated at a first time point, and down-regulated at a second time point in a converter population relative to a nonconverter population.

66. The method of claim 1, wherein a first biomarker in said biomarker profile is down-regulated at a first time point, and up-regulated at a second time point in a converter population relative to a nonconverter population.

67. The method of claim 1, wherein at least five biomarkers in said biomarker profile are down-regulated at a first time point, and up-regulated at a second time point in a converter population relative to a nonconverter population.

68. The method of claim 1, wherein the test subject has a likelihood of developing sepsis within 4 to 8 hours.

69. The method of claim 1, wherein the test subject has a likelihood of developing sepsis within 8 to 12 hours.

70. The method of claim 1, wherein the test subject has a likelihood of developing sepsis within 12 to 24 hours.

71. The method of claim 1, wherein the test subject has a likelihood of developing sepsis within 24 to 36 hours.

72. The method of claim 1, wherein the test subject has a likelihood of developing sepsis within 36 to 48 hours.

73. The method of claim 1, wherein the test subject has a likelihood of developing sepsis within 48 to 72 hours.

74. A method of diagnosing sepsis in a test subject, comprising:
evaluating whether a plurality of features in a biomarker profile of the test subject satisfies a first value set, wherein satisfying the first value set predicts that the test subject is likely to develop sepsis, wherein the plurality of features correspond to a plurality of biomarkers, the plurality of biomarkers comprising at least two biomarkers listed in any one of Tables 1, 4, 5, 6, 7, 12, 13, 14, 15, 16, 17, 18, 19, 20, and 21 wherein, when the plurality of biomarkers comprises complement component C3 and complement component C4, the plurality of biomarkers comprises three or more biomarkers.

75. A microarray comprising a plurality of probe spots, wherein at least twenty percent of the probe spots in the plurality of probe spots correspond to a plurality of biomarkers listed in Table 1, 4, 5, 6, 7, 12, 13, 14, 15, 16, 17, 18, 19, or 20.

76. A kit for predicting the development of sepsis in a test subject, the kit comprising a plurality of antibodies that specifically bind a plurality of biomarkers listed in Table 1, 4, 5, 6, 7, 12, 13, 14, 15, 16, 17, 18, 19, or 20.

77. A computer program product for use in conjunction with a computer system, wherein the computer program product comprises a computer readable storage medium and a computer program mechanism embedded therein, the computer program mechanism comprising:
instructions for evaluating whether a plurality of features in a biomarker profile of a test subject at risk for developing sepsis satisfies a first value set, wherein satisfying the first value set predicts that the test subject is likely to develop sepsis, and wherein the plurality of features are measurable aspects of a plurality of biomarkers, the plurality of biomarkers comprising at least two biomarkers listed in Table 1, 4, 5, 6, 7, 12, 13, 14, 15, 16, 17, 18, 19 20 and/or 21, wherein, when the plurality of biomarkers comprises complement component C3 and complement component C4, the plurality of biomarkers comprises three or more biomarkers.

78. A computer comprising:
a central processing unit;
a memory coupled to the central processing unit, the memory storing:
instructions for evaluating whether a plurality of features in a biomarker profile of a test subject at risk for developing sepsis satisfies a first value set, wherein satisfying the first value set predicts that the test subject is likely to develop sepsis, and wherein the plurality of features are measurable aspects of a plurality of biomarkers, the plurality of biomarkers comprising at least two biomarkers listed in Table 1, 4, 5, 6, 7, 12, 13, 14, 15, 16, 17, 18, 19 and/or 20, wherein, when the plurality of biomarkers comprises complement component C3 and complement component C4, the plurality of biomarkers comprises three or more biomarkers.

79. A computer system for determining whether a subject is likely to develop sepsis, the computer system comprising:
a central processing unit; and
a memory, coupled to the central processing unit, the memory storing:
instructions for obtaining a biomarker profile of a test subject, wherein said biomarker profile comprises a plurality of features and wherein the plurality of features are measurable aspects of a plurality of biomarkers, the plurality of biomarkers comprising at least two biomarkers listed in any one of Tables 1, 4, 5, 6, 7, 12, 13, 14, 15, 16, 17, 18, 19, 20, and 21, wherein, when the plurality of biomarkers comprises complement component C3 and complement component C4, the plurality of biomarkers comprises three or more biomarkers;
instructions for transmitting the biomarker profile to a remote computer, wherein the remote computer includes instructions for evaluating whether the plurality of features in the biomarker profile of the test subject satisfies a first value set, wherein satisfying the first value set predicts that the test subject is likely to develop sepsis; and
instructions for receiving a determination, from the remote computer, as to whether the plurality of features in the biomarker profile of the test subject satisfies the first value set; and
instructions for reporting whether the plurality of features in the biomarker profile of the test subject satisfies the first value set.

80. The method of claim 1, wherein a first biomarker in said biomarker profile is up-regulated in a converter population relative to a nonconverter population.

81. The method of claim 1, wherein at least five biomarkers in said biomarker profile are up-regulated in a converter population relative to a nonconverter population.

82. The method of claim 1, wherein a first biomarker in said biomarker profile is down-regulated in a converter population relative to a nonconverter population.

83. The method of claim 1, wherein at least five biomarkers in said biomarker profile are down-regulated in a converter population relative to a nonconverter population.
